SEQUENCE LISTING (407 sequences)

SEQ ID NO: 1 - protein, 1391 aa, synthetic construct
  REGION 1..1391: note = Synthetic
MDKPKKKRKVKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 2 - DNA, 4176 bp, synthetic construct
  misc_feature 1..4176: note = Synthetic
  source 1..4176: mol_type = other DNA
atggacaagcccaagaaaaagcggaaagtgaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtga

SEQ ID NO: 3 - protein, 7 aa, synthetic construct
  REGION 1..7: note = Synthetic
PKKKRKV

SEQ ID NO: 4 - protein, 7 aa, synthetic construct
  REGION 1..7: note = Synthetic
PKKKRRV

SEQ ID NO: 5 - protein, 16 aa, synthetic construct
  REGION 1..16: note = Synthetic
KRPAATKKAGQAKKKK

SEQ ID NO: 6 - RNA, 16 nt, synthetic construct
  misc_feature 1..16: note = Synthetic
  source 1..16: mol_type = other RNA
gttttagagctatgct

SEQ ID NO: 7 - RNA, 22 nt, synthetic construct
  misc_feature 1..22: note = Synthetic
  source 1..22: mol_type = other RNA
gttttagagctatgctgttttg

SEQ ID NO: 8 - RNA, 67 nt, synthetic construct
  misc_feature 1..67: note = Synthetic
  source 1..67: mol_type = other RNA
agcatagcaagttaaaataaggctagtccgttatcaacttgaaaaagtggcaccgagtcggtgcttt

SEQ ID NO: 9 - RNA, 72 nt, synthetic construct
  misc_feature 1..72: note = Synthetic
  source 1..72: mol_type = other RNA
aaacagcatagcaagttaaaataaggctagtccgttatcaacttgaaaaagtggcaccgagtcggtgctttt

SEQ ID NO: 10 - RNA, 82 nt, synthetic construct
  misc_feature 1..82: note = Synthetic
  source 1..82: mol_type = other RNA
gttggaaccattcaaaacagcatagcaagttaaaataaggctagtccgttatcaacttgaaaaagtggcaccgagtcggtgc

SEQ ID NO: 11 - RNA, 77 nt, synthetic construct
  misc_feature 1..77: note = Synthetic
  source 1..77: mol_type = other RNA
gttttagagctagaaatagcaagttaaaataaggctagtccgttatcaacttgaaaaagtggcaccgagtcggtgct

SEQ ID NO: 12 - RNA, 82 nt, synthetic construct; identical to SEQ ID NO: 10
  misc_feature 1..82: note = Synthetic
  source 1..82: mol_type = other RNA

SEQ ID NO: 13 - RNA, 76 nt, synthetic construct
  misc_feature 1..76: note = Synthetic
  source 1..76: mol_type = other RNA
gttttagagctagaaatagcaagttaaaataaggctagtccgttatcaacttgaaaaagtggcaccgagtcggtgc

SEQ ID NO: 14 - RNA, 86 nt, synthetic construct
  misc_feature 1..86: note = Synthetic
  source 1..86: mol_type = other RNA
gtttaagagctatgctggaaacagcatagcaagtttaaataaggctagtccgttatcaacttgaaaaagtggcaccgagtcggtgc

SEQ ID NO: 15 - RNA, 83 nt, synthetic construct
  misc_feature 1..83: note = Synthetic
  source 1..83: mol_type = other RNA
gttttagagctagaaatagcaagttaaaataaggctagtccgttatcaacttgaaaaagtggcaccgagtcggtgcttttttt

SEQ ID NO: 16 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 17 - RNA, 92 nt, synthetic construct
  misc_feature 1..92: note = Synthetic
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 18 - RNA, 68 nt, synthetic construct
  misc_feature 1..68: note = Synthetic
  source 1..68: mol_type = other RNA
gttttagagctagaaatagcaagttaaaataaggctagtccgttatcaacttggcaccgagtcggtgc

SEQ ID NO: 19-21 - skipped (3 entries)

SEQ ID NO: 22 - DNA, 20 bp, synthetic construct
  misc_feature 1..20: note = Synthetic
  source 1..20: mol_type = other DNA
gcttacatttgccacaggcg

SEQ ID NO: 23 - DNA, 20 bp, synthetic construct
  misc_feature 1..20: note = Synthetic
  source 1..20: mol_type = other DNA
ggcaccatctgcgccgagct

SEQ ID NO: 24 - DNA, 20 bp, synthetic construct
  misc_feature 1..20: note = Synthetic
  source 1..20: mol_type = other DNA
ggtccagcaaatactagatt

SEQ ID NO: 25 - RNA, 20 nt, synthetic construct
  misc_feature 1..20: note = Synthetic
  source 1..20: mol_type = other RNA
gcttacatttgccacaggcg

SEQ ID NO: 26 - RNA, 20 nt, synthetic construct
  misc_feature 1..20: note = Synthetic
  source 1..20: mol_type = other RNA
ggcaccatctgcgccgagct

SEQ ID NO: 27 - RNA, 20 nt, synthetic construct
  misc_feature 1..20: note = Synthetic
  source 1..20: mol_type = other RNA
ggtccagcaaatactagatt

SEQ ID NO: 28 - RNA, 103 nt, synthetic construct
  misc_feature 1..103: note = Synthetic
  source 1..103: mol_type = other RNA
gcttacatttgccacaggcggttttagagctagaaatagcaagttaaaataaggctagtccgttatcaacttgaaaaagtggcaccgagtcggtgcttttttt

SEQ ID NO: 29 - RNA, 103 nt, synthetic construct
  misc_feature 1..103: note = Synthetic
  source 1..103: mol_type = other RNA
ggcaccatctgcgccgagctgttttagagctagaaatagcaagttaaaataaggctagtccgttatcaacttgaaaaagtggcaccgagtcggtgcttttttt

SEQ ID NO: 30 - RNA, 103 nt, synthetic construct
  misc_feature 1..103: note = Synthetic
  source 1..103: mol_type = other RNA
ggtccagcaaatactagattgttttagagctagaaatagcaagttaaaataaggctagtccgttatcaacttgaaaaagtggcaccgagtcggtgcttttttt

SEQ ID NO: 31 - protein, 18 aa, synthetic construct
  REGION 1..18: note = Synthetic
EGRGSLLTCGDVEENPGP

SEQ ID NO: 32 - protein, 19 aa, synthetic construct
  REGION 1..19: note = Synthetic
ATNFSLLKQAGDVEENPGP

SEQ ID NO: 33 - protein, 20 aa, synthetic construct
  REGION 1..20: note = Synthetic
QCTNYALLKLAGDVESNPGP

SEQ ID NO: 34 - protein, 22 aa, synthetic construct
  REGION 1..22: note = Synthetic
VKQTLNFDLLKLAGDVESNPGP

SEQ ID NO: 35 - DNA, 20 bp, synthetic construct
  misc_feature 1..20: note = Synthetic
  source 1..20: mol_type = other DNA
accacgcctatgtagacaac

SEQ ID NO: 36 - DNA, 20 bp, synthetic construct
  misc_feature 1..20: note = Synthetic
  source 1..20: mol_type = other DNA
gccaaactcaaaccagtccg

SEQ ID NO: 37 - DNA, 20 bp, synthetic construct
  misc_feature 1..20: note = Synthetic
  source 1..20: mol_type = other DNA
ttggcaaatcgtgttggctg

SEQ ID NO: 38 - DNA, 20 bp, synthetic construct
  misc_feature 1..20: note = Synthetic
  source 1..20: mol_type = other DNA
ccagtttatcgtagagcaca

SEQ ID NO: 39 - DNA, 296 bp, Homo sapiens
  source 1..296: mol_type = genomic DNA
aaaggggggatcttaaatttggtctagccaaagccactaggcactgaaaggaacagttccccttttgccttccttctaaatggtacagggttgcaacactctgcttggctctgtgcttacatttgccacaggcggggaagatcagaactccccagcgatgtgtactggccagtgagtgctgccagggcaaagttgataaactaaagaaatgatgactaaacaaactattaaccaagtcctaatctctgcttgcctcctccatcattagaaccagaagaagattagagccctgtgtg

SEQ ID NO: 40 - DNA, 297 bp, Homo sapiens
  source 1..297: mol_type = genomic DNA
cctgagcaaacccagagcccagccctccctccccctgcagcctgagggcagggaccatggcaagcccttctcctacagccccagtgcctcagcagcctcattttcggggacgtgccagctcattttctggtctgttttgtggtgtggcaacagcctcgaatcacagcagcctgaacagcccagtaaatatttacacaaacctggggctggcaggccctctcctttgtggggctgccctgctgaaagggaggggagccaggccggctcagtcccagctcggcgcagatggtgcctgca

SEQ ID NO: 41 - DNA, 291 bp, Homo sapiens
  source 1..291: mol_type = genomic DNA
tgcttttatatttcttctagtacatgagcgatagtgtattttttggcctttcatgtgaataagtctgacacggacaagtactggaacaaagcattcccagttctctccgtgaaaaatctgctgacacctgaactggcttcaattggaatttgttcttggctgatctttacatgacctttgaaatcctggcatccgacctgcctcgggcttgggctccatggctctaagcctggctgcttcagggactctgtcctcccaatctagtatttgctggacctctctgcctgtctt

SEQ ID NO: 42 - DNA, 20 bp, synthetic construct
  misc_feature 1..20: note = Synthetic
  source 1..20: mol_type = other DNA
actggccagtacacatcgct

SEQ ID NO: 43 - DNA, 20 bp, synthetic construct
  misc_feature 1..20: note = Synthetic
  source 1..20: mol_type = other DNA
ctggcacgtccccgaaaatg

SEQ ID NO: 44 - DNA, 20 bp, synthetic construct
  misc_feature 1..20: note = Synthetic
  source 1..20: mol_type = other DNA
atccgacctgcctcgggctt

SEQ ID NO: 45 - RNA, 20 nt, synthetic construct
  misc_feature 1..20: note = Synthetic
  source 1..20: mol_type = other RNA
actggccagtacacatcgct

SEQ ID NO: 46 - RNA, 20 nt, synthetic construct
  misc_feature 1..20: note = Synthetic
  source 1..20: mol_type = other RNA
ctggcacgtccccgaaaatg

SEQ ID NO: 47 - RNA, 20 nt, synthetic construct
  misc_feature 1..20: note = Synthetic
  source 1..20: mol_type = other RNA
atccgacctgcctcgggctt

SEQ ID NO: 48 - RNA, 103 nt, synthetic construct
  misc_feature 1..103: note = Synthetic
  source 1..103: mol_type = other RNA
actggccagtacacatcgctgttttagagctagaaatagcaagttaaaataaggctagtccgttatcaacttgaaaaagtggcaccgagtcggtgcttttttt

SEQ ID NO: 49 - RNA, 103 nt, synthetic construct
  misc_feature 1..103: note = Synthetic
  source 1..103: mol_type = other RNA
ctggcacgtccccgaaaatggttttagagctagaaatagcaagttaaaataaggctagtccgttatcaacttgaaaaagtggcaccgagtcggtgcttttttt

SEQ ID NO: 50 - RNA, 103 nt, synthetic construct
  misc_feature 1..103: note = Synthetic
  source 1..103: mol_type = other RNA
atccgacctgcctcgggcttgttttagagctagaaatagcaagttaaaataaggctagtccgttatcaacttgaaaaagtggcaccgagtcggtgcttttttt

SEQ ID NO: 51 - DNA, 20 bp, synthetic construct
  misc_feature 1..20: note = Synthetic
  source 1..20: mol_type = other DNA
cgttatcgagtattgggcga

SEQ ID NO: 52 - DNA, 20 bp, synthetic construct
  misc_feature 1..20: note = Synthetic
  source 1..20: mol_type = other DNA
acgttatcgagtattgggcg

SEQ ID NO: 53 - DNA, 20 bp, synthetic construct
  misc_feature 1..20: note = Synthetic
  source 1..20: mol_type = other DNA
gttatcgagtattgggcgag

SEQ ID NO: 54 - DNA, 20 bp, synthetic construct
  misc_feature 1..20: note = Synthetic
  source 1..20: mol_type = other DNA
atcatgacgttatcgagtat

SEQ ID NO: 55 - DNA, 20 bp, synthetic construct
  misc_feature 1..20: note = Synthetic
  source 1..20: mol_type = other DNA
gtagccgtcacttattagat

SEQ ID NO: 56 - DNA, 20 bp, synthetic construct
  misc_feature 1..20: note = Synthetic
  source 1..20: mol_type = other DNA
tcatgacgttatcgagtatt

SEQ ID NO: 57 - DNA, 20 bp, synthetic construct
  misc_feature 1..20: note = Synthetic
  source 1..20: mol_type = other DNA
tatcttgtacgatagggtca

SEQ ID NO: 58 - DNA, 20 bp, synthetic construct
  misc_feature 1..20: note = Synthetic
  source 1..20: mol_type = other DNA
actggccagtacacatcgct

SEQ ID NO: 59 - DNA, 20 bp, synthetic construct
  misc_feature 1..20: note = Synthetic
  source 1..20: mol_type = other DNA
attgttaattgtagtccggg

SEQ ID NO: 60 - DNA, 20 bp, synthetic construct
  misc_feature 1..20: note = Synthetic
  source 1..20: mol_type = other DNA
aactccccagcgatgtgtac

SEQ ID NO: 61 - DNA, 20 bp, synthetic construct
  misc_feature 1..20: note = Synthetic
  source 1..20: mol_type = other DNA
cgcatgatgcttgctctcac

SEQ ID NO: 62 - DNA, 20 bp, synthetic construct
  misc_feature 1..20: note = Synthetic
  source 1..20: mol_type = other DNA
gttaaaagacgggattcctt

SEQ ID NO: 63 - DNA, 20 bp, synthetic construct
  misc_feature 1..20: note = Synthetic
  source 1..20: mol_type = other DNA
gtataggtactaacactccc

SEQ ID NO: 64 - DNA, 20 bp, synthetic construct
  misc_feature 1..20: note = Synthetic
  source 1..20: mol_type = other DNA
ttgtacgatagggtcaagga

SEQ ID NO: 65 - DNA, 20 bp, synthetic construct
  misc_feature 1..20: note = Synthetic
  source 1..20: mol_type = other DNA
atgcaatgtgctgatccgga

SEQ ID NO: 66 - DNA, 20 bp, synthetic construct
  misc_feature 1..20: note = Synthetic
  source 1..20: mol_type = other DNA
tcgagtattgggcgaggggt

SEQ ID NO: 67 - DNA, 20 bp, synthetic construct
  misc_feature 1..20: note = Synthetic
  source 1..20: mol_type = other DNA
catgcaatgtgctgatccgg

SEQ ID NO: 68 - DNA, 20 bp, synthetic construct
  misc_feature 1..20: note = Synthetic
  source 1..20: mol_type = other DNA
cttgtacgatagggtcaagg

SEQ ID NO: 69 - DNA, 20 bp, synthetic construct
  misc_feature 1..20: note = Synthetic
  source 1..20: mol_type = other DNA
ctggccagtacacatcgctg

SEQ ID NO: 70 - DNA, 20 bp, synthetic construct
  misc_feature 1..20: note = Synthetic
  source 1..20: mol_type = other DNA
gcatcgaaggacactaaagc

SEQ ID NO: 71 - DNA, 20 bp, synthetic construct
  misc_feature 1..20: note = Synthetic
  source 1..20: mol_type = other DNA
tgcaatgtgctgatccggag

SEQ ID NO: 72 - DNA, 20 bp, synthetic construct
  misc_feature 1..20: note = Synthetic
  source 1..20: mol_type = other DNA
gagatcaatcatgtactagg

SEQ ID NO: 73 - DNA, 20 bp, synthetic construct
  misc_feature 1..20: note = Synthetic
  source 1..20: mol_type = other DNA
cacgctaacataatattccc

SEQ ID NO: 74 - DNA, 20 bp, synthetic construct
  misc_feature 1..20: note = Synthetic
  source 1..20: mol_type = other DNA
acttacaataagactcctgc

SEQ ID NO: 75 - DNA, 20 bp, synthetic construct
  misc_feature 1..20: note = Synthetic
  source 1..20: mol_type = other DNA
cactggccagtacacatcgc

SEQ ID NO: 76 - DNA, 20 bp, synthetic construct
  misc_feature 1..20: note = Synthetic
  source 1..20: mol_type = other DNA
gctagaggacatatcctctt

SEQ ID NO: 77 - DNA, 20 bp, synthetic construct
  misc_feature 1..20: note = Synthetic
  source 1..20: mol_type = other DNA
caaggttggtggtttccggc

SEQ ID NO: 78 - DNA, 20 bp, synthetic construct
  misc_feature 1..20: note = Synthetic
  source 1..20: mol_type = other DNA
cgcattgttaattgtagtcc

SEQ ID NO: 79 - DNA, 20 bp, synthetic construct
  misc_feature 1..20: note = Synthetic
  source 1..20: mol_type = other DNA
ctgggaatattatgttagcg

SEQ ID NO: 80 - DNA, 20 bp, synthetic construct
  misc_feature 1..20: note = Synthetic
  source 1..20: mol_type = other DNA
gcacggacccgtttacggag

SEQ ID NO: 81 - DNA, 20 bp, synthetic construct
  misc_feature 1..20: note = Synthetic
  source 1..20: mol_type = other DNA
acgctacggaagtcaactgc

SEQ ID NO: 82 - DNA, 20 bp, synthetic construct
  misc_feature 1..20: note = Synthetic
  source 1..20: mol_type = other DNA
cacggacccgtttacggaga

SEQ ID NO: 83 - DNA, 20 bp, synthetic construct
  misc_feature 1..20: note = Synthetic
  source 1..20: mol_type = other DNA
aatttgcacggacccgttta

SEQ ID NO: 84 - DNA, 20 bp, synthetic construct
  misc_feature 1..20: note = Synthetic
  source 1..20: mol_type = other DNA
tcgggcggcactgaacgccc

SEQ ID NO: 85 - DNA, 20 bp, synthetic construct
  misc_feature 1..20: note = Synthetic
  source 1..20: mol_type = other DNA
aaattccacagacgcgcacc

SEQ ID NO: 86 - DNA, 20 bp, synthetic construct
  misc_feature 1..20: note = Synthetic
  source 1..20: mol_type = other DNA
gccgggttctgcgtacacat

SEQ ID NO: 87 - DNA, 20 bp, synthetic construct
  misc_feature 1..20: note = Synthetic
  source 1..20: mol_type = other DNA
atctggacccgtgcgccagc

SEQ ID NO: 88 - DNA, 20 bp, synthetic construct
  misc_feature 1..20: note = Synthetic
  source 1..20: mol_type = other DNA
agatcgcatgcacgcccagc

SEQ ID NO: 89 - DNA, 20 bp, synthetic construct
  misc_feature 1..20: note = Synthetic
  source 1..20: mol_type = other DNA
gtgtagtcaaccctgcccga

SEQ ID NO: 90 - DNA, 20 bp, synthetic construct
  misc_feature 1..20: note = Synthetic
  source 1..20: mol_type = other DNA
aacgggtccgtgcaaattca

SEQ ID NO: 91 - DNA, 20 bp, synthetic construct
  misc_feature 1..20: note = Synthetic
  source 1..20: mol_type = other DNA
tgcagcacgcagttcttgcg

SEQ ID NO: 92 - DNA, 20 bp, synthetic construct
  misc_feature 1..20: note = Synthetic
  source 1..20: mol_type = other DNA
tgggcaccttgattcggtgc

SEQ ID NO: 93 - DNA, 20 bp, synthetic construct
  misc_feature 1..20: note = Synthetic
  source 1..20: mol_type = other DNA
aggtctcttgttgcacccga

SEQ ID NO: 94 - DNA, 20 bp, synthetic construct
  misc_feature 1..20: note = Synthetic
  source 1..20: mol_type = other DNA
ctggcacgtccccgaaaatg

SEQ ID NO: 95 - DNA, 20 bp, synthetic construct
  misc_feature 1..20: note = Synthetic
  source 1..20: mol_type = other DNA
cattgtacgactaagattga

SEQ ID NO: 96 - DNA, 20 bp, synthetic construct
  misc_feature 1..20: note = Synthetic
  source 1..20: mol_type = other DNA
gttcagtgccgcccgacaga

SEQ ID NO: 97 - DNA, 20 bp, synthetic construct
  misc_feature 1..20: note = Synthetic
  source 1..20: mol_type = other DNA
tgtgtagtcaaccctgcccg

SEQ ID NO: 98 - DNA, 20 bp, synthetic construct
  misc_feature 1..20: note = Synthetic
  source 1..20: mol_type = other DNA
ggcacacctgacgatgtcaa

SEQ ID NO: 99 - DNA, 20 bp, synthetic construct
  misc_feature 1..20: note = Synthetic
  source 1..20: mol_type = other DNA
cggccaccagcttttgccgg

SEQ ID NO: 100 - DNA, 20 bp, synthetic construct
  misc_feature 1..20: note = Synthetic
  source 1..20: mol_type = other DNA
gggactctggccctaacgtg

SEQ ID NO: 101 - DNA, 20 bp, synthetic construct
  misc_feature 1..20: note = Synthetic
  source 1..20: mol_type = other DNA
aggtgcactaggtagactag

SEQ ID NO: 102 - DNA, 20 bp, synthetic construct
  misc_feature 1..20: note = Synthetic
  source 1..20: mol_type = other DNA
aggccccctttgacatcgtc

SEQ ID NO: 103 - DNA, 20 bp, synthetic construct
  misc_feature 1..20: note = Synthetic
  source 1..20: mol_type = other DNA
aggcaccatctgcgccgagc

SEQ ID NO: 104 - DNA, 20 bp, synthetic construct
  misc_feature 1..20: note = Synthetic
  source 1..20: mol_type = other DNA
gcgcacgggtccagatggag

SEQ ID NO: 105 - DNA, 20 bp, synthetic construct
  misc_feature 1..20: note = Synthetic
  source 1..20: mol_type = other DNA
ctggataccagctggcgcac

SEQ ID NO: 106 - DNA, 20 bp, synthetic construct
  misc_feature 1..20: note = Synthetic
  source 1..20: mol_type = other DNA
ggtactggtggcaaacgcta

SEQ ID NO: 107 - DNA, 20 bp, synthetic construct
  misc_feature 1..20: note = Synthetic
  source 1..20: mol_type = other DNA
aggtgcgcgtctgtggaatt

SEQ ID NO: 108 - DNA, 20 bp, synthetic construct
  misc_feature 1..20: note = Synthetic
  source 1..20: mol_type = other DNA
gcccatgtgtacgcagaacc

SEQ ID NO: 109 - DNA, 20 bp, synthetic construct
  misc_feature 1..20: note = Synthetic
  source 1..20: mol_type = other DNA
acccgctagaatctagtcat

SEQ ID NO: 110 - DNA, 20 bp, synthetic construct
  misc_feature 1..20: note = Synthetic
  source 1..20: mol_type = other DNA
aagtaggacgttcccgcata

SEQ ID NO: 111 - DNA, 20 bp, synthetic construct
  misc_feature 1..20: note = Synthetic
  source 1..20: mol_type = other DNA
atccgacctgcctcgggctt

SEQ ID NO: 112 - DNA, 20 bp, synthetic construct
  misc_feature 1..20: note = Synthetic
  source 1..20: mol_type = other DNA
tcgagagcaagacaacgcaa

SEQ ID NO: 113 - DNA, 20 bp, synthetic construct
  misc_feature 1..20: note = Synthetic
  source 1..20: mol_type = other DNA
cacttgaagattccggatga

SEQ ID NO: 114 - DNA, 20 bp, synthetic construct
  misc_feature 1..20: note = Synthetic
  source 1..20: mol_type = other DNA
ctaccagtgggcctaaataa

SEQ ID NO: 115 - DNA, 20 bp, synthetic construct
  misc_feature 1..20: note = Synthetic
  source 1..20: mol_type = other DNA
taagtgtcagcttaccaacg

SEQ ID NO: 116 - DNA, 20 bp, synthetic construct
  misc_feature 1..20: note = Synthetic
  source 1..20: mol_type = other DNA
ggggccaatagcaaactaac

SEQ ID NO: 117 - DNA, 20 bp, synthetic construct
  misc_feature 1..20: note = Synthetic
  source 1..20: mol_type = other DNA
ccgaatctatcagtgcctgg

SEQ ID NO: 118 - DNA, 20 bp, synthetic construct
  misc_feature 1..20: note = Synthetic
  source 1..20: mol_type = other DNA
cgagcatatatgctagattt

SEQ ID NO: 119 - DNA, 20 bp, synthetic construct
  misc_feature 1..20: note = Synthetic
  source 1..20: mol_type = other DNA
gtcggatgccaggatttcaa

SEQ ID NO: 120 - DNA, 20 bp, synthetic construct
  misc_feature 1..20: note = Synthetic
  source 1..20: mol_type = other DNA
tagaactcacgacatagggc

SEQ ID NO: 121 - DNA, 20 bp, synthetic construct
  misc_feature 1..20: note = Synthetic
  source 1..20: mol_type = other DNA
tttaactaaggttgccgaaa

SEQ ID NO: 122 - DNA, 20 bp, synthetic construct
  misc_feature 1..20: note = Synthetic
  source 1..20: mol_type = other DNA
gaacggatgcaacaagacca

SEQ ID NO: 123 - DNA, 20 bp, synthetic construct
  misc_feature 1..20: note = Synthetic
  source 1..20: mol_type = other DNA
gatgtgctaacatcctcccg

SEQ ID NO: 124 - DNA, 20 bp, synthetic construct
  misc_feature 1..20: note = Synthetic
  source 1..20: mol_type = other DNA
agtttctttatcacgatcat

SEQ ID NO: 125 - DNA, 20 bp, synthetic construct
  misc_feature 1..20: note = Synthetic
  source 1..20: mol_type = other DNA
ggaacggatgcaacaagacc

SEQ ID NO: 126 - DNA, 20 bp, synthetic construct
  misc_feature 1..20: note = Synthetic
  source 1..20: mol_type = other DNA
tcctatgactagattctagc

SEQ ID NO: 127 - DNA, 20 bp, synthetic construct
  misc_feature 1..20: note = Synthetic
  source 1..20: mol_type = other DNA
atgccattatttaggcccac

SEQ ID NO: 128 - DNA, 20 bp, synthetic construct
  misc_feature 1..20: note = Synthetic
  source 1..20: mol_type = other DNA
agtctgacacggacaagtac

SEQ ID NO: 129 - DNA, 20 bp, synthetic construct
  misc_feature 1..20: note = Synthetic
  source 1..20: mol_type = other DNA
catccgacctgcctcgggct

SEQ ID NO: 130 - DNA, 20 bp, synthetic construct
  misc_feature 1..20: note = Synthetic
  source 1..20: mol_type = other DNA
tcagcactggtacatatatt

SEQ ID NO: 131 - DNA, 20 bp, synthetic construct
  misc_feature 1..20: note = Synthetic
  source 1..20: mol_type = other DNA
agtccgaatctatcagtgcc

SEQ ID NO: 132 - DNA, 20 bp, synthetic construct
  misc_feature 1..20: note = Synthetic
  source 1..20: mol_type = other DNA
atataggatcccaacaatat

SEQ ID NO: 133 - DNA, 20 bp, synthetic construct
  misc_feature 1..20: note = Synthetic
  source 1..20: mol_type = other DNA
ccagcaaatactagattggg

SEQ ID NO: 134 - DNA, 20 bp, synthetic construct
  misc_feature 1..20: note = Synthetic
  source 1..20: mol_type = other DNA
actccatattcttccccggg

SEQ ID NO: 135 - DNA, 20 bp, synthetic construct
  misc_feature 1..20: note = Synthetic
  source 1..20: mol_type = other DNA
aacgcacatcgtttatatgg

SEQ ID NO: 136 - DNA, 20 bp, synthetic construct
  misc_feature 1..20: note = Synthetic
  source 1..20: mol_type = other DNA
actaacaggatgcatgcaag

SEQ ID NO: 137 - DNA, 20 bp, synthetic construct
  misc_feature 1..20: note = Synthetic
  source 1..20: mol_type = other DNA
taggacgttcccgcataagg

SEQ ID NO: 138 - DNA, 20 bp, synthetic construct
  misc_feature 1..20: note = Synthetic
  source 1..20: mol_type = other DNA
gggaccgatttggccgttaa

SEQ ID NO: 139 - DNA, 20 bp, synthetic construct
  misc_feature 1..20: note = Synthetic
  source 1..20: mol_type = other DNA
gtaaccattaacggccaaat

SEQ ID NO: 140 - DNA, 20 bp, synthetic construct
  misc_feature 1..20: note = Synthetic
  source 1..20: mol_type = other DNA
cacggcccttcgattgttga

SEQ ID NO: 141 - DNA, 20 bp, synthetic construct
  misc_feature 1..20: note = Synthetic
  source 1..20: mol_type = other DNA
aaggatcgttaggtgatcgt

SEQ ID NO: 142 - DNA, 20 bp, synthetic construct
  misc_feature 1..20: note = Synthetic
  source 1..20: mol_type = other DNA
tacctaccggtcatatatcc

SEQ ID NO: 143 - DNA, 20 bp, synthetic construct
  misc_feature 1..20: note = Synthetic
  source 1..20: mol_type = other DNA
cacgaggacgattaccatat

SEQ ID NO: 144 - DNA, 20 bp, synthetic construct
  misc_feature 1..20: note = Synthetic
  source 1..20: mol_type = other DNA
aggatcgttaggtgatcgtc

SEQ ID NO: 145 - DNA, 20 bp, synthetic construct
  misc_feature 1..20: note = Synthetic
  source 1..20: mol_type = other DNA
cacgggacgctattctaaac

SEQ ID NO: 146 - DNA, 20 bp, synthetic construct
  misc_feature 1..20: note = Synthetic
  source 1..20: mol_type = other DNA
ccataatcaggtcgtttatc

SEQ ID NO: 147 - DNA, 20 bp, synthetic construct
  misc_feature 1..20: note = Synthetic
  source 1..20: mol_type = other DNA
atcggtcccataatagggtg

SEQ ID NO: 148 - DNA, 20 bp, synthetic construct
  misc_feature 1..20: note = Synthetic
  source 1..20: mol_type = other DNA
ggccaaatcggtcccataat

SEQ ID NO: 149 - DNA, 20 bp, synthetic construct
  misc_feature 1..20: note = Synthetic
  source 1..20: mol_type = other DNA
ctcggtttactaggacaaag

SEQ ID NO: 150 - DNA, 20 bp, synthetic construct
  misc_feature 1..20: note = Synthetic
  source 1..20: mol_type = other DNA
atagcgtcccgtgatgtttt

SEQ ID NO: 151 - DNA, 20 bp, synthetic construct
  misc_feature 1..20: note = Synthetic
  source 1..20: mol_type = other DNA
tcagtattgtaccgctttag

SEQ ID NO: 152 - DNA, 20 bp, synthetic construct
  misc_feature 1..20: note = Synthetic
  source 1..20: mol_type = other DNA
aatcggtcccataatagggt

SEQ ID NO: 153 - DNA, 20 bp, synthetic construct
  misc_feature 1..20: note = Synthetic
  source 1..20: mol_type = other DNA
accctattatgggaccgatt

SEQ ID NO: 154 - DNA, 20 bp, synthetic construct
  misc_feature 1..20: note = Synthetic
  source 1..20: mol_type = other DNA
tgcacttgcccgtgtaacac

SEQ ID NO: 155 - DNA, 20 bp, synthetic construct
  misc_feature 1..20: note = Synthetic
  source 1..20: mol_type = other DNA
gatctaccatcaacaatcga

SEQ ID NO: 156 - DNA, 20 bp, synthetic construct
  misc_feature 1..20: note = Synthetic
  source 1..20: mol_type = other DNA
caccaggatatatgaccggt

SEQ ID NO: 157 - DNA, 20 bp, synthetic construct
  misc_feature 1..20: note = Synthetic
  source 1..20: mol_type = other DNA
ggtggctacgttgggcccac

SEQ ID NO: 158 - DNA, 20 bp, synthetic construct
  misc_feature 1..20: note = Synthetic
  source 1..20: mol_type = other DNA
gccaaatcggtcccataata

SEQ ID NO: 159 - DNA, 20 bp, synthetic construct
  misc_feature 1..20: note = Synthetic
  source 1..20: mol_type = other DNA
atcagttataacagcctagc

SEQ ID NO: 160 - DNA, 20 bp, synthetic construct
  misc_feature 1..20: note = Synthetic
  source 1..20: mol_type = other DNA
ccgcttgcactagagggtcc

SEQ ID NO: 161 - DNA, 20 bp, synthetic construct
  misc_feature 1..20: note = Synthetic
  source 1..20: mol_type = other DNA
gaccctctagtgcaagcggc

SEQ ID NO: 162 - DNA, 20 bp, synthetic construct
  misc_feature 1..20: note = Synthetic
  source 1..20: mol_type = other DNA
gatagttactttactctccg

SEQ ID NO: 163 - DNA, 20 bp, synthetic construct
  misc_feature 1..20: note = Synthetic
  source 1..20: mol_type = other DNA
atcgaagggccgtgttaaga

SEQ ID NO: 164 - DNA, 20 bp, synthetic construct
  misc_feature 1..20: note = Synthetic
  source 1..20: mol_type = other DNA
aaagaggcttctcaacccgc

SEQ ID NO: 165 - DNA, 20 bp, synthetic construct
  misc_feature 1..20: note = Synthetic
  source 1..20: mol_type = other DNA
acggaccatcatgcacggca

SEQ ID NO: 166 - DNA, 20 bp, synthetic construct
  misc_feature 1..20: note = Synthetic
  source 1..20: mol_type = other DNA
gtcccccgtgccctagatcc

SEQ ID NO: 167 - DNA, 20 bp, synthetic construct
  misc_feature 1..20: note = Synthetic
  source 1..20: mol_type = other DNA
tcgaaggaccaaactcatag

SEQ ID NO: 168 - DNA, 20 bp, synthetic construct
  misc_feature 1..20: note = Synthetic
  source 1..20: mol_type = other DNA
gtacgacccataaaagtgcc

SEQ ID NO: 169 - DNA, 20 bp, synthetic construct
  misc_feature 1..20: note = Synthetic
  source 1..20: mol_type = other DNA
ctgtgaatacgccttggcac

SEQ ID NO: 170 - DNA, 20 bp, synthetic construct
  misc_feature 1..20: note = Synthetic
  source 1..20: mol_type = other DNA
ccttcaacagtcccgcccat

SEQ ID NO: 171 - DNA, 20 bp, synthetic construct
  misc_feature 1..20: note = Synthetic
  source 1..20: mol_type = other DNA
cgtgtgcagcccggtgccga

SEQ ID NO: 172 - DNA, 20 bp, synthetic construct
  misc_feature 1..20: note = Synthetic
  source 1..20: mol_type = other DNA
cacaaacgatgggtagtttc

SEQ ID NO: 173 - DNA, 20 bp, synthetic construct
  misc_feature 1..20: note = Synthetic
  source 1..20: mol_type = other DNA
tcgcacactaaacgaggaac

SEQ ID NO: 174 - DNA, 20 bp, synthetic construct
  misc_feature 1..20: note = Synthetic
  source 1..20: mol_type = other DNA
atacgccttggcactggatc

SEQ ID NO: 175 - DNA, 20 bp, synthetic construct
  misc_feature 1..20: note = Synthetic
  source 1..20: mol_type = other DNA
tgccacgggccttcggcacc

SEQ ID NO: 176 - DNA, 20 bp, synthetic construct
  misc_feature 1..20: note = Synthetic
  source 1..20: mol_type = other DNA
ggaactgtgatccgtgccac

SEQ ID NO: 177 - DNA, 20 bp, synthetic construct
  misc_feature 1..20: note = Synthetic
  source 1..20: mol_type = other DNA
aggctactatgatgcggtag

SEQ ID NO: 178 - DNA, 20 bp, synthetic construct
  misc_feature 1..20: note = Synthetic
  source 1..20: mol_type = other DNA
gtttgatcgcacactaaacg

SEQ ID NO: 179 - DNA, 20 bp, synthetic construct
  misc_feature 1..20: note = Synthetic
  source 1..20: mol_type = other DNA
catgctttcccgcaagcgtt

SEQ ID NO: 180 - DNA, 20 bp, synthetic construct
  misc_feature 1..20: note = Synthetic
  source 1..20: mol_type = other DNA
cggatgtgaccaaacgcttg

SEQ ID NO: 181 - DNA, 20 bp, synthetic construct
  misc_feature 1..20: note = Synthetic
  source 1..20: mol_type = other DNA
gtgtgccccgtttattggtg

SEQ ID NO: 182 - DNA, 20 bp, synthetic construct
  misc_feature 1..20: note = Synthetic
  source 1..20: mol_type = other DNA
agcccggtgccgaaggcccg

SEQ ID NO: 183 - DNA, 20 bp, synthetic construct
  misc_feature 1..20: note = Synthetic
  source 1..20: mol_type = other DNA
ttctagaggatcaatactcg

SEQ ID NO: 184 - DNA, 20 bp, synthetic construct
  misc_feature 1..20: note = Synthetic
  source 1..20: mol_type = other DNA
gggtcggcttattaaagaat

SEQ ID NO: 185 - DNA, 20 bp, synthetic construct
  misc_feature 1..20: note = Synthetic
  source 1..20: mol_type = other DNA
ttaataagccgacccctatg

SEQ ID NO: 186 - DNA, 20 bp, synthetic construct
  misc_feature 1..20: note = Synthetic
  source 1..20: mol_type = other DNA
gccggtcttcctagtacaac

SEQ ID NO: 187 - DNA, 20 bp, synthetic construct
  misc_feature 1..20: note = Synthetic
  source 1..20: mol_type = other DNA
gcaaccttctttccgagtag

SEQ ID NO: 188 - DNA, 20 bp, synthetic construct
  misc_feature 1..20: note = Synthetic
  source 1..20: mol_type = other DNA
gttgctgattggagccggca

SEQ ID NO: 189 - DNA, 20 bp, synthetic construct
  misc_feature 1..20: note = Synthetic
  source 1..20: mol_type = other DNA
gggtcgtacctatgtctctc

SEQ ID NO: 190 - DNA, 20 bp, synthetic construct
  misc_feature 1..20: note = Synthetic
  source 1..20: mol_type = other DNA
cttgcgggaaagcatgctac

SEQ ID NO: 191 - DNA, 20 bp, synthetic construct
  misc_feature 1..20: note = Synthetic
  source 1..20: mol_type = other DNA
tgtgttggcgctgcctcgac

SEQ ID NO: 192 - DNA, 20 bp, synthetic construct
  misc_feature 1..20: note = Synthetic
  source 1..20: mol_type = other DNA
cttcaacagtcccgcccatg

SEQ ID NO: 193 - DNA, 20 bp, synthetic construct
  misc_feature 1..20: note = Synthetic
  source 1..20: mol_type = other DNA
cccatgggcgggactgttga

SEQ ID NO: 194 - DNA, 20 bp, synthetic construct
  misc_feature 1..20: note = Synthetic
  source 1..20: mol_type = other DNA
gccctagtgcatattggtta

SEQ ID NO: 195 - DNA, 20 bp, synthetic construct
  misc_feature 1..20: note = Synthetic
  source 1..20: mol_type = other DNA
tatggctaccggaactctgt

SEQ ID NO: 196 - DNA, 20 bp, synthetic construct
  misc_feature 1..20: note = Synthetic
  source 1..20: mol_type = other DNA
ccggtagccataattgctcc

SEQ ID NO: 197 - DNA, 20 bp, synthetic construct
  misc_feature 1..20: note = Synthetic
  source 1..20: mol_type = other DNA
gccatgtgtgccccgtttat

SEQ ID NO: 198 - DNA, 20 bp, synthetic construct
  misc_feature 1..20: note = Synthetic
  source 1..20: mol_type = other DNA
atcaccggtgccaaagggcc

SEQ ID NO: 199 - DNA, 20 bp, synthetic construct
  misc_feature 1..20: note = Synthetic
  source 1..20: mol_type = other DNA
tctacgctatcccatactga

SEQ ID NO: 200 - DNA, 20 bp, synthetic construct
  misc_feature 1..20: note = Synthetic
  source 1..20: mol_type = other DNA
atgtaatcaccggtgccaaa

SEQ ID NO: 201 - DNA, 20 bp, synthetic construct
  misc_feature 1..20: note = Synthetic
  source 1..20: mol_type = other DNA
atagtgactatgcagtgcga

SEQ ID NO: 202 - DNA, 20 bp, synthetic construct
  misc_feature 1..20: note = Synthetic
  source 1..20: mol_type = other DNA
gccccctatcttaaatttat

SEQ ID NO: 203 - DNA, 20 bp, synthetic construct
  misc_feature 1..20: note = Synthetic
  source 1..20: mol_type = other DNA
tggtgtgcccttacatcatt

SEQ ID NO: 204 - DNA, 20 bp, synthetic construct
  misc_feature 1..20: note = Synthetic
  source 1..20: mol_type = other DNA
ggattccaccggaggcaaac

SEQ ID NO: 205 - DNA, 20 bp, synthetic construct
  misc_feature 1..20: note = Synthetic
  source 1..20: mol_type = other DNA
gcaggcacccaatgatgtaa

SEQ ID NO: 206 - DNA, 20 bp, synthetic construct
  misc_feature 1..20: note = Synthetic
  source 1..20: mol_type = other DNA
atgaaagtacgtacaaactg

SEQ ID NO: 207 - DNA, 20 bp, synthetic construct
  misc_feature 1..20: note = Synthetic
  source 1..20: mol_type = other DNA
gatctagggctggatcactt

SEQ ID NO: 208 - DNA, 20 bp, synthetic construct
  misc_feature 1..20: note = Synthetic
  source 1..20: mol_type = other DNA
tagccactagcatgtttaat

SEQ ID NO: 209 - DNA, 20 bp, synthetic construct
  misc_feature 1..20: note = Synthetic
  source 1..20: mol_type = other DNA
agaatcctgtttgcctccgg

SEQ ID NO: 210 - DNA, 20 bp, synthetic construct
  misc_feature 1..20: note = Synthetic
  source 1..20: mol_type = other DNA
agcagttcagtataccttaa

SEQ ID NO: 211 - DNA, 20 bp, synthetic construct
  misc_feature 1..20: note = Synthetic
  source 1..20: mol_type = other DNA
ttaagatagggggcatgttg

SEQ ID NO: 212 - DNA, 20 bp, synthetic construct
  misc_feature 1..20: note = Synthetic
  source 1..20: mol_type = other DNA
aatgtaatcaccggtgccaa

SEQ ID NO: 213 - DNA, 20 bp, synthetic construct
  misc_feature 1..20: note = Synthetic
  source 1..20: mol_type = other DNA
agatccatggattccaccgg

SEQ ID NO: 214 - DNA, 20 bp, synthetic construct
  misc_feature 1..20: note = Synthetic
  source 1..20: mol_type = other DNA
tcctgtcaagctgtacacac

SEQ ID NO: 215 - DNA, 20 bp, synthetic construct
  misc_feature 1..20: note = Synthetic
  source 1..20: mol_type = other DNA
gaaaacccttgccagtagtt

SEQ ID NO: 216 - DNA, 20 bp, synthetic construct
  misc_feature 1..20: note = Synthetic
  source 1..20: mol_type = other DNA
agatatcacctatggtagct

SEQ ID NO: 217 - DNA, 20 bp, synthetic construct
  misc_feature 1..20: note = Synthetic
  source 1..20: mol_type = other DNA
catccaattaaacatgctag

SEQ ID NO: 218 - DNA, 20 bp, synthetic construct
  misc_feature 1..20: note = Synthetic
  source 1..20: mol_type = other DNA
ttgcaattacttagatgtac

SEQ ID NO: 219 - DNA, 20 bp, synthetic construct
  misc_feature 1..20: note = Synthetic
  source 1..20: mol_type = other DNA
gctataatcactcaatgtgt

SEQ ID NO: 220 - DNA, 20 bp, synthetic construct
  misc_feature 1..20: note = Synthetic
  source 1..20: mol_type = other DNA
gttagattcctaggtttcta

SEQ ID NO: 221 - DNA, 20 bp, synthetic construct
  misc_feature 1..20: note = Synthetic
  source 1..20: mol_type = other DNA
aagaccccccactccagggc

SEQ ID NO: 222 - DNA, 20 bp, synthetic construct
  misc_feature 1..20: note = Synthetic
  source 1..20: mol_type = other DNA
gacctacagttaatggttta

SEQ ID NO: 223 - DNA, 20 bp, synthetic construct
  misc_feature 1..20: note = Synthetic
  source 1..20: mol_type = other DNA
ggcaggcacccaatgatgta

SEQ ID NO: 224 - DNA, 20 bp, synthetic construct
  misc_feature 1..20: note = Synthetic
  source 1..20: mol_type = other DNA
ttgtggagggcaaatcacac

SEQ ID NO: 225 - DNA, 20 bp, synthetic construct
  misc_feature 1..20: note = Synthetic
  source 1..20: mol_type = other DNA
cttgctgattctggaatacg

SEQ ID NO: 226 - DNA, 20 bp, synthetic construct
  misc_feature 1..20: note = Synthetic
  source 1..20: mol_type = other DNA
tttgcctccggtggaatcca

SEQ ID NO: 227 - DNA, 20 bp, synthetic construct
  misc_feature 1..20: note = Synthetic
  source 1..20: mol_type = other DNA
ggaatccatggatctaagtt

SEQ ID NO: 228 - RNA, 20 nt, synthetic construct
  misc_feature 1..20: note = Synthetic
  source 1..20: mol_type = other RNA
cgttatcgagtattgggcga

SEQ ID NO: 229 - RNA, 20 nt, synthetic construct
  misc_feature 1..20: note = Synthetic
  source 1..20: mol_type = other RNA
acgttatcgagtattgggcg

SEQ ID NO: 230 - RNA, 20 nt, synthetic construct
  misc_feature 1..20: note = Synthetic
  source 1..20: mol_type = other RNA
gttatcgagtattgggcgag

SEQ ID NO: 231 - RNA, 20 nt, synthetic construct
  misc_feature 1..20: note = Synthetic
  source 1..20: mol_type = other RNA
atcatgacgttatcgagtat

SEQ ID NO: 232 - RNA, 20 nt, synthetic construct
  misc_feature 1..20: note = Synthetic
  source 1..20: mol_type = other RNA
gtagccgtcacttattagat

SEQ ID NO: 233 - RNA, 20 nt, synthetic construct
  misc_feature 1..20: note = Synthetic
  source 1..20: mol_type = other RNA
tcatgacgttatcgagtatt

SEQ ID NO: 234 - RNA, 20 nt, synthetic construct
  misc_feature 1..20: note = Synthetic
  source 1..20: mol_type = other RNA
tatcttgtacgatagggtca

SEQ ID NO: 235 - RNA, 20 nt, synthetic construct
  misc_feature 1..20: note = Synthetic
  source 1..20: mol_type = other RNA
actggccagtacacatcgct

SEQ ID NO: 236 - RNA, 20 nt, synthetic construct
  misc_feature 1..20: note = Synthetic
  source 1..20: mol_type = other RNA
attgttaattgtagtccggg

SEQ ID NO: 237 - RNA, 20 nt, synthetic construct
  misc_feature 1..20: note = Synthetic
  source 1..20: mol_type = other RNA
aactccccagcgatgtgtac

SEQ ID NO: 238 - RNA, 20 nt, synthetic construct
  misc_feature 1..20: note = Synthetic
  source 1..20: mol_type = other RNA
cgcatgatgcttgctctcac

SEQ ID NO: 239 - RNA, 20 nt, synthetic construct
  misc_feature 1..20: note = Synthetic
  source 1..20: mol_type = other RNA
gttaaaagacgggattcctt

SEQ ID NO: 240 - RNA, 20 nt, synthetic construct
  misc_feature 1..20: note = Synthetic
  source 1..20: mol_type = other RNA
gtataggtactaacactccc

SEQ ID NO: 241 - RNA, 20 nt, synthetic construct
  misc_feature 1..20: note = Synthetic
  source 1..20: mol_type = other RNA
ttgtacgatagggtcaagga

SEQ ID NO: 242 - RNA, 20 nt, synthetic construct
  misc_feature 1..20: note = Synthetic
  source 1..20: mol_type = other RNA
atgcaatgtgctgatccgga

SEQ ID NO: 243 - RNA, 20 nt, synthetic construct
  misc_feature 1..20: note = Synthetic
  source 1..20: mol_type = other RNA
tcgagtattgggcgaggggt

SEQ ID NO: 244 - RNA, 20 nt, synthetic construct
  misc_feature 1..20: note = Synthetic
  source 1..20: mol_type = other RNA
catgcaatgtgctgatccgg

SEQ ID NO: 245 - RNA, 20 nt, synthetic construct
  misc_feature 1..20: note = Synthetic
  source 1..20: mol_type = other RNA
cttgtacgatagggtcaagg

SEQ ID NO: 246 - RNA, 20 nt, synthetic construct
  misc_feature 1..20: note = Synthetic
  source 1..20: mol_type = other RNA
ctggccagtacacatcgctg

SEQ ID NO: 247 - RNA, 20 nt, synthetic construct
  misc_feature 1..20: note = Synthetic
  source 1..20: mol_type = other RNA
gcatcgaaggacactaaagc

SEQ ID NO: 248 - RNA, 20 nt, synthetic construct
  misc_feature 1..20: note = Synthetic
  source 1..20: mol_type = other RNA
tgcaatgtgctgatccggag

SEQ ID NO: 249 - RNA, 20 nt, synthetic construct
  misc_feature 1..20: note = Synthetic
  source 1..20: mol_type = other RNA
gagatcaatcatgtactagg

SEQ ID NO: 250 - RNA, 20 nt, synthetic construct
  misc_feature 1..20: note = Synthetic
  source 1..20: mol_type = other RNA
cacgctaacataatattccc

SEQ ID NO: 251 - RNA, 20 nt, synthetic construct
  misc_feature 1..20: note = Synthetic
  source 1..20: mol_type = other RNA
acttacaataagactcctgc

SEQ ID NO: 252 - RNA, 20 nt, synthetic construct
  misc_feature 1..20: note = Synthetic
  source 1..20: mol_type = other RNA
cactggccagtacacatcgc

SEQ ID NO: 253 - RNA, 20 nt, synthetic construct
  misc_feature 1..20: note = Synthetic
  source 1..20: mol_type = other RNA
gctagaggacatatcctctt

SEQ ID NO: 254 - RNA, 20 nt, synthetic construct
  misc_feature 1..20: note = Synthetic
  source 1..20: mol_type = other RNA
caaggttggtggtttccggc

SEQ ID NO: 255 - RNA, 20 nt, synthetic construct
  misc_feature 1..20: note = Synthetic
  source 1..20: mol_type = other RNA
cgcattgttaattgtagtcc

SEQ ID NO: 256 - RNA, 20 nt, synthetic construct
  misc_feature 1..20: note = Synthetic
  source 1..20: mol_type = other RNA
ctgggaatattatgttagcg

SEQ ID NO: 257 - RNA, 20 nt, synthetic construct
  misc_feature 1..20: note = Synthetic
  source 1..20: mol_type = other RNA
gcacggacccgtttacggag

SEQ ID NO: 258 - RNA, 20 nt, synthetic construct
  misc_feature 1..20: note = Synthetic
  source 1..20: mol_type = other RNA
acgctacggaagtcaactgc

SEQ ID NO: 259 - RNA, 20 nt, synthetic construct
  misc_feature 1..20: note = Synthetic
  source 1..20: mol_type = other RNA
cacggacccgtttacggaga

SEQ ID NO: 260 - RNA, 20 nt, synthetic construct
  misc_feature 1..20: note = Synthetic
  source 1..20: mol_type = other RNA
aatttgcacggacccgttta

SEQ ID NO: 261 - RNA, 20 nt, synthetic construct
  misc_feature 1..20: note = Synthetic
  source 1..20: mol_type = other RNA
tcgggcggcactgaacgccc

SEQ ID NO: 262 - RNA, 20 nt, synthetic construct
  misc_feature 1..20: note = Synthetic
  source 1..20: mol_type = other RNA
aaattccacagacgcgcacc

SEQ ID NO: 263 - RNA, 20 nt, synthetic construct
  misc_feature 1..20: note = Synthetic
  source 1..20: mol_type = other RNA
gccgggttctgcgtacacat

SEQ ID NO: 264 - RNA, 20 nt, synthetic construct
  misc_feature 1..20: note = Synthetic
  source 1..20: mol_type = other RNA
atctggacccgtgcgccagc

SEQ ID NO: 265 - RNA, 20 nt, synthetic construct
  misc_feature 1..20: note = Synthetic
  source 1..20: mol_type = other RNA
agatcgcatgcacgcccagc

SEQ ID NO: 266 - RNA, 20 nt, synthetic construct
  misc_feature 1..20: note = Synthetic
  source 1..20: mol_type = other RNA
gtgtagtcaaccctgcccga

SEQ ID NO: 267 - RNA, 20 nt, synthetic construct
  misc_feature 1..20: note = Synthetic
  source 1..20: mol_type = other RNA
aacgggtccgtgcaaattca

SEQ ID NO: 268 - RNA, 20 nt, synthetic construct
  misc_feature 1..20: note = Synthetic
  source 1..20: mol_type = other RNA
tgcagcacgcagttcttgcg

SEQ ID NO: 269 - RNA, 20 nt, synthetic construct
  misc_feature 1..20: note = Synthetic
  source 1..20: mol_type = other RNA
tgggcaccttgattcggtgc

SEQ ID NO: 270 - RNA, 20 nt, synthetic construct
  misc_feature 1..20: note = Synthetic
  source 1..20: mol_type = other RNA
aggtctcttgttgcacccga

SEQ ID NO: 271 - RNA, 20 nt, synthetic construct
  misc_feature 1..20: note = Synthetic
  source 1..20: mol_type = other RNA
ctggcacgtccccgaaaatg

SEQ ID NO: 272 - RNA, 20 nt, synthetic construct
  misc_feature 1..20: note = Synthetic
  source 1..20: mol_type = other RNA
cattgtacgactaagattga

SEQ ID NO: 273 - RNA, 20 nt, synthetic construct
  misc_feature 1..20: note = Synthetic
  source 1..20: mol_type = other RNA
gttcagtgccgcccgacaga

SEQ ID NO: 274 - RNA, 20 nt, synthetic construct
  misc_feature 1..20: note = Synthetic
  source 1..20: mol_type = other RNA
tgtgtagtcaaccctgcccg

SEQ ID NO: 275 - RNA, 20 nt, synthetic construct
  misc_feature 1..20: note = Synthetic
  source 1..20: mol_type = other RNA
ggcacacctgacgatgtcaa

SEQ ID NO: 276 - RNA, 20 nt, synthetic construct
  misc_feature 1..20: note = Synthetic
  source 1..20: mol_type = other RNA
cggccaccagcttttgccgg

SEQ ID NO: 277 - RNA, 20 nt, synthetic construct
  misc_feature 1..20: note = Synthetic
  source 1..20: mol_type = other RNA
gggactctggccctaacgtg

SEQ ID NO: 278 - RNA, 20 nt, synthetic construct
  misc_feature 1..20: note = Synthetic
  source 1..20: mol_type = other RNA
aggtgcactaggtagactag

SEQ ID NO: 279 - RNA, 20 nt, synthetic construct
  misc_feature 1..20: note = Synthetic
  source 1..20: mol_type = other RNA
aggccccctttgacatcgtc

SEQ ID NO: 280 - RNA, 20 nt, synthetic construct
  misc_feature 1..20: note = Synthetic
  source 1..20: mol_type = other RNA
aggcaccatctgcgccgagc

SEQ ID NO: 281 - RNA, 20 nt, synthetic construct
  misc_feature 1..20: note = Synthetic
  source 1..20: mol_type = other RNA
gcgcacgggtccagatggag

SEQ ID NO: 282 - RNA, 20 nt, synthetic construct
  misc_feature 1..20: note = Synthetic
  source 1..20: mol_type = other RNA
ctggataccagctggcgcac

SEQ ID NO: 283 - RNA, 20 nt, synthetic construct
  misc_feature 1..20: note = Synthetic
  source 1..20: mol_type = other RNA
ggtactggtggcaaacgcta

SEQ ID NO: 284 - RNA, 20 nt, synthetic construct
  misc_feature 1..20: note = Synthetic
  source 1..20: mol_type = other RNA
aggtgcgcgtctgtggaatt

SEQ ID NO: 285 - RNA, 20 nt, synthetic construct
  misc_feature 1..20: note = Synthetic
  source 1..20: mol_type = other RNA
gcccatgtgtacgcagaacc

SEQ ID NO: 286 - RNA, 20 nt, synthetic construct
  misc_feature 1..20: note = Synthetic
  source 1..20: mol_type = other RNA
acccgctagaatctagtcat

SEQ ID NO: 287 - RNA, 20 nt, synthetic construct
  misc_feature 1..20: note = Synthetic
  source 1..20: mol_type = other RNA
aagtaggacgttcccgcata

SEQ ID NO: 288 - RNA, 20 nt, synthetic construct
  misc_feature 1..20: note = Synthetic
  source 1..20: mol_type = other RNA
atccgacctgcctcgggctt

SEQ ID NO: 289 - RNA, 20 nt, synthetic construct
  misc_feature 1..20: note = Synthetic
  source 1..20: mol_type = other RNA
tcgagagcaagacaacgcaa

SEQ ID NO: 290 - RNA, 20 nt, synthetic construct
  misc_feature 1..20: note = Synthetic
  source 1..20: mol_type = other RNA
cacttgaagattccggatga

SEQ ID NO: 291 - RNA, 20 nt, synthetic construct
  misc_feature 1..20: note = Synthetic
  source 1..20: mol_type = other RNA
ctaccagtgggcctaaataa

SEQ ID NO: 292 - RNA, 20 nt, synthetic construct
  misc_feature 1..20: note = Synthetic
  source 1..20: mol_type = other RNA
taagtgtcagcttaccaacg

SEQ ID NO: 293 - RNA, 20 nt, synthetic construct
  misc_feature 1..20: note = Synthetic
  source 1..20: mol_type = other RNA
ggggccaatagcaaactaac

SEQ ID NO: 294 - RNA, 20 nt, synthetic construct
  misc_feature 1..20: note = Synthetic
  source 1..20: mol_type = other RNA
ccgaatctatcagtgcctgg

SEQ ID NO: 295 - RNA, 20 nt, synthetic construct
  misc_feature 1..20: note = Synthetic
  source 1..20: mol_type = other RNA
cgagcatatatgctagattt

SEQ ID NO: 296 - RNA, 20 nt, synthetic construct
  misc_feature 1..20: note = Synthetic
  source 1..20: mol_type = other RNA
gtcggatgccaggatttcaa

SEQ ID NO: 297 - RNA, 20 nt, synthetic construct
  misc_feature 1..20: note = Synthetic
  source 1..20: mol_type = other RNA
tagaactcacgacatagggc

SEQ ID NO: 298 - RNA, 20 nt, synthetic construct
  misc_feature 1..20: note = Synthetic
  source 1..20: mol_type = other RNA
tttaactaaggttgccgaaa

SEQ ID NO: 299 - RNA, 20 nt, synthetic construct
  misc_feature 1..20: note = Synthetic
  source 1..20: mol_type = other RNA
gaacggatgcaacaagacca

SEQ ID NO: 300 - RNA, 20 nt, synthetic construct
  misc_feature 1..20: note = Synthetic
  source 1..20: mol_type = other RNA
gatgtgctaacatcctcccg

SEQ ID NO: 301 - RNA, 20 nt, synthetic construct
  misc_feature 1..20: note = Synthetic
  source 1..20: mol_type = other RNA
agtttctttatcacgatcat

SEQ ID NO: 302 - RNA, 20 nt, synthetic construct
  misc_feature 1..20: note = Synthetic
  source 1..20: mol_type = other RNA
ggaacggatgcaacaagacc

SEQ ID NO: 303 - RNA, 20 nt, synthetic construct
  misc_feature 1..20: note = Synthetic
  source 1..20: mol_type = other RNA
tcctatgactagattctagc

SEQ ID NO: 304 - RNA, 20 nt, synthetic construct
  misc_feature 1..20: note = Synthetic
  source 1..20: mol_type = other RNA
atgccattatttaggcccac

SEQ ID NO: 305 - RNA, 20 nt, synthetic construct
  misc_feature 1..20: note = Synthetic
  source 1..20: mol_type = other RNA
agtctgacacggacaagtac

SEQ ID NO: 306 - RNA, 20 nt, synthetic construct
  misc_feature 1..20: note = Synthetic
  source 1..20: mol_type = other RNA
catccgacctgcctcgggct

SEQ ID NO: 307 - RNA, 20 nt, synthetic construct
  misc_feature 1..20: note = Synthetic
  source 1..20: mol_type = other RNA
tcagcactggtacatatatt

SEQ ID NO: 308 - RNA, 20 nt, synthetic construct
  misc_feature 1..20: note = Synthetic
  source 1..20: mol_type = other RNA
agtccgaatctatcagtgcc

SEQ ID NO: 309 - RNA, 20 nt, synthetic construct
  misc_feature 1..20: note = Synthetic
  source 1..20: mol_type = other RNA
atataggatcccaacaatat

SEQ ID NO: 310 - RNA, 20 nt, synthetic construct
  misc_feature 1..20: note = Synthetic
  source 1..20: mol_type = other RNA
ccagcaaatactagattggg

SEQ ID NO: 311 - RNA, 20 nt, synthetic construct
  misc_feature 1..20: note = Synthetic
  source 1..20: mol_type = other RNA
actccatattcttccccggg

SEQ ID NO: 312 - RNA, 20 nt, synthetic construct
  misc_feature 1..20: note = Synthetic
  source 1..20: mol_type = other RNA
aacgcacatcgtttatatgg

SEQ ID NO: 313 - RNA, 20 nt, synthetic construct
  misc_feature 1..20: note = Synthetic
  source 1..20: mol_type = other RNA
actaacaggatgcatgcaag

SEQ ID NO: 314 - RNA, 20 nt, synthetic construct
  misc_feature 1..20: note = Synthetic
  source 1..20: mol_type = other RNA
taggacgttcccgcataagg

SEQ ID NO: 315 - RNA, 20 nt, synthetic construct
  misc_feature 1..20: note = Synthetic
  source 1..20: mol_type = other RNA
gggaccgatttggccgttaa

SEQ ID NO: 316 - RNA, 20 nt, synthetic construct
  misc_feature 1..20: note = Synthetic
  source 1..20: mol_type = other RNA
gtaaccattaacggccaaat

SEQ ID NO: 317 - RNA, 20 nt, synthetic construct
  misc_feature 1..20: note = Synthetic
  source 1..20: mol_type = other RNA
cacggcccttcgattgttga

SEQ ID NO: 318 - RNA, 20 nt, synthetic construct
  misc_feature 1..20: note = Synthetic
  source 1..20: mol_type = other RNA
aaggatcgttaggtgatcgt

SEQ ID NO: 319 - RNA, 20 nt, synthetic construct
  misc_feature 1..20: note = Synthetic
  source 1..20: mol_type = other RNA
tacctaccggtcatatatcc

SEQ ID NO: 320 - RNA, 20 nt, synthetic construct
  misc_feature 1..20: note = Synthetic
  source 1..20: mol_type = other RNA
cacgaggacgattaccatat

SEQ ID NO: 321 - RNA, 20 nt, synthetic construct
  misc_feature 1..20: note = Synthetic
  source 1..20: mol_type = other RNA
aggatcgttaggtgatcgtc

SEQ ID NO: 322 - RNA, 20 nt, synthetic construct
  misc_feature 1..20: note = Synthetic
  source 1..20: mol_type = other RNA
cacgggacgctattctaaac

SEQ ID NO: 323 - RNA, 20 nt, synthetic construct
  misc_feature 1..20: note = Synthetic
  source 1..20: mol_type = other RNA
ccataatcaggtcgtttatc

SEQ ID NO: 324 - RNA, 20 nt, synthetic construct
  misc_feature 1..20: note = Synthetic
  source 1..20: mol_type = other RNA
atcggtcccataatagggtg

SEQ ID NO: 325 - RNA, 20 nt, synthetic construct
  misc_feature 1..20: note = Synthetic
  source 1..20: mol_type = other RNA
ggccaaatcggtcccataat

SEQ ID NO: 326 - RNA, 20 nt, synthetic construct
  misc_feature 1..20: note = Synthetic
  source 1..20: mol_type = other RNA
ctcggtttactaggacaaag

SEQ ID NO: 327 - RNA, 20 nt, synthetic construct
  misc_feature 1..20: note = Synthetic
  source 1..20: mol_type = other RNA
atagcgtcccgtgatgtttt

SEQ ID NO: 328 - RNA, 20 nt, synthetic construct
  misc_feature 1..20: note = Synthetic
  source 1..20: mol_type = other RNA
tcagtattgtaccgctttag

SEQ ID NO: 329 - RNA, 20 nt, synthetic construct
  misc_feature 1..20: note = Synthetic
  source 1..20: mol_type = other RNA
aatcggtcccataatagggt

SEQ ID NO: 330 - RNA, 20 nt, synthetic construct
  misc_feature 1..20: note = Synthetic
  source 1..20: mol_type = other RNA
accctattatgggaccgatt

SEQ ID NO: 331 - RNA, 20 nt, synthetic construct
  misc_feature 1..20: note = Synthetic
  source 1..20: mol_type = other RNA
tgcacttgcccgtgtaacac

SEQ ID NO: 332 - RNA, 20 nt, synthetic construct
  misc_feature 1..20: note = Synthetic
  source 1..20: mol_type = other RNA
gatctaccatcaacaatcga

SEQ ID NO: 333 - RNA, 20 nt, synthetic construct
  misc_feature 1..20: note = Synthetic
  source 1..20: mol_type = other RNA
caccaggatatatgaccggt

SEQ ID NO: 334 - RNA, 20 nt, synthetic construct
  misc_feature 1..20: note = Synthetic
  source 1..20: mol_type = other RNA
ggtggctacgttgggcccac

SEQ ID NO: 335 - RNA, 20 nt, synthetic construct
  misc_feature 1..20: note = Synthetic
  source 1..20: mol_type = other RNA
gccaaatcggtcccataata

SEQ ID NO: 336 - RNA, 20 nt, synthetic construct
  misc_feature 1..20: note = Synthetic
  source 1..20: mol_type = other RNA
atcagttataacagcctagc

SEQ ID NO: 337 - RNA, 20 nt, synthetic construct
  misc_feature 1..20: note = Synthetic
  source 1..20: mol_type = other RNA
ccgcttgcactagagggtcc

SEQ ID NO: 338 - RNA, 20 nt, synthetic construct
  misc_feature 1..20: note = Synthetic
  source 1..20: mol_type = other RNA
gaccctctagtgcaagcggc

SEQ ID NO: 339 - RNA, 20 nt, synthetic construct
  misc_feature 1..20: note = Synthetic
  source 1..20: mol_type = other RNA
gatagttactttactctccg

SEQ ID NO: 340 - RNA, 20 nt, synthetic construct
  misc_feature 1..20: note = Synthetic
  source 1..20: mol_type = other RNA
atcgaagggccgtgttaaga

SEQ ID NO: 341 - RNA, 20 nt, synthetic construct
  misc_feature 1..20: note = Synthetic
  source 1..20: mol_type = other RNA
aaagaggcttctcaacccgc

SEQ ID NO: 342 - RNA, 20 nt, synthetic construct
  misc_feature 1..20: note = Synthetic
  source 1..20: mol_type = other RNA
acggaccatcatgcacggca

SEQ ID NO: 343 - RNA, 20 nt, synthetic construct
  misc_feature 1..20: note = Synthetic
  source 1..20: mol_type = other RNA
gtcccccgtgccctagatcc

SEQ ID NO: 344 - RNA, 20 nt, synthetic construct
  misc_feature 1..20: note = Synthetic
  source 1..20: mol_type = other RNA
tcgaaggaccaaactcatag

SEQ ID NO: 345 - RNA, 20 nt, synthetic construct
  misc_feature 1..20: note = Synthetic
  source 1..20: mol_type = other RNA
gtacgacccataaaagtgcc

SEQ ID NO: 346 - RNA, 20 nt, synthetic construct
  misc_feature 1..20: note = Synthetic
  source 1..20: mol_type = other RNA
ctgtgaatacgccttggcac

SEQ ID NO: 347 - RNA, 20 nt, synthetic construct
  misc_feature 1..20: note = Synthetic
  source 1..20: mol_type = other RNA
ccttcaacagtcccgcccat

SEQ ID NO: 348 - RNA, 20 nt, synthetic construct
  misc_feature 1..20: note = Synthetic
  source 1..20: mol_type = other RNA
cgtgtgcagcccggtgccga

SEQ ID NO: 349 - RNA, 20 nt, synthetic construct
  misc_feature 1..20: note = Synthetic
  source 1..20: mol_type = other RNA
cacaaacgatgggtagtttc

SEQ ID NO: 350 - RNA, 20 nt, synthetic construct
  misc_feature 1..20: note = Synthetic
  source 1..20: mol_type = other RNA
tcgcacactaaacgaggaac

SEQ ID NO: 351 - RNA, 20 nt, synthetic construct
  misc_feature 1..20: note = Synthetic
  source 1..20: mol_type = other RNA
atacgccttggcactggatc

SEQ ID NO: 352 - RNA, 20 nt, synthetic construct
  misc_feature 1..20: note = Synthetic
  source 1..20: mol_type = other RNA
tgccacgggccttcggcacc

SEQ ID NO: 353 - RNA, 20 nt, synthetic construct
  misc_feature 1..20: note = Synthetic
  source 1..20: mol_type = other RNA
ggaactgtgatccgtgccac

SEQ ID NO: 354 - RNA, 20 nt, synthetic construct
  misc_feature 1..20: note = Synthetic
  source 1..20: mol_type = other RNA
aggctactatgatgcggtag

SEQ ID NO: 355 - RNA, 20 nt, synthetic construct
  misc_feature 1..20: note = Synthetic
  source 1..20: mol_type = other RNA
gtttgatcgcacactaaacg

SEQ ID NO: 356 - RNA, 20 nt, synthetic construct
  misc_feature 1..20: note = Synthetic
  source 1..20: mol_type = other RNA
catgctttcccgcaagcgtt

SEQ ID NO: 357 - RNA, 20 nt, synthetic construct
  misc_feature 1..20: note = Synthetic
  source 1..20: mol_type = other RNA
cggatgtgaccaaacgcttg

SEQ ID NO: 358 - RNA, 20 nt, synthetic construct
  misc_feature 1..20: note = Synthetic
  source 1..20: mol_type = other RNA
gtgtgccccgtttattggtg

SEQ ID NO: 359 - RNA, 20 nt, synthetic construct
  misc_feature 1..20: note = Synthetic
  source 1..20: mol_type = other RNA
agcccggtgccgaaggcccg

SEQ ID NO: 360 - RNA, 20 nt, synthetic construct
  misc_feature 1..20: note = Synthetic
  source 1..20: mol_type = other RNA
ttctagaggatcaatactcg

SEQ ID NO: 361 - RNA, 20 nt, synthetic construct
  misc_feature 1..20: note = Synthetic
  source 1..20: mol_type = other RNA
gggtcggcttattaaagaat

SEQ ID NO: 362 - RNA, 20 nt, synthetic construct
  misc_feature 1..20: note = Synthetic
  source 1..20: mol_type = other RNA
ttaataagccgacccctatg

SEQ ID NO: 363 - RNA, 20 nt, synthetic construct
  misc_feature 1..20: note = Synthetic
  source 1..20: mol_type = other RNA
gccggtcttcctagtacaac

SEQ ID NO: 364 - RNA, 20 nt, synthetic construct
  misc_feature 1..20: note = Synthetic
  source 1..20: mol_type = other RNA
gcaaccttctttccgagtag

SEQ ID NO: 365 - RNA, 20 nt, synthetic construct
  misc_feature 1..20: note = Synthetic
  source 1..20: mol_type = other RNA
gttgctgattggagccggca

SEQ ID NO: 366 - RNA, 20 nt, synthetic construct
  misc_feature 1..20: note = Synthetic
  source 1..20: mol_type = other RNA
gggtcgtacctatgtctctc

SEQ ID NO: 367 - RNA, 20 nt, synthetic construct
  misc_feature 1..20: note = Synthetic
  source 1..20: mol_type = other RNA
cttgcgggaaagcatgctac

SEQ ID NO: 368 - RNA, 20 nt, synthetic construct
  misc_feature 1..20: note = Synthetic
  source 1..20: mol_type = other RNA
tgtgttggcgctgcctcgac

SEQ ID NO: 369 - RNA, 20 nt, synthetic construct
  misc_feature 1..20: note = Synthetic
  source 1..20: mol_type = other RNA
cttcaacagtcccgcccatg

SEQ ID NO: 370 - RNA, 20 nt, synthetic construct
  misc_feature 1..20: note = Synthetic
  source 1..20: mol_type = other RNA
cccatgggcgggactgttga

SEQ ID NO: 371 - RNA, 20 nt, synthetic construct
  misc_feature 1..20: note = Synthetic
  source 1..20: mol_type = other RNA
gccctagtgcatattggtta

SEQ ID NO: 372 - RNA, 20 nt, synthetic construct
  misc_feature 1..20: note = Synthetic
  source 1..20: mol_type = other RNA
tatggctaccggaactctgt

SEQ ID NO: 373 - RNA, 20 nt, synthetic construct
  misc_feature 1..20: note = Synthetic
  source 1..20: mol_type = other RNA
ccggtagccataattgctcc

SEQ ID NO: 374 - RNA, 20 nt, synthetic construct
  misc_feature 1..20: note = Synthetic
  source 1..20: mol_type = other RNA
gccatgtgtgccccgtttat

SEQ ID NO: 375 - RNA, 20 nt, synthetic construct
  misc_feature 1..20: note = Synthetic
  source 1..20: mol_type = other RNA
atcaccggtgccaaagggcc

SEQ ID NO: 376 - RNA, 20 nt, synthetic construct
  misc_feature 1..20: note = Synthetic
  source 1..20: mol_type = other RNA
tctacgctatcccatactga

SEQ ID NO: 377 - RNA, 20 nt, synthetic construct
  misc_feature 1..20: note = Synthetic
  source 1..20: mol_type = other RNA
atgtaatcaccggtgccaaa

SEQ ID NO: 378 - RNA, 20 nt, synthetic construct
  misc_feature 1..20: note = Synthetic
  source 1..20: mol_type = other RNA
atagtgactatgcagtgcga

SEQ ID NO: 379 - RNA, 20 nt, synthetic construct
  misc_feature 1..20: note = Synthetic
  source 1..20: mol_type = other RNA
gccccctatcttaaatttat

SEQ ID NO: 380 - RNA, 20 nt, synthetic construct
  misc_feature 1..20: note = Synthetic
  source 1..20: mol_type = other RNA
tggtgtgcccttacatcatt

SEQ ID NO: 381 - RNA, 20 nt, synthetic construct
  misc_feature 1..20: note = Synthetic
  source 1..20: mol_type = other RNA
ggattccaccggaggcaaac

SEQ ID NO: 382 - RNA, 20 nt, synthetic construct
  misc_feature 1..20: note = Synthetic
  source 1..20: mol_type = other RNA
gcaggcacccaatgatgtaa

SEQ ID NO: 383 - RNA, 20 nt, synthetic construct
  misc_feature 1..20: note = Synthetic
  source 1..20: mol_type = other RNA
atgaaagtacgtacaaactg

SEQ ID NO: 384 - RNA, 20 nt, synthetic construct
  misc_feature 1..20: note = Synthetic
  source 1..20: mol_type = other RNA
gatctagggctggatcactt

SEQ ID NO: 385 - RNA, 20 nt, synthetic construct
  misc_feature 1..20: note = Synthetic
  source 1..20: mol_type = other RNA
tagccactagcatgtttaat

SEQ ID NO: 386 - RNA, 20 nt, synthetic construct
  misc_feature 1..20: note = Synthetic
  source 1..20: mol_type = other RNA
agaatcctgtttgcctccgg

SEQ ID NO: 387 - RNA, 20 nt, synthetic construct
  misc_feature 1..20: note = Synthetic
  source 1..20: mol_type = other RNA
agcagttcagtataccttaa

SEQ ID NO: 388 - RNA, 20 nt, synthetic construct
  misc_feature 1..20: note = Synthetic
  source 1..20: mol_type = other RNA
ttaagatagggggcatgttg

SEQ ID NO: 389 - RNA, 20 nt, synthetic construct
  misc_feature 1..20: note = Synthetic
  source 1..20: mol_type = other RNA
aatgtaatcaccggtgccaa

SEQ ID NO: 390 - RNA, 20 nt, synthetic construct
  misc_feature 1..20: note = Synthetic
  source 1..20: mol_type = other RNA
agatccatggattccaccgg

SEQ ID NO: 391 - RNA, 20 nt, synthetic construct
  misc_feature 1..20: note = Synthetic
  source 1..20: mol_type = other RNA
tcctgtcaagctgtacacac

SEQ ID NO: 392 - RNA, 20 nt, synthetic construct
  misc_feature 1..20: note = Synthetic
  source 1..20: mol_type = other RNA
gaaaacccttgccagtagtt

SEQ ID NO: 393 - RNA, 20 nt, synthetic construct
  misc_feature 1..20: note = Synthetic
  source 1..20: mol_type = other RNA
agatatcacctatggtagct

SEQ ID NO: 394 - RNA, 20 nt, synthetic construct
  misc_feature 1..20: note = Synthetic
  source 1..20: mol_type = other RNA
catccaattaaacatgctag

SEQ ID NO: 395 - RNA, 20 nt, synthetic construct
  misc_feature 1..20: note = Synthetic
  source 1..20: mol_type = other RNA
ttgcaattacttagatgtac

SEQ ID NO: 396 - RNA, 20 nt, synthetic construct
  misc_feature 1..20: note = Synthetic
  source 1..20: mol_type = other RNA
gctataatcactcaatgtgt

SEQ ID NO: 397 - RNA, 20 nt, synthetic construct
  misc_feature 1..20: note = Synthetic
  source 1..20: mol_type = other RNA
gttagattcctaggtttcta

SEQ ID NO: 398 - RNA, 20 nt, synthetic construct
  misc_feature 1..20: note = Synthetic
  source 1..20: mol_type = other RNA
aagaccccccactccagggc

SEQ ID NO: 399 - RNA, 20 nt, synthetic construct
  misc_feature 1..20: note = Synthetic
  source 1..20: mol_type = other RNA
gacctacagttaatggttta

SEQ ID NO: 400 - RNA, 20 nt, synthetic construct
  misc_feature 1..20: note = Synthetic
  source 1..20: mol_type = other RNA
ggcaggcacccaatgatgta

SEQ ID NO: 401 - RNA, 20 nt, synthetic construct
  misc_feature 1..20: note = Synthetic
  source 1..20: mol_type = other RNA
ttgtggagggcaaatcacac

SEQ ID NO: 402 - RNA, 20 nt, synthetic construct
  misc_feature 1..20: note = Synthetic
  source 1..20: mol_type = other RNA
cttgctgattctggaatacg

SEQ ID NO: 403 - RNA, 20 nt, synthetic construct
  misc_feature 1..20: note = Synthetic
  source 1..20: mol_type = other RNA
tttgcctccggtggaatcca

SEQ ID NO: 404 - RNA, 20 nt, synthetic construct
  misc_feature 1..20: note = Synthetic
  source 1..20: mol_type = other RNA
ggaatccatggatctaagtt

SEQ ID NO: 405 - DNA, 1005 bp, Mus musculus
  source 1..1005: mol_type = genomic DNA
ctgctatactcatgggcacgaggacgattaccatattggctcctgcgggttgagaagcctctttgttaataattgagtcagctaaattttattcctcagggtccagagcaggactccatatccaacatggtgaagaacaaaatcttggaggacagacagttgacaagtagagggaaattgctgacagagtcagctgggacttaatggaatccacttacaatagggatgggggctgtggcccaggaagcacctctgaggtgtgacctgtgtcacatctgggcacccctgtgagctggggagaacctcaagatggacctgagaaggcagcccaggagatgccaagatgccaagaggcattaaagaggatccacaacattgtgtcacttcatgaatttccccaagtttaacaaagactttttctaataacagcaatgtttttaaaagctctgaagtttagagaaagtcttttttcccccttctttctcaatttttacagtgttgcaaccatgcgtccttgctcaagtctgcccgggcagcaaagatgggcactttccagttgagtgtctgccatcaagagctgccagggcagaagctggtaaggatggctcaacaatccaccagatcctcatcttttcttgcctccttctgacatttagcccagaagtgaactccgggagcctcatgtctcaagaactagttgttattattacagcacactctctattattagagcaggaagaaacttgcatgtgcaggaaaaacaaggcccgacgatcacctaacgatccttctacagagtatgtgttttgcagccctgctcacaccaccttgggttcccagtgatattaacaaggtcttgtctcttctgggggtcatgagaagcagtgatttagctcaattattctagtcccacagggttgtaggaaacgcacgtgcatctctttcattagtttgaattcctttccactcgtgatttgaactcaggttacctactgccattgtcca

SEQ ID NO: 406 - DNA, 1001 bp, Mus musculus
  source 1..1001: mol_type = genomic DNA
gacacaataccgtcatgtaaccacataagcacaaggtctcattatcacatgtggcaaacgtatgtgcatgcggcccaccattactgtacccagttttcagtacccagtccaagagaatgttagagccttcttgagactagcttacaatagatgcttttgtattccttgctgacgagacccaaaaagcacccaggaagcactaccacccccacaggttctcaggattagtgggacagtcacaccactgacacacacagcagggtatcccgtgccattggagatgagagcaaagctaatcaactcctactgcccatgtcctgcttactccaaaagcatggtgcctgagaacaggtagtgttaatcaggcaagctggactgccgcttgcagctcctcaggtgggcacacacctctcctccccatgggcgggactgttgaaggttcagggattccatctcactgggagagcaggacccagcatccctcctctgcaacctgcagtactggcgatgtgactggcttctacatgccagggcctctgatgctcattttcaggagtatgccaattcactttaaagcttgttttatgatgcgtcaacagcccagatcacagtagtccaaacagcccagtaaatatttacacaaacctgggctccactggaattttcttctttgcagttgatgggcctctcaactgtctgtctgcatggatggccctctcaactgtttgcctggcggacctctcctctgtctggctaaggagttcttttctgtgtggctaactgtgaggggaggaggcttaggcctggatcagtactcaccatatacccttcctgcaaaaggctgtgggttgtaaagcacagggaagcccatgactgatttttgagcttgaaaatacgtgggagtgaccccgagtattgatcctctagaaaccgcaacaaaaggcctccaccaagccatgcttgtcatacatatgtgtgctgtacactgaagcatgagacat

SEQ ID NO: 407 - DNA, 985 bp, Mus musculus
  source 1..985: mol_type = genomic DNA
gtaataataacaggaaaagctttttaacagcaggaatttttcctaaaatgattttctactgagccttgcttattgaatcatcattgtagattttaatctaaggcaaagtcctctcataaagatcatctgctagttatagcttgtcccatgatggctcctgacagaagcaggtaggttttcctgttttatacacacacacacacacacacacacacacacacacacacacacacacacacacaaatcagataatataaagatggttcagtcaacaatgattttatcctttaaatcttcaaggtaaatgtattttgttaatttctaagagaataatgccttagtcctattctcctttctcccatgctataaactaagcttttctttgtaaattccccttatgtttcttcttgtacatgagctttatgttttatccaaatttgtgtccttttcccctcttctatgtgaatagcacacacatgctagaatacagcattccctgtgctctctgtggagaatctgctgacacctgaagtggcttctattggaatttgttcttggctgatctttacatgacctttgaaatcctggcatctgacctgctttgagcttggactccatggctctaagcctgccttctgcagggacattctcctctccatctcatattttctgccttaaccctctggaagccaaaatgtgagaagataagtcatttctagtcctctttgtcagccttcaccttctgagaatgtggaaataagcaaataaaatatatgggaaaatgcaaagataaacacctcttcttattggctattttgtcccaacaaaactaacatgctgagagctatcttctccaagggagaagacttggataaaaaacaaaatatcaggaactgggagatctttggtatttggaaataaaaagaaactcagctaaaaatcttattacttaatgaattattatttttcttacaaattttttttt

(19) United States
(12) Patent Application Publication
(10) Pub. No.: US 2025/0332287 A1
(43) Pub. Date: Oct. 30, 2025

(54) IDENTIFICATION OF TISSUE-SPECIFIC EXTRAGENIC SAFE HARBORS FOR GENE THERAPY APPROACHES

(71) Applicant: Regeneron Pharmaceuticals, Inc., Tarrytown, NY (US)

(72) Inventors: Ciro Bonetti, New York, NY (US); Guochun Gong, Pleasantville, NY (US); Jing He, Tarrytown, NY (US); Jinrui Liu, White Plains, NY (US); Gregg Warshaw, Bayside, NY (US); Eric Chiao, New Fairfield, CT (US); Brian Zambrowicz, Sleepy Hollow, NY (US)

(21) Appl. No.: 18/860,774

(22) PCT Filed: Apr. 28, 2023
(86) PCT No.: PCT/US2023/066343

§ 371 Date: Oct. 28, 2024

Related U.S. Application Data
(60) Provisional application No. 63/336,663, filed on Apr. 29, 2022.

(51) Int. Cl. A61K 48/00; C12N 9/22; C12N 15/10; C12N 15/11

(52) U.S. Cl. CPC A61K 48/0058; A61K 48/0041; C12N 9/226; C12N 15/1082; C12N 15/11; C12N 15/111; C12N 2310/20

(57) ABSTRACT

Compositions and methods for inserting a nucleic acid encoding a product of interest into a genomic safe harbor locus in a cell, a population of cells, or a subject or for expressing a nucleic acid encoding a product of interest from a genomic safe harbor locus in a cell, a population of cells, or a subject are provided. Also provided are cells or populations of cells comprising a nucleic acid construct comprising a coding sequence for a product of interest inserted into a genomic safe harbor locus. Also provided are methods of identifying genomic safe harbor loci for use in specific cell or tissue types.

### CROSS-REFERENCE TO RELATED APPLICATIONS

This application claims the benefit of U.S. Application No. 63/336,663, filed Apr. 29, 2022, which is herein incorporated by reference in its entirety for all purposes.

### REFERENCE TO A SEQUENCE LISTING SUBMITTED AS AN XML FILE VIA EFS WEB

The Sequence Listing written in file 591806SEQLIST.xml is 504 kilobytes, was created on Apr. 27, 2023, and is hereby incorporated by reference.

### BACKGROUND

Current gene therapy approaches rely on episomal expression of transgenes and/or insertion in specific genomic loci. The episomal approach has proven limited for the liver due to dilution or silencing. Integration in a specific locus allows for sustained expression of a transgene. However, this approach is still to be proven effective and safe in human settings. Canonical genomic safe harbor loci in humans, such as AAVS1, CCR5, and Rosa26, are all intragenic and are less explored than mouse genomic safe harbor loci. In addition, different tissues have different chromatin states for a defined locus, so canonical genomic safe harbors can be silenced in some tissues. Thus, there is a need for tissue-specific genomic safe harbor loci.

### SUMMARY

Compositions and methods for inserting a nucleic acid encoding a product of interest into a genomic safe harbor locus in a cell, a population of cells, or a subject or for expressing a nucleic acid encoding a product of interest from a genomic safe harbor locus in a cell, a population of cells, or a subject are provided. Also provided are cells or populations of cells comprising a nucleic acid construct comprising a coding sequence for a product of interest inserted into a genomic safe harbor locus. Also provided are methods of identifying genomic safe harbor loci for use in specific cell or tissue types.

In one aspect, provided are methods of integrating a nucleic acid construct into a genomic safe harbor locus in a cell (e.g., mammalian cell), such as a human cell, methods of expressing a product of interest from a genomic safe harbor locus in a cell (e.g., mammalian cell), such as a human cell, methods of integrating a nucleic acid construct into a genomic safe harbor locus in a cell (e.g., mammalian cell) in a subject (e.g., mammalian subject), such as in a human cell in a human subject, and methods of expressing a product of interest from a genomic safe harbor locus in a cell (e.g., mammalian cell) in a subject (e.g., mammalian subject), such as a human cell in a human subject.

Methods of integrating a nucleic acid construct into a genomic safe harbor locus in a cell (e.g., mammalian cell), such as a human cell are provided. Such methods can comprise administering to the cell (e.g., human cell): (a) a nuclease agent or one or more nucleic acids encoding the nuclease agent, wherein the nuclease agent targets a nuclease target site in the genomic safe harbor locus, wherein the genomic safe harbor locus is selected from the following genomic locations: (i) genomic coordinates of about 77460242 to about 77460537 on human chromosome 13; (ii) genomic coordinates of about 170031084 to about 170031382 on human chromosome 6; and (iii) genomic coordinates of about 25207412 to about 25207703 on human chromosome 9; and (b) the nucleic acid construct, wherein the nucleic acid construct comprises a nucleic acid operably linked to a promoter, wherein the nucleic acid encodes a product of interest, wherein the nuclease agent cleaves the nuclease target site, and the nucleic acid construct is inserted into the genomic safe harbor locus. Also provided are methods of expressing a product of interest from a genomic safe harbor locus in a cell (e.g., mammalian cell), such as a human cell. Such methods can comprise administering to the cell (e.g., human cell): (a) a nuclease agent or one or more nucleic acids encoding the nuclease agent, wherein the nuclease agent targets a nuclease target site in the genomic safe harbor locus, wherein the genomic safe harbor locus is selected from the following genomic locations: (i) genomic coordinates of about 77460242 to about 77460537 on human chromosome 13; (ii) genomic coordinates of about 170031084 to about 170031382 on human chromosome 6; and (iii) genomic coordinates of about 25207412 to about 25207703 on human chromosome 9; and (b) a nucleic acid construct, wherein the nucleic acid construct comprises a nucleic acid operably linked to a promoter, wherein the nucleic acid encodes the product of interest, wherein the nuclease agent cleaves the nuclease target site, the nucleic acid construct is inserted into the genomic safe harbor locus to create a modified genomic safe harbor locus, and the product of interest is expressed from the modified genomic safe harbor locus. In some such methods, the cell (e.g., human cell) is a liver cell. In some such methods, the cell (e.g., human cell) is a hepatocyte. In some such methods, the cell (e.g., human cell) is in vitro or ex vivo. In some such methods, the cell (e.g., human cell) is in vivo in a subject. Also provided are methods of integrating a nucleic acid construct into a genomic safe harbor locus in a cell (e.g., mammalian cell) in a subject (e.g., mammalian subject), such as in a human cell in a human subject. Such methods can comprise administering to the subject (e.g., human subject): (a) a nuclease agent or one or more nucleic acids encoding the nuclease agent, wherein the nuclease agent targets a nuclease target site in the genomic safe harbor locus, wherein the genomic safe harbor locus is selected from the following genomic locations: (i) genomic coordinates of about 77460242 to about 77460537 on human chromosome 13; (ii) genomic coordinates of about 170031084 to about 170031382 on human chromosome 6; and (iii) genomic coordinates of about 25207412 to about 25207703 on human chromosome 9; and (b) the nucleic acid construct, wherein the nucleic acid construct comprises a nucleic acid operably linked to a promoter, wherein the nucleic acid encodes a product of interest, wherein the nuclease agent cleaves the nuclease target site, and the nucleic acid construct is inserted into the genomic safe harbor locus. Also provided are methods of expressing a product of interest from a genomic safe harbor locus in a cell (e.g., mammalian cell) in a subject (e.g., mammalian subject), such as a human cell in a human subject. Such methods can comprise administering to the subject (e.g., human subject): (a) a nuclease agent or one or more nucleic acids encoding the nuclease agent, wherein the nuclease agent targets a nuclease target site in the genomic safe harbor locus, wherein the genomic safe harbor locus is selected from the following genomic locations: (i) genomic coordinates of about 77460242 to about 77460537 on human chromosome 13; (ii) genomic coordinates of about 170031084 to about 170031382 on human chromosome 6; and (iii) genomic coordinates of about 25207412 to about 25207703 on human chromosome 9; and (b) a nucleic acid construct, wherein the nucleic acid construct comprises a nucleic acid operably linked to a promoter, wherein the nucleic acid encodes the product of interest, wherein the nuclease agent cleaves the nuclease target site, the nucleic acid construct is inserted into the genomic safe harbor locus to create a modified genomic safe harbor locus, and the product of interest is expressed from the modified genomic safe harbor locus. In some such methods, the cell (e.g., human cell) is a liver cell. In some such methods, the cell (e.g., human cell) is a hepatocyte.

In some such methods, the genomic safe harbor locus is selected from the following genomic locations: (i) human chromosome 13, coordinates 77460242-77460537; (ii) human chromosome 6, coordinates 170031084-170031382; and (iii) human chromosome 9, coordinates 25207412-25207703. In some such methods, the genomic safe harbor locus is genomic coordinates of about 77460242 to about 77460537 on human chromosome 13. In some such methods, the genomic safe harbor locus is human chromosome 13, coordinates 77460242-77460537 or comprises, consists essentially of, or consists of the sequence set forth in SEQ ID NO: 39. In some such methods, the genomic safe harbor locus is genomic coordinates of about 170031084 to about 170031382 on human chromosome 6. In some such methods, the genomic safe harbor locus is human chromosome 6, coordinates 170031084-170031382 or comprises, consists essentially of, or consists of the sequence set forth in SEQ ID NO: 40. In some such methods, the genomic safe harbor locus is genomic coordinates of about 25207412 to about 25207703 on human chromosome 9. In some such methods, the genomic safe harbor locus is human chromosome 9, coordinates 25207412-25207703 or comprises, consists essentially of, or consists of the sequence set forth in SEQ ID NO: 41.

In some such methods, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the method comprises administering the guide RNA in the form of RNA. In some such methods, the guide RNA comprises at least one modification. In some such methods, the at least one modification comprises a 2′-O-methyl-modified nucleotide. In some such methods, the at least one modification comprises a phosphorothioate bond between nucleotides. In some such methods, the guide RNA is a single guide RNA (sgRNA). In some such methods, the Cas protein is a Cas9 protein. In some such methods, the Cas protein is a CasX protein. In some such methods, the Cas protein is a CasD protein. In some such methods, the Cas protein is a Cpf1 protein. In some such methods, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such methods, the Cas protein is derived from a Streptococcus pyogenes Cas9 protein. In some such methods, the nucleic acid encoding the Cas protein is codon-optimized for expression in a mammalian cell or a human cell. In some such methods, the method comprises administering the nucleic acid encoding the Cas protein, wherein the nucleic acid comprises an mRNA encoding the Cas protein. In some such methods, the mRNA encoding the Cas protein comprises at least one modification. In some such methods, the Cas protein or the nucleic acid encoding the Cas protein and the guide RNA or the one or more DNAs encoding the guide RNA are associated with a lipid nanoparticle. In some such methods, the genomic safe harbor locus is selected from the following genomic locations: (i) human chromosome 13, coordinates 77460242-77460537; (ii) human chromosome 6, coordinates 170031084-170031382; and (iii) human chromosome 9, coordinates 25207412-25207703. In some such methods, the genomic safe harbor locus is genomic coordinates of about 77460242 to about 77460537 on human chromosome 13. In some such methods, the genomic safe harbor locus is human chromosome 13, coordinates 77460242-77460537 or comprises, consists essentially of, or consists of the sequence set forth in SEQ ID NO: 39. In some such methods, (I) the DNA-targeting segment comprises at least 17, at least 18, at least 19, or at least 20 contiguous nucleotides of the sequence set forth in any one of SEQ ID NOS: 25, 45, and 228-256; and/or (II) the DNA-targeting segment is at least 90% or at least 95% identical to the sequence set forth in any one of SEQ ID NOS: 25, 45, and 228-256; and/or (III) the DNA-targeting segment comprises any one of SEQ ID NOS: 25, 45, and 228-256; and/or (IV) the DNA-targeting segment consists of any one of SEQ ID NOS: 25, 45, and 228-256. In some such methods, (I) the DNA-targeting segment comprises at least 17, at least 18, at least 19, or at least 20 contiguous nucleotides of the sequence set forth in any one of SEQ ID NOS: 25, 45, 235, 237, and 246; and/or (II) the DNA-targeting segment is at least 90% or at least 95% identical to the sequence set forth in any one of SEQ ID NOS: 25, 45, 235, 237, and 246; and/or (III) the DNA-targeting segment comprises any one of SEQ ID NOS: 25, 45, 235, 237, and 246; and/or (IV) the DNA-targeting segment consists of any one of SEQ ID NOS: 25, 45, 235, 237, and 246. In some such methods, (I) the DNA-targeting segment comprises at least 17, at least 18, at least 19, or at least 20 contiguous nucleotides of the sequence set forth in SEQ ID NO: 25; and/or (II) the DNA-targeting segment is at least 90% or at least 95% identical to the sequence set forth in SEQ ID NO: 25. In some such methods, the DNA-targeting segment comprises SEQ ID NO: 25. In some such methods, the DNA-targeting segment consists of SEQ ID NO: 25. In some such methods, (I) the DNA-targeting segment comprises at least 17, at least 18, at least 19, or at least 20 contiguous nucleotides of the sequence set forth in SEQ ID NO: 45; and/or (II) the DNA-targeting segment is at least 90% or at least 95% identical to the sequence set forth in SEQ ID NO: 45. In some such methods, the DNA-targeting segment comprises SEQ ID NO: 45. In some such methods, the DNA-targeting segment consists of SEQ ID NO: 45. In some such methods, the genomic safe harbor locus is genomic coordinates of about 170031084 to about 170031382 on human chromosome 6. In some such methods, the genomic safe harbor locus is human chromosome 6, coordinates 170031084-170031382 or comprises, consists essentially of, or consists of the sequence set forth in SEQ ID NO: 40. In some such methods, (I) the DNA-targeting segment comprises at least 17, at least 18, at least 19, or at least 20 contiguous nucleotides of the sequence set forth in any one of SEQ ID NOS: 26, 46, and 257-285; and/or (II) the DNA-targeting segment is at least 90% or at least 95% identical to the sequence set forth in any one of SEQ ID NOS: 26, 46, and 257-285; and/or (III) the DNA-targeting segment comprises any one of SEQ ID NOS: 26, 46, and 257-285; and/or (IV) the DNA-targeting segment consists of any one of SEQ ID NOS: 26, 46, and 257-285. In some such methods, (I) the DNA-targeting segment comprises at least 17, at least 18, at least 19, or at least 20 contiguous nucleotides of the sequence set forth in any one of SEQ ID NOS: 26, 46, 268, 271, and 280; and/or (II) the DNA-targeting segment is at least 90% or at least 95% identical to the sequence set forth in any one of SEQ ID NOS: 26, 46, 268, 271, and 280; and/or (III) the DNA-targeting segment comprises any one of SEQ ID NOS: 26, 46, 268, 271, and 280; and/or (IV) the DNA-targeting segment consists of any one of SEQ ID NOS: 26, 46, 268, 271, and 280. In some such methods, (I) the DNA-targeting segment comprises at least 17, at least 18, at least 19, or at least 20 contiguous nucleotides of the sequence set forth in SEQ ID NO: 26; and/or (II) the DNA-targeting segment is at least 90% or at least 95% identical to the sequence set forth in SEQ ID NO: 26. In some such methods, the DNA-targeting segment comprises SEQ ID NO: 26. In some such methods, the DNA-targeting segment consists of SEQ ID NO: 26. In some such methods, (I) the DNA-targeting segment comprises at least 17, at least 18, at least 19, or at least 20 contiguous nucleotides of the sequence set forth in SEQ ID NO: 46; and/or (II) the DNA-targeting segment is at least 90% or at least 95% identical to the sequence set forth in SEQ ID NO: 46. In some such methods, the DNA-targeting segment comprises SEQ ID NO: 46. In some such methods, the DNA-targeting segment consists of SEQ ID NO: 46. In some such methods, the genomic safe harbor locus is genomic coordinates of about 25207412 to about 25207703 on human chromosome 9. In some such methods, the genomic safe harbor locus is human chromosome 9, coordinates 25207412-25207703 or comprises, consists essentially of, or consists of the sequence set forth in SEQ ID NO: 41. In some such methods, (I) the DNA-targeting segment comprises at least 17, at least 18, at least 19, or at least 20 contiguous nucleotides of the sequence set forth in any one of SEQ ID NOS: 27, 47, and 286-314; and/or (II) the DNA-targeting segment is at least 90% or at least 95% identical to the sequence set forth in any one of SEQ ID NOS: 27, 47, and 286-314; and/or (III) the DNA-targeting segment comprises any one of SEQ ID NOS: 27, 47, and 286-314; and/or (IV) the DNA-targeting segment consists of any one of SEQ ID NOS: 27, 47, and 286-314. In some such methods, (I) the DNA-targeting segment comprises at least 17, at least 18, at least 19, or at least 20 contiguous nucleotides of the sequence set forth in any one of SEQ ID NOS: 27, 47, 288, 296, 305, 306, and 310; and/or (II) the DNA-targeting segment is at least 90% or at least 95% identical to the sequence set forth in any one of SEQ ID NOS: 27, 47, 288, 296, 305, 306, and 310; and/or (III) the DNA-targeting segment comprises any one of SEQ ID NOS: 27, 47, 288, 296, 305, 306, and 310; and/or (IV) the DNA-targeting segment consists of any one of SEQ ID NOS: 27, 47, 288, 296, 305, 306, and 310. In some such methods, (I) the DNA-targeting segment comprises at least 17, at least 18, at least 19, or at least 20 contiguous nucleotides of the sequence set forth in SEQ ID NO: 27; and/or (II) the DNA-targeting segment is at least 90% or at least 95% identical to the sequence set forth in SEQ ID NO: 27. In some such methods, the DNA-targeting segment comprises SEQ ID NO: 27. In some such methods, the DNA-targeting segment consists of SEQ ID NO: 27. In some such methods, (I) the DNA-targeting segment comprises at least 17, at least 18, at least 19, or at least 20 contiguous nucleotides of the sequence set forth in SEQ ID NO: 47; and/or (II) the DNA-targeting segment is at least 90% or at least 95% identical to the sequence set forth in SEQ ID NO: 47. In some such methods, the DNA-targeting segment comprises SEQ ID NO: 47. In some such methods, the DNA-targeting segment consists of SEQ ID NO: 47.

Methods of integrating a nucleic acid construct into a genomic safe harbor locus in a mouse cell are also provided. Some such methods comprise administering to the mouse cell: (a) a nuclease agent or one or more nucleic acids encoding the nuclease agent, wherein the nuclease agent targets a nuclease target site in the genomic safe harbor locus, wherein the genomic safe harbor locus is selected from the following genomic locations: (i) genomic coordinates of about 103,450,397 to about 103,451,396 on mouse chromosome 14; (ii) genomic coordinates of about 15,226,387 to about 15,227,386 on mouse chromosome 17; and (iii) genomic coordinates of about 92,827,563 to about 92,828,592 on mouse chromosome 4; and (b) the nucleic acid construct, wherein the nucleic acid construct comprises a nucleic acid operably linked to a promoter, wherein the nucleic acid encodes a product of interest, wherein the nuclease agent cleaves the nuclease target site, and the nucleic acid construct is inserted into the genomic safe harbor locus. Also provided are methods of expressing a product of interest from a genomic safe harbor locus in a mouse cell. Some such methods comprise administering to the mouse cell: (a) a nuclease agent or one or more nucleic acids encoding the nuclease agent, wherein the nuclease agent targets a nuclease target site in the genomic safe harbor locus, wherein the genomic safe harbor locus is selected from the following genomic locations: (i) genomic coordinates of about 103,450,397 to about 103,451,396 on mouse chromosome 14; (ii) genomic coordinates of about 15,226,387 to about 15,227,386 on mouse chromosome 17; and (iii) genomic coordinates of about 92,827,563 to about 92,828,592 on mouse chromosome 4; and (b) a nucleic acid construct, wherein the nucleic acid construct comprises a nucleic acid operably linked to a promoter, wherein the nucleic acid encodes the product of interest, wherein the nuclease agent cleaves the nuclease target site, the nucleic acid construct is inserted into the genomic safe harbor locus to create a modified genomic safe harbor locus, and the product of interest is expressed from the modified genomic safe harbor locus. In some such methods, the mouse cell is a liver cell. In some such methods, the mouse cell is a hepatocyte. In some such methods, the mouse cell is in vitro or ex vivo. In some such methods, the mouse cell is in vivo in a subject. Also provided are methods of integrating a nucleic acid construct into a genomic safe harbor locus in a mouse cell in a mouse subject. Some such methods comprise administering to the mouse subject: (a) a nuclease agent or one or more nucleic acids encoding the nuclease agent, wherein the nuclease agent targets a nuclease target site in the genomic safe harbor locus, wherein the genomic safe harbor locus is selected from the following genomic locations: (i) genomic coordinates of about 103,450,397 to about 103,451,396 on mouse chromosome 14; (ii) genomic coordinates of about 15,226,387 to about 15,227,386 on mouse chromosome 17; and (iii) genomic coordinates of about 92,827,563 to about 92,828,592 on mouse chromosome 4; and (b) the nucleic acid construct, wherein the nucleic acid construct comprises a nucleic acid operably linked to a promoter, wherein the nucleic acid encodes a product of interest, wherein the nuclease agent cleaves the nuclease target site, and the nucleic acid construct is inserted into the genomic safe harbor locus. Also provided are methods of expressing a product of interest from a genomic safe harbor locus in a mouse cell in a mouse subject. Some such methods comprise administering to the mouse subject: (a) a nuclease agent or one or more nucleic acids encoding the nuclease agent, wherein the nuclease agent targets a nuclease target site in the genomic safe harbor locus, wherein the genomic safe harbor locus is selected from the following genomic locations: (i) genomic coordinates of about 103,450,397 to about 103,451,396 on mouse chromosome 14; (ii) genomic coordinates of about 15,226,387 to about 15,227,386 on mouse chromosome 17; and (iii) genomic coordinates of about 92,827,563 to about 92,828,592 on mouse chromosome 4; and (b) a nucleic acid construct, wherein the nucleic acid construct comprises a nucleic acid operably linked to a promoter, wherein the nucleic acid encodes the product of interest, wherein the nuclease agent cleaves the nuclease target site, the nucleic acid construct is inserted into the genomic safe harbor locus to create a modified genomic safe harbor locus, and the product of interest is expressed from the modified genomic safe harbor locus. In some such methods, the mouse cell is a liver cell. In some such methods, the mouse cell is a hepatocyte.

In some such methods, the genomic safe harbor locus is selected from the following genomic locations: (i) mouse chromosome 14, coordinates 103,450,397-103,451,396; (ii) mouse chromosome 17, coordinates 15,226,387-15,227,386; and (iii) mouse chromosome 4, coordinates 92,827,563-92,828,592. In some such methods, the genomic safe harbor locus is genomic coordinates of about 103,450,397 to about 103,451,396 on mouse chromosome 14. In some such methods, the genomic safe harbor locus is mouse chromosome 14, coordinates 103,450,397-103,451,396 or comprises, consists essentially of, or consists of the sequence set forth in SEQ ID NO: 405. In some such methods, the genomic safe harbor locus is genomic coordinates of about 15,226,387 to about 15,227,386 on mouse chromosome 17. In some such methods, the genomic safe harbor locus is mouse chromosome 17, coordinates 15,226,387-15,227,386 or comprises, consists essentially of, or consists of the sequence set forth in SEQ ID NO: 406. In some such methods, the genomic safe harbor locus is genomic coordinates of about 92,827,563 to about 92,828,592 on mouse chromosome 4. In some such methods, the genomic safe harbor locus is mouse chromosome 4, coordinates 92,827,563-92,828,592 or comprises, consists essentially of, or consists of the sequence set forth in SEQ ID NO: 407.

In some such methods, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the method comprises administering the guide RNA in the form of RNA. In some such methods, the guide RNA comprises at least one modification. In some such methods, the at least one modification comprises a 2′-O-methyl-modified nucleotide. In some such methods, the at least one modification comprises a phosphorothioate bond between nucleotides. In some such methods, the guide RNA is a single guide RNA (sgRNA). In some such methods, the Cas protein is a Cas9 protein. In some such methods, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such methods, the Cas protein is derived from a Streptococcus pyogenes Cas9 protein. In some such methods, the nucleic acid encoding the Cas protein is codon-optimized for expression in a mammalian cell or a mouse cell. In some such methods, the method comprises administering the nucleic acid encoding the Cas protein, wherein the nucleic acid comprises an mRNA encoding the Cas protein. In some such methods, the mRNA encoding the Cas protein comprises at least one modification. In some such methods, the Cas protein or the nucleic acid encoding the Cas protein and the guide RNA or the one or more DNAs encoding the guide RNA are associated with a lipid nanoparticle. In some such methods, the genomic safe harbor locus is selected from the following genomic locations: (i) mouse chromosome 14, coordinates 103,450,397-103,451,396; (ii) mouse chromosome 17, coordinates 15,226,387-15,227,386; and (iii) mouse chromosome 4, coordinates 92,827,563-92,828,592. In some such methods, the genomic safe harbor locus is genomic coordinates of about 103,450,397 to about 103,451,396 on mouse chromosome 14. In some such methods, the genomic safe harbor locus is mouse chromosome 14, coordinates 103,450,397-103,451,396 or comprises, consists essentially of, or consists of the sequence set forth in SEQ ID NO: 405. In some such methods, (I) the DNA-targeting segment comprises at least 17, at least 18, at least 19, or at least 20 contiguous nucleotides of the sequence set forth in any one of SEQ ID NOS: 315-344; and/or (II) the DNA-targeting segment is at least 90% or at least 95% identical to the sequence set forth in any one of SEQ ID NOS: 315-344; and/or (III) the DNA-targeting segment comprises any one of SEQ ID NOS: 315-344; and/or (IV) the DNA-targeting segment consists of any one of SEQ ID NOS: 315-344. In some such methods, (I) the DNA-targeting segment comprises at least 17, at least 18, at least 19, or at least 20 contiguous nucleotides of the sequence set forth in any one of SEQ ID NOS: 318, 320, 321, and 341; and/or (II) the DNA-targeting segment is at least 90% or at least 95% identical to the sequence set forth in any one of SEQ ID NOS: 318, 320, 321, and 341; and/or (III) the DNA-targeting segment comprises any one of SEQ ID NOS: 318, 320, 321, and 341; and/or (IV) the DNA-targeting segment consists of any one of SEQ ID NOS: 318, 320, 321, and 341. In some such methods, the genomic safe harbor locus is genomic coordinates of about 15,226,387 to about 15,227,386 on mouse chromosome 17. In some such methods, the genomic safe harbor locus is mouse chromosome 17, coordinates 15,226,387-15,227,386 or comprises, consists essentially of, or consists of the sequence set forth in SEQ ID NO: 406. In some such methods, (I) the DNA-targeting segment comprises at least 17, at least 18, at least 19, or at least 20 contiguous nucleotides of the sequence set forth in any one of SEQ ID NOS: 345-374; and/or (II) the DNA-targeting segment is at least 90% or at least 95% identical to the sequence set forth in any one of SEQ ID NOS: 345-374; and/or (III) the DNA-targeting segment comprises any one of SEQ ID NOS: 345-374; and/or (IV) the DNA-targeting segment consists of any one of SEQ ID NOS: 345-374. In some such methods, (I) the DNA-targeting segment comprises at least 17, at least 18, at least 19, or at least 20 contiguous nucleotides of the sequence set forth in any one of SEQ ID NOS: 347, 360, 369, and 370; and/or (II) the DNA-targeting segment is at least 90% or at least 95% identical to the sequence set forth in any one of SEQ ID NOS: 347, 360, 369, and 370; and/or (III) the DNA-targeting segment comprises any one of SEQ ID NOS: 347, 360, 369, and 370; and/or (IV) the DNA-targeting segment consists of any one of SEQ ID NOS: 347, 360, 369, and 370. In some such methods, the genomic safe harbor locus is genomic coordinates of about 92,827,563 to about 92,828,592 on mouse chromosome 4. In some such methods, the genomic safe harbor locus is mouse chromosome 4, coordinates 92,827,563-92,828,592 or comprises, consists essentially of, or consists of the sequence set forth in SEQ ID NO: 407. In some such methods, (I) the DNA-targeting segment comprises at least 17, at least 18, at least 19, or at least 20 contiguous nucleotides of the sequence set forth in any one of SEQ ID NOS: 375-404; and/or (II) the DNA-targeting segment is at least 90% or at least 95% identical to the sequence set forth in any one of SEQ ID NOS: 375-404; and/or (III) the DNA-targeting segment comprises any one of SEQ ID NOS: 375-404; and/or (IV) the DNA-targeting segment consists of any one of SEQ ID NOS: 375-404. In some such methods, (I) the DNA-targeting segment comprises at least 17, at least 18, at least 19, or at least 20 contiguous nucleotides of the sequence set forth in any one of SEQ ID NOS: 379, 380, and 388; and/or (II) the DNA-targeting segment is at least 90% or at least 95% identical to the sequence set forth in any one of SEQ ID NOS: 379, 380, and 388; and/or (III) the DNA-targeting segment comprises any one of SEQ ID NOS: 379, 380, and 388; and/or (IV) the DNA-targeting segment consists of any one of SEQ ID NOS: 379, 380, and 388.

In some such methods, the nucleic acid construct is administered simultaneously with the nuclease agent or the one or more nucleic acids encoding the nuclease agent. In some such methods, the nucleic acid construct is not administered simultaneously with the nuclease agent or the one or more nucleic acids encoding the nuclease agent. In some such methods, the nucleic acid construct is administered prior to the nuclease agent or the one or more nucleic acids encoding the nuclease agent. In some such methods, the nucleic acid construct is administered after the nuclease agent or the one or more nucleic acids encoding the nuclease agent.

In some such methods, the product of interest is a polypeptide of interest. In some such methods, the polypeptide of interest comprises a therapeutic polypeptide. In some such methods, the polypeptide of interest is a secreted polypeptide. In some such methods, the polypeptide of interest is an intracellular polypeptide.

In some such methods, the promoter is active in liver cells. In some such methods, the promoter is a tissue-specific promoter. In some such methods, the promoter is a constitutive promoter. In some such methods, the promoter is an inducible promoter.

In some such methods, the nucleic acid construct does not comprise a homology arm. In some such methods, the nucleic acid construct is inserted into the target genomic locus via non-homologous end joining. In some such methods, the nucleic acid construct comprises homology arms. In some such methods, the nucleic acid construct is inserted into the target genomic locus via homology-directed repair. In some such methods, the nucleic acid construct is single-stranded DNA or double-stranded DNA. In some such methods, the nucleic acid construct is single-stranded DNA.

In some such methods, the nucleic acid construct is in a nucleic acid vector or a lipid nanoparticle. In some such methods, the nucleic acid construct is in the nucleic acid vector. In some such methods, the nucleic acid vector is a viral vector. In some such methods, the nucleic acid vector is an adeno-associated viral (AAV) vector. In some such methods, the AAV vector is a single-stranded AAV (ssAAV) vector. In some such methods, the AAV vector is derived from an AAV8 vector, an AAV3B vector, an AAV5 vector, an AAV6 vector, an AAV7 vector, an AAV9 vector, an AAVrh.74 vector, an AAV-DJ vector, or an AAVhu.37 vector. In some such methods, the AAV vector is a recombinant AAV8 (rAAV8) vector. In some such methods, the AAV vector is a single-stranded rAAV8 vector.

In another aspect, provided are cells (e.g., mammalian cells, such as human cells) made by any of the above methods. In another aspect, provided are cells (e.g., mammalian cells, such as human cells) comprising a nucleic acid construct integrated into a genomic safe harbor locus. In some such cells, the nucleic acid construct comprises a nucleic acid operably linked to a promoter, wherein the nucleic acid encodes a product of interest, and wherein the genomic safe harbor locus is selected from the following genomic locations: (i) genomic coordinates of about 77460242 to about 77460537 on human chromosome 13; (ii) genomic coordinates of about 170031084 to about 170031382 on human chromosome 6; and (iii) genomic coordinates of about 25207412 to about 25207703 on human chromosome 9. In some such cells, the nucleic acid construct comprises a nucleic acid operably linked to a promoter, wherein the nucleic acid encodes a product of interest, and wherein the genomic safe harbor locus is selected from the following genomic locations: (i) genomic coordinates of about 103,450,397 to about 103,451,396 on mouse chromosome 14; (ii) genomic coordinates of about 15,226,387 to about 15,227,386 on mouse chromosome 17; and (iii) genomic coordinates of about 92,827,563 to about 92,828,592 on mouse chromosome 4.

In some such cells, the cell is a human cell. In some such cells, the cell is a mouse cell. In some such cells, the cell is a liver cell (e.g., human liver cell). In some such cells, the cell is a hepatocyte (e.g., human hepatocyte).

In some such cells, the product of interest is expressed. In some such cells, the product of interest is a polypeptide of interest. In some such cells, the polypeptide of interest comprises a therapeutic polypeptide. In some such cells, the polypeptide of interest is a secreted polypeptide. In some such cells, the polypeptide of interest is an intracellular polypeptide. In some such cells, the promoter is active in liver cells. In some such cells, the promoter is a tissue-specific promoter. In some such cells, the promoter is a constitutive promoter. In some such cells, the promoter is an inducible promoter.

In some such cells, the genomic safe harbor locus is selected from the following genomic locations: (i) human chromosome 13, coordinates 77460242-77460537; (ii) human chromosome 6, coordinates 170031084-170031382; and (iii) human chromosome 9, coordinates 25207412-25207703. In some such cells, the genomic safe harbor locus is genomic coordinates of about 77460242 to about 77460537 on human chromosome 13. In some such cells, the genomic safe harbor locus is human chromosome 13, coordinates 77460242-77460537 or comprises, consists essentially of, or consists of the sequence set forth in SEQ ID NO: 39. In some such cells, the genomic safe harbor locus is genomic coordinates of about 170031084 to about 170031382 on human chromosome 6. In some such cells, the genomic safe harbor locus is human chromosome 6, coordinates 170031084-170031382 or comprises, consists essentially of, or consists of the sequence set forth in SEQ ID NO: 40. In some such cells, the genomic safe harbor locus is genomic coordinates of about 25207412 to about 25207703 on human chromosome 9. In some such cells, the genomic safe harbor locus is human chromosome 9, coordinates 25207412-25207703 or comprises, consists essentially of, or consists of the sequence set forth in SEQ ID NO: 41.

In some such cells, the genomic safe harbor locus is selected from the following genomic locations: (i) mouse chromosome 14, coordinates 103,450,397-103,451,396; (ii) mouse chromosome 17, coordinates 15,226,387-15,227,386; and (iii) mouse chromosome 4, coordinates 92,827,563-92,828,592. In some such cells, the genomic safe harbor locus is genomic coordinates of about 103,450,397 to about 103,451,396 on mouse chromosome 14. In some such cells, the genomic safe harbor locus is mouse chromosome 14, coordinates 103,450,397-103,451,396 or comprises, consists essentially of, or consists of the sequence set forth in SEQ ID NO: 405. In some such cells, the genomic safe harbor locus is genomic coordinates of about 15,226,387 to about 15,227,386 on mouse chromosome 17. In some such cells, the genomic safe harbor locus is mouse chromosome 17, coordinates 15,226,387-15,227,386 or comprises, consists essentially of, or consists of the sequence set forth in SEQ ID NO: 406. In some such cells, the genomic safe harbor locus is genomic coordinates of about 92,827,563 to about 92,828,592 on mouse chromosome 4. In some such cells, the genomic safe harbor locus is mouse chromosome 4, coordinates 92,827,563-92,828,592 or comprises, consists essentially of, or consists of the sequence set forth in SEQ ID NO: 407.

In another aspect, provided are compositions comprising a guide RNA or a DNA encoding a guide RNA, wherein the guide RNA comprises a DNA-targeting segment that targets a guide RNA target sequence in a genomic safe harbor locus and a protein-binding segment that binds to a Cas protein, wherein the genomic safe harbor locus is selected from the following genomic locations: (i) genomic coordinates of about 77460242 to about 77460537 on human chromosome 13; (ii) genomic coordinates of about 170031084 to about 170031382 on human chromosome 6; and (iii) genomic coordinates of about 25207412 to about 25207703 on human chromosome 9. In another aspect, provided are compositions comprising a guide RNA or a DNA encoding a guide RNA, wherein the guide RNA comprises a DNA-targeting segment that targets a guide RNA target sequence in a genomic safe harbor locus and a protein-binding segment that binds to a Cas protein, wherein the genomic safe harbor locus is selected from the following genomic locations: (i) genomic coordinates of about 103,450,397 to about 103,451,396 on mouse chromosome 14; (ii) genomic coordinates of about 15,226,387 to about 15,227,386 on mouse chromosome 17; and (iii) genomic coordinates of about 92,827,563 to about 92,828,592 on mouse chromosome 4.

In some such compositions, the genomic safe harbor locus is selected from the following genomic locations: (i) human chromosome 13, coordinates 77460242-77460537; (ii) human chromosome 6, coordinates 170031084-170031382; and (iii) human chromosome 9, coordinates 25207412-25207703. In some such compositions, the genomic safe harbor locus is genomic coordinates of about 77460242 to about 77460537 on human chromosome 13. In some such compositions, the genomic safe harbor locus is human chromosome 13, coordinates 77460242-77460537 or comprises, consists essentially of, or consists of the sequence set forth in SEQ ID NO: 39. In some such compositions, (I) the DNA-targeting segment comprises at least 17, at least 18, at least 19, or at least 20 contiguous nucleotides of the sequence set forth in any one of SEQ ID NOS: 25, 45, and 228-256; and/or (II) the DNA-targeting segment is at least 90% or at least 95% identical to the sequence set forth in any one of SEQ ID NOS: 25, 45, and 228-256; and/or (III) the DNA-targeting segment comprises any one of SEQ ID NOS: 25, 45, and 228-256; and/or (IV) the DNA-targeting segment consists of any one of SEQ ID NOS: 25, 45, and 228-256. In some such compositions, (I) the DNA-targeting segment comprises at least 17, at least 18, at least 19, or at least 20 contiguous nucleotides of the sequence set forth in any one of SEQ ID NOS: 25, 45, 235, 237, and 246; and/or (II) the DNA-targeting segment is at least 90% or at least 95% identical to the sequence set forth in any one of SEQ ID NOS: 25, 45, 235, 237, and 246; and/or (III) the DNA-targeting segment comprises any one of SEQ ID NOS: 25, 45, 235, 237, and 246; and/or (IV) the DNA-targeting segment consists of any one of SEQ ID NOS: 25, 45, 235, 237, and 246. In some such compositions, (I) the DNA-targeting segment comprises at least 17, at least 18, at least 19, or at least 20 contiguous nucleotides of the sequence set forth in SEQ ID NO: 25; and/or (II) the DNA-targeting segment is at least 90% or at least 95% identical to the sequence set forth in SEQ ID NO: 25. In some such compositions, the DNA-targeting segment comprises SEQ ID NO: 25. In some such compositions, the DNA-targeting segment consists of SEQ ID NO: 25. In some such compositions, (I) the DNA-targeting segment comprises at least 17, at least 18, at least 19, or at least 20 contiguous nucleotides of the sequence set forth in SEQ ID NO: 45; and/or (II) the DNA-targeting segment is at least 90% or at least 95% identical to the sequence set forth in SEQ ID NO: 45. In some such compositions, the DNA-targeting segment comprises SEQ ID NO: 45. In some such compositions, the DNA-targeting segment consists of SEQ ID NO: 45. In some such compositions, the genomic safe harbor locus is genomic coordinates of about 170031084 to about 170031382 on human chromosome 6. In some such compositions, the genomic safe harbor locus is human chromosome 6, coordinates 170031084-170031382 or comprises, consists essentially of, or consists of the sequence set forth in SEQ ID NO: 40. In some such compositions, (I) the DNA-targeting segment comprises at least 17, at least 18, at least 19, or at least 20 contiguous nucleotides of the sequence set forth in any one of SEQ ID NOS: 26, 46, and 257-285; and/or (II) the DNA-targeting segment is at least 90% or at least 95% identical to the sequence set forth in any one of SEQ ID NOS: 26, 46, and 257-285; and/or (III) the DNA-targeting segment comprises any one of SEQ ID NOS: 26, 46, and 257-285; and/or (IV) the DNA-targeting segment consists of any one of SEQ ID NOS: 26, 46, and 257-285. In some such compositions, (I) the DNA-targeting segment comprises at least 17, at least 18, at least 19, or at least 20 contiguous nucleotides of the sequence set forth in any one of SEQ ID NOS: 26, 46, 268, 271, and 280; and/or (II) the DNA-targeting segment is at least 90% or at least 95% identical to the sequence set forth in any one of SEQ ID NOS: 26, 46, 268, 271, and 280; and/or (III) the DNA-targeting segment comprises any one of SEQ ID NOS: 26, 46, 268, 271, and 280; and/or (IV) the DNA-targeting segment consists of any one of SEQ ID NOS: 26, 46, 268, 271, and 280. In some such compositions, (I) the DNA-targeting segment comprises at least 17, at least 18, at least 19, or at least 20 contiguous nucleotides of the sequence set forth in SEQ ID NO: 26; and/or (II) the DNA-targeting segment is at least 90% or at least 95% identical to the sequence set forth in SEQ ID NO: 26. In some such compositions, the DNA-targeting segment comprises SEQ ID NO: 26. In some such compositions, the DNA-targeting segment consists of SEQ ID NO: 26. In some such compositions, (I) the DNA-targeting segment comprises at least 17, at least 18, at least 19, or at least 20 contiguous nucleotides of the sequence set forth in SEQ ID NO: 46; and/or (II) the DNA-targeting segment is at least 90% or at least 95% identical to the sequence set forth in SEQ ID NO: 46. In some such compositions, the DNA-targeting segment comprises SEQ ID NO: 46. In some such compositions, the DNA-targeting segment consists of SEQ ID NO: 46. In some such compositions, the genomic safe harbor locus is genomic coordinates of about 25207412 to about 25207703 on human chromosome 9. In some such compositions, the genomic safe harbor locus is human chromosome 9, coordinates 25207412-25207703 or comprises, consists essentially of, or consists of the sequence set forth in SEQ ID NO: 41. In some such compositions, (I) the DNA-targeting segment comprises at least 17, at least 18, at least 19, or at least 20 contiguous nucleotides of the sequence set forth in any one of SEQ ID NOS: 27, 47, and 286-314; and/or (II) the DNA-targeting segment is at least 90% or at least 95% identical to the sequence set forth in any one of SEQ ID NOS: 27, 47, and 286-314; and/or (III) the DNA-targeting segment comprises any one of SEQ ID NOS: 27, 47, and 286-314; and/or (IV) the DNA-targeting segment consists of any one of SEQ ID NOS: 27, 47, and 286-314. In some such compositions, (I) the DNA-targeting segment comprises at least 17, at least 18, at least 19, or at least 20 contiguous nucleotides of the sequence set forth in any one of SEQ ID NOS: 27, 47, 288, 296, 305, 306, and 310; and/or (II) the DNA-targeting segment is at least 90% or at least 95% identical to the sequence set forth in any one of SEQ TD NOS: 27, 47, 288, 296, 305, 306, and 310; and/or (III) the DNA-targeting segment comprises any one of SEQ ID NOS: 27, 47, 288, 296, 305, 306, and 310; and/or (IV) the DNA-targeting segment consists of any one of SEQ ID NOS: 27, 47, 288, 296, 305, 306, and 310. In some such compositions, (I) the DNA-targeting segment comprises at least 17, at least 18, at least 19, or at least 20 contiguous nucleotides of the sequence set forth in SEQ ID NO: 27; and/or (II) the DNA-targeting segment is at least 90% or at least 95% identical to the sequence set forth in SEQ ID NO: 27. In some such compositions, the DNA-targeting segment comprises SEQ ID NO: 27. In some such compositions, the DNA-targeting segment consists of SEQ ID NO: 27. In some such compositions, (I) the DNA-targeting segment comprises at least 17, at least 18, at least 19, or at least 20 contiguous nucleotides of the sequence set forth in SEQ ID NO: 47; and/or (II) the DNA-targeting segment is at least 90% or at least 95% identical to the sequence set forth in SEQ ID NO: 47. In some such compositions, the DNA-targeting segment comprises SEQ ID NO: 47. In some such compositions, the DNA-targeting segment consists of SEQ ID NO: 47.

In some such compositions, the genomic safe harbor locus is selected from the following genomic locations: (i) mouse chromosome 14, coordinates 103,450,397-103,451,396; (ii) mouse chromosome 17, coordinates 15,226,387-15,227,386; and (iii) mouse chromosome 4, coordinates 92,827,563-92,828,592. In some such compositions, the genomic safe harbor locus is genomic coordinates of about 103,450,397 to about 103,451,396 on mouse chromosome 14. In some such compositions, the genomic safe harbor locus is mouse chromosome 14, coordinates 103,450,397-103,451,396 or comprises, consists essentially of, or consists of the sequence set forth in SEQ ID NO: 405. In some such compositions, (I) the DNA-targeting segment comprises at least 17, at least 18, at least 19, or at least 20 contiguous nucleotides of the sequence set forth in any one of SEQ ID NOS: 315-344; and/or (II) the DNA-targeting segment is at least 90% or at least 95% identical to the sequence set forth in any one of SEQ ID NOS: 315-344; and/or (III) the DNA-targeting segment comprises any one of SEQ ID NOS: 315-344; and/or (IV) the DNA-targeting segment consists of any one of SEQ ID NOS: 315-344. In some such compositions, (I) the DNA-targeting segment comprises at least 17, at least 18, at least 19, or at least 20 contiguous nucleotides of the sequence set forth in any one of SEQ ID NOS: 318, 320, 321, and 341; and/or (II) the DNA-targeting segment is at least 90% or at least 95% identical to the sequence set forth in any one of SEQ ID NOS: 318, 320, 321, and 341; and/or (III) the DNA-targeting segment comprises any one of SEQ ID NOS: 318, 320, 321, and 341; and/or (IV) the DNA-targeting segment consists of any one of SEQ ID NOS: 318, 320, 321, and 341. In some such compositions, the genomic safe harbor locus is genomic coordinates of about 15,226,387 to about 15,227,386 on mouse chromosome 17. In some such compositions, the genomic safe harbor locus is mouse chromosome 17, coordinates 15,226,387-15,227,386 or comprises, consists essentially of, or consists of the sequence set forth in SEQ ID NO: 406. In some such compositions, (I) the DNA-targeting segment comprises at least 17, at least 18, at least 19, or at least 20 contiguous nucleotides of the sequence set forth in any one of SEQ ID NOS: 345-374; and/or (II) the DNA-targeting segment is at least 90% or at least 95% identical to the sequence set forth in any one of SEQ ID NOS: 345-374; and/or (III) the DNA-targeting segment comprises any one of SEQ ID NOS: 345-374; and/or (IV) the DNA-targeting segment consists of any one of SEQ ID NOS: 345-374. In some such compositions, (I) the DNA-targeting segment comprises at least 17, at least 18, at least 19, or at least 20 contiguous nucleotides of the sequence set forth in any one of SEQ ID NOS: 347, 360, 369, and 370; and/or (II) the DNA-targeting segment is at least 90% or at least 95% identical to the sequence set forth in any one of SEQ ID NOS: 347, 360, 369, and 370; and/or (III) the DNA-targeting segment comprises any one of SEQ ID NOS: 347, 360, 369, and 370; and/or (IV) the DNA-targeting segment consists of any one of SEQ ID NOS: 347, 360, 369, and 370. In some such compositions, the genomic safe harbor locus is genomic coordinates of about 92,827,563 to about 92,828,592 on mouse chromosome 4. In some such compositions, the genomic safe harbor locus is mouse chromosome 4, coordinates 92,827,563-92,828,592 or comprises, consists essentially of, or consists of the sequence set forth in SEQ ID NO: 407. In some such compositions, (I) the DNA-targeting segment comprises at least 17, at least 18, at least 19, or at least 20 contiguous nucleotides of the sequence set forth in any one of SEQ ID NOS: 375-404; and/or (II) the DNA-targeting segment is at least 90% or at least 95% identical to the sequence set forth in any one of SEQ ID NOS: 375-404; and/or (III) the DNA-targeting segment comprises any one of SEQ ID NOS: 375-404; and/or (IV) the DNA-targeting segment consists of any one of SEQ ID NOS: 375-404. In some such compositions, (I) the DNA-targeting segment comprises at least 17, at least 18, at least 19, or at least 20 contiguous nucleotides of the sequence set forth in any one of SEQ ID NOS: 379, 380, and 388; and/or (II) the DNA-targeting segment is at least 90% or at least 95% identical to the sequence set forth in any one of SEQ ID NOS: 379, 380, and 388; and/or (III) the DNA-targeting segment comprises any one of SEQ ID NOS: 379, 380, and 388; and/or (IV) the DNA-targeting segment consists of any one of SEQ ID NOS: 379, 380, and 388.

In some such compositions, the composition comprises the DNA encoding the guide RNA. In some such compositions, the DNA encoding the guide RNA is in a nucleic acid vector. In some such compositions, the nucleic acid vector is a viral vector. In some such compositions, the nucleic acid vector is an adeno-associated viral (AAV) vector. In some such compositions, the AAV vector is a single-stranded AAV (ssAAV) vector. In some such compositions, the AAV vector is derived from an AAV8 vector, an AAV3B vector, an AAV5 vector, an AAV6 vector, an AAV7 vector, an AAV9 vector, an AAVrh.74 vector, an AAV-DJ vector, or an AAVhu.37 vector. In some such compositions, the AAV vector is a recombinant AAV8 (rAAV8) vector. In some such compositions, the AAV vector is a single-stranded rAAV8 vector. In some such compositions, the composition comprises the guide RNA in the form of RNA. In some such compositions, the guide RNA comprises at least one modification. In some such compositions, the at least one modification comprises a 2′-O-methyl-modified nucleotide. In some such compositions, the at least one modification comprises a phosphorothioate bond between nucleotides. In some such compositions, the guide RNA is a single guide RNA (sgRNA).

In some such compositions, the composition further comprises the Cas protein or a nucleic acid encoding the Cas protein. In some such compositions, the composition comprises the Cas protein. In some such compositions, the composition comprises the nucleic acid encoding the Cas protein. In some such compositions, the nucleic acid encoding the Cas protein is codon-optimized for expression in a mammalian cell or a human cell. In some such compositions, the nucleic acid encoding the Cas protein comprises a DNA encoding the Cas protein. In some such compositions, the DNA encoding the guide RNA is in a nucleic acid vector. In some such compositions, the nucleic acid vector is a viral vector. In some such compositions, the nucleic acid vector is an adeno-associated viral (AAV) vector. In some such compositions, the AAV vector is a single-stranded AAV (ssAAV) vector. In some such compositions, the AAV vector is derived from an AAV8 vector, an AAV3B vector, an AAV5 vector, an AAV6 vector, an AAV7 vector, an AAV9 vector, an AAVrh.74 vector, an AAV-DJ vector, or an AAVhu.37 vector. In some such compositions, the AAV vector is a recombinant AAV8 (rAAV8) vector. In some such compositions, the AAV vector is a single-stranded rAAV8 vector. In some such compositions, the nucleic acid encoding the Cas protein comprises an mRNA encoding the Cas protein. In some such compositions, the mRNA encoding the Cas protein comprises at least one modification. In some such compositions, the Cas protein or the nucleic acid encoding the Cas protein and the guide RNA or the one or more DNAs encoding the guide RNA are associated with a lipid nanoparticle. In some such compositions, the Cas protein is a Cas9 protein. In some such compositions, the Cas protein is a CasX protein. In some such compositions, the Cas protein is a CasD protein. In some such compositions, the Cas protein is a Cpf1 protein. In some such compositions, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such compositions, the Cas protein is derived from a Streptococcus pyogenes Cas9 protein.

In some such compositions, the composition further comprises a nucleic acid construct, wherein the nucleic acid construct comprises a nucleic acid operably linked to a promoter, wherein the nucleic acid encodes a product of interest. In some such compositions, the product of interest is a polypeptide of interest. In some such compositions, the polypeptide of interest comprises a therapeutic polypeptide. In some such compositions, the polypeptide of interest is a secreted polypeptide. In some such compositions, the polypeptide of interest is an intracellular polypeptide. In some such compositions, the promoter is active in liver cells. In some such compositions, the promoter is a tissue-specific promoter. In some such compositions, the promoter is a constitutive promoter. In some such compositions, the promoter is an inducible promoter. In some such compositions, the nucleic acid construct does not comprise a homology arm. In some such compositions, the nucleic acid construct comprises homology arms. In some such compositions, the nucleic acid construct is single-stranded DNA or double-stranded DNA. In some such compositions, the nucleic acid construct is single-stranded DNA.

In some such compositions, the nucleic acid construct is in a nucleic acid vector or a lipid nanoparticle. In some such compositions, the nucleic acid construct is in the nucleic acid vector. In some such compositions, the nucleic acid vector is a viral vector. In some such compositions, the nucleic acid vector is an adeno-associated viral (AAV) vector. In some such compositions, the AAV vector is a single-stranded AAV (ssAAV) vector. In some such compositions, the AAV vector is derived from an AAV8 vector, an AAV3B vector, an AAV5 vector, an AAV6 vector, an AAV7 vector, an AAV9 vector, an AAVrh.74 vector, an AAV-DJ vector, or an AAVhu.37 vector. In some such compositions, the AAV vector is a recombinant AAV8 (rAAV8) vector. In some such compositions, the AAV vector is a single-stranded rAAV8 vector.

In another aspect, provided are nucleic acids comprising a genomic safe harbor locus comprising an integrated nucleic acid construct. In some such nucleic acids, the nucleic acid construct comprises a nucleic acid operably linked to a promoter, the nucleic acid encodes a product of interest, and the genomic safe harbor locus is selected from the following genomic locations: (i) genomic coordinates of about 77460242 to about 77460537 on human chromosome 13; (ii) genomic coordinates of about 170031084 to about 170031382 on human chromosome 6; and (iii) genomic coordinates of about 25207412 to about 25207703 on human chromosome 9. In some such nucleic acids, the nucleic acid construct comprises a nucleic acid operably linked to a promoter, the nucleic acid encodes a product of interest, and the genomic safe harbor locus is selected from the following genomic locations: (i) genomic coordinates of about 103,450,397 to about 103,451,396 on mouse chromosome 14; (ii) genomic coordinates of about 15,226,387 to about 15,227,386 on mouse chromosome 17; and (iii) genomic coordinates of about 92,827,563 to about 92,828,592 on mouse chromosome 4.

In some such nucleic acids, the product of interest is a polypeptide of interest. In some such nucleic acids, the polypeptide of interest comprises a therapeutic polypeptide. In some such nucleic acids, the polypeptide of interest is a secreted polypeptide. In some such nucleic acids, the polypeptide of interest is an intracellular polypeptide. In some such nucleic acids, the promoter is active in liver cells. In some such nucleic acids, the promoter is a tissue-specific promoter. In some such nucleic acids, the promoter is a constitutive promoter. In some such nucleic acids, the promoter is an inducible promoter.

In some such nucleic acids, the genomic safe harbor locus is selected from the following genomic locations: (i) human chromosome 13, coordinates 77460242-77460537; (ii) human chromosome 6, coordinates 170031084-170031382; and (iii) human chromosome 9, coordinates 25207412-25207703. In some such nucleic acids, the genomic safe harbor locus is genomic coordinates of about 77460242 to about 77460537 on human chromosome 13. In some such nucleic acids, the genomic safe harbor locus is human chromosome 13, coordinates 77460242-77460537 or comprises, consists essentially of, or consists of the sequence set forth in SEQ ID NO: 39. In some such nucleic acids, the genomic safe harbor locus is genomic coordinates of about 170031084 to about 170031382 on human chromosome 6. In some such nucleic acids, the genomic safe harbor locus is human chromosome 6, coordinates 170031084-170031382 or comprises, consists essentially of, or consists of the sequence set forth in SEQ ID NO: 40. In some such nucleic acids, the genomic safe harbor locus is genomic coordinates of about 25207412 to about 25207703 on human chromosome 9. In some such nucleic acids, the genomic safe harbor locus is human chromosome 9, coordinates 25207412-25207703 or comprises, consists essentially of, or consists of the sequence set forth in SEQ ID NO: 41.

In some such nucleic acids, the genomic safe harbor locus is selected from the following genomic locations: (i) mouse chromosome 14, coordinates 103,450,397-103,451,396; (ii) mouse chromosome 17, coordinates 15,226,387-15,227,386; and (iii) mouse chromosome 4, coordinates 92,827,563-92,828,592. In some such nucleic acids, the genomic safe harbor locus is genomic coordinates of about 103,450,397 to about 103,451,396 on mouse chromosome 14. In some such nucleic acids, the genomic safe harbor locus is mouse chromosome 14, coordinates 103,450,397-103,451,396 or comprises, consists essentially of, or consists of the sequence set forth in SEQ ID NO: 405. In some such nucleic acids, the genomic safe harbor locus is genomic coordinates of about 15,226,387 to about 15,227,386 on mouse chromosome 17. In some such nucleic acids, the genomic safe harbor locus is mouse chromosome 17, coordinates 15,226,387-15,227,386 or comprises, consists essentially of, or consists of the sequence set forth in SEQ ID NO: 406. In some such nucleic acids, the genomic safe harbor locus is genomic coordinates of about 92,827,563 to about 92,828,592 on mouse chromosome 4. In some such nucleic acids, the genomic safe harbor locus is mouse chromosome 4, coordinates 92,827,563-92,828,592 or comprises, consists essentially of, or consists of the sequence set forth in SEQ ID NO: 407.

In another aspect, provided are methods of identifying one or more genomic safe harbor loci in a tissue or cell type of interest. Some such methods comprise: (a) identifying accessible genomic loci in the tissue or cell type of interest; (b) selecting genomic loci identified in step (a) based on safety criteria, functional silencing criteria, and/or structural accessibility criteria; and (c) selecting genomic loci identified in step (b) based on guide RNA availability, efficacy, and specificity. In some such methods, step (a) comprises identifying accessible genomic loci using an assay for transposase-accessible chromatin with high-throughput sequencing. In some such methods, step (a) comprises identifying accessible genomic loci using DNase I hypersensitive sites sequencing. In some such methods, step (a) comprises identifying accessible genomic loci using an assay for transposase-accessible chromatin with high-throughput sequencing and DNase I hypersensitive sites sequencing. In some such methods, step (b) comprises selecting genomic loci identified in step (a) based on safety criteria, functional silencing criteria, and structural accessibility criteria. In some such methods, the safety criteria in step (b) comprise selecting genomic loci only if they are more than 300 kb from any cancer-related gene, more than 300 kb from any miRNA or small RNA, and more than 50 kb from the 5′ end of any gene. In some such methods, the functional silencing criteria in step (b) comprise selecting genomic loci only if they are more than 50 kb from any replication origin and more than 50 kb from any ultra-conserved elements. In some such methods, the structural accessibility criteria in step (b) comprise selecting genomic loci only if they are not in copy number variable regions. In some such methods, efficacy in step (c) comprises editing efficiency in the tissue or cell type of interest. In some such methods, the method further comprises analyzing the chromatin environment of the genomic loci selected in step (c) for markers to disqualify any genomic locus that is in a region predicted to be a regulatory region, a heterochromatin region, a region participating in chromatin three-dimensional organization, or transcriptionally active region. In some such methods, the markers for the regulatory region comprise H3K4me1, H3K27ac, and H3K4me3. In some such methods, the markers for the heterochromatin region comprise H3K9me3. In some such methods, the markers for the region participating in chromatin three-dimensional organization comprise CTCF. In some such methods, the markers for the transcriptionally active region comprise H3K36me3, PolR2A, RNASeq−, and RNASeq+. In some such methods, step (a) comprises identifying accessible genomic loci using an assay for transposase-accessible chromatin with high-throughput sequencing and DNase I hypersensitive sites sequencing, wherein step (b) comprises selecting genomic loci identified in step (a) based on safety criteria, functional silencing criteria, and structural accessibility criteria, wherein the safety criteria in step (b) comprise selecting genomic loci only if they are more than 300 kb from any cancer-related gene, more than 300 kb from any miRNA or small RNA, and more than 50 kb from the 5′ end of any gene, wherein the functional silencing criteria in step (b) comprise selecting genomic loci only if they are more than 50 kb from any replication origin and more than 50 kb from any ultra-conserved elements, and wherein the structural accessibility criteria in step (b) comprise selecting genomic loci only if they are not in copy number variable regions, and wherein the method further comprises analyzing the chromatin environment of the genomic loci selected in step (c) for markers to disqualify any genomic locus that is in a region predicted to be a regulatory region, a heterochromatin region, a region participating in chromatin three-dimensional organization, or a transcriptionally active region, wherein the markers for the regulatory region comprise H3K4me1, H3K27ac, and H3K4me3, wherein the markers for the heterochromatin region comprise H3K9me3, wherein the markers for the region participating in chromatin three-dimensional organization comprise CTCF, and wherein the markers for the transcriptionally active region comprise H3K36me3, PolR2A, RNASeq−, and RNASeq+. In some such methods, the method is for identifying one or more genomic safe harbor loci in a human tissue or cell type of interest. In some such methods, the tissue or cell type of interest is liver. In some such methods, the tissue or cell type of interest is hematopoietic cells.

### BRIEF DESCRIPTION OF THE FIGURES

FIG. 1 shows a systematic approach used to identify liver-specific, extragenic, genomic safe harbor loci.

FIG. 2 shows editing efficiency of 33 gRNAs covering 20 loci following screening in primary human hepatocytes from three different donors. Editing efficiency of control gRNAs targeting AAVS1, ROSA26, and CCR5 are also shown.

FIGS. 3A-3F show manual curation of six potential liver-specific, extragenic, genomic safe harbor loci (L-SH4, L-SHT 1, L-SH17, L-SH5, L-SH18, and L-SH20, respectively) to analyze the chromatin environment based on Chip Seq data for chromatin marks to disqualify from the analysis any potential safe harbor that was falling in regions predicted to be regulatory regions (H3K4me1, H3K27ac, H3K4me3), heterochromatin regions (H3K9me3), or participating into chromatin organization (CTCF signals).

FIGS. 4A and 4B show editing efficiency at the L-SH5, L-SH18, and L-SH20 genomic loci in primary human hepatocytes in 96-well plates 96 hours following transfection of 100 ng Cas9 mRNA and 25 nM sgRNA (FIG. 4A) or 96 hours following administration of Cas9 mRNA and sgRNA via lipid nanoparticles (dose of 1 g/mL) (FIG. 4B). To assess editing efficiency, next-generation sequencing (NGS) was used to determine the percentage of cells with insertions/deletions (indels).

FIG. 5 shows editing efficiency at the L-SH5, L-SH18, and L-SH20 genomic loci in HepG2 cells following LNP-mediated delivery of Cas9 mRNA and sgRNA and co-delivery of AAV-DJ comprising a firefly luciferase (FLuc) coding sequence driven by a CMV promoter. To assess editing efficiency, NGS was used to determine the percentage of cells with insertions/deletions (indels).

FIG. 6 shows FLuc signal in HepG2 cells following LNP-mediated delivery of Cas9 mRNA and sgRNA (targeting L-SH5, L-SH18, or L-SH20) and delivery of AAV-DJ harboring an FLuc coding sequence driven by a CMV promoter. Negative controls included an untreated sample, an AAV-DJ only samples (no integration), and a sample in which the sgRNA was a non-targeting sgRNA (no integration). After 23 passages, the episomal AAV-DJ FLuc is diluted out and only integrated AAV-DJ in the safe harbors is maintained.

FIG. 7 shows editing efficiency at the L-SH5, L-SH18, and L-SH20 genomic loci in primary human hepatocytes cells following delivery of AAV-DJ harboring an FLuc coding sequence driven by a CMV promoter and 1 μg/mL of LNP comprising Cas9 mRNA and sgRNA. To assess editing efficiency, NGS was used to determine the percentage of cells with insertions/deletions (indels).

FIG. 8 shows FLuc signal in primary human hepatocytes following delivery of 1 g/mL of LNP comprising Cas9 mRNA and sgRNA (targeting L-SH5, L-SH18, or L-SH20) and AAV-DJ harboring an FLuc coding sequence driven by a CMV promoter at a multiplicity of infection (MOI) of 10³, 10⁴, or 10⁵. A sample in which the sgRNA was a non-targeting sgRNA was used as a control. FLuc signal was assessed 72 hours after delivery of the CRISPR/Cas9 and the FLuc nucleic acid construct.

FIG. 9 shows a schematic for testing the sgRNAs targeting L-SH5, L-SH18, and L-SH20 for CRISPR/Cas9-mediated insertion of a CMV-FLuc donor in a humanized liver mouse model.

FIG. 10 shows a transgene (FLuc) driven by a CMV promoter to be inserted into human primary hepatocytes with an AAV-DJ vector.

FIG. 11 shows a schematic for testing the safety profile of targeting potential safe harbor loci in a humanized liver mouse model.

FIG. 12 shows levels of human albumin (hAlb) detected by a serum ELISA from immunodeficient FRG mice 25 weeks post engraftment with primary human hepatocytes.

FIG. 13 shows long term expression of FLuc in a humanized liver mouse model. IVIS imaging was performed to assay for FLuc expression in FRG mice 12 months after engraftment with primary human hepatocytes. Nucleic acid constructs for the insertion of the FLuc transgene into potential safe harbor loci L-SH5, L-SH18, and L-SH20 were delivered to the primary human hepatocytes with an AAV-DJ vector. Images were rearranged from the IVIS analysis.

FIGS. 14A-14E show safety in targeting safe harbor loci L-SH5, L-SH18, and L-SH20 in a humanized liver mouse model. No overt dysregulation of liver enzymes was observed in the serum of immunodeficient FRG mice following engraftment with primary human hepatocytes. Liver markers ALT (FIG. 14A), AST (FIG. 14B), and ALP (FIG. 14C) were consistent among treatment groups. Bilirubin levels (FIG. 14D) were reduced in the treatment groups. Body weight remained consistent between treatment groups (FIG. 14E).

FIG. 15 shows the liver tissue of humanized liver mice stained for H&E, human FAH, human ASGR1, and Ki67. No significant staining was observed with H&E or Ki67, a marker of proliferation in the liver, suggesting no tumorigenesis or active oncogenic transformation. Staining for human FAH and human ASGR1 indicates a high degree of humanization of the mouse livers.

FIG. 16 shows an alignment blocks in between the human chromosome region containing the human safe harbor locus L-SH5 (indicated by the arrow) and the corresponding mouse chromosome's block with same alignment order.

FIG. 17 shows an alignment blocks in between the human chromosome region containing the human safe harbor locus L-SH18 (indicated by the arrow) and the corresponding mouse chromosome's block with same alignment order.

FIG. 18 shows an alignment blocks in between the human chromosome region containing the human safe harbor locus L-SH20 (indicated by the arrow) and the corresponding mouse chromosome's block with same alignment order.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known.

The term “isolated” with respect to cells, tissues (e.g., liver samples), proteins, and nucleic acids includes cells, tissues (e.g., liver samples), proteins, and nucleic acids that are relatively purified with respect to other bacterial, viral, cellular, or other components that may normally be present in situ, up to and including a substantially pure preparation of the cells, tissues (e.g., liver samples), proteins, and nucleic acids. The term “isolated” also includes cells, tissues (e.g., liver samples), proteins, and nucleic acids that have no naturally occurring counterpart, have been chemically synthesized and are thus substantially uncontaminated by other cells, tissues (e.g., liver samples), proteins, and nucleic acids, or has been separated or purified from most other components (e.g., cellular components) with which they are naturally accompanied (e.g., other cellular proteins, polynucleotides, or cellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous Rosa26 sequence of a human refers to a native Rosa26 sequence that naturally occurs at the Rosa26 locus in the human.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” (i.e., “codon optimized” sequences) takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a polypeptide of interest can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, a “Rosa26 locus” may refer to the specific location of a Rosa26 gene, Rosa26 DNA sequence, or Rosa26 position on a chromosome of the genome of an organism that has been identified as to where such a sequence resides. A “Rosa26 locus” may comprise a regulatory element of a Rosa26 gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a human cell, a human liver cell, or a human liver hepatocyte). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing a upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, it is understood that when the maximum amount of a value is represented by 100% (e.g., 100% inhibition) that the value is limited by the method of detection. For example, 100% inhibition is understood as inhibition to a level below the level of detection of the assay.

Unless otherwise apparent from the context, the term “about” encompasses values: 5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

In the event of a conflict between a sequence in the application and an indicated accession number or position in an accession number, the sequence in the application predominates.

### DETAILED DESCRIPTION

### I. Overview

Current gene therapy approaches rely on episomal expression of transgenes and/or insertion in specific genomic loci. The episomal approach has proven limited for the liver due to dilution or silencing. Integration in a specific locus allows for sustained expression of a transgene. However, this approach is still to be proven effective and safe in human settings. Canonical genomic safe harbor loci in humans, such as AAVS1, CCR5, and Rosa26, are all intragenic and are less explored than mouse genomic safe harbor loci. In addition, different tissues have different chromatin states for a defined locus, so canonical genomic safe harbors can be silenced in some tissues. The canonical genomic safe harbor loci in humans all have additional drawbacks. Methylation mechanisms can silence transgene in the AAVS1 locus in some cell lineages, knockout of CCR5 can lead to increased susceptibility to infection with West Nile virus and Japanese encephalitis, and the human Rosa26 locus is less explored than the mouse ortholog. Thus, there is a need for tissue-specific genomic safe harbor loci.

Compositions and methods for inserting a nucleic acid encoding a product of interest into a genomic safe harbor locus in a cell, a population of cells, or a subject (e.g., a subject in need thereof) or for expressing a nucleic acid encoding a product of interest from a genomic safe harbor locus in a cell, a population of cells, or a subject (e.g., a subject in need thereof) are provided. Also provided are cells or populations of cells or subjects comprising a nucleic acid construct comprising a coding sequence for a product of interest inserted into a genomic safe harbor locus. Also provided herein are methods of identifying genomic safe harbor loci (e.g., extragenic genomic safe harbor loci) for use in specific cell or tissue types.

II. Compositions for Inserting Nucleic Acid Constructs into a Genomic Safe Harbor Locus and for Expressing Products of Interest from a Genomic Safe Harbor Locus in Cells and Subjects

Provided herein are nucleic acid constructs and compositions that allow insertion of a coding sequence for a product of interest into a genomic safe harbor locus and/or expression of the coding sequence for the product of interest from the genomic safe harbor locus. The nucleic acid constructs and compositions can be used in methods for integration into a genomic safe harbor locus and/or expression from a genomic safe harbor locus in a cell or a subject. Also provided are nuclease agents (e.g., targeting a genomic safe harbor locus) or nucleic acids encoding nuclease agents to facilitate integration of the nucleic acid constructs into a genomic safe harbor locus. Also provided are nuclease agents targeting near or within a genomic safe harbor locus or nucleic acids encoding nuclease agents to facilitate integration of the nucleic acid constructs into a genomic safe harbor locus.

### A. Genomic Safe Harbor Loci Methods of Identifying Genomic Safe Harbor Loci

Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes.

Target genomic loci used herein can be genomic safe harbor loci. Genomic safe harbor loci include chromosomal loci where transgenes or other exogenous nucleic acid inserts can be stably and reliably expressed in tissues of interest without overtly altering cell behavior or phenotype (i.e., without any deleterious effects on the host cell). For example, the genomic safe harbor locus can be one in which expression of the inserted gene sequence is not perturbed by any read-through expression from neighboring genes. For example, genomic safe harbor loci can include chromosomal loci where exogenous DNA can integrate and function in a predictable manner without adversely affecting endogenous gene structure or expression. The genomic safe harbor loci can be targeted with high efficiency, and safe harbor loci can be disrupted with no overt phenotype. Genomic safe harbor loci can include extragenic regions or intragenic regions such as, for example, loci within genes that are non-essential, dispensable, or able to be disrupted without overt phenotypic consequences.

A genomic safe harbor locus described herein can be a genomic locus that, when targeted for integration in a subject, does not cause changes in liver functionality. A genomic safe harbor locus described herein can be a genomic locus that, when targeted for integration in a subject, does not cause changes in alanine aminotransferase (alanine transaminase or ALT) levels. A genomic safe harbor locus described herein can be a genomic locus that, when targeted for integration in a subject, does not cause changes in aspartate aminotransferase (AST) levels. A genomic safe harbor locus described herein can be a genomic locus that, when targeted for integration in a subject, does not cause changes in alkaline phosphatase (ALP) levels. A genomic safe harbor locus described herein can be a genomic locus that, when targeted for integration in a subject, does not cause changes in body weight. A genomic safe harbor locus described herein can be a genomic locus that, when targeted for integration in a subject, does not cause changes in proliferation such as in a target organ such as the liver (e.g., as assessed by Ki67 staining). A genomic safe harbor locus described herein can be a genomic locus that, when targeted for integration in a subject, does not cause oncogenic transformation such as in a target organ such as the liver (e.g., as assessed by H&E staining).

A genomic safe harbor locus described herein can be a genomic locus with an open chromatin configuration in the liver such that exogenous nucleic acid inserts can be stably and reliably expressed in the liver. Alternatively, a genomic safe harbor locus can be a genomic locus with an open chromatin configuration in another tissue or cell type (e.g., hematopoietic cells, such as hematopoietic stem cells, T cells, B cells, and/or macrophages) such that exogenous nucleic acid inserts can be stably and reliably expressed in that tissue or cell type.

A genomic safe harbor locus described herein can be an extragenic genomic safe harbor locus (i.e., occurring outside of a gene). In a specific example, a genomic safe harbor locus described herein is an extragenic genomic safe harbor locus with an open chromatin configuration in the liver.

In a specific example, the genomic safe harbor locus can be one that is more than 300 kb from any cancer-related gene (e.g., to prevent insertional oncogenesis), more than 300 kb from any miRNA or small RNA (e.g., to preserve regulation of gene expression and cellular development), more than 50 kb from the 5′ end of any gene (e.g., to avoid perturbing endogenous gene expression), more than 50 kb from any replication origin, more than 50 kb from any ultra-conserved elements (e.g., non-coding intragenic or intergenic regions that are completely conserved in human, mouse, and rat genomes), outside of copy number variable regions, and in open chromatin (as determined, e.g., by ATAC-Seq analysis (e.g., in human liver biopsy samples)). In addition, the genomic safe harbor locus can be one that does not overlap with regions predicted to be regulatory regions (e.g., H3K4me1, H3K27ac, and/or H3K4me3 markers), heterochromatin regions (e.g., H3K9me3 marker), or participating into chromatin organization (e.g., CTCF signals).

For example, a method of identifying a genomic safe harbor locus (e.g., an extragenic genomic safe harbor locus) can comprise: (a) identifying accessible genomic loci (i.e., chromatin sites) in a tissue or cell type of interest (e.g., relying on ATAC-Seq data sets); (b) filtering out loci identified in step (a) based on safety criteria, functional silencing criteria, and/or structural accessibility criteria; and (c) filtering out loci identified in step (b) based on gRNA availability, efficacy (editing efficiency), and specificity (off-target analysis). Such methods can further comprise analyzing the chromatin environment for chromatin marks to disqualify from the analysis any potential safe harbor that is falling in regions predicted to be regulatory regions (e.g., H3K4me1, H3K27ac, and/or H3K4me3), heterochromatin regions (e.g., H3K9me3), or participating in chromatin three-dimensional organization (e.g., CTCF signals).

Eukaryotic chromatin is tightly packaged into an array of nucleosomes, each consisting of a histone octamer core wrapped around DNA and separated by linker DNA. The nucleosomal core consists of histone proteins that can be post-translationally altered by covalent modifications or replaced by histone variants. Positioning of nucleosomes throughout a genome has a significant regulatory function by modifying the in vivo availability of binding sites to transcription factors and the general transcription machinery and thus affecting DNA-dependent processes such as transcription, DNA repair, replication, and recombination. Accessible genomic loci are regions of open chromatin. Open chromatin regions are nucleosome-depleted regions that can be bound by protein factors and can play various roles in DNA replication, nuclear organization, and gene transcription. Step (a) can comprise, for example, identifying accessible genomic loci using an assay for transposase-accessible chromatin, such as ATAC-Seq analysis. ATAC-Seq stands for Assay for Transposase-Accessible Chromatin with high-throughput sequencing. See, e.g., Buenrostro et al. (2013) Nat. Methods 10(12):1213-1218 and Buenrostro et al. (2015) Curr. Protoc. Mol. Biol. 109:21.29.1-21.29.9, each of which is herein incorporated by reference in its entirety for all purposes. The ATAC-Seq method relies on next-generation sequencing (NGS) library construction using the hyperactive transposase Tn5. NGS adapters are loaded onto the transposase, which allows simultaneous fragmentation of chromatin and integration of those adapters into open chromatin regions. The library that is generated can be sequenced by NGS, and the regions of the genome with open or accessible chromatin are analyzed using bioinformatics. As a first step, cells are harvested. After harvesting, cells are lysed with a nonionic detergent to yield pure nuclei. The resulting chromatin is then fragmented and simultaneously tagmented with sequencing adapters using the Tn5 transposase to generate the ATAC-Seq library. After purification, the library can be amplified by PCR using barcoded primers. The resulting library can then be analyzed by qPCR or next-generation sequencing. ATAC-seq identifies accessible DNA regions by probing open chromatin with hyperactive mutant Tn5 Transposase that inserts sequencing adapters into open regions of the genome. While naturally occurring transposases have a low level of activity, ATAC-seq employs the mutated hyperactive transposase. In a process called tagmentation, Tn5 transposase cleaves and tags double-stranded DNA with sequencing adaptors. The tagged DNA fragments are then purified, PCR-amplified, and sequenced using next-generation sequencing. Sequencing reads can then be used to infer regions of increased accessibility as well as to map regions of transcription factor binding sites and nucleosome positions. The number of reads for a region correlate with how open that chromatin is, at single nucleotide resolution.

Step (a) can also comprise, for example, identifying accessible genomic loci using DNase I hypersensitive sites sequencing (DNase-Seq). DNase-seq is a method used to identify the location of regulatory regions based on the genome-wide sequencing of regions sensitive to cleavage by DNase I. This method utilizes DNase I to selectively digest nucleosome-depleted DNA, whereas DNA regions tightly wrapped in nucleosome and higher order structures are more resistant. The high-throughput method identifies DNase I hypersensitive sites across the whole genome by capturing DNase-digested fragments and sequencing them by high-throughput next generation sequencing.

In step (b), safety criteria can include selecting genomic loci only if they are more than 300 kb from any cancer-related gene (e.g., to prevent insertional oncogenesis), more than 300 kb from any miRNA or small RNA (e.g., to preserve regulation of gene expression and cellular development), and/or more than 50 kb from the 5′ end of any gene (e.g., to avoid perturbing endogenous gene expression). Functional silencing criteria can include selecting genomic loci only if they are more than 50 kb from any replication origin and/or more than 50 kb from any ultra-conserved elements (e.g., non-coding intragenic or intergenic regions that are completely conserved in human, mouse, and rat genomes). Structural accessibility criteria can include selecting genomic loci only if they are not in copy number variable regions.

In step (c), loci can be filtered based on gRNA availability, efficacy (editing efficiency), and specificity (off-target analysis). gRNA availability means there are suitable target sequences for guide RNAs, taking into account PAM requirements. Efficacy means editing efficiency of a gRNA in the tissue or cell type of interest. Any suitable threshold of editing efficiency can be set. For example, a locus or gRNA can be selected if the editing efficiency is at least about 10%, at least about 11%, at least about 12%, at least about 13%, at least about 14%, at least about 15%, at least about 16%, at least about 17%, at least about 18%, at least about 19%, or at least about 20%. In one example, gRNA efficacy is measured in primary cells (e.g., primary hepatocytes). In another example, gRNA efficacy is measured in a tissue of interest in vivo. In a specific example, gRNA efficacy is measured in primary cells from multiple different donors (e.g., primary hepatocytes from multiple different donors, such as two or three different donors). Any suitable threshold for gRNA specificity can be used. For example, a guide RNA can be selected if there are no other sequences in the genome that are a perfect match or have only one mismatch with the guide RNA target sequence. In another example, a guide RNA can be selected if there are no other sequences in the genome that are a perfect match or have only one or two mismatches with the guide RNA target sequence.

Such methods can also comprise analyzing the chromatin environment for markers (e.g., signals or chromatin marks) to disqualify from the analysis any potential safe harbor that is falling in regions predicted to be regulatory regions (e.g., H3K4me1, H3K27ac, and/or H3K4me3), heterochromatin regions (e.g., H3K9me3), participating into chromatin organization (e.g., CTCF signals), or regions having transcriptional activity (e.g., H3K36me3, PolR2A, RNASeq−, and RNASeq+). For example, ChIP-Seq data on transcription factor binding, genome-wide DNA methylation, promoter/enhancer signatures inferred by histone marks, and chromatin accessibility can be used. Post-translational modifications on histone tails are closely correlated to transcriptional states. For example, trimethylation of histone H3 lysine 4 (H3K4me3) marks active gene promoters. Monomethylation on lysine 4 of histone 3 (H3K4me1) is a mark that has been linked to enhancers. Identifying regions enriched for H3K4me1 and depleted in H3K4me3, or regions enriched for both H3K4me1 and H3K27ac, have proven to be feasible methods for enhancer discovery. H3K27ac is an activation mark distinguishing active from primed enhancers. H3K9me3 marks regions subject to long-term repression. The primary role of CTCF is thought to be in regulating the 3D structure of chromatin. CTCF binds together strands of DNA, thus forming chromatin loops, and anchors DNA to cellular structures like the nuclear lamina. It also defines the boundaries between active and heterochromatic DNA. Because the three-dimensional structure of DNA influences the regulation of genes, CTCF's activity influences the expression of genes. CTCF is thought to be a primary part of the activity of insulators, sequences that block the interaction between enhancers and promoters. CTCF binding has also been shown to promote and repress gene expression. It is unknown whether CTCF affects gene expression solely through its looping activity, or if it has some other, unknown, activity. H3K36me3 indicates gene bodies, to show experimentally that there is no transcriptional unit being interfered with. PolR2A indicates transcriptional activity, and is used to show there is no transcript coming from the region. RNASeq− indicates transcriptional activity on the minus strand of DNA, and RNASeq+ indicates transcriptional activity on the plus strand of DNA, and both are used to show there is no transcript coming from the region. RNA-Seq (RNA sequencing) is a sequencing technique that uses next-generation sequencing (NGS) to reveal the presence and quantity of RNA in a biological sample. The mRNA is extracted from the sample, fragmented and copied into stable ds-cDNA. The ds-cDNA is sequenced using high-throughput, short-read sequencing methods. These sequences can then be aligned to a reference genome sequence to reconstruct which genome regions were being transcribed.

In some embodiments, integration of a nucleic acid construct into a genomic safe harbor loci as described herein does not cause liver toxicity. In some embodiments, integration of a nucleic acid construct into a genomic safe harbor loci as described herein does not expression changes in adjacent genes. In some embodiments, integration of a nucleic acid construct into a genomic safe harbor loci as described herein does not cause liver toxicity and does not expression changes in adjacent genes.

In a specific example, the genomic safe harbor locus is selected from the following genomic locations: (i) human chromosome 13, coordinates 77460242-77460537 (referred to herein as L-SH5) or a corresponding region (e.g., orthologous or syntenic region) in a non-human animal, non-human mammal (e.g., non-human primate), or rodent, such as a rat or a mouse; (ii) human chromosome 6, coordinates 170031084-170031382 (referred to herein as L-SH18) or a corresponding region (e.g., orthologous or syntenic region) in a non-human animal, non-human mammal (e.g., non-human primate), or rodent, such as a rat or a mouse; and (iii) human chromosome 9, coordinates 25207412-25207703 (referred to herein as L-SH20) or a corresponding region (e.g., orthologous or syntenic region) in a non-human animal, non-human mammal (e.g., non-human primate), or rodent, such as a rat or a mouse. Throughout this application, the referenced genomic coordinates are based on genomic annotations in the GRCh38 (also referred to as hg38) assembly of the human genome from the Genome Reference Consortium, available at the National Center for Biotechnology Information website. Exemplary sequences of L-SH5, L-SH18, and L-SH20 based on genomic annotations in the GRCh38 (also referred to as hg38) assembly of the human genome from the Genome Reference Consortium are set forth in SEQ ID NOS: 39, 40, and 41, respectively. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

In a specific example, the genomic safe harbor locus is selected from the following genomic coordinates: (i) about 77460242 to about 77460537 on human chromosome 13 (corresponds to L-SH5) or a corresponding region (e.g., orthologous or syntenic region) in a non-human animal, non-human mammal (e.g., non-human primate), or rodent, such as a rat or a mouse; (ii) about 170031084 to about 170031382 on human chromosome 6 (corresponds to L-SH18) or a corresponding region (e.g., orthologous or syntenic region) in a non-human animal, non-human mammal (e.g., non-human primate), or rodent, such as a rat or a mouse; and (iii) about 25207412 to about 25207703 on human chromosome 9 (corresponds to L-SH20) or a corresponding region (e.g., orthologous or syntenic region) in a non-human animal, non-human mammal (e.g., non-human primate), or rodent, such as a rat or a mouse. The term “about” when referring to genomic coordinates means±20 base pairs. In other examples, the genomic safe harbor locus is near the regions identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In one specific example, the genomic safe harbor locus is human L-SH5 (chromosome 13, coordinates 77460242-77460537) or a corresponding region (e.g., orthologous or syntenic region) in a non-human animal, non-human mammal (e.g., non-human primate), or rodent, such as a rat or a mouse. For example, the genomic safe harbor locus can comprise the sequence set forth in SEQ ID NO: 39 or variants thereof which are located at the same position, or genetic locus, on a chromosome in humans or orthologous or syntenic regions in a non-human animal, non-human mammal (e.g., non-human primate), or rodent, such as a rat or a mouse. Syntenic regions are derived from a single ancestral genomic region. For example, syntenic regions can be from different organisms and are derived from speciation.

In another specific example, the genomic safe harbor locus is human L-SH18 (chromosome 6, coordinates 170031084-170031382) or a corresponding region (e.g., orthologous or syntenic region) in a non-human animal, non-human mammal (e.g., non-human primate), or rodent, such as a rat or a mouse. For example, the genomic safe harbor locus can comprise the sequence set forth in SEQ ID NO: 40 or variants thereof which are located at the same position, or genetic locus, on a chromosome in humans or orthologous or syntenic regions in a non-human animal, non-human mammal (e.g., non-human primate), or rodent, such as a rat or a mouse.

In another specific example, the genomic safe harbor locus is human L-SH20 (chromosome 9, coordinates 25207412-25207703) or a corresponding region (e.g., orthologous or syntenic region) in a non-human animal, non-human mammal (e.g., non-human primate), or rodent, such as a rat or a mouse. For example, the genomic safe harbor locus can comprise the sequence set forth in SEQ ID NO: 41 or variants thereof which are located at the same position, or genetic locus, on a chromosome in humans or orthologous or syntenic regions in a non-human animal, non-human mammal (e.g., non-human primate), or rodent, such as a rat or a mouse.

In one specific example, the genomic safe harbor locus corresponds to human L-SH5 (coordinates of about 77460242 to about 77460537 on chromosome 13) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to human L-SH18 (coordinates of about 170031084 to about 170031382 on chromosome 6) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means ±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to human L-SH20 (coordinates of about 25207412 to about 25207703 on chromosome 9) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In a specific example, the genomic safe harbor locus is selected from the following genomic locations: (i) mouse chromosome 14, coordinates 103,450,397-103,451,396 (referred to herein as mouse L-SH5) or a corresponding region (e.g., orthologous or syntenic region) in a non-human animal, non-human mammal (e.g., non-human primate), or rodent, such as a rat; (ii) mouse chromosome 17, coordinates 15,226,387-15,227,386 (referred to herein as mouse L-SH18) or a corresponding region (e.g., orthologous or syntenic region) in a non-human animal, non-human mammal (e.g., non-human primate), or rodent, such as a rat; and (iii) mouse chromosome 4, coordinates 92,827,563-92,828,592 (referred to herein as mouse L-SH20) or a corresponding region (e.g., orthologous or syntenic region) in a non-human animal, non-human mammal (e.g., non-human primate), or rodent, such as a rat. Throughout this application, the referenced genomic coordinates are based on genomic annotations in the GRCm38 (also referred to as mm10) assembly of the mouse genome from the Genome Reference Consortium, available at the National Center for Biotechnology Information website. Exemplary sequences of L-SH5, L-SH18, and L-SH20 based on genomic annotations in the GRCm38 (also referred to as mm10) assembly of the mouse genome from the Genome Reference Consortium are set forth in SEQ ID NOS: 405, 406, and 407, respectively. Tools and methods for converting genomic coordinates between one assembly and another are known in the art and can be used to convert the genomic coordinates provided herein to the corresponding coordinates in another assembly of the mouse genome, including conversion to an earlier assembly generated by the same institution or using the same algorithm, and conversion an assembly generated by a different institution or algorithm. Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

In a specific example, the genomic safe harbor locus is selected from the following genomic coordinates: (i) about 103,450,397 to about 103,451,396 on mouse chromosome 14 (corresponds to mouse L-SH5) or a corresponding region (e.g., orthologous or syntenic region) in a non-human animal, non-human mammal (e.g., non-human primate), or rodent, such as a rat; (ii) about 15,226,387 to about 15,227,386 on mouse chromosome 17 (corresponds to mouse L-SH18) or a corresponding region (e.g., orthologous or syntenic region) in a non-human animal, non-human mammal (e.g., non-human primate), or rodent, such as a rat; and (iii) about 92,827,563 to about 92,828,592 on mouse chromosome 4 (corresponds to mouse L-SH20) or a corresponding region (e.g., orthologous or syntenic region) in a non-human animal, non-human mammal (e.g., non-human primate), or rodent, such as a rat. The term “about” when referring to genomic coordinates means±20 base pairs. In other examples, the genomic safe harbor locus is near the regions identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In one specific example, the genomic safe harbor locus is mouse L-SH5 (chromosome 14, coordinates 103,450,397-103,451,396) or a corresponding region (e.g., orthologous or syntenic region) in a non-human animal, non-human mammal (e.g., non-human primate), or rodent, such as a rat. For example, the genomic safe harbor locus can comprise the sequence set forth in SEQ ID NO: 405 or variants thereof which are located at the same position, or genetic locus, on a chromosome in mice or orthologous or syntenic regions in a human or non-human animal, non-human mammal (e.g., non-human primate), or rodent, such as a rat. Syntenic regions are derived from a single ancestral genomic region. For example, syntenic regions can be from different organisms and are derived from speciation.

In another specific example, the genomic safe harbor locus is mouse L-SH18 (chromosome 17, coordinates 15,226,387-15,227,386) or a corresponding region (e.g., orthologous or syntenic region) in a non-human animal, non-human mammal (e.g., non-human primate), or rodent, such as a rat. For example, the genomic safe harbor locus can comprise the sequence set forth in SEQ ID NO: 406 or variants thereof which are located at the same position, or genetic locus, on a chromosome in mice or orthologous or syntenic regions in a human or non-human animal, non-human mammal (e.g., non-human primate), or rodent, such as a rat.

In another specific example, the genomic safe harbor locus is mouse L-SH20 (chromosome 4, coordinates 92,827,563-92,828,592) or a corresponding region (e.g., orthologous or syntenic region) in a non-human animal, non-human mammal (e.g., non-human primate), or rodent, such as a rat. For example, the genomic safe harbor locus can comprise the sequence set forth in SEQ ID NO: 407 or variants thereof which are located at the same position, or genetic locus, on a chromosome in mice or orthologous or syntenic regions in a human or non-human animal, non-human mammal (e.g., non-human primate), or rodent, such as a rat.

In one specific example, the genomic safe harbor locus corresponds to mouse L-SH5 (coordinates of about 103,450,397 to about 103,451,396 on mouse chromosome 14) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to mouse L-SH18 (coordinates of about 15,226,387 to about 15,227,386 on mouse chromosome 17) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to mouse L-SH20 (coordinates of about 92,827,563 to about 92,828,592 on mouse chromosome 4) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

### B. Nucleic Acid Constructs Encoding a Product of Interest

The compositions and methods described herein include the use of a nucleic acid construct that comprises a coding sequence for a product of interest (e.g., a polypeptide of interest) operably linked to a promoter. Such nucleic acid constructs can be for insertion into a target genomic locus (e.g., a genomic safe harbor locus as described elsewhere herein) or into a cleavage site created by a nuclease agent or CRISPR/Cas system as disclosed elsewhere herein. The term cleavage site includes a DNA sequence at which a nick or double-strand break is created by a nuclease agent (e.g., a Cas9 protein complexed with a guide RNA). In some embodiments, a double-stranded break is created by a Cas9 protein complexed with a guide RNA, e.g., a SpCas9 protein complexed with a SpCas9 guide RNA.

The length of the nucleic acid constructs disclosed herein can vary. The construct can be, for example, from about 1 kb to about 5 kb, such as from about 1 kb to about 4.5 kb or about 1 kb to about 4 kb. An exemplary nucleic acid construct is between about 1 kb to about 5 kb in length or between about 1 kb to about 4 kb in length. Alternatively, a nucleic acid construct can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, a nucleic acid construct can be, for example, no more than 5 kb, no more than 4.5 kb, no more than 4 kb, no more than 3.5 kb, no more than 3 kb, or no more than 2.5 kb in length.

The constructs can comprise deoxyribonucleic acid (DNA) or ribonucleic acid (RNA), can be single-stranded, double-stranded, or partially single-stranded and partially double-stranded, and can be introduced into a host cell in linear or circular (e.g., minicircle) form. See, e.g., US 2010/0047805, US 2011/0281361, and US 2011/0207221, each of which is herein incorporated by reference in their entirety for all purposes. If introduced in linear form, the ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in their entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters, and genes encoding antibiotic resistance. A construct may omit viral elements. Moreover, constructs can be introduced as a naked nucleic acid, can be introduced as a nucleic acid complexed with an agent such as a liposome or poloxamer, or can be delivered by viruses (e.g., adenovirus, adeno-associated virus (AAV), herpesvirus, retrovirus, or lentivirus).

The constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

The constructs comprise a promoter and/or enhancer that drives expression of the product of interest, for example a constitutive promoter or an inducible or tissue-specific (e.g., liver-specific) promoter that drives expression of the product of interest in an episome or upon integration. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. For example, the promoter may be a CMV promoter or a truncated CMV promoter. In another example, the promoter may be an EF1a promoter. Promoters suitable for liver can include, for example, albumin (ALB) promoters or transthyretin (TTR) promoters. Suitable enhancers for liver can include, for example, SERPINA1 enhancers. Non-limiting exemplary inducible promoters include those inducible by heat shock, light, chemicals, peptides, metals, steroids, antibiotics, or alcohol. The inducible promoter may be one that has a low basal (non-induced) expression level, such as the Tet-On® promoter (Clontech).

In some examples, the nucleic acid construct works in homology-independent insertion of a nucleic acid that encodes a product of interest (e.g., polypeptide of interest). Such nucleic acid constructs can work, for example, in non-dividing cells (e.g., cells in which non-homologous end joining (NHEJ), not homologous recombination (HR), is the primary mechanism by which double-stranded DNA breaks are repaired) or dividing cells (e.g., actively dividing cells). Such constructs can be, for example, homology-independent donor constructs. In preferred embodiments, promoters and other regulatory sequences are appropriate for use in humans, e.g., recognized by regulatory factors in human cells, e.g., in human liver cells, and acceptable to regulatory authorities for use in humans.

The constructs disclosed herein can be modified to include or exclude any suitable structural feature as needed for any particular use and/or that confers one or more desired function. For example, some constructs disclosed herein do not comprise a homology arm. Some constructs disclosed herein are capable of insertion into a target genomic locus or a cut site in a target DNA sequence for a nuclease agent (e.g., capable of insertion into a genomic safe harbor locus) by non-homologous end joining. For example, such constructs can be inserted into a blunt end double-strand break following cleavage with a nuclease agent (e.g., CRISPR/Cas system, e.g., a SpyCas9 CRISPR/Cas system) as disclosed herein. In a specific example, the construct can be delivered via AAV and can be capable of insertion by non-homologous end joining (e.g., the construct does not comprise a homology arm).

In a particular example, the construct can be inserted via homology-independent targeted integration. For example, the nucleic acid construct or the product of interest coding sequence (e.g., the polypeptide of interest coding sequence) and the promoter in the construct can be flanked on each side by a target site for a nuclease agent (e.g., the same target site as in the target DNA sequence for targeted insertion (e.g., in a genomic safe harbor locus), and the same nuclease agent being used to cleave the target DNA sequence for targeted insertion). The nuclease agent can then cleave the flanking target sites. In a specific example, the construct is delivered by AAV-mediated delivery, and cleavage of the flanking target sites can remove the inverted terminal repeats (ITRs) of the AAV. In some instances, the target DNA sequence for targeted insertion (e.g., target DNA sequence in a genomic safe harbor locus such as a gRNA target sequence including the flanking protospacer adjacent motif) is no longer present if the product of interest coding sequence (e.g., the polypeptide of interest coding sequence) and promoter are inserted into the cut site or target DNA sequence in one orientation but it is reformed if the product of interest coding sequence (e.g., the polypeptide of interest coding sequence) and promoter are inserted into the cut site or target DNA sequence in the opposite orientation.

The constructs disclosed herein can comprise a polyadenylation sequence or polyadenylation tail sequence (e.g., downstream or 3′ of a product of interest coding sequence). Methods of designing a suitable polyadenylation tail sequence are well-known. The polyadenylation tail sequence can be encoded, for example, as a “poly-A” stretch downstream of the product of interest coding sequence. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. In one example, the polyadenylation signal is a simian virus 40 (SV40) late polyadenylation signal. In another example, the polyadenylation signal is a bovine growth hormone (BGH) polyadenylation signal.

### (1) Products of Interest and Polypeptides of Interest

Any product of interest may be encoded by the nucleic acid constructs disclosed herein. For example, the product of interest can be a therapeutic product of interest, such as a therapeutic RNA or a therapeutic polypeptide.

In one example, the product of interest is an RNA of interest, such as an miRNA, an antisense oligonucleotide, an RNAi agent, or a guide RNA for use in a CRISPR/Cas system. For example, the RNA of interest can be a therapeutic RNA.

An “RNAi agent” is a composition that comprises a small double-stranded RNA or RNA-like (e.g., chemically modified RNA) oligonucleotide molecule capable of facilitating degradation or inhibition of translation of a target RNA, such as messenger RNA (mRNA), in a sequence-specific manner. The oligonucleotide in the RNAi agent is a polymer of linked nucleosides, each of which can be independently modified or unmodified. RNAi agents operate through the RNA interference mechanism (i.e., inducing RNA interference through interaction with the RNA interference pathway machinery (RNA-induced silencing complex or RISC) of mammalian cells). While it is believed that RNAi agents, as that term is used herein, operate primarily through the RNA interference mechanism, the disclosed RNAi agents are not bound by or limited to any particular pathway or mechanism of action. RNAi agents disclosed herein comprise a sense strand and an antisense strand, and include, but are not limited to, short interfering RNAs (siRNAs), double-stranded RNAs (dsRNA), micro RNAs (miRNAs), short hairpin RNAs (shRNA), and dicer substrates. The antisense strand of the RNAi agents described herein is at least partially complementary to a sequence (i.e., a succession or order of nucleobases or nucleotides, described with a succession of letters using standard nomenclature) in the target RNA.

Single-stranded ASOs and RNA interference (RNAi) share a fundamental principle in that an oligonucleotide binds a target RNA through Watson-Crick base pairing. Without wishing to be bound by theory, during RNAi, a small RNA duplex (RNAi agent) associates with the RNA-induced silencing complex (RISC), one strand (the passenger strand) is lost, and the remaining strand (the guide strand) cooperates with RISC to bind complementary RNA. Argonaute 2 (Ago2), the catalytic component of the RISC, then cleaves the target RNA. The guide strand is always associated with either the complementary sense strand or a protein (RISC). In contrast, an ASO must survive and function as a single strand. ASOs bind to the target RNA and block ribosomes or other factors, such as splicing factors, from binding the RNA or recruit proteins such as nucleases. Different modifications and target regions are chosen for ASOs based on the desired mechanism of action. A gapmer is an ASO oligonucleotide containing 2-5 chemically modified nucleotides (e.g. LNA or 2′-MOE) on each terminus flanking a central 8-10 base gap of DNA. After binding the target RNA, the DNA-RNA hybrid acts substrate for RNase H.

In another example, the product of interest is a polypeptide of interest. In one example, the polypeptide of interest is a therapeutic polypeptide. For example, the therapeutic polypeptides can be a polypeptide that is lacking or deficient in a subject. In one example, the polypeptide of interest is an enzyme.

In one example, a polypeptide of interest is an antibody or an antigen-binding protein. In another example, a polypeptide of interest is an exogenous T cell receptor or a chimeric antigen receptor (CAR). In another example, a polypeptide of interest is a Cas protein (e.g., Cas9) for use in a CRISPR/Cas system.

An “antigen-binding protein” as disclosed herein includes any protein that binds to an antigen. Examples of antigen-binding proteins include an antibody, an antigen-binding fragment of an antibody, a multi-specific antibody (e.g., a bi-specific antibody), an scFv, a bis-scFv, a diabody, a triabody, a tetrabody, a V-NAR, a VHH, a VL, a F(ab), a F(ab)₂, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes).

The term “antibody” includes immunoglobulin molecules comprising four polypeptide chains, two heavy (H) chains and two light (L) chains inter-connected by disulfide bonds. Each heavy chain comprises a heavy chain variable domain and a heavy chain constant region (C_{H}). The heavy chain constant region comprises three domains: C_{H}1, C_{H}2 and C_{H}3. Each light chain comprises a light chain variable domain and a light chain constant region (C_{L}). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3). The term “high affinity” antibody refers to an antibody that has a K_{D} with respect to its target epitope about of 10⁻⁹ M or lower (e.g., about 1×10⁻⁹ M, 1×10⁻¹⁰ M, 1×10¹¹ M, or about 1×10⁻¹²M). In one embodiment, K_{D} is measured by surface plasmon resonance, e.g., BIACORE™; in another embodiment, K_{D} is measured by ELISA.

An antigen-binding protein or antibody can be, for example, a neutralizing antigen-binding protein or antibody or a broadly neutralizing antigen-binding protein or antibody. A neutralizing antibody is an antibody that defends a cell from an antigen or infectious body by neutralizing any effect it has biologically. Broadly-neutralizing antibodies (bNAbs) affect multiple strains of a particular bacteria or virus. For example, broadly neutralizing antibodies can focus on conserved functional targets, attacking a vulnerable site on conserved bacterial or viral proteins (e.g., a vulnerable site on the influenza viral protein hemagglutinin). Antibodies developed by the immune system upon infection or vaccination tend to focus on easily accessible loops on the bacterial or viral surface, which often have great sequence and conformational variability. This is a problem for two reasons: the bacteria or virus population can quickly evade these antibodies, and the antibodies are attacking portions of the protein that are not essential for function. Broadly neutralizing antibodies-termed “broadly” because they attack many strains of the bacteria or virus, and “neutralizing” because they attack key functional sites in the bacteria or virus and block infection—can overcome these problems. Unfortunately, however, these antibodies usually come too late and do not provide effective protection from the disease.

The antigen-binding proteins disclosed herein can target any antigen. The term “antigen” refers to a substance, whether an entire molecule or a domain within a molecule, which is capable of eliciting production of antibodies with binding specificity to that substance. The term antigen also includes substances, which in wild type host organisms would not elicit antibody production by virtue of self-recognition, but can elicit such a response in a host animal with appropriate genetic engineering to break immunological tolerance.

As one example, the targeted antigen can be a disease-associated antigen. The term “disease-associated antigen” refers to an antigen whose presence is correlated with the occurrence or progression of a particular disease. For example, the antigen can be in a disease-associated protein (i.e., a protein whose expression is correlated with the occurrence or progression of the disease). Optionally, a disease-associated protein can be a protein that is expressed in a particular type of disease but is not normally expressed in healthy adult tissue (i.e., a protein with disease-specific expression or disease-restricted expression). However, a disease-associated protein does not have to have disease-specific or disease-restricted expression.

As one example, a disease-associated antigen can be a cancer-associated antigen. The term “cancer-associated antigen” refers to an antigen whose presence is correlated with the occurrence or progression of one or more types of cancer. For example, the antigen can be in a cancer-associated protein (i.e., a protein whose expression is correlated with the occurrence or progression of one or more types of cancer). For example, a cancer-associated protein can be an oncogenic protein (i.e., a protein with activity that can contribute to cancer progression, such as proteins that regulate cell growth), or it can be a tumor-suppressor protein (i.e., a protein that typically acts to alleviate the potential for cancer formation, such as through negative regulation of the cell cycle or by promoting apoptosis). Optionally, a cancer-associated protein can be a protein that is expressed in a particular type of cancer but is not normally expressed in healthy adult tissue (i.e., a protein with cancer-specific expression, cancer-restricted expression, tumor-specific expression, or tumor-restricted expression). However, a cancer-associated protein does not have to have cancer-specific, cancer-restricted, tumor-specific, or tumor-restricted expression. Examples of proteins that are considered cancer-specific or cancer-restricted are cancer testis antigens or oncofetal antigens. Cancer testis antigens (CTAs) are a large family of tumor-associated antigens expressed in human tumors of different histological origin but not in normal tissue, except for male germ cells. In cancer, these developmental antigens can be re-expressed and can serve as a locus of immune activation. Oncofetal antigens (OFAs) are proteins that are typically present only during fetal development but are found in adults with certain kinds of cancer.

As another example, a disease-associated antigen can be an infectious-disease-associated antigen. The term “infectious-disease-associated antigen” refers to an antigen whose presence is correlated with the occurrence or progression of a particular infectious disease. For example, the antigen can be in an infectious-disease-associated protein (i.e., a protein whose expression is correlated with the occurrence or progression of the infectious disease). Optionally, an infectious-disease-associated protein can be a protein that is expressed in a particular type of infectious disease but is not normally expressed in healthy adult tissue (i.e., a protein with infectious-disease-specific expression or infectious-disease-restricted expression). However, an infectious-disease-associated protein does not have to have infectious-disease-specific or infectious-disease-restricted expression. For example, the antigen can be a viral antigen or a bacterial antigen. Such antigens include, for example, molecular structures on the surface of viruses or bacteria (e.g., viral proteins or bacterial proteins) that are recognized by the immune system and are capable of triggering an immune response.

The term “epitope” refers to a site on an antigen to which an antigen-binding protein (e.g., antibody) binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996), herein incorporated by reference in its entirety for all purposes.

The term “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1 domain, a hinge, a C_{H}2 domain, and a C_{H}3 domain. A functional fragment of a heavy chain includes a fragment that is capable of specifically recognizing an epitope (e.g., recognizing the epitope with a K_{D} in the micromolar, nanomolar, or picomolar range), that is capable of expressing and secreting from a cell, and that comprises at least one CDR. Heavy chain variable domains are encoded by variable region nucleotide sequence, which generally comprises V_{H}, D_{H}, and J_{H} segments derived from a repertoire of V_{H}, D_{H}, and J_{H} segments present in the germline. Sequences, locations and nomenclature for V, D, and J heavy chain segments for various organisms can be found in IMGT database, which is accessible via the internet on the world wide web (www) at the URL “imgt.org.”

The term “light chain” includes an immunoglobulin light chain sequence from any organism, and unless otherwise specified includes human kappa (κ) and lambda (λ) light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region amino acid sequence. Light chain variable domains are encoded by the light chain variable region nucleotide sequence, which generally comprises light chain V_{L} and light chain J_{L} gene segments, derived from a repertoire of light chain V and J gene segments present in the germline. Sequences, locations and nomenclature for light chain V and J gene segments for various organisms can be found in IM4GT database, which is accessible via the internet on the world wide web (www) at the URL “imgt.org.” Light chains include those, e.g., that do not selectively bind either a first or a second epitope selectively bound by the epitope-binding protein in which they appear. Light chains also include those that bind and recognize, or assist the heavy chain with binding and recognizing, one or more epitopes selectively bound by the epitope-binding protein in which they appear.

The term “complementary determining region” or “CDR,” as used herein, includes an amino acid sequence encoded by a nucleic acid sequence of an organism's immunoglobulin genes that normally (i.e., in a wild type animal) appears between two framework regions in a variable region of a light or a heavy chain of an immunoglobulin molecule (e.g., an antibody or a T cell receptor). A CDR can be encoded by, for example, a germline sequence or a rearranged sequence, and, for example, by a naïve or a mature B cell or a T cell. A CDR can be somatically mutated (e.g., vary from a sequence encoded in an animal's germline), humanized, and/or modified with amino acid substitutions, additions, or deletions. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as a result of splicing or connecting the sequences (e.g., V-D-J recombination to form a heavy chain CDR3.

The term “unrearranged” includes the state of an immunoglobulin locus wherein V gene segments and J gene segments (for heavy chains, D gene segments as well) are maintained separately but are capable of being joined to form a rearranged V(D)J gene that comprises a single V, (D), J of the V(D)J repertoire. The term “rearranged” includes a configuration of a heavy chain or light chain immunoglobulin locus wherein a V segment is positioned immediately adjacent to a D-J or J segment in a conformation encoding essentially a complete V_{H} or V_{L} domain, respectively.

The antigen-binding protein can be a single-chain antigen-binding protein such as an scFv. Alternatively, the antigen-binding protein is not a single-chain antigen-binding protein. For example, the antigen-binding protein can include separate light and heavy chains. The heavy chain coding sequence can be upstream of the light chain coding sequence, or the light chain coding sequence can be upstream of the heavy chain coding sequence. In one specific example, the heavy chain coding sequence is upstream of the light chain coding sequence. For example, the heavy chain coding sequence can comprise V_{H}, D_{H}, and J_{H} segments, and the light chain coding sequence can comprise light chain V_{L} and light chain J_{L} gene segments. The antigen-binding protein coding sequence can be operably linked to an exogenous promoter in the nucleic acid construct. Likewise, the antigen-binding protein coding sequence in the nucleic acid construct can include an exogenous signal sequence for secretion. In a specific example, the antigen-binding protein comprises separate light and heavy chains, and each chain is operably linked to separate exogenous signal sequences.

Signal sequences (i.e., N-terminal signal sequences) mediate targeting of nascent secretory and membrane proteins to the endoplasmic reticulum (ER) in a signal recognition particle (SRP)-dependent manner. Usually, signal sequences are cleaved off co-translationally so that signal peptides and mature proteins are generated. Examples of exogenous signal sequences or signal peptides that can be used include, for example, the signal sequence/peptide from mouse albumin, human albumin, mouse ROR1, human ROR1, human azurocidin, Cricetulus griseus Ig kappa chain V III region MOPC 63 like, and human Ig kappa chain V III region VG. Any other known signal sequence/peptide can also be used. In a specific example, an ROR1 signal sequence is used.

One or more of the nucleic acids in the antigen-binding-protein coding sequence (e.g., a heavy chain coding sequence and a light chain coding sequence) can be together in a multicistronic expression construct. For example, a nucleic acid encoding a heavy chain and a light chain can be together in a bicistronic expression construct. Multicistronic expression vectors simultaneously express two or more separate proteins from the same mRNA (i.e., a transcript produced from the same promoter). Suitable strategies for multicistronic expression of proteins include, for example, the use of a 2A peptide and the use of an internal ribosome entry site (TRES). As one example, such multicistronic vectors can use one or more internal ribosome entry sites (IRES) to allow for initiation of translation from an internal region of an mRNA. As another example, such multicistronic vectors can use one or more 2A peptides. These peptides are small “self-cleaving” peptides, generally having a length of 18-22 amino acids and produce equimolar levels of multiple genes from the same mRNA. Ribosomes skip the synthesis of a glycyl-prolyl peptide bond at the C-terminus of a 2A peptide, leading to the “cleavage” between a 2A peptide and its immediate downstream peptide. See, e.g., Kim et al. (2011) PLoS One 6(4): e18556, herein incorporated by reference in its entirety for all purposes. The “cleavage” occurs between the glycine and proline residues found on the C-terminus, meaning the upstream cistron will have a few additional residues added to the end, while the downstream cistron will start with the proline. As a result, the “cleaved-off” downstream peptide has proline at its N-terminus. 2A-mediated cleavage is a universal phenomenon in all eukaryotic cells. 2A peptides have been identified from picornaviruses, insect viruses and type C rotaviruses. See, e.g., Szymczak et al. (2005) Expert Opin Biol Ther 5:627-638, herein incorporated by reference in its entirety for all purposes. Examples of 2A peptides that can be used include Thosea asigna virus 2A (T2A); porcine teschovirus-1 2A (P2A); equine rhinitis A virus (ERAV) 2A (E2A); and FMDV 2A (F2A). Exemplary T2A, P2A, E2A, and F2A sequences include the following: T2A (EGRGSLLTCGDVEENPGP; SEQ ID NO: 31); P2A (ATNFSLLKQAGDVEENPGP; SEQ ID NO: 32); E2A (QCTNYALLKLAGDVESNPGP; SEQ ID NO: 33); and F2A (VKQTLNFDLLKLAGDVESNPGP; SEQ ID NO: 34). GSG residues can be added to the 5′ end of any of these peptides to improve cleavage efficiency.

In some nucleic acid constructs, a nucleic acid encoding a furin cleavage site is included between the light chain coding sequence and the heavy chain coding sequence. In some nucleic acid constructs, a nucleic acid encoding a linker (e.g., GSG) is included between the light chain coding sequence and the heavy chain coding sequence (e.g., directly upstream of the 2A peptide coding sequence). For example, a furin cleavage site can be included upstream of a 2A peptide, with both the furin cleavage site and the 2A peptide being located between the light chain and the heavy chain (i.e., upstream chain—furin cleavage site—2A peptide—downstream chain). During translation, a first cleavage event will occur at the 2A peptide sequence. However, most of the 2A peptide will remain attached as a remnant to the C-terminus of the upstream chain (e.g., light chain if the light chain is upstream of the heavy chain, or heavy chain if the heavy chain is upstream of the light chain), with one amino acid added to the N-terminus of the downstream chain (or the N-terminus of a signal sequence, if a signal sequence is included upstream of the downstream chain). A second cleavage event, initiated at the furin cleavage site, yields the upstream chain without the 2A remnants in order to obtain a more native heavy chain or light chain by post-translational processing.

The term “chimeric antigen receptor” (CAR) refers to molecules that combine a binding domain against a component present on the target cell, for example an antibody-based specificity for a desired antigen, with a T cell receptor-activating intracellular domain to generate a chimeric protein that exhibits a specific anti-target cellular immune activity. For example, CARs can comprise an extracellular single chain antibody-binding domain (scFv) fused to the intracellular signaling domain of the T cell antigen receptor complex zeta chain, and have the ability, when expressed in T cells, to redirect antigen recognition based on the monoclonal antibody's specificity.

The polypeptide of interest can be a secreted polypeptide (e.g., a protein that is secreted by the cell and/or is functionally active as a soluble extracellular protein). Alternatively, the polypeptide of interest can be an intracellular polypeptide (e.g., a protein that is not secreted by the cell and is functionally active within the cell, including soluble cytosolic polypeptides).

The polypeptide of interest can be a wild type polypeptide. Alternatively, the polypeptide of interest can be a variant or mutant polypeptide.

In one example, the polypeptide of interest is a liver protein (e.g., a protein that is, endogenously produced in the liver and/or functionally active in the liver). In another example, the polypeptide of interest can be a circulating protein that is produced by the liver. In another example, the polypeptide of interest can be a non-liver protein.

The polypeptide of interest can be an exogenous polypeptide. An “exogenous” polypeptide coding sequence can refer to a coding sequence that has been introduced from an exogenous source to a site within a host cell genome (e.g., at a genomic locus such as a genomic safe harbor locus described herein). That is, the exogenous polypeptide coding sequence is exogenous with respect to its insertion site, and the polypeptide of interest expressed from such an exogenous coding sequence is referred to as an exogenous polypeptide. The exogenous coding sequence can be naturally-occurring or engineered, and can be wild type or a variant. The exogenous coding sequence may include nucleotide sequences other than the sequence that encodes the exogenous polypeptide (e.g., an internal ribosomal entry site). The exogenous coding sequence can be a coding sequence that occurs naturally in the host genome, as a wild type or a variant (e.g., mutant). For example, although the host cell contains the coding sequence of interest (as a wild type or as a variant), the same coding sequence or variant thereof can be introduced as an exogenous source (e.g., for expression at a locus that is highly expressed). The exogenous coding sequence can also be a coding sequence that is not naturally occurring in the host genome, or that expresses an exogenous polypeptide that does not naturally occur in the host genome. An exogenous coding sequence can include an exogenous nucleic acid sequence (e.g., a nucleic acid sequence is not endogenous to the recipient cell), or may be exogenous with respect to its insertion site and/or with respect to its recipient cell.

The coding sequence for the polypeptide of interest can be codon-optimized for expression in a host cell. For example, the coding sequence can be codon optimized or may use one or more alternative codons for one or more amino acids of the polypeptide of interest (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

### (2) Vectors

The nucleic acid constructs disclosed herein can be provided in a vector for expression or for integration into and expression from a target genomic locus (e.g., a genomic safe harbor locus). A vector can comprise additional sequences such as, for example, replication origins, promoters, and genes encoding antibiotic resistance. A vector can also comprise nuclease agent components as disclosed elsewhere herein. For example, a vector can comprise a nucleic acid construct encoding a product of interest (e.g., polypeptide of interest), a CRISPR/Cas system (nucleic acids encoding Cas protein and gRNA), one or more components of a CRISPR/Cas system, or a combination thereof (e.g., a nucleic acid construct and a gRNA). In some cases, a vector comprising a nucleic acid construct encoding a product of interest (e.g., polypeptide of interest) does not comprise any components of the nuclease agents described herein (e.g., does not comprise a nucleic acid encoding a Cas protein and does not comprise a nucleic acid encoding a gRNA). Some such vectors comprise homology arms corresponding to target sites in the target genomic locus. Other such vectors do not comprise any homology arms.

Some vectors may be circular. Alternatively, the vector may be linear. The vector can be packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper components to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In rAAV genomes, a gene expression cassette can be placed between ITR sequences. Typically, rAAV genome cassettes comprise of a promoter to drive expression of a transgene, followed by a polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, AAV-DJ, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

### C. Nuclease Agents and CRISPR/Cas Systems

The methods and compositions disclosed herein can utilize nuclease agents such as Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems or components of such systems to modify a target genomic locus in a target locus such as a genomic safe harbor locus for insertion of a nucleic acid construct as disclosed herein. Generally, the nuclease agents involve the use of engineered cleavage systems to induce a double strand break or a nick (i.e., a single strand break) in a nuclease target site. Cleavage or nicking can occur through the use of specific nucleases such as engineered ZFNs, TALENs, or CRISPR/Cas systems with an engineered guide RNA to guide specific cleavage or nicking of the nuclease target site. Any nuclease agent that induces a nick or double-strand break at a desired target sequence can be used in the methods and compositions disclosed herein. The nuclease agent can be used to create a site of insertion at a desired locus (genomic safe harbor locus) within a host genome, at which site the nucleic acid construct is inserted to express the product of interest (e.g., polypeptide of interest). The product of interest (e.g., polypeptide of interest) may be exogenous with respect to its insertion site or locus, such as an extragenic genomic safe harbor locus from which product of interest (e.g., polypeptide of interest) is not normally expressed.

In one example, the nuclease agent is a CRISPR/Cas system. In another example, the nuclease agent comprises one or more ZFNs. In yet another example, the nuclease agent comprises one or more TALENs. In a specific example, the CRISPR/Cas systems or components of such systems target a genomic safe harbor locus as described elsewhere herein within a cell. In a more specific example, the CRISPR/Cas systems or components of such systems target a L-SH5, L-SH18, or L-SH20 genomic safe harbor locus (e.g., a human L-SH5, L-SH18, or L-SH20 genomic safe harbor locus) as described herein within a cell. In a more specific example, the CRISPR/Cas systems or components of such systems target a human L-SH5, L-SH18, or L-SH20 genomic safe harbor locus as described herein within a cell. In a more specific example, the CRISPR/Cas systems or components of such systems target a mouse L-SH5, L-SH18, or L-SH20 genomic safe harbor locus as described herein within a cell.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting a genomic safe harbor locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus.

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### (1) Target Genomic Loci

Any target genomic locus capable of expressing a gene can be used, such as a genomic safe harbor locus as described elsewhere herein. Target genomic loci used herein can be genomic safe harbor loci. Genomic safe harbor loci include chromosomal loci where transgenes or other exogenous nucleic acid inserts can be stably and reliably expressed in tissues of interest without overtly altering cell behavior or phenotype (i.e., without any deleterious effects on the host cell). For example, the genomic safe harbor locus can be one in which expression of the inserted gene sequence is not perturbed by any read-through expression from neighboring genes. For example, genomic safe harbor loci can include chromosomal loci where exogenous DNA can integrate and function in a predictable manner without adversely affecting endogenous gene structure or expression. The genomic safe harbor loci can be targeted with high efficiency, and safe harbor loci can be disrupted with no overt phenotype. Genomic safe harbor loci can include extragenic regions or intragenic regions such as, for example, loci within genes that are non-essential, dispensable, or able to be disrupted without overt phenotypic consequences.

A genomic safe harbor locus described herein can be a genomic locus that, when targeted for integration in a subject, does not cause changes in liver functionality. A genomic safe harbor locus described herein can be a genomic locus that, when targeted for integration in a subject, does not cause changes in alanine aminotransferase (alanine transaminase or ALT) levels. A genomic safe harbor locus described herein can be a genomic locus that, when targeted for integration in a subject, does not cause changes in aspartate aminotransferase (AST) levels. A genomic safe harbor locus described herein can be a genomic locus that, when targeted for integration in a subject, does not cause changes in alkaline phosphatase (ALP) levels. A genomic safe harbor locus described herein can be a genomic locus that, when targeted for integration in a subject, does not cause changes in body weight. A genomic safe harbor locus described herein can be a genomic locus that, when targeted for integration in a subject, does not cause changes in proliferation such as in a target organ such as the liver (e.g., as assessed by Ki67 staining). A genomic safe harbor locus described herein can be a genomic locus that, when targeted for integration in a subject, does not cause oncogenic transformation such as in a target organ such as the liver (e.g., as assessed by H&E staining).

A genomic safe harbor locus described herein can be a genomic locus with an open chromatin configuration in the liver such that exogenous nucleic acid inserts can be stably and reliably expressed in the liver. Alternatively, a genomic safe harbor locus can be a genomic locus with an open chromatin configuration in another tissue or cell type (e.g., hematopoietic cells, such as hematopoietic stem cells, T cells, B cells, and/or macrophages) such that exogenous nucleic acid inserts can be stably and reliably expressed in that tissue or cell type.

A genomic safe harbor locus described herein can be an extragenic genomic safe harbor locus (i.e., occurring outside of a gene). In a specific example, a genomic safe harbor locus described herein is an extragenic genomic safe harbor locus with an open chromatin configuration in the liver.

In a specific example, the genomic safe harbor locus can be one that is more than 300 kb from any cancer-related gene (e.g., to prevent insertional oncogenesis), more than 300 kb from any miRNA or small RNA (e.g., to preserve regulation of gene expression and cellular development), more than 50 kb from the 5′ end of any gene (e.g., to avoid perturbing endogenous gene expression), more than 50 kb from any replication origin, more than 50 kb from any ultra-conserved elements (e.g., non-coding intragenic or intergenic regions that are completely conserved in human, mouse, and rat genomes), outside of copy number variable regions, and in open chromatin (as determined, e.g., by ATAC-Seq analysis (e.g., in human liver biopsy samples)). In addition, the genomic safe harbor locus can be one that does not overlap with regions predicted to be regulatory regions (e.g., H3K4me1, H3K27ac, and/or H3K4me3 markers), heterochromatin regions (e.g., H3K9me3 marker), or participating into chromatin organization (e.g., CTCF signals).

In a specific example, the genomic safe harbor locus is selected from the following genomic locations: (i) human chromosome 13, coordinates 77460242-77460537 (referred to herein as L-SH5) or a corresponding region (e.g., orthologous or syntenic region) in a non-human animal, non-human mammal (e.g., non-human primate), or rodent, such as a rat or a mouse; (ii) human chromosome 6, coordinates 170031084-170031382 (referred to herein as L-SH18) or a corresponding region (e.g., orthologous or syntenic region) in a non-human animal, non-human mammal (e.g., non-human primate), or rodent, such as a rat or a mouse; and (iii) human chromosome 9, coordinates 25207412-25207703 (referred to herein as L-SH20) or a corresponding region (e.g., orthologous or syntenic region) in a non-human animal, non-human mammal (e.g., non-human primate), or rodent, such as a rat or a mouse.

In a specific example, the genomic safe harbor locus is selected from the following genomic coordinates: (i) about 77460242 to about 77460537 on human chromosome 13 (corresponds to L-SH5) or a corresponding region (e.g., orthologous or syntenic region) in a non-human animal, non-human mammal (e.g., non-human primate), or rodent, such as a rat or a mouse; (ii) about 170031084 to about 170031382 on human chromosome 6 (corresponds to L-SH18) or a corresponding region (e.g., orthologous or syntenic region) in a non-human animal, non-human mammal (e.g., non-human primate), or rodent, such as a rat or a mouse; and (iii) about 25207412 to about 25207703 on human chromosome 9 (corresponds to L-SH20) or a corresponding region (e.g., orthologous or syntenic region) in a non-human animal, non-human mammal (e.g., non-human primate), or rodent, such as a rat or a mouse. The term “about” when referring to genomic coordinates means ±20 base pairs. In other examples, the genomic safe harbor locus is near the regions identified by the above coordinates. The term “near” when referring to genomic coordinates means ±5 kb, ±4 kb, ±3 kb, ±2 kb, ±1 kb, ±0.5 kb, ±0.4 kb, ±0.3 kb, ±0.2 kb, or ±0.1 kb.

In one specific example, the genomic safe harbor locus is human L-SH5 (chromosome 13, coordinates 77460242-77460537) or a corresponding region (e.g., orthologous or syntenic region) in a non-human animal, non-human mammal (e.g., non-human primate), or rodent, such as a rat or a mouse. Syntenic regions are derived from a single ancestral genomic region. For example, syntenic regions can be from different organisms and are derived from speciation.

In another specific example, the genomic safe harbor locus is human L-SH18 (chromosome 6, coordinates 170031084-170031382) or a corresponding region (e.g., orthologous or syntenic region) in a non-human animal, non-human mammal (e.g., non-human primate), or rodent, such as a rat or a mouse.

In another specific example, the genomic safe harbor locus is human L-SH20 (chromosome 9, coordinates 25207412-25207703) or a corresponding region (e.g., orthologous or syntenic region) in a non-human animal, non-human mammal (e.g., non-human primate), or rodent, such as a rat or a mouse.

In one specific example, the genomic safe harbor locus corresponds to human L-SH5 (coordinates of about 77460242 to about 77460537 on chromosome 13) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means±20 base pairs. In other examples, the genomic safe harbor locus is near the region identified by the above coordinates. The term “near” when referring to genomic coordinates means ±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to human L-SH18 (coordinates of about 170031084 to about 170031382 on chromosome 6) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means ±20 base pairs. In other examples, the genomic safe harbor locus is near the region identified by the above coordinates. The term “near” when referring to genomic coordinates means ±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to human L-SH20 (coordinates of about 25207412 to about 25207703 on chromosome 9) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In a specific example, the genomic safe harbor locus is selected from the following genomic locations: (i) mouse chromosome 14, coordinates 103,450,397-103,451,396 (referred to herein as mouse L-SH5) or a corresponding region (e.g., orthologous or syntenic region) in a non-human animal, non-human mammal (e.g., non-human primate), or rodent, such as a rat; (ii) mouse chromosome 17, coordinates 15,226,387-15,227,386 (referred to herein as mouse L-SH18) or a corresponding region (e.g., orthologous or syntenic region) in a non-human animal, non-human mammal (e.g., non-human primate), or rodent, such as a rat; and (iii) mouse chromosome 4, coordinates 92,827,563-92,828,592 (referred to herein as mouse L-SH20) or a corresponding region (e.g., orthologous or syntenic region) in a non-human animal, non-human mammal (e.g., non-human primate), or rodent, such as a rat.

In a specific example, the genomic safe harbor locus is selected from the following genomic coordinates: (i) about 103,450,397 to about 103,451,396 on mouse chromosome 14 (corresponds to mouse L-SH5) or a corresponding region (e.g., orthologous or syntenic region) in a non-human animal, non-human mammal (e.g., non-human primate), or rodent, such as a rat; (ii) about 15,226,387 to about 15,227,386 on mouse chromosome 17 (corresponds to mouse L-SH18) or a corresponding region (e.g., orthologous or syntenic region) in a non-human animal, non-human mammal (e.g., non-human primate), or rodent, such as a rat; and (iii) about 92,827,563 to about 92,828,592 on mouse chromosome 4 (corresponds to mouse L-SH20) or a corresponding region (e.g., orthologous or syntenic region) in a non-human animal, non-human mammal (e.g., non-human primate), or rodent, such as a rat. The term “about” when referring to genomic coordinates means±20 base pairs. In other examples, the genomic safe harbor locus is near the regions identified by the above coordinates. The term “near” when referring to genomic coordinates means ±5 kb, ±4 kb, ±3 kb, ±2 kb, ±1 kb, ±0.5 kb, ±0.4 kb, ±0.3 kb, ±0.2 kb, or ±0.1 kb.

In one specific example, the genomic safe harbor locus is mouse L-SH5 (chromosome 14, coordinates 103,450,397-103,451,396) or a corresponding region (e.g., orthologous or syntenic region) in a non-human animal, non-human mammal (e.g., non-human primate), or rodent, such as a rat. Syntenic regions are derived from a single ancestral genomic region. For example, syntenic regions can be from different organisms and are derived from speciation.

In another specific example, the genomic safe harbor locus is mouse L-SH18 (chromosome 17, coordinates 15,226,387-15,227,386) or a corresponding region (e.g., orthologous or syntenic region) in a non-human animal, non-human mammal (e.g., non-human primate), or rodent, such as a rat.

In another specific example, the genomic safe harbor locus is mouse L-SH20 (chromosome 4, coordinates 92,827,563-92,828,592) or a corresponding region (e.g., orthologous or syntenic region) in a non-human animal, non-human mammal (e.g., non-human primate), or rodent, such as a rat.

In one specific example, the genomic safe harbor locus corresponds to mouse L-SH5 (coordinates of about 103,450,397 to about 103,451,396 on mouse chromosome 14) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to mouse L-SH18 (coordinates of about 15,226,387 to about 15,227,386 on mouse chromosome 17) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to mouse L-SH20 (coordinates of about 92,827,563 to about 92,828,592 on mouse chromosome 4) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

### (2) Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. An exemplary SpCas9 protein sequence is set forth in SEQ ID NO: 1 (encoded by the DNA sequence set forth in SEQ ID NO: 2). Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes.

Another example of a Cas protein is a Cpf1 (CRISPR from Prevotella and Francisella 1; Cas12a) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3).759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC20171, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number AOQ7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasD (CasPhi or Cas12j), which is uniquely found in bacteriophages. Cas4 is less than 1000 amino acids in size (e.g., 700-800 amino acids). CasΦ cleavage generates staggered 5′ overhangs. A single RuvC active site in Cas(is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and Cas(D generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasD) proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas protein can be fused to a cleavage domain. See WO 2014/089290, herein incorporated by reference in its entirety for all purposesCas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 3) or PKKKRRV (SEQ ID NO: 4). The NLS may be a bipartite sequence, such as the NLS of nucleoplasmin, KRPAATKKAGQAKKKK (SEQ ID NO: 5). In a specific example, a single PKKKRKV (SEQ ID NO: 3) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, ZsYellow1), blue fluorescent proteins (e.g., eBFP, eBFP2, Azurite, mKalama1, GFPuv, Sapphire, T-sapphire), cyan fluorescent proteins (e.g., eCFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, Si, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med Chem. 5(1):41-55; Duckworth et al. (2007) Angew. Chem. Int. Ed. Engl. 46(46):8819-8822; Schaeffer and Dixon (2009) Australian J Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in a human cell, a human liver cell, or a human hepatocyte. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery. In preferred embodiments, promotors are accepted by regulatory authorities for use in humans. In certain embodiments, promotors drive expression in a liver cell.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV8-mediated delivery). For example, the nuclease agent can be CRISPR/Cas9, and a Cas9 mRNA and a gRNA (e.g., targeting a human L-SH5, L-SH18, or L-SH20 genomic safe harbor locus as described herein) can be delivered via LNP-mediated delivery or AAV-mediated delivery. For example, the nuclease agent can be CRISPR/Cas9, and a Cas9 mRNA and a gRNA (e.g., targeting a mouse L-SH5, L-SH18, or L-SH20 genomic safe harbor locus as described herein) can be delivered via LNP-mediated delivery or AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. mRNA encoding Cas proteins can also be capped. Cas mRNAs can further comprise a poly-adenylated (poly-A or poly(A) or poly-adenine) tail. For example, a Cas mRNA can include a modification to one or more nucleosides within the mRNA, the Cas mRNA can be capped, and the Cas mRNA can comprise a poly(A) tail.

### (3) Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasqD, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 6) or GUUUUAGAGCUAUGCUGUUUUG (SEQ ID NO: 7). Any of the DNA-targeting segments disclosed herein can be joined to the 5′ end of SEQ ID NO: 6 or 7 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of

(SEQ ID NO: 8)
AGCAUAGCAAGUUAAAAUAAGGCUAGUCCGUUAUCAACUUGAAAA
AGUGGCACCGAGUCGGUGCUUU,
(SEQ ID NO: 9)
AAACAGCAUAGCAAGUUAAAAUAAGGCUAGUCCGUUAUCAACUUG
AAAAAGUGGCACCGAGUCGGUGCUUUU,
or
(SEQ ID NO: 10)
GUUGGAACCAUUCAAAACAGCAUAGCAAGUUAAAAUAAGGCUAGU
CCGUUAUCAACUUGAAAAAGUGGCACCGAGUCGGUGC

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As one example, a guide RNA targeting a genomic safe harbor locus described herein can comprise a DNA-targeting segment (i.e., guide sequence) comprising, consisting essentially of, or consisting of the sequence (DNA-targeting segment) set forth in any one of SEQ ID NOS: 25-27, 45-47, and 228-314. Alternatively, a guide RNA targeting a genomic safe harbor locus described herein can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 25-27, 45-47, and 228-314. Alternatively, a guide RNA targeting a genomic safe harbor locus described herein can comprise a DNA-targeting segment that is at least 75%, at least 80%, at least 85%, at least 90%, or at least 95% identical to the sequence (DNA-targeting segment) set forth in any one of SEQ ID NOS: 25-27, 45-47, and 228-314. Alternatively a guide RNA targeting a genomic safe harbor locus described herein can comprise a DNA-targeting segment that is at least 90% or at least 95% identical to the sequence (DNA-targeting segment) set forth in any one of SEQ ID NOS: 25-27, 45-47, and 228-314. Alternatively, a guide RNA targeting a genomic safe harbor locus described herein can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 25-27, 45-47, and 228-314. Alternatively, a guide RNA targeting a genomic safe harbor locus described herein can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 25-27, 45-47, and 228-314. Alternatively, a guide RNA targeting a genomic safe harbor locus described herein can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 25-27, 45-47, and 228-314. Alternatively, a guide RNA targeting a genomic safe harbor locus described herein can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 25-27, 45-47, and 228-314.

As one example, a guide RNA targeting a genomic safe harbor locus described herein can comprise a DNA-targeting segment (i.e., guide sequence) comprising, consisting essentially of, or consisting of the sequence (DNA-targeting segment) set forth in any one of SEQ ID NOS: 315-404. Alternatively, a guide RNA targeting a genomic safe harbor locus described herein can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 315-404. Alternatively, a guide RNA targeting a genomic safe harbor locus described herein can comprise a DNA-targeting segment that is at least 75%, at least 80%, at least 85%, at least 90%, or at least 95% identical to the sequence (DNA-targeting segment) set forth in any one of SEQ ID NOS: 315-404. Alternatively a guide RNA targeting a genomic safe harbor locus described herein can comprise a DNA-targeting segment that is at least 90% or at least 95% identical to the sequence (DNA-targeting segment) set forth in any one of SEQ ID NOS: 315-404. Alternatively, a guide RNA targeting a genomic safe harbor locus described herein can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 315-404. Alternatively, a guide RNA targeting a genomic safe harbor locus described herein can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 315-404. Alternatively, a guide RNA targeting a genomic safe harbor locus described herein can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 315-404. Alternatively, a guide RNA targeting a genomic safe harbor locus described herein can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 315-404.

As one example, a guide RNA targeting a genomic safe harbor locus described herein can comprise a DNA-targeting segment (i.e., guide sequence) comprising, consisting essentially of, or consisting of the sequence (DNA-targeting segment) set forth in any one of SEQ ID NOS: 25-27 and 45-47. As one example, a guide RNA targeting a genomic safe harbor locus described herein can comprise a DNA-targeting segment (i.e., guide sequence) comprising, consisting essentially of, or consisting of the sequence (DNA-targeting segment) set forth in any one of SEQ ID NOS: 25-27. Alternatively, a guide RNA targeting a genomic safe harbor locus described herein can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 25-27 and 45-47. Alternatively, a guide RNA targeting a genomic safe harbor locus described herein can comprise a DNA-targeting segment that is at least 75%, at least 80%, at least 85%, at least 90%, or at least 95% identical to the sequence (DNA-targeting segment) set forth in any one of SEQ ID NOS: 25-27 and 45-47. Alternatively a guide RNA targeting a genomic safe harbor locus described herein can comprise a DNA-targeting segment that is at least 90% or at least 95% identical to the sequence (DNA-targeting segment) set forth in any one of SEQ ID NOS: 25-27 and 45-47. Alternatively, a guide RNA targeting a genomic safe harbor locus described herein can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 25-27 and 45-47. Alternatively, a guide RNA targeting a genomic safe harbor locus described herein can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 25-27 and 45-47. Alternatively, a guide RNA targeting a genomic safe harbor locus described herein can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 25-27 and 45-47. Alternatively, a guide RNA targeting a genomic safe harbor locus described herein can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 25-27 and 45-47.

As another example, a guide RNA targeting human L-SH5 (chromosome 13, coordinates 77460242-77460537) can comprise a DNA-targeting segment (i.e., guide sequence) comprising, consisting essentially of, or consisting of the sequence (DNA-targeting segment) set forth in any one of SEQ ID NOS: 25, 45, and 228-256. Alternatively, a guide RNA targeting human L-SH5 (chromosome 13, coordinates 77460242-77460537) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 25, 45, and 228-256. Alternatively, a guide RNA targeting human L-SH5 (chromosome 13, coordinates 77460242-77460537) can comprise a DNA-targeting segment that is at least 75%, at least 80%, at least 85%, at least 90%, or at least 95% identical to the sequence (DNA-targeting segment) set forth in any one of SEQ ID NOS: 25, 45, and 228-256. Alternatively, a guide RNA targeting human L-SH5 (chromosome 13, coordinates 77460242-77460537) can comprise a DNA-targeting segment that is at least 90% or at least 95% identical to the sequence (DNA-targeting segment) set forth in any one of SEQ ID NOS: 25, 45, and 228-256. Alternatively, a guide RNA targeting human L-SH5 (chromosome 13, coordinates 77460242-77460537) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 25, 45, and 228-256. Alternatively, a guide RNA targeting human L-SH5 (chromosome 13, coordinates 77460242-77460537) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 25, 45, and 228-256. Alternatively, a guide RNA targeting human L-SH5 (chromosome 13, coordinates 77460242-77460537) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 25, 45, and 228-256. Alternatively, a guide RNA targeting human L-SH5 (chromosome 13, coordinates 77460242-77460537) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 25, 45, and 228-256.

As another example, a guide RNA targeting human L-SH5 (chromosome 13, coordinates 77460242-77460537) can comprise a DNA-targeting segment (i.e., guide sequence) comprising, consisting essentially of, or consisting of the sequence (DNA-targeting segment) set forth in any one of SEQ ID NOS: 25, 45, 235, 237, and 246. Alternatively, a guide RNA targeting human L-SH5 (chromosome 13, coordinates 77460242-77460537) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 25, 45, 235, 237, and 246. Alternatively, a guide RNA targeting human L-SH5 (chromosome 13, coordinates 77460242-77460537) can comprise a DNA-targeting segment that is at least 75%, at least 80%, at least 85%, at least 90%, or at least 95% identical to the sequence (DNA-targeting segment) set forth in any one of SEQ ID NOS: 25, 45, 235, 237, and 246. Alternatively, a guide RNA targeting human L-SH5 (chromosome 13, coordinates 77460242-77460537) can comprise a DNA-targeting segment that is at least 90% or at least 95% identical to the sequence (DNA-targeting segment) set forth in any one of SEQ ID NOS: 25, 45, 235, 237, and 246. Alternatively, a guide RNA targeting human L-SHS (chromosome 13, coordinates 77460242-77460537) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 25, 45, 235, 237, and 246. Alternatively, a guide RNA targeting human L-SH5 (chromosome 13, coordinates 77460242-77460537) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 25, 45, 235, 237, and 246. Alternatively, a guide RNA targeting human L-SH5 (chromosome 13, coordinates 77460242-77460537) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 25, 45, 235, 237, and 246. Alternatively, a guide RNA targeting human L-SH5 (chromosome 13, coordinates 77460242-77460537) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 25, 45, 235, 237, and 246.

As another example, a guide RNA targeting human L-SH5 (chromosome 13, coordinates 77460242-77460537) can comprise a DNA-targeting segment (i.e., guide sequence) comprising, consisting essentially of, or consisting of the sequence (DNA-targeting segment) set forth in SEQ ID NO: 25 or 45. As another example, a guide RNA targeting human L-SH5 (chromosome 13, coordinates 77460242-77460537) can comprise a DNA-targeting segment (i.e., guide sequence) comprising, consisting essentially of, or consisting of the sequence (DNA-targeting segment) set forth in SEQ ID NO: 25. As another example, a guide RNA targeting human L-SH5 (chromosome 13, coordinates 77460242-77460537) can comprise a DNA-targeting segment (i.e., guide sequence) comprising, consisting essentially of, or consisting of the sequence (DNA-targeting segment) set forth in SEQ ID NO: 45. Alternatively, a guide RNA targeting human L-SH5 (chromosome 13, coordinates 77460242-77460537) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 25 or 45. Alternatively, a guide RNA targeting human L-SH5 (chromosome 13, coordinates 77460242-77460537) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 25. Alternatively, a guide RNA targeting human L-SH5 (chromosome 13, coordinates 77460242-77460537) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 45. Alternatively, a guide RNA targeting human L-SH5 (chromosome 13, coordinates 77460242-77460537) can comprise a DNA-targeting segment that is at least 75%, at least 80%, at least 85%, at least 90%, or at least 95% identical to the sequence (DNA-targeting segment) set forth in SEQ ID NO: 25 or 45. Alternatively, a guide RNA targeting human L-SH5 (chromosome 13, coordinates 77460242-77460537) can comprise a DNA-targeting segment that is at least 75%, at least 80%, at least 85%, at least 90%, or at least 95% identical to the sequence (DNA-targeting segment) set forth in SEQ ID NO: 25. Alternatively, a guide RNA targeting human L-SH5 (chromosome 13, coordinates 77460242-77460537) can comprise a DNA-targeting segment that is at least 75%, at least 80%, at least 85%, at least 90%, or at least 95% identical to the sequence (DNA-targeting segment) set forth in SEQ ID NO: 45. Alternatively, a guide RNA targeting human L-SHS (chromosome 13, coordinates 77460242-77460537) can comprise a DNA-targeting segment that is at least 90% or at least 95% identical to the sequence (DNA-targeting segment) set forth in SEQ ID NO: 25 or 45. Alternatively, a guide RNA targeting human L-SH5 (chromosome 13, coordinates 77460242-77460537) can comprise a DNA-targeting segment that is at least 90% or at least 95% identical to the sequence (DNA-targeting segment) set forth in SEQ ID NO: 25. Alternatively, a guide RNA targeting human L-SH5 (chromosome 13, coordinates 77460242-77460537) can comprise a DNA-targeting segment that is at least 90% or at least 95% identical to the sequence (DNA-targeting segment) set forth in SEQ ID NO: 45. Alternatively, a guide RNA targeting human L-SH5 (chromosome 13, coordinates 77460242-77460537) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 25 or 45. Alternatively, a guide RNA targeting human L-SH5 (chromosome 13, coordinates 77460242-77460537) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 25. Alternatively, a guide RNA targeting human L-SH5 (chromosome 13, coordinates 77460242-77460537) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 45. Alternatively, a guide RNA targeting human L-SH5 (chromosome 13, coordinates 77460242-77460537) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 25 or 45. Alternatively, a guide RNA targeting human L-SHS (chromosome 13, coordinates 77460242-77460537) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 25. Alternatively, a guide RNA targeting human L-SH5 (chromosome 13, coordinates 77460242-77460537) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 45. Alternatively, a guide RNA targeting human L-SH5 (chromosome 13, coordinates 77460242-77460537) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 25 or 45. Alternatively, a guide RNA targeting human L-SH5 (chromosome 13, coordinates 77460242-77460537) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 25. Alternatively, a guide RNA targeting human L-SH5 (chromosome 13, coordinates 77460242-77460537) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 45. Alternatively, a guide RNA targeting human L-SH5 (chromosome 13, coordinates 77460242-77460537) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 25 or 45. Alternatively, a guide RNA targeting human L-SH5 (chromosome 13, coordinates 77460242-77460537) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 25. Alternatively, a guide RNA targeting human L-SH5 (chromosome 13, coordinates 77460242-77460537) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 45.

As another example, a guide RNA targeting mouse L-SH5 (chromosome 14, coordinates 103,450,397-103,451,396) can comprise a DNA-targeting segment (i.e., guide sequence) comprising, consisting essentially of, or consisting of the sequence (DNA-targeting segment) set forth in any one of SEQ ID NOS: 315-344. Alternatively, a guide RNA targeting mouse L-SH5 (chromosome 14, coordinates 103,450,397-103,451,396) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 315-344. Alternatively, a guide RNA targeting mouse L-SH5 (chromosome 14, coordinates 103,450,397-103,451,396) can comprise a DNA-targeting segment that is at least 75%, at least 80%, at least 85%, at least 90%, or at least 95% identical to the sequence (DNA-targeting segment) set forth in any one of SEQ ID NOS: 315-344. Alternatively, a guide RNA targeting mouse L-SH5 (chromosome 14, coordinates 103,450,397-103,451,396) can comprise a DNA-targeting segment that is at least 90% or at least 95% identical to the sequence (DNA-targeting segment) set forth in any one of SEQ ID NOS: 315-344. Alternatively, a guide RNA targeting mouse L-SH5 (chromosome 14, coordinates 103,450,397-103,451,396) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 315-344. Alternatively, a guide RNA targeting mouse L-SH5 (chromosome 14, coordinates 103,450,397-103,451,396) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 315-344. Alternatively, a guide RNA targeting mouse L-SH5 (chromosome 14, coordinates 103,450,397-103,451,396) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 315-344. Alternatively, a guide RNA targeting mouse L-SH5 (chromosome 14, coordinates 103,450,397-103,451,396) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 315-344.

As another example, a guide RNA targeting mouse L-SH5 (chromosome 14, coordinates 103,450,397-103,451,396) can comprise a DNA-targeting segment (i.e., guide sequence) comprising, consisting essentially of, or consisting of the sequence (DNA-targeting segment) set forth in any one of SEQ ID NOS: 318, 320, 321, and 341. Alternatively, a guide RNA targeting mouse L-SH5 (chromosome 14, coordinates 103,450,397-103,451,396) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 318, 320, 321, and 341. Alternatively, a guide RNA targeting mouse L-SH5 (chromosome 14, coordinates 103,450,397-103,451,396) can comprise a DNA-targeting segment that is at least 75%, at least 80%, at least 85%, at least 90%, or at least 95% identical to the sequence (DNA-targeting segment) set forth in any one of SEQ ID NOS: 318, 320, 321, and 341. Alternatively, a guide RNA targeting mouse L-SH5 (chromosome 14, coordinates 103,450,397-103,451,396) can comprise a DNA-targeting segment that is at least 90% or at least 95% identical to the sequence (DNA-targeting segment) set forth in any one of SEQ ID NOS: 318, 320, 321, and 341. Alternatively, a guide RNA targeting mouse L-SH5 (chromosome 14, coordinates 103,450,397-103,451,396) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 318, 320, 321, and 341. Alternatively, a guide RNA targeting mouse L-SH5 (chromosome 14, coordinates 103,450,397-103,451,396) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 318, 320, 321, and 341. Alternatively, a guide RNA targeting mouse L-SH5 (chromosome 14, coordinates 103,450,397-103,451,396) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 318, 320, 321, and 341. Alternatively, a guide RNA targeting mouse L-SH5 (chromosome 14, coordinates 103,450,397-103,451,396) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 318, 320, 321, and 341.

As another example, a guide RNA targeting human L-SH18 (chromosome 6, coordinates 170031084-170031382) can comprise a DNA-targeting segment (i.e., guide sequence) comprising, consisting essentially of, or consisting of the sequence (DNA-targeting segment) set forth in any one of SEQ ID NOS: 26, 46, and 257-285. Alternatively, a guide RNA targeting human L-SH18 (chromosome 6, coordinates 170031084-170031382) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 26, 46, and 257-285. Alternatively, a guide RNA targeting human L-SH18 (chromosome 6, coordinates 170031084-170031382) can comprise a DNA-targeting segment that is at least 75%, at least 80%, at least 85%, at least 90%, or at least 95% identical to the sequence (DNA-targeting segment) set forth in any one of SEQ ID NOS: 26, 46, and 257-285. Alternatively, a guide RNA targeting human L-SH18 (chromosome 6, coordinates 170031084-170031382) can comprise a DNA-targeting segment that is at least 90% or at least 95% identical to the sequence (DNA-targeting segment) set forth in any one of SEQ ID NOS: 26, 46, and 257-285. Alternatively, a guide RNA targeting human L-SH18 (chromosome 6, coordinates 170031084-170031382) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 26, 46, and 257-285. Alternatively, a guide RNA targeting human L-SH18 (chromosome 6, coordinates 170031084-170031382) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 26, 46, and 257-285. Alternatively, a guide RNA targeting human L-SH18 (chromosome 6, coordinates 170031084-170031382) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 26, 46, and 257-285. Alternatively, a guide RNA targeting human L-SH18 (chromosome 6, coordinates 170031084-170031382) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 26, 46, and 257-285.

As another example, a guide RNA targeting human L-SH18 (chromosome 6, coordinates 170031084-170031382) can comprise a DNA-targeting segment (i.e., guide sequence) comprising, consisting essentially of, or consisting of the sequence (DNA-targeting segment) set forth in any one of SEQ ID NOS: 26, 46, 268, 271, and 280. Alternatively, a guide RNA targeting human L-SH18 (chromosome 6, coordinates 170031084-170031382) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 26, 46, 268, 271, and 280. Alternatively, a guide RNA targeting human L-SH18 (chromosome 6, coordinates 170031084-170031382) can comprise a DNA-targeting segment that is at least 75%, at least 80%, at least 85%, at least 90%, or at least 95% identical to the sequence (DNA-targeting segment) set forth in any one of SEQ ID NOS: 26, 46, 268, 271, and 280. Alternatively, a guide RNA targeting human L-SH18 (chromosome 6, coordinates 170031084-170031382) can comprise a DNA-targeting segment that is at least 90% or at least 95% identical to the sequence (DNA-targeting segment) set forth in any one of SEQ ID NOS: 26, 46, 268, 271, and 280. Alternatively, a guide RNA targeting human L-SH18 (chromosome 6, coordinates 170031084-170031382) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 26, 46, 268, 271, and 280. Alternatively, a guide RNA targeting human L-SH18 (chromosome 6, coordinates 170031084-170031382) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 26, 46, 268, 271, and 280. Alternatively, a guide RNA targeting human L-SH18 (chromosome 6, coordinates 170031084-170031382) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 26, 46, 268, 271, and 280. Alternatively, a guide RNA targeting human L-SH18 (chromosome 6, coordinates 170031084-170031382) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 26, 46, 268, 271, and 280.

As another example, a guide RNA targeting human L-SH18 (chromosome 6, coordinates 170031084-170031382) can comprise a DNA-targeting segment (i.e., guide sequence) comprising, consisting essentially of, or consisting of the sequence (DNA-targeting segment) set forth in SEQ ID NO: 26 or 46. As another example, a guide RNA targeting human L-SH18 (chromosome 6, coordinates 170031084-170031382) can comprise a DNA-targeting segment (i.e., guide sequence) comprising, consisting essentially of, or consisting of the sequence (DNA-targeting segment) set forth in SEQ ID NO: 26. As another example, a guide RNA targeting human L-SH18 (chromosome 6, coordinates 170031084-170031382) can comprise a DNA-targeting segment (i.e., guide sequence) comprising, consisting essentially of, or consisting of the sequence (DNA-targeting segment) set forth in SEQ ID NO: 46. Alternatively, a guide RNA targeting human L-SH18 (chromosome 6, coordinates 170031084-170031382) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 26 or 46. Alternatively, a guide RNA targeting human L-SH18 (chromosome 6, coordinates 170031084-170031382) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 26. Alternatively, a guide RNA targeting human L-SH18 (chromosome 6, coordinates 170031084-170031382) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 46. Alternatively, a guide RNA targeting human L-SH18 (chromosome 6, coordinates 170031084-170031382) can comprise a DNA-targeting segment that is at least 75%, at least 80%, at least 85%, at least 90%, or at least 95% identical to the sequence (DNA-targeting segment) set forth in SEQ ID NO: 26 or 46. Alternatively, a guide RNA targeting human L-SH18 (chromosome 6, coordinates 170031084-170031382) can comprise a DNA-targeting segment that is at least 75%, at least 80%, at least 85%, at least 90%, or at least 95% identical to the sequence (DNA-targeting segment) set forth in SEQ ID NO: 26. Alternatively, a guide RNA targeting human L-SH18 (chromosome 6, coordinates 170031084-170031382) can comprise a DNA-targeting segment that is at least 75%, at least 80%, at least 85%, at least 90%, or at least 95% identical to the sequence (DNA-targeting segment) set forth in SEQ ID NO: 46. Alternatively, a guide RNA targeting human L-SH18 (chromosome 6, coordinates 170031084-170031382) can comprise a DNA-targeting segment that is at least 90% or at least 95% identical to the sequence (DNA-targeting segment) set forth in SEQ ID NO: 26 or 46. Alternatively, a guide RNA targeting human L-SH18 (chromosome 6, coordinates 170031084-170031382) can comprise a DNA-targeting segment that is at least 90% or at least 95% identical to the sequence (DNA-targeting segment) set forth in SEQ ID NO: 26. Alternatively, a guide RNA targeting human L-SH18 (chromosome 6, coordinates 170031084-170031382) can comprise a DNA-targeting segment that is at least 90% or at least 95% identical to the sequence (DNA-targeting segment) set forth in SEQ ID NO: 46. Alternatively, a guide RNA targeting human L-SH18 (chromosome 6, coordinates 170031084-170031382) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 26 or 46. Alternatively, a guide RNA targeting human L-SH18 (chromosome 6, coordinates 170031084-170031382) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 26. Alternatively, a guide RNA targeting human L-SH18 (chromosome 6, coordinates 170031084-170031382) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 46. Alternatively, a guide RNA targeting human L-SH18 (chromosome 6, coordinates 170031084-170031382) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 26 or 46. Alternatively, a guide RNA targeting human L-SH18 (chromosome 6, coordinates 170031084-170031382) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 26. Alternatively, a guide RNA targeting human L-SH18 (chromosome 6, coordinates 170031084-170031382) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 46. Alternatively, a guide RNA targeting human L-SH18 (chromosome 6, coordinates 170031084-170031382) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 26 or 46. Alternatively, a guide RNA targeting human L-SH18 (chromosome 6, coordinates 170031084-170031382) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 26. Alternatively, a guide RNA targeting human L-SH18 (chromosome 6, coordinates 170031084-170031382) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 46. Alternatively, a guide RNA targeting human L-SH18 (chromosome 6, coordinates 170031084-170031382) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 26 or 46. Alternatively, a guide RNA targeting human L-SH18 (chromosome 6, coordinates 170031084-170031382) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 26. Alternatively, a guide RNA targeting human L-SH18 (chromosome 6, coordinates 170031084-170031382) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 46.

As another example, a guide RNA targeting mouse L-SH18 (chromosome 17, coordinates 15,226,387-15,227,386) can comprise a DNA-targeting segment (i.e., guide sequence) comprising, consisting essentially of, or consisting of the sequence (DNA-targeting segment) set forth in any one of SEQ ID NOS: 345-374. Alternatively, a guide RNA targeting mouse L-SH18 (chromosome 17, coordinates 15,226,387-15,227,386) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 345-374. Alternatively, a guide RNA targeting mouse L-SH18 (chromosome 17, coordinates 15,226,387-15,227,386) can comprise a DNA-targeting segment that is at least 75%, at least 80%, at least 85%, at least 90%, or at least 95% identical to the sequence (DNA-targeting segment) set forth in any one of SEQ ID NOS: 345-374. Alternatively, a guide RNA targeting mouse L-SH18 (chromosome 17, coordinates 15,226,387-15,227,386) can comprise a DNA-targeting segment that is at least 90% or at least 95% identical to the sequence (DNA-targeting segment) set forth in any one of SEQ ID NOS: 345-374. Alternatively, a guide RNA targeting mouse L-SH18 (chromosome 17, coordinates 15,226,387-15,227,386) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 345-374. Alternatively, a guide RNA targeting mouse L-SH18 (chromosome 17, coordinates 15,226,387-15,227,386) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 345-374. Alternatively, a guide RNA targeting mouse L-SH18 (chromosome 17, coordinates 15,226,387-15,227,386) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 345-374. Alternatively, a guide RNA targeting mouse L-SH18 (chromosome 17, coordinates 15,226,387-15,227,386) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 345-374.

As another example, a guide RNA targeting mouse L-SH18 (chromosome 17, coordinates 15,226,387-15,227,386) can comprise a DNA-targeting segment (i.e., guide sequence) comprising, consisting essentially of, or consisting of the sequence (DNA-targeting segment) set forth in any one of SEQ ID NOS: 347, 360, 369, and 370. Alternatively, a guide RNA targeting mouse L-SH18 (chromosome 17, coordinates 15,226,387-15,227,386) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 347, 360, 369, and 370. Alternatively, a guide RNA targeting mouse L-SH18 (chromosome 17, coordinates 15,226,387-15,227,386) can comprise a DNA-targeting segment that is at least 75%, at least 80%, at least 85%, at least 90%, or at least 95% identical to the sequence (DNA-targeting segment) set forth in any one of SEQ ID NOS: 347, 360, 369, and 370. Alternatively, a guide RNA targeting mouse L-SH18 (chromosome 17, coordinates 15,226,387-15,227,386) can comprise a DNA-targeting segment that is at least 90% or at least 95% identical to the sequence (DNA-targeting segment) set forth in any one of SEQ ID NOS: 347, 360, 369, and 370. Alternatively, a guide RNA targeting mouse L-SH18 (chromosome 17, coordinates 15,226,387-15,227,386) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 347, 360, 369, and 370. Alternatively, a guide RNA targeting mouse L-SH18 (chromosome 17, coordinates 15,226,387-15,227,386) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 347, 360, 369, and 370. Alternatively, a guide RNA targeting mouse L-SH18 (chromosome 17, coordinates 15,226,387-15,227,386) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 347, 360, 369, and 370. Alternatively, a guide RNA targeting mouse L-SH18 (chromosome 17, coordinates 15,226,387-15,227,386) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 347, 360, 369, and 370.

As another example, a guide RNA targeting human L-SH20 (chromosome 9, coordinates 25207412-25207703) can comprise a DNA-targeting segment (i.e., guide sequence) comprising, consisting essentially of, or consisting of the sequence (DNA-targeting segment) set forth in any one of SEQ ID NOS: 27, 47, and 286-314. Alternatively, a guide RNA targeting human L-SH20 (chromosome 9, coordinates 25207412-25207703) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 27, 47, and 286-314. Alternatively, a guide RNA targeting human L-SH20 (chromosome 9, coordinates 25207412-25207703) can comprise a DNA-targeting segment that is at least 75%, at least 80%, at least 85%, at least 90%, or at least 95% identical to the sequence (DNA-targeting segment) set forth in any one of SEQ ID NOS: 27, 47, and 286-314. Alternatively, a guide RNA targeting human L-SH20 (chromosome 9, coordinates 25207412-25207703) can comprise a DNA-targeting segment that is at least 90% or at least 95% identical to the sequence (DNA-targeting segment) set forth in any one of SEQ ID NOS: 27, 47, and 286-314. Alternatively, a guide RNA targeting human L-SH20 (chromosome 9, coordinates 25207412-25207703) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 27, 47, and 286-314. Alternatively, a guide RNA targeting human L-SH20 (chromosome 9, coordinates 25207412-25207703) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 27, 47, and 286-314. Alternatively, a guide RNA targeting human L-SH20 (chromosome 9, coordinates 25207412-25207703) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 27, 47, and 286-314. Alternatively, a guide RNA targeting human L-SH20 (chromosome 9, coordinates 25207412-25207703) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 27, 47, and 286-314.

As another example, a guide RNA targeting human L-SH20 (chromosome 9, coordinates 25207412-25207703) can comprise a DNA-targeting segment (i.e., guide sequence) comprising, consisting essentially of, or consisting of the sequence (DNA-targeting segment) set forth in any one of SEQ ID NOS: 27, 47, 288, 296, 305, 306, and 310. Alternatively, a guide RNA targeting human L-SH20 (chromosome 9, coordinates 25207412-25207703) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 27, 47, 288, 296, 305, 306, and 310. Alternatively, a guide RNA targeting human L-SH20 (chromosome 9, coordinates 25207412-25207703) can comprise a DNA-targeting segment that is at least 75%, at least 80%, at least 85%, at least 90%, or at least 95% identical to the sequence (DNA-targeting segment) set forth in any one of SEQ ID NOS: 27, 47, 288, 296, 305, 306, and 310. Alternatively, a guide RNA targeting human L-SH20 (chromosome 9, coordinates 25207412-25207703) can comprise a DNA-targeting segment that is at least 90% or at least 95% identical to the sequence (DNA-targeting segment) set forth in any one of SEQ ID NOS: 27, 47, 288, 296, 305, 306, and 310. Alternatively, a guide RNA targeting human L-SH20 (chromosome 9, coordinates 25207412-25207703) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 27, 47, 288, 296, 305, 306, and 310. Alternatively, a guide RNA targeting human L-SH20 (chromosome 9, coordinates 25207412-25207703) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 27, 47, 288, 296, 305, 306, and 310. Alternatively, a guide RNA targeting human L-SH20 (chromosome 9, coordinates 25207412-25207703) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 27, 47, 288, 296, 305, 306, and 310. Alternatively, a guide RNA targeting human L-SH20 (chromosome 9, coordinates 25207412-25207703) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 27, 47, 288, 296, 305, 306, and 310.

As another example, a guide RNA targeting human L-SH20 (chromosome 9, coordinates 25207412-25207703) can comprise a DNA-targeting segment (i.e., guide sequence) comprising, consisting essentially of, or consisting of the sequence (DNA-targeting segment) set forth in SEQ ID NO: 27 or 47. As another example, a guide RNA targeting human L-SH20 (chromosome 9, coordinates 25207412-25207703) can comprise a DNA-targeting segment (i.e., guide sequence) comprising, consisting essentially of, or consisting of the sequence (DNA-targeting segment) set forth in SEQ ID NO: 27. As another example, a guide RNA targeting human L-SH20 (chromosome 9, coordinates 25207412-25207703) can comprise a DNA-targeting segment (i.e., guide sequence) comprising, consisting essentially of, or consisting of the sequence (DNA-targeting segment) set forth in SEQ ID NO: 47. Alternatively, a guide RNA targeting human L-SH20 (chromosome 9, coordinates 25207412-25207703) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 27 or 47. Alternatively, a guide RNA targeting human L-SH20 (chromosome 9, coordinates 25207412-25207703) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 27. Alternatively, a guide RNA targeting human L-SH20 (chromosome 9, coordinates 25207412-25207703) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 47. Alternatively, a guide RNA targeting human L-SH20 (chromosome 9, coordinates 25207412-25207703) can comprise a DNA-targeting segment that is at least 75%, at least 80%, at least 85%, at least 90%, or at least 95% identical to the sequence (DNA-targeting segment) set forth in SEQ ID NO: 27 or 47. Alternatively, a guide RNA targeting human L-SH20 (chromosome 9, coordinates 25207412-25207703) can comprise a DNA-targeting segment that is at least 75%, at least 80%, at least 85%, at least 90%, or at least 95% identical to the sequence (DNA-targeting segment) set forth in SEQ ID NO: 27. Alternatively, a guide RNA targeting human L-SH20 (chromosome 9, coordinates 25207412-25207703) can comprise a DNA-targeting segment that is at least 75%, at least 80%, at least 85%, at least 90%, or at least 95% identical to the sequence (DNA-targeting segment) set forth in SEQ ID NO: 47. Alternatively, a guide RNA targeting human L-SH20 (chromosome 9, coordinates 25207412-25207703) can comprise a DNA-targeting segment that is at least 90% or at least 95% identical to the sequence (DNA-targeting segment) set forth in SEQ ID NO: 27 or 47. Alternatively, a guide RNA targeting human L-SH20 (chromosome 9, coordinates 25207412-25207703) can comprise a DNA-targeting segment that is at least 90% or at least 95% identical to the sequence (DNA-targeting segment) set forth in SEQ ID NO: 27. Alternatively, a guide RNA targeting human L-SH20 (chromosome 9, coordinates 25207412-25207703) can comprise a DNA-targeting segment that is at least 90% or at least 95% identical to the sequence (DNA-targeting segment) set forth in SEQ ID NO: 47. Alternatively, a guide RNA targeting human L-SH20 (chromosome 9, coordinates 25207412-25207703) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 27 or 47. Alternatively, a guide RNA targeting human L-SH20 (chromosome 9, coordinates 25207412-25207703) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 27. Alternatively, a guide RNA targeting human L-SH20 (chromosome 9, coordinates 25207412-25207703) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 47. Alternatively, a guide RNA targeting human L-SH20 (chromosome 9, coordinates 25207412-25207703) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 27 or 47. Alternatively, a guide RNA targeting human L-SH20 (chromosome 9, coordinates 25207412-25207703) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 27. Alternatively, a guide RNA targeting human L-SH20 (chromosome 9, coordinates 25207412-25207703) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 47. Alternatively, a guide RNA targeting human L-SH20 (chromosome 9, coordinates 25207412-25207703) can comprise a DNA-targeting segment comprising, consisting essentially of, or consisting of a sequence that differs by no more than 3, no more than 2, or no more than 1 nucleotide from the sequence (DNA-targeting segment) set forth in SEQ TD NO: 27 or 47. Alternatively, a guide RNA targeting human L-SH20 (chromosome 9, coordinates 25207412-25207703) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 27. Alternatively, a guide RNA targeting human L-SH20 (chromosome 9, coordinates 25207412-25207703) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 47. Alternatively, a guide RNA targeting human L-SH20 (chromosome 9, coordinates 25207412-25207703) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 27 or 47. Alternatively, a guide RNA targeting human L-SH20 (chromosome 9, coordinates 25207412-25207703) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 27. Alternatively, a guide RNA targeting human L-SH20 (chromosome 9, coordinates 25207412-25207703) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 47.

As another example, a guide RNA targeting mouse L-SH20 (chromosome 4, coordinates 92,827,563-92,828,592) can comprise a DNA-targeting segment (i.e., guide sequence) comprising, consisting essentially of, or consisting of the sequence (DNA-targeting segment) set forth in any one of SEQ ID NOS: 375-404. Alternatively, a guide RNA targeting mouse L-SH20 (chromosome 4, coordinates 92,827,563-92,828,592) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 375-404. Alternatively, a guide RNA targeting mouse L-SH20 (chromosome 4, coordinates 92,827,563-92,828,592) can comprise a DNA-targeting segment that is at least 75%, at least 80%, at least 85%, at least 90%, or at least 95% identical to the sequence (DNA-targeting segment) set forth in any one of SEQ ID NOS: 375-404. Alternatively, a guide RNA targeting mouse L-SH20 (chromosome 4, coordinates 92,827,563-92,828,592) can comprise a DNA-targeting segment that is at least 90% or at least 95% identical to the sequence (DNA-targeting segment) set forth in any one of SEQ ID NOS: 375-404. Alternatively, a guide RNA targeting mouse L-SH20 (chromosome 4, coordinates 92,827,563-92,828,592) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 375-404. Alternatively, a guide RNA targeting mouse L-SH20 (chromosome 4, coordinates 92,827,563-92,828,592) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 375-404. Alternatively, a guide RNA targeting mouse L-SH20 (chromosome 4, coordinates 92,827,563-92,828,592) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 375-404. Alternatively, a guide RNA targeting mouse L-SH20 (chromosome 4, coordinates 92,827,563-92,828,592) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 375-404.

As another example, a guide RNA targeting mouse L-SH20 (chromosome 4, coordinates 92,827,563-92,828,592) can comprise a DNA-targeting segment (i.e., guide sequence) comprising, consisting essentially of, or consisting of the sequence (DNA-targeting segment) set forth in any one of SEQ ID NOS: 379, 380, and 388. Alternatively, a guide RNA targeting mouse L-SH20 (chromosome 4, coordinates 92,827,563-92,828,592) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 379, 380, and 388. Alternatively, a guide RNA targeting mouse L-SH20 (chromosome 4, coordinates 92,827,563-92,828,592) can comprise a DNA-targeting segment that is at least 75%, at least 80%, at least 85%, at least 90%, or at least 95% identical to the sequence (DNA-targeting segment) set forth in any one of SEQ ID NOS: 379, 380, and 388. Alternatively, a guide RNA targeting mouse L-SH20 (chromosome 4, coordinates 92,827,563-92,828,592) can comprise a DNA-targeting segment that is at least 90% or at least 95% identical to the sequence (DNA-targeting segment) set forth in any one of SEQ ID NOS: 379, 380, and 388. Alternatively, a guide RNA targeting mouse L-SH20 (chromosome 4, coordinates 92,827,563-92,828,592) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 379, 380, and 388. Alternatively, a guide RNA targeting mouse L-SH20 (chromosome 4, coordinates 92,827,563-92,828,592) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 379, 380, and 388. Alternatively, a guide RNA targeting mouse L-SH20 (chromosome 4, coordinates 92,827,563-92,828,592) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 379, 380, and 388. Alternatively, a guide RNA targeting mouse L-SH20 (chromosome 4, coordinates 92,827,563-92,828,592) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 379, 380, and 388.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of:

(version 1; SEQ ID NO: 11)
GUUUUAGAGCUAGAAAUAGCAAGUUAAAAUAAGGCUAGUCCGUUA
UCAACUUGAAAAAGUGGCACCGAGUCGGUGCU;
(version 2; SEQ ID NO: 12)
GUUGGAACCAUUCAAAACAGCAUAGCAAGUUAAAAUAAGGCUAGU
CCGUUAUCAACUUGAAAAAGUGGCACCGAGUCGGUGC;
(version 3; SEQ ID NO: 13)
GUUUUAGAGCUAGAAAUAGCAAGUUAAAAUAAGGCUAGUCCGUUA
UCAACUUGAAAAAGUGGCACCGAGUCGGUGC;
and
(version 4; SEQ ID NO: 14)
GUUUAAGAGCUAUGCUGGAAACAGCAUAGCAAGUUUAAAUAAGGC
UAGUCCGUUAUCAACUUGAAAAAGUGGCACCGAGUCGGUGC;
(version 5; SEQ ID NO: 15)
GUUUUAGAGCUAGAAAUAGCAAGUUAAAAUAAGGCUAGUCCGUUA
UCAACUUGAAAAAGUGGCACCGAGUCGGUGCUUUUUUU;
(version 6; SEQ ID NO: 16)
GUUUUAGAGCUAGAAAUAGCAAGUUAAAAUAAGGCUAGUCCGUUA
UCAACUUGAAAAAGUGGCACCGAGUCGGUGCUUUU;
(version 7; SEQ ID NO: 17)
GUUUAAGAGCUAUGCUGGAAACAGCAUAGCAAGUUUAAAUAAGGC
UAGUCCGUUAUCAACUUGAAAAAGUGGCACCGAGUCGGUGCUUUU
UU;
(version 8; SEQ ID NO: 18)
orGUUUUAGAGCUAGAAAUAGCAAGUUAAAAUAAGGCUAGUCCGUUA
UCAACUUGGCACCGAGUCGGUGC.

In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such as those listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification. Some gRNAs comprise a 5′ end modification and a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in a human cell, a human liver cell, or a human hepatocyte. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

As one example, a guide RNA targeting a genomic safe harbor locus as described herein can comprise, consist essentially of, or consist of the sequence set forth in any one of SEQ ID NOS: 28-30 or 48-50. Alternatively, a guide RNA targeting a genomic safe harbor locus as described herein can comprise, consist essentially of, or consist of a sequence that is at least 75%, at least 80%, at least 85%, at least 90%, or at least 95% identical to the DNA-targeting segment set forth in any one of SEQ ID NOS: 28-30 or 48-50. Alternatively, a guide RNA targeting a genomic safe harbor locus as described herein can comprise, consist essentially of, or consist of a sequence that is at least 90% or at least 95% identical to the DNA-targeting segment set forth in any one of SEQ ID NOS: 28-30 or 48-50. Alternatively, a guide RNA targeting a genomic safe harbor locus as described herein can comprise, consist essentially of, or consist of a sequence that differs by no more than 3, no more than 2, or no more than 1 nucleotide from the sequence set forth in any one of SEQ ID NOS: 28-30 or 48-50.

As another example, a guide RNA targeting human L-SH5 (chromosome 13, coordinates 77460242-77460537) can comprise, consist essentially of, or consist of the sequence set forth in SEQ ID NO: 28 or 48. As another example, a guide RNA targeting human L-SH5 (chromosome 13, coordinates 77460242-77460537) can comprise, consist essentially of, or consist of the sequence set forth in SEQ ID NO: 28. As another example, a guide RNA targeting human L-SH5 (chromosome 13, coordinates 77460242-77460537) can comprise, consist essentially of, or consist of the sequence set forth in SEQ ID NO: 48. Alternatively, a guide RNA targeting human L-SH5 (chromosome 13, coordinates 77460242-77460537) can comprise, consist essentially of, or consist of a sequence that is at least 75%, at least 80%, at least 85%, at least 90%, or at least 95% identical to the DNA-targeting segment set forth in SEQ ID NO: 28 or 48. Alternatively, a guide RNA targeting human L-SH5 (chromosome 13, coordinates 77460242-77460537) can comprise, consist essentially of, or consist of a sequence that is at least 75%, at least 80%, at least 85%, at least 90%, or at least 95% identical to the DNA-targeting segment set forth in SEQ ID NO: 28. Alternatively, a guide RNA targeting human L-SH5 (chromosome 13, coordinates 77460242-77460537) can comprise, consist essentially of, or consist of a sequence that is at least 75%, at least 80%, at least 85%, at least 90%, or at least 95% identical to the DNA-targeting segment set forth in SEQ ID NO: 48. Alternatively, a guide RNA targeting human L-SH5 (chromosome 13, coordinates 77460242-77460537) can comprise, consist essentially of, or consist of a sequence that is at least 90% or at least 95% identical to the DNA-targeting segment set forth in SEQ ID NO: 28 or 48. Alternatively, a guide RNA targeting human L-SH5 (chromosome 13, coordinates 77460242-77460537) can comprise, consist essentially of, or consist of a sequence that is at least 90% or at least 95% identical to the DNA-targeting segment set forth in SEQ ID NO: 28. Alternatively, a guide RNA targeting human L-SH5 (chromosome 13, coordinates 77460242-77460537) can comprise, consist essentially of, or consist of a sequence that is at least 90% or at least 95% identical to the DNA-targeting segment set forth in SEQ ID NO: 48. Alternatively, a guide RNA targeting human L-SH5 (chromosome 13, coordinates 77460242-77460537) can comprise, consist essentially of, or consist of a sequence that differs by no more than 3, no more than 2, or no more than 1 nucleotide from the sequence set forth in SEQ ID NO: 28 or 48. Alternatively, a guide RNA targeting human L-SH5 (chromosome 13, coordinates 77460242-77460537) can comprise, consist essentially of, or consist of a sequence that differs by no more than 3, no more than 2, or no more than 1 nucleotide from the sequence set forth in SEQ ID NO: 28. Alternatively, a guide RNA targeting human L-SH5 (chromosome 13, coordinates 77460242-77460537) can comprise, consist essentially of, or consist of a sequence that differs by no more than 3, no more than 2, or no more than 1 nucleotide from the sequence set forth in SEQ ID NO: 48.

As another example, a guide RNA targeting human L-SH18 (chromosome 6, coordinates 170031084-170031382) can comprise, consist essentially of, or consist of the sequence set forth in SEQ ID NO: 29 or 49. As another example, a guide RNA targeting human L-SH18 (chromosome 6, coordinates 170031084-170031382) can comprise, consist essentially of, or consist of the sequence set forth in SEQ ID NO: 29. As another example, a guide RNA targeting human L-SH18 (chromosome 6, coordinates 170031084-170031382) can comprise, consist essentially of, or consist of the sequence set forth in SEQ ID NO: 49. Alternatively, a guide RNA targeting human L-SH18 (chromosome 6, coordinates 170031084-170031382) can comprise, consist essentially of, or consist of a sequence that is at least 75%, at least 80%, at least 85%, at least 90%, or at least 95% identical to the DNA-targeting segment set forth in SEQ ID NO: 29 or 49. Alternatively, a guide RNA targeting human L-SH18 (chromosome 6, coordinates 170031084-170031382) can comprise, consist essentially of, or consist of a sequence that is at least 75%, at least 80%, at least 85%, at least 90%, or at least 95% identical to the DNA-targeting segment set forth in SEQ ID NO: 29. Alternatively, a guide RNA targeting human L-SH18 (chromosome 6, coordinates 170031084-170031382) can comprise, consist essentially of, or consist of a sequence that is at least 75%, at least 80%, at least 85%, at least 90%, or at least 95% identical to the DNA-targeting segment set forth in SEQ ID NO: 49. Alternatively, a guide RNA targeting human L-SH18 (chromosome 6, coordinates 170031084-170031382) can comprise, consist essentially of, or consist of a sequence that is at least 90% or at least 95% identical to the DNA-targeting segment set forth in SEQ ID NO: 29 or 49. Alternatively, a guide RNA targeting human L-SH18 (chromosome 6, coordinates 170031084-170031382) can comprise, consist essentially of, or consist of a sequence that is at least 90% or at least 95% identical to the DNA-targeting segment set forth in SEQ ID NO: 29. Alternatively, a guide RNA targeting human L-SH18 (chromosome 6, coordinates 170031084-170031382) can comprise, consist essentially of, or consist of a sequence that is at least 90% or at least 95% identical to the DNA-targeting segment set forth in SEQ ID NO: 49. Alternatively, a guide RNA targeting human L-SH18 (chromosome 6, coordinates 170031084-170031382) can comprise, consist essentially of, or consist of a sequence that differs by no more than 3, no more than 2, or no more than 1 nucleotide from the sequence set forth in SEQ ID NO: 29 or 49. Alternatively, a guide RNA targeting human L-SH18 (chromosome 6, coordinates 170031084-170031382) can comprise, consist essentially of, or consist of a sequence that differs by no more than 3, no more than 2, or no more than 1 nucleotide from the sequence set forth in SEQ ID NO: 29. Alternatively, a guide RNA targeting human L-SH18 (chromosome 6, coordinates 170031084-170031382) can comprise, consist essentially of, or consist of a sequence that differs by no more than 3, no more than 2, or no more than 1 nucleotide from the sequence set forth in SEQ ID NO: 49.

As another example, a guide RNA targeting human L-SH20 (chromosome 9, coordinates 25207412-25207703) can comprise, consist essentially of, or consist of the sequence set forth in SEQ ID NO: 30 or 50. As another example, a guide RNA targeting human L-SH20 (chromosome 9, coordinates 25207412-25207703) can comprise, consist essentially of, or consist of the sequence set forth in SEQ ID NO: 30. As another example, a guide RNA targeting human L-SH20 (chromosome 9, coordinates 25207412-25207703) can comprise, consist essentially of, or consist of the sequence set forth in SEQ ID NO: 50. Alternatively, a guide RNA targeting human L-SH20 (chromosome 9, coordinates 25207412-25207703) can comprise, consist essentially of, or consist of a sequence that is at least 75%, at least 80%, at least 85%, at least 90%, or at least 95% identical to the DNA-targeting segment set forth in SEQ ID NO: 30 or 50. Alternatively, a guide RNA targeting human L-SH20 (chromosome 9, coordinates 25207412-25207703) can comprise, consist essentially of, or consist of a sequence that is at least 75%, at least 80%, at least 85%, at least 90%, or at least 95% identical to the DNA-targeting segment set forth in SEQ ID NO: 30. Alternatively, a guide RNA targeting human L-SH20 (chromosome 9, coordinates 25207412-25207703) can comprise, consist essentially of, or consist of a sequence that is at least 75%, at least 80%, at least 85%, at least 90%, or at least 95% identical to the DNA-targeting segment set forth in SEQ ID NO: 50. Alternatively, a guide RNA targeting human L-SH20 (chromosome 9, coordinates 25207412-25207703) can comprise, consist essentially of, or consist of a sequence that is at least 90% or at least 95% identical to the DNA-targeting segment set forth in SEQ ID NO: 30 or 50. Alternatively, a guide RNA targeting human L-SH20 (chromosome 9, coordinates 25207412-25207703) can comprise, consist essentially of, or consist of a sequence that is at least 90% or at least 95% identical to the DNA-targeting segment set forth in SEQ ID NO: 30. Alternatively, a guide RNA targeting human L-SH20 (chromosome 9, coordinates 25207412-25207703) can comprise, consist essentially of, or consist of a sequence that is at least 90% or at least 95% identical to the DNA-targeting segment set forth in SEQ ID NO: 50. Alternatively, a guide RNA targeting human L-SH20 (chromosome 9, coordinates 25207412-25207703) can comprise, consist essentially of, or consist of a sequence that differs by no more than 3, no more than 2, or no more than 1 nucleotide from the sequence set forth in SEQ ID NO: 30 or 50. Alternatively, a guide RNA targeting human L-SH20 (chromosome 9, coordinates 25207412-25207703) can comprise, consist essentially of, or consist of a sequence that differs by no more than 3, no more than 2, or no more than 1 nucleotide from the sequence set forth in SEQ ID NO: 30. Alternatively, a guide RNA targeting human L-SH20 (chromosome 9, coordinates 25207412-25207703) can comprise, consist essentially of, or consist of a sequence that differs by no more than 3, no more than 2, or no more than 1 nucleotide from the sequence set forth in SEQ ID NO: 50.

### (4) Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of Cas(D, the PAM can have the sequence 5′-TBN-3′, where B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 19) or N₂₀NGG (SEQ ID NO: 20). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 21) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 19-21, including the 5′ G or GG and the 3′ GG or NGG. Yet other guide RNA target sequences plus PAMs can have between 14 and 20 nucleotides in length of SEQ ID NOS: 19-21.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g. Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

The guide RNA target sequence can also be selected to minimize off-target modification or avoid off-target effects (e.g., by avoiding two or fewer mismatches to off-target genomic sequences).

As one example, a guide RNA targeting a genomic safe harbor locus as described herein can target the guide RNA target sequence set forth in any one of SEQ ID NOS: 22-24, 42-44, and 51-137. As another example, a guide RNA targeting in a genomic safe harbor locus as described herein can target at least 17, at least 18, at least 19, or at least 20 contiguous nucleotides of the guide RNA target sequence set forth in any one of SEQ ID NOS: 22-24, 42-44, and 51-137.

As one example, a guide RNA targeting a genomic safe harbor locus as described herein can target the guide RNA target sequence set forth in any one of SEQ ID NOS: 138-227. As another example, a guide RNA targeting in a genomic safe harbor locus as described herein can target at least 17, at least 18, at least 19, or at least 20 contiguous nucleotides of the guide RNA target sequence set forth in any one of SEQ ID NOS: 138-227.

As one example, a guide RNA targeting a genomic safe harbor locus as described herein can target the guide RNA target sequence set forth in any one of SEQ ID NOS: 22-24 or 42-44. As another example, a guide RNA targeting in a genomic safe harbor locus as described herein can target at least 17, at least 18, at least 19, or at least 20 contiguous nucleotides of the guide RNA target sequence set forth in any one of SEQ ID NOS: 22-24 or 42-44.

As another example, a guide RNA targeting human L-SH5 (chromosome 13, coordinates 77460242-77460537) can target the guide RNA target sequence set forth in any one of SEQ ID NOS: 22, 42, and 51-79. As another example, a guide RNA targeting human L-SH5 (chromosome 13, coordinates 77460242-77460537) can target at least 17, at least 18, at least 19, or at least 20 contiguous nucleotides of the guide RNA target sequence set forth in any one of SEQ ID NOS: 22, 42, and 51-79.

As another example, a guide RNA targeting human L-SH5 (chromosome 13, coordinates 77460242-77460537) can target the guide RNA target sequence set forth in any one of SEQ ID NOS: 22, 42, 58, 60, and 69. As another example, a guide RNA targeting human L-SH5 (chromosome 13, coordinates 77460242-77460537) can target at least 17, at least 18, at least 19, or at least 20 contiguous nucleotides of the guide RNA target sequence set forth in any one of SEQ ID NOS: 22, 42, 58, 60, and 69.

As another example, a guide RNA targeting human L-SH5 (chromosome 13, coordinates 77460242-77460537) can target the guide RNA target sequence set forth in SEQ ID NO: 22 or 42. As another example, a guide RNA targeting human L-SH5 (chromosome 13, coordinates 77460242-77460537) can target the guide RNA target sequence set forth in SEQ ID NO: 22. As another example, a guide RNA targeting human L-SH5 (chromosome 13, coordinates 77460242-77460537) can target the guide RNA target sequence set forth in SEQ ID NO: 42. As another example, a guide RNA targeting human L-SH5 (chromosome 13, coordinates 77460242-77460537) can target at least 17, at least 18, at least 19, or at least 20 contiguous nucleotides of the guide RNA target sequence set forth in SEQ ID NO: 22 or 42. As another example, a guide RNA targeting human L-SH5 (chromosome 13, coordinates 77460242-77460537) can target at least 17, at least 18, at least 19, or at least 20 contiguous nucleotides of the guide RNA target sequence set forth in SEQ ID NO: 22. As another example, a guide RNA targeting human L-SH5 (chromosome 13, coordinates 77460242-77460537) can target at least 17, at least 18, at least 19, or at least 20 contiguous nucleotides of the guide RNA target sequence set forth in SEQ ID NO: 42.

As another example, a guide RNA targeting mouse L-SH5 (chromosome 14, coordinates 103,450,397-103,451,396) can target the guide RNA target sequence set forth in any one of SEQ ID NOS: 138-167. As another example, a guide RNA targeting mouse L-SH5 (chromosome 14, coordinates 103,450,397-103,451,396) can target at least 17, at least 18, at least 19, or at least 20 contiguous nucleotides of the guide RNA target sequence set forth in any one of SEQ ID NOS: 138-167.

As another example, a guide RNA targeting mouse L-SH5 (chromosome 14, coordinates 103,450,397-103,451,396) can target the guide RNA target sequence set forth in any one of SEQ ID NOS: 141, 143, 144, and 164. As another example, a guide RNA targeting mouse L-SH5 (chromosome 14, coordinates 103,450,397-103,451,396) can target at least 17, at least 18, at least 19, or at least 20 contiguous nucleotides of the guide RNA target sequence set forth in any one of SEQ ID NOS: 141, 143, 144, and 164.

As another example, a guide RNA targeting human L-SH18 (chromosome 6, coordinates 170031084-170031382) can target the guide RNA target sequence set forth in any one of SEQ ID NOS: 23, 43, and 80-108. As another example, a guide RNA targeting human L-SH18 (chromosome 6, coordinates 170031084-170031382) can target at least 17, at least 18, at least 19, or at least 20 contiguous nucleotides of the guide RNA target sequence set forth in any one of SEQ ID NOS: 23, 43, and 80-108.

As another example, a guide RNA targeting human L-SH18 (chromosome 6, coordinates 170031084-170031382) can target the guide RNA target sequence set forth in any one of SEQ ID NOS: 23, 43, 91, 94, and 103. As another example, a guide RNA targeting human L-SH18 (chromosome 6, coordinates 170031084-170031382) can target at least 17, at least 18, at least 19, or at least 20 contiguous nucleotides of the guide RNA target sequence set forth in any one of SEQ ID NOS: 23, 43, 91, 94, and 103.

As another example, a guide RNA targeting human L-SH18 (chromosome 6, coordinates 170031084-170031382) can target the guide RNA target sequence set forth in SEQ ID NO: 23 or 43. As another example, a guide RNA targeting human L-SH18 (chromosome 6, coordinates 170031084-170031382) can target the guide RNA target sequence set forth in SEQ ID NO: 23. As another example, a guide RNA targeting human L-SH18 (chromosome 6, coordinates 170031084-170031382) can target the guide RNA target sequence set forth in SEQ ID NO: 43. As another example, a guide RNA targeting human L-SH18 (chromosome 6, coordinates 170031084-170031382) can target at least 17, at least 18, at least 19, or at least 20 contiguous nucleotides of the guide RNA target sequence set forth in SEQ ID NO: 23 or 43. As another example, a guide RNA targeting human L-SH18 (chromosome 6, coordinates 170031084-170031382) can target at least 17, at least 18, at least 19, or at least 20 contiguous nucleotides of the guide RNA target sequence set forth in SEQ ID NO: 23. As another example, a guide RNA targeting human L-SH18 (chromosome 6, coordinates 170031084-170031382) can target at least 17, at least 18, at least 19, or at least 20 contiguous nucleotides of the guide RNA target sequence set forth in SEQ ID NO: 43.

As another example, a guide RNA targeting mouse L-SH18 (chromosome 17, coordinates 15,226,387-15,227,386) can target the guide RNA target sequence set forth in any one of SEQ ID NOS: 168-197. As another example, a guide RNA targeting mouse L-SH18 (chromosome 17, coordinates 15,226,387-15,227,386) can target at least 17, at least 18, at least 19, or at least 20 contiguous nucleotides of the guide RNA target sequence set forth in any one of SEQ ID NOS: 168-197.

As another example, a guide RNA targeting mouse L-SH18 (chromosome 17, coordinates 15,226,387-15,227,386) can target the guide RNA target sequence set forth in any one of SEQ ID NOS: 170, 183, 192, and 193. As another example, a guide RNA targeting mouse L-SH18 (chromosome 17, coordinates 15,226,387-15,227,386) can target at least 17, at least 18, at least 19, or at least 20 contiguous nucleotides of the guide RNA target sequence set forth in any one of SEQ ID NOS: 170, 183, 192, and 193.

As another example, a guide RNA targeting human L-SH20 (chromosome 9, coordinates 25207412-25207703) can target the guide RNA target sequence set forth in any one of SEQ ID NOS: 24, 44, and 109-137. As another example, a guide RNA targeting human L-SH20 (chromosome 9, coordinates 25207412-25207703) can target at least 17, at least 18, at least 19, or at least 20 contiguous nucleotides of the guide RNA target sequence set forth in any one of SEQ ID NOS: 24, 44, and 109-137.

As another example, a guide RNA targeting human L-SH20 (chromosome 9, coordinates 25207412-25207703) can target the guide RNA target sequence set forth in any one of SEQ ID NOS: 24, 44, 111, 119, 128, 129, and 133. As another example, a guide RNA targeting human L-SH20 (chromosome 9, coordinates 25207412-25207703) can target at least 17, at least 18, at least 19, or at least 20 contiguous nucleotides of the guide RNA target sequence set forth in any one of SEQ ID NOS: 24, 44, 111, 119, 128, 129, and 133.

As another example, a guide RNA targeting human L-SH20 (chromosome 9, coordinates 25207412-25207703) can target the guide RNA target sequence set forth in SEQ ID NO: 24 or 44. As another example, a guide RNA targeting human L-SH20 (chromosome 9, coordinates 25207412-25207703) can target the guide RNA target sequence set forth in SEQ ID NO: 24. As another example, a guide RNA targeting human L-SH20 (chromosome 9, coordinates 25207412-25207703) can target the guide RNA target sequence set forth in SEQ ID NO: 44. As another example, a guide RNA targeting human L-SH20 (chromosome 9, coordinates 25207412-25207703) can target at least 17, at least 18, at least 19, or at least 20 contiguous nucleotides of the guide RNA target sequence set forth in SEQ ID NO: 24 or 44. As another example, a guide RNA targeting human L-SH20 (chromosome 9, coordinates 25207412-25207703) can target at least 17, at least 18, at least 19, or at least 20 contiguous nucleotides of the guide RNA target sequence set forth in SEQ ID NO: 24. As another example, a guide RNA targeting human L-SH20 (chromosome 9, coordinates 25207412-25207703) can target at least 17, at least 18, at least 19, or at least 20 contiguous nucleotides of the guide RNA target sequence set forth in SEQ ID NO: 44.

As another example, a guide RNA targeting mouse L-SH20 (chromosome 4, coordinates 92,827,563-92,828,592) can target the guide RNA target sequence set forth in any one of SEQ ID NOS: 198-227. As another example, a guide RNA targeting mouse L-SH20 (chromosome 4, coordinates 92,827,563-92,828,592) can target at least 17, at least 18, at least 19, or at least 20 contiguous nucleotides of the guide RNA target sequence set forth in any one of SEQ ID NOS: 198-227.

As another example, a guide RNA targeting mouse L-SH20 (chromosome 4, coordinates 92,827,563-92,828,592) can target the guide RNA target sequence set forth in any one of SEQ ID NOS: 202, 203, and 211. As another example, a guide RNA targeting mouse L-SH20 (chromosome 4, coordinates 92,827,563-92,828,592) can target at least 17, at least 18, at least 19, or at least 20 contiguous nucleotides of the guide RNA target sequence set forth in any one of SEQ ID NOS: 202, 203, and 211.

### (5) Lipid Nanoparticles Comprising Nuclease Agents

Lipid nanoparticles comprising the nuclease agents (e.g., CRISPR/Cas systems) are also provided. The lipid nanoparticles can alternatively or additionally comprise a nucleic acid construct encoding a product of interest (e.g., polypeptide of interest) as disclosed herein. For example, the lipid nanoparticles can comprise a nuclease agent (e.g., CRISPR/Cas system), can comprise a nucleic acid construct encoding a product of interest (e.g., polypeptide of interest), or can comprise both a nuclease agent (e.g., a CRISPR/Cas system) and a nucleic acid construct encoding a product of interest (e.g., polypeptide of interest). Regarding CRISPR/Cas systems, the lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. See, e.g., WO 2016/010840 A1 and WO 2017/173054 A1, each of which is herein incorporated by reference in its entirety for all purposes. An exemplary lipid nanoparticle can comprise a cationic lipid and one or more other components.

In some LNPs, the cargo can comprise Cas mRNA (e.g., Cas9 mRNA) and gRNA. The Cas mRNA and gRNAs can be in different ratios. In some LNPs, the cargo can comprise a nucleic acid construct encoding a product of interest (e.g., polypeptide of interest) and gRNA. The nucleic acid construct encoding a product of interest (e.g., polypeptide of interest) and gRNAs can be in different ratios.

Examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

### (6) Vectors Comprising Nuclease Agents

The nuclease agents disclosed herein (e.g., ZFN, TALEN, or CRISPR/Cas) can be provided in a vector for expression. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper components to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In rAAV genomes, a gene expression cassette can be placed between ITR sequences. Typically, rAAV genome cassettes comprise of a promoter to drive expression of a transgene, followed by a polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, AAV-DJ, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas nuclease, such as Cas9, and DNA encoding one or more guide RNAs (e.g., DNA encoding a guide RNA, or DNA encoding two or more guide RNAs). In certain AAVs, the cargo can include a nucleic acid construct encoding a product of interest (e.g., polypeptide of interest). In certain AAVs, the cargo can include a nucleic acid (e.g., DNA) encoding a Cas nuclease, such as Cas9, a DNA encoding a guide RNA (or multiple guide RNAs), and a nucleic acid construct encoding a product of interest (e.g., polypeptide of interest).

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., rAAV8-mediated delivery). For example, a Cas9 mRNA and a gRNA can be delivered via LNP-mediated delivery, or DNA encoding Cas9 and DNA encoding a gRNA can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity). D. Cells or Animals or Genomes or Nucleic Acids

Cells or animals (i.e., subjects) comprising any of the above compositions (e.g., nucleic acid construct encoding a product of interest (e.g., polypeptide of interest), nuclease agents, vectors, lipid nanoparticles, or any combination thereof) are also provided herein. Such cells or animals (or genomes) can be produced by the methods disclosed herein. For example, the cells or animals can comprise any of the nucleic acid constructs encoding a product of interest (e.g., polypeptide of interest) described herein, any of the nuclease agents disclosed herein, or both.

In some such cells or animals or genomes, the nucleic acid construct encoding a product of interest (e.g., polypeptide of interest) can be genomically integrated at a target genomic locus (e.g., a genomic safe harbor locus), such that the product of interest (e.g., polypeptide of interest) encoded by the nucleic acid construct is expressed in the cell, animal, or genome. For example, if the nucleic acid construct encoding a product of interest (e.g., polypeptide of interest) is integrated into a genomic safe harbor locus, the product of interest (e.g., polypeptide of interest) can be expressed from the genomic safe harbor locus. In one specific example, the genomic safe harbor locus is L-SH5 (human chromosome 13, coordinates 77460242-77460537). In another specific example, the genomic safe harbor locus is L-SH18 (human chromosome 6, coordinates 170031084-170031382). In another specific example, the genomic safe harbor locus is L-SH20 (human chromosome 9, coordinates 25207412-25207703).

In a specific example, the genomic safe harbor locus is selected from the following genomic locations: (i) human chromosome 13, coordinates 77460242-77460537 (referred to herein as L-SH5) or a corresponding region (e.g., orthologous or syntenic region) in a non-human animal, non-human mammal (e.g., non-human primate), or rodent, such as a rat or a mouse; (ii) human chromosome 6, coordinates 170031084-170031382 (referred to herein as L-SH18) or a corresponding region (e.g., orthologous or syntenic region) in a non-human animal, non-human mammal (e.g., non-human primate), or rodent, such as a rat or a mouse; and (iii) human chromosome 9, coordinates 25207412-25207703 (referred to herein as L-SH20) or a corresponding region (e.g., orthologous or syntenic region) in a non-human animal, non-human mammal (e.g., non-human primate), or rodent, such as a rat or a mouse.

In a specific example, the genomic safe harbor locus is selected from the following genomic coordinates: (i) about 77460242 to about 77460537 on human chromosome 13 (corresponds to L-SH5) or a corresponding region (e.g., orthologous or syntenic region) in a non-human animal, non-human mammal (e.g., non-human primate), or rodent, such as a rat or a mouse; (ii) about 170031084 to about 170031382 on human chromosome 6 (corresponds to L-SH18) or a corresponding region (e.g., orthologous or syntenic region) in a non-human animal, non-human mammal (e.g., non-human primate), or rodent, such as a rat or a mouse; and (iii) about 25207412 to about 25207703 on human chromosome 9 (corresponds to L-SH20) or a corresponding region (e.g., orthologous or syntenic region) in a non-human animal, non-human mammal (e.g., non-human primate), or rodent, such as a rat or a mouse. The term “about” when referring to genomic coordinates means±20 base pairs. In other examples, the genomic safe harbor locus is near the regions identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In one specific example, the genomic safe harbor locus is human L-SH5 (chromosome 13, coordinates 77460242-77460537) or a corresponding region (e.g., orthologous or syntenic region) in a non-human animal, non-human mammal (e.g., non-human primate), or rodent, such as a rat or a mouse. For example, the genomic safe harbor locus can comprise the sequence set forth in SEQ ID NO: 39 or variants thereof which are located at the same position, or genetic locus, on a chromosome in humans or orthologous or syntenic regions in a non-human animal, non-human mammal (e.g., non-human primate), or rodent, such as a rat or a mouse. Syntenic regions are derived from a single ancestral genomic region. For example, syntenic regions can be from different organisms and are derived from speciation.

In another specific example, the genomic safe harbor locus is human L-SH18 (chromosome 6, coordinates 170031084-170031382) or a corresponding region (e.g., orthologous or syntenic region) in a non-human animal, non-human mammal (e.g., non-human primate), or rodent, such as a rat or a mouse. For example, the genomic safe harbor locus can comprise the sequence set forth in SEQ ID NO: 40 or variants thereof which are located at the same position, or genetic locus, on a chromosome in humans or orthologous or syntenic regions in a non-human animal, non-human mammal (e.g., non-human primate), or rodent, such as a rat or a mouse.

In another specific example, the genomic safe harbor locus is human L-SH20 (chromosome 9, coordinates 25207412-25207703) or a corresponding region (e.g., orthologous or syntenic region) in a non-human animal, non-human mammal (e.g., non-human primate), or rodent, such as a rat or a mouse. For example, the genomic safe harbor locus can comprise the sequence set forth in SEQ ID NO: 41 or variants thereof which are located at the same position, or genetic locus, on a chromosome in humans or orthologous or syntenic regions in a non-human animal, non-human mammal (e.g., non-human primate), or rodent, such as a rat or a mouse.

In one specific example, the genomic safe harbor locus corresponds to human L-SH5 (coordinates of about 77460242 to about 77460537 on chromosome 13) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to human L-SH18 (coordinates of about 170031084 to about 170031382 on chromosome 6) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means ±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to human L-SH20 (coordinates of about 25207412 to about 25207703 on chromosome 9) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In some such cells or animals or genomes, the nucleic acid construct encoding a product of interest (e.g., polypeptide of interest) can be genomically integrated at a target genomic locus (e.g., a genomic safe harbor locus), such that the product of interest (e.g., polypeptide of interest) encoded by the nucleic acid construct is expressed in the cell, animal, or genome. For example, if the nucleic acid construct encoding a product of interest (e.g., polypeptide of interest) is integrated into a genomic safe harbor locus, the product of interest (e.g., polypeptide of interest) can be expressed from the genomic safe harbor locus. In one specific example, the genomic safe harbor locus is mouse L-SH5 (mouse chromosome 14, coordinates 103,450,397-103,451,396). In another specific example, the genomic safe harbor locus is mouse L-SH18 (mouse chromosome 17, coordinates 15,226,387-15,227,386). In another specific example, the genomic safe harbor locus is mouse L-SH20 (mouse chromosome 4, coordinates 92,827,563-92,828,592).

In a specific example, the genomic safe harbor locus is selected from the following genomic locations: (i) mouse chromosome 14, coordinates 103,450,397-103,451,396 (referred to herein as mouse L-SH5) or a corresponding region (e.g., orthologous or syntenic region) in a non-human animal, non-human mammal (e.g., non-human primate), or rodent, such as a rat; (ii) mouse chromosome 17, coordinates 15,226,387-15,227,386 (referred to herein as mouse L-SH18) or a corresponding region (e.g., orthologous or syntenic region) in a non-human animal, non-human mammal (e.g., non-human primate), or rodent, such as a rat; and (iii) mouse chromosome 4, coordinates 92,827,563-92,828,592 (referred to herein as mouse L-SH20) or a corresponding region (e.g., orthologous or syntenic region) in a non-human animal, non-human mammal (e.g., non-human primate), or rodent, such as a rat.

In a specific example, the genomic safe harbor locus is selected from the following genomic coordinates: (i) about 103,450,397 to about 103,451,396 on mouse chromosome 14 (corresponds to mouse L-SH5) or a corresponding region (e.g., orthologous or syntenic region) in a non-human animal, non-human mammal (e.g., non-human primate), or rodent, such as a rat; (ii) about 15,226,387 to about 15,227,386 on mouse chromosome 17 (corresponds to mouse L-SH18) or a corresponding region (e.g., orthologous or syntenic region) in a non-human animal, non-human mammal (e.g., non-human primate), or rodent, such as a rat; and (iii) about 92,827,563 to about 92,828,592 on mouse chromosome 4 (corresponds to mouse L-SH20) or a corresponding region (e.g., orthologous or syntenic region) in a non-human animal, non-human mammal (e.g., non-human primate), or rodent, such as a rat. The term “about” when referring to genomic coordinates means±20 base pairs. In other examples, the genomic safe harbor locus is near the regions identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In one specific example, the genomic safe harbor locus is mouse L-SH5 (chromosome 14, coordinates 103,450,397-103,451,396) or a corresponding region (e.g., orthologous or syntenic region) in a non-human animal, non-human mammal (e.g., non-human primate), or rodent, such as a rat. For example, the genomic safe harbor locus can comprise the sequence set forth in SEQ ID NO: 405 or variants thereof which are located at the same position, or genetic locus, on a chromosome in mice or orthologous or syntenic regions in a human or non-human animal, non-human mammal (e.g., non-human primate), or rodent, such as a rat. Syntenic regions are derived from a single ancestral genomic region. For example, syntenic regions can be from different organisms and are derived from speciation.

In another specific example, the genomic safe harbor locus is mouse L-SH18 (chromosome 17, coordinates 15,226,387-15,227,386) or a corresponding region (e.g., orthologous or syntenic region) in a non-human animal, non-human mammal (e.g., non-human primate), or rodent, such as a rat. For example, the genomic safe harbor locus can comprise the sequence set forth in SEQ ID NO: 406 or variants thereof which are located at the same position, or genetic locus, on a chromosome in mice or orthologous or syntenic regions in a human or non-human animal, non-human mammal (e.g., non-human primate), or rodent, such as a rat.

In another specific example, the genomic safe harbor locus is mouse L-SH20 (chromosome 4, coordinates 92,827,563-92,828,592) or a corresponding region (e.g., orthologous or syntenic region) in a non-human animal, non-human mammal (e.g., non-human primate), or rodent, such as a rat. For example, the genomic safe harbor locus can comprise the sequence set forth in SEQ ID NO: 407 or variants thereof which are located at the same position, or genetic locus, on a chromosome in mice or orthologous or syntenic regions in a human or non-human animal, non-human mammal (e.g., non-human primate), or rodent, such as a rat.

In one specific example, the genomic safe harbor locus corresponds to mouse L-SH5 (coordinates of about 103,450,397 to about 103,451,396 on mouse chromosome 14) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to mouse L-SH18 (coordinates of about 15,226,387 to about 15,227,386 on mouse chromosome 17) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to mouse L-SH20 (coordinates of about 92,827,563 to about 92,828,592 on mouse chromosome 4) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

The target genomic locus at which the nucleic acid construct is stably integrated can be heterozygous for the nucleic acid construct encoding a product of interest (e.g., polypeptide of interest) or homozygous for the nucleic acid construct encoding a product of interest (e.g., polypeptide of interest). A diploid organism has two alleles at each genetic locus. Each pair of alleles represents the genotype of a specific genetic locus. Genotypes are described as homozygous if there are two identical alleles at a particular locus and as heterozygous if the two alleles differ.

The cells or genomes can be from any suitable species, such as eukaryotic cells or eukaryotes, or mammalian cells or mammals (e.g., non-human mammalian cells or non-human mammals, or human cells or humans). A mammal can be, for example, a non-human mammal, a human, a rodent, a rat, a mouse, or a hamster. Other non-human mammals include, for example, non-human primates, e.g., monkeys and apes. The term “non-human” excludes humans. Examples include, but are not limited to, human cells/humans, rodent cells/rodents, mouse cells/mice, rat cells/rats, and non-human primate cells/non-human primates. In a specific example, the cell is a human cell or the animal is a human. Likewise, cells can be any suitable type of cell. In a specific example, the cell is a liver cell such as a hepatocyte (e.g., a human liver cell or human hepatocyte).

The cells can be isolated cells (e.g., in vitro), ex vivo cells, or can be in vivo within an animal (i.e., in a subject). In one example, the cells are in vitro or ex vivo. In another example, the cells are in vivo within a subject. The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatocytes (e.g., human hepatocytes).

The cells provided herein can be normal, healthy cells, or can be diseased or mutant-bearing cells. For example, the cells can have a deficiency of the product of interest (e.g., polypeptide of interest) or can be from a subject with deficiency of the product of interest (e.g., polypeptide of interest).

The cells provided herein can be dividing cells (e.g., actively dividing cells). Alternatively, the cells provided herein can be non-dividing cells.

Also provided nucleic acids comprising any of the nucleic acid constructs disclosed herein integrated into a target genomic locus (e.g., genomic safe harbor locus as disclosed elsewhere herein). The nucleic acid construct can comprise a nucleic acid operably linked to a promoter, wherein the nucleic acid encodes a product of interest. The genomic safe harbor locus can be selected, for example, from the following genomic locations: (i) human chromosome 13, coordinates 77460242-77460537 (referred to herein as L-SH5) or a corresponding region (e.g., orthologous or syntenic region) in a non-human animal, non-human mammal (e.g., non-human primate), or rodent, such as a rat or a mouse; (ii) human chromosome 6, coordinates 170031084-170031382 (referred to herein as L-SH18) or a corresponding region (e.g., orthologous or syntenic region) in a non-human animal, non-human mammal (e.g., non-human primate), or rodent, such as a rat or a mouse; and (iii) human chromosome 9, coordinates 25207412-25207703 (referred to herein as L-SH20) or a corresponding region (e.g., orthologous or syntenic region) in a non-human animal, non-human mammal (e.g., non-human primate), or rodent, such as a rat or a mouse.

The genomic safe harbor locus can also selected from the following genomic coordinates: (i) about 77460242 to about 77460537 on human chromosome 13 (corresponds to L-SH5) or a corresponding region (e.g., orthologous or syntenic region) in a non-human animal, non-human mammal (e.g., non-human primate), or rodent, such as a rat or a mouse; (ii) about 170031084 to about 170031382 on human chromosome 6 (corresponds to L-SH18) or a corresponding region (e.g., orthologous or syntenic region) in a non-human animal, non-human mammal (e.g., non-human primate), or rodent, such as a rat or a mouse; and (iii) about 25207412 to about 25207703 on human chromosome 9 (corresponds to L-SH20) or a corresponding region (e.g., orthologous or syntenic region) in a non-human animal, non-human mammal (e.g., non-human primate), or rodent, such as a rat or a mouse. The term “about” when referring to genomic coordinates means±20 base pairs. In other examples, the genomic safe harbor locus is near the regions identified by the above coordinates. The term “near” when referring to genomic coordinates means 5 kb, ±4 kb, ±3 kb, ±2 kb, ±1 kb, ±0.5 kb, ±0.4 kb, ±0.3 kb, ±0.2 kb, or ±0.1 kb.

Also provided nucleic acids comprising any of the nucleic acid constructs disclosed herein integrated into a target genomic locus (e.g., genomic safe harbor locus as disclosed elsewhere herein). The nucleic acid construct can comprise a nucleic acid operably linked to a promoter, wherein the nucleic acid encodes a product of interest. The genomic safe harbor locus can be selected, for example, from the following genomic locations: (i) mouse chromosome 14, coordinates 103,450,397-103,451,396 (referred to herein as mouse L-SH5) or a corresponding region (e.g., orthologous or syntenic region) in a non-human animal, non-human mammal (e.g., non-human primate), or rodent, such as a rat; (ii) mouse chromosome 17, coordinates 15,226,387-15,227,386 (referred to herein as mouse L-SH18) or a corresponding region (e.g., orthologous or syntenic region) in a non-human animal, non-human mammal (e.g., non-human primate), or rodent, such as a rat; and (iii) mouse chromosome 4, coordinates 92,827,563-92,828,592 (referred to herein as mouse L-SH20) or a corresponding region (e.g., orthologous or syntenic region) in a non-human animal, non-human mammal (e.g., non-human primate), or rodent, such as a rat.

The genomic safe harbor locus can also selected from the following genomic coordinates: (i) about 103,450,397 to about 103,451,396 on mouse chromosome 14 (corresponds to mouse L-SH5) or a corresponding region (e.g., orthologous or syntenic region) in a non-human animal, non-human mammal (e.g., non-human primate), or rodent, such as a rat; (ii) about 15,226,387 to about 15,227,386 on mouse chromosome 17 (corresponds to mouse L-SH18) or a corresponding region (e.g., orthologous or syntenic region) in a non-human animal, non-human mammal (e.g., non-human primate), or rodent, such as a rat; and (iii) about 92,827,563 to about 92,828,592 on mouse chromosome 4 (corresponds to mouse L-SH20) or a corresponding region (e.g., orthologous or syntenic region) in a non-human animal, non-human mammal (e.g., non-human primate), or rodent, such as a rat. The term “about” when referring to genomic coordinates means±20 base pairs. In other examples, the genomic safe harbor locus is near the regions identified by the above coordinates. The term “near” when referring to genomic coordinates means 5 kb, ±4 kb, ±3 kb, ±2 kb, ±1 kb, ±0.5 kb, ±0.4 kb, ±0.3 kb, ±0.2 kb, or ±0.1 kb.

The product of interest can be any product of interest disclosed elsewhere herein. For example, the product of interest can be a polypeptide of interest, such as a therapeutic polypeptide, a secreted polypeptide, or an intracellular polypeptide.

The promoter can be any promoter disclosed elsewhere herein. For example, the promoter can be active in liver cells, can be a tissue-specific promoter, can be a constitutive promoter, or can be an inducible promoter.

In one specific example, the genomic safe harbor locus is human chromosome 13, coordinates 77460242-77460537 (referred to herein as L-SH5) or a corresponding region (e.g., orthologous or syntenic region) in a non-human animal, non-human mammal (e.g., non-human primate), or rodent, such as a rat or a mouse.

In one specific example, the genomic safe harbor locus is human chromosome 6, coordinates 170031084-170031382 (referred to herein as L-SH18) or a corresponding region (e.g., orthologous or syntenic region) in a non-human animal, non-human mammal (e.g., non-human primate), or rodent, such as a rat or a mouse.

In one specific example, the genomic safe harbor locus is human chromosome 9, coordinates 25207412-25207703 (referred to herein as L-SH20) or a corresponding region (e.g., orthologous or syntenic region) in a non-human animal, non-human mammal (e.g., non-human primate), or rodent, such as a rat or a mouse.

In one specific example, the genomic safe harbor locus corresponds to human L-SH5 (coordinates of about 77460242 to about 77460537 on chromosome 13) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±kb, ±0.5 kb, ±0.4 kb, ±0.3 kb, ±0.2 kb, or ±0.1 kb.

In another specific example, the genomic safe harbor locus corresponds to human L-SH18 (coordinates of about 170031084 to about 170031382 on chromosome 6) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means ±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to human L-SH20 (coordinates of about 25207412 to about 25207703 on chromosome 9) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In one specific example, the genomic safe harbor locus is mouse chromosome 14, coordinates 103,450,397-103,451,396 (referred to herein as mouse L-SH5) or a corresponding region (e.g., orthologous or syntenic region) in a non-human animal, non-human mammal (e.g., non-human primate), or rodent, such as a rat.

In one specific example, the genomic safe harbor locus is mouse chromosome 17, coordinates 15,226,387-15,227,386 (referred to herein as mouse L-SH18) or a corresponding region (e.g., orthologous or syntenic region) in a non-human animal, non-human mammal (e.g., non-human primate), or rodent, such as a rat.

In one specific example, the genomic safe harbor locus is mouse chromosome 4, coordinates 92,827,563-92,828,592 (referred to herein as mouse L-SH20) or a corresponding region (e.g., orthologous or syntenic region) in a non-human animal, non-human mammal (e.g., non-human primate), or rodent, such as a rat.

In one specific example, the genomic safe harbor locus corresponds to mouse L-SH5 (coordinates of about 103,450,397 to about 103,451,396 on mouse chromosome 14) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to mouse L-SH18 (coordinates of about 15,226,387 to about 15,227,386 on mouse chromosome 17) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to mouse L-SH20 (coordinates of about 92,827,563 to about 92,828,592 on mouse chromosome 4) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

### III. Methods for Introducing, Integrating, or Expressing a Nucleic Acid Encoding a Product of Interest in Cells or Subjects

The nucleic acid constructs and compositions disclosed herein can be used in methods of inserting or integrating a nucleic acid encoding a product of interest (e.g., a polypeptide of interest) into a target genomic locus (e.g., a genomic safe harbor locus as described elsewhere herein) or methods of expressing a product of interest (e.g., a polypeptide of interest) in a cell, in a population of cells, or in a subject (e.g., a subject in need thereof).

In one example, provided herein are methods of introducing a nucleic acid construct into a cell or a population of cells, such as a cell or a population of cells in a subject (e.g., a subject in need thereof). The nucleic acid construct can comprise a nucleic acid operably linked to a promoter (e.g., a promoter active in the cell or population of cells), wherein the nucleic acid encodes a product of interest (e.g., a polypeptide of interest). Such methods can comprise administering any of the nucleic acid constructs described herein (or any of the compositions comprising a nucleic acid construct described herein, including, for example, vectors or lipid nanoparticles) to the cell, the population of cells, or the subject (e.g., a subject in need thereof). In some methods, the nucleic acid construct or composition comprising the nucleic acid construct can be administered together with a nuclease agent (simultaneously or sequentially in any order) described herein. The nuclease agent can cleave a nuclease target sequence within a target genomic locus (e.g., genomic safe harbor locus) (e.g., to create a cleavage site), and the nucleic acid construct can be inserted into the target genomic locus (e.g., into the cleavage site) to create a modified target genomic locus. The product of interest (e.g., a polypeptide of interest) can be expressed from the modified target genomic locus. In one example, the nuclease agent is a CRISPR/Cas system, the cell or subject is a human cell (e.g., a human liver cell) or a human subject, and the genomic safe harbor locus is selected from the following genomic locations: (i) chromosome 13, coordinates 77460242-77460537; (ii) chromosome 6, coordinates 170031084-170031382; and (iii) chromosome 9, coordinates 25207412-25207703. In one example, the nuclease agent is a CRISPR/Cas system, the cell or subject is a mouse cell (e.g., a mouse liver cell) or a mouse subject, and the genomic safe harbor locus is selected from the following genomic locations: (i) chromosome 14, coordinates 103,450,397-103,451,396; (ii) chromosome 17, coordinates 15,226,387-15,227,386; and (iii) chromosome 4, coordinates 92,827,563-92,828,592. Alternatively, the cell or subject is a non-human animal cell (e.g., non-human animal liver cell) or subject, and the genomic safe harbor locus is selected from the corresponding genomic locations in the non-human animal. In such methods, the guide RNA can bind to the Cas protein and target the Cas protein to the guide RNA target sequence in the genomic safe harbor locus, the Cas protein can cleave the guide RNA target sequence (e.g., to create a cleavage site), the nucleic acid construct can be inserted into the genomic safe harbor locus (e.g., into the cleavage site) to create a modified the genomic safe harbor locus, and the product of interest (e.g., polypeptide of interest) can be expressed from the modified genomic safe harbor locus. In one specific example, the genomic safe harbor locus is human L-SH5 (chromosome 13, coordinates 77460242-77460537) or a corresponding region (e.g., orthologous or syntenic region) in a non-human animal, non-human mammal (e.g., non-human primate), or rodent, such as a rat or a mouse. In another specific example, the genomic safe harbor locus is human L-SH18 (chromosome 6, coordinates 170031084-170031382) or a corresponding region (e.g., orthologous or syntenic region) in a non-human animal, non-human mammal (e.g., non-human primate), or rodent, such as a rat or a mouse. In another specific example, the genomic safe harbor locus is human L-SH20 (chromosome 9, coordinates 25207412-25207703) or a corresponding region (e.g., orthologous or syntenic region) in a non-human animal, non-human mammal (e.g., non-human primate), or rodent, such as a rat or a mouse. In one specific example, the genomic safe harbor locus is mouse L-SH5 (chromosome 14, coordinates 103,450,397-103,451,396) or a corresponding region (e.g., orthologous or syntenic region) in a non-human animal, non-human mammal (e.g., non-human primate), or rodent, such as a rat. In another specific example, the genomic safe harbor locus is mouse L-SH18 (chromosome 17, coordinates 15,226,387-15,227,386) or a corresponding region (e.g., orthologous or syntenic region) in a non-human animal, non-human mammal (e.g., non-human primate), or rodent, such as a rat. In another specific example, the genomic safe harbor locus is mouse L-SH20 (chromosome 4, coordinates 92,827,563-92,828,592) or a corresponding region (e.g., orthologous or syntenic region) in a non-human animal, non-human mammal (e.g., non-human primate), or rodent, such as a rat.

In one example, provided herein are methods of inserting a nucleic acid construct into a target genomic locus (e.g., genomic safe harbor locus) in a cell or a population of cells, such as a cell or a population of cells in a subject (e.g., a subject in need thereof). The nucleic acid construct can comprise a nucleic acid operably linked to a promoter (e.g., a promoter active in the cell or population of cells), wherein the nucleic acid encodes a product of interest (e.g. a polypeptide of interest). Such methods can comprise administering any of the nucleic acid constructs described herein (or any of the compositions comprising a nucleic acid construct described herein, including, for example, vectors or lipid nanoparticles) to the cell, the population of cells, or the subject (e.g., a subject in need thereof). In some methods, the nucleic acid construct or composition comprising the nucleic acid construct can be administered together with a nuclease agent (simultaneously or sequentially in any order) described herein. The nuclease agent can cleave a nuclease target sequence within a target genomic locus (e.g., genomic safe harbor locus) (e.g., to create a cleavage site), and the nucleic acid construct can be inserted into the target genomic locus (e.g., into the cleavage site) to create a modified target genomic locus. The product of interest (e.g., polypeptide of interest) can be expressed from the modified target genomic locus. In one example, the nuclease agent is a CRISPR/Cas system, the cell or subject is a human cell (e.g., a human liver cell) or a human subject, and the genomic safe harbor locus is selected from the following genomic locations: (i) chromosome 13, coordinates 77460242-77460537; (ii) chromosome 6, coordinates 170031084-170031382; and (iii) chromosome 9, coordinates 25207412-25207703. In one example, the nuclease agent is a CRISPR/Cas system, the cell or subject is a mouse cell (e.g., a mouse liver cell) or a mouse subject, and the genomic safe harbor locus is selected from the following genomic locations: (i) chromosome 14, coordinates 103,450,397-103,451,396; (ii) chromosome 17, coordinates 15,226,387-15,227,386; and (iii) chromosome 4, coordinates 92,827,563-92,828,592. Alternatively, the cell or subject is a non-human animal cell (e.g., non-human animal liver cell) or subject, and the genomic safe harbor locus is selected from the corresponding genomic locations in the non-human animal. In such methods, the guide RNA can bind to the Cas protein and target the Cas protein to the guide RNA target sequence in the genomic safe harbor locus, the Cas protein can cleave the guide RNA target sequence (e.g., to create a cleavage site), the nucleic acid construct can be inserted into the genomic safe harbor locus (e.g., into the cleavage site) to create a modified genomic safe harbor locus, and the product of interest (e.g., polypeptide of interest) can be expressed from the modified genomic safe harbor locus. In one specific example, the genomic safe harbor locus is human L-SH5 (chromosome 13, coordinates 77460242-77460537) or a corresponding region (e.g., orthologous or syntenic region) in a non-human animal, non-human mammal (e.g., non-human primate), or rodent, such as a rat or a mouse. In another specific example, the genomic safe harbor locus is human L-SH18 (chromosome 6, coordinates 170031084-170031382) or a corresponding region (e.g., orthologous or syntenic region) in a non-human animal, non-human mammal (e.g., non-human primate), or rodent, such as a rat or a mouse. In another specific example, the genomic safe harbor locus is human L-SH20 (chromosome 9, coordinates 25207412-25207703) or a corresponding region (e.g., orthologous or syntenic region) in a non-human animal, non-human mammal (e.g., non-human primate), or rodent, such as a rat or a mouse. In one specific example, the genomic safe harbor locus is mouse L-SH5 (chromosome 14, coordinates 103,450,397-103,451,396) or a corresponding region (e.g., orthologous or syntenic region) in a non-human animal, non-human mammal (e.g., non-human primate), or rodent, such as a rat. In another specific example, the genomic safe harbor locus is mouse L-SH18 (chromosome 17, coordinates 15,226,387-15,227,386) or a corresponding region (e.g., orthologous or syntenic region) in a non-human animal, non-human mammal (e.g., non-human primate), or rodent, such as a rat. In another specific example, the genomic safe harbor locus is mouse L-SH20 (chromosome 4, coordinates 92,827,563-92,828,592) or a corresponding region (e.g., orthologous or syntenic region) in a non-human animal, non-human mammal (e.g., non-human primate), or rodent, such as a rat.

In another example, provided herein are methods of expressing a product of interest (e.g., polypeptide of interest) from a target genomic locus (e.g., genomic safe harbor locus) in a cell, a population of cells, or a subject (e.g., a subject in need thereof). The nucleic acid constructs can comprise a nucleic acid operably linked to a promoter (e.g., a promoter active in the cell or population of cells), wherein the nucleic acid encodes a product of interest (e.g., a polypeptide of interest). Such methods can comprise administering any of the nucleic acid constructs described herein (or any of the compositions comprising a nucleic acid construct described herein, including, for example, vectors or lipid nanoparticles) to the cell, the population of cells, or the subject (e.g., a subject in need thereof). In some methods, the nucleic acid construct can be administered together (simultaneously or sequentially in any order) with a nuclease agent described herein. The nuclease agent can cleave a nuclease target sequence within a target genomic locus (e.g., genomic safe harbor locus) (e.g., to create a cleavage site), the nucleic acid construct can be inserted into the target genomic locus (e.g., into the cleavage site) to create a modified target genomic locus, and the product of interest (e.g., polypeptide of interest) can be expressed from the modified target genomic locus. In one example, the nuclease agent is a CRISPR/Cas system, the cell or subject is a human cell (e.g., a human liver cell) or a human subject, and the genomic safe harbor locus is selected from the following genomic locations: (i) chromosome 13, coordinates 77460242-77460537; (ii) chromosome 6, coordinates 170031084-170031382; and (iii) chromosome 9, coordinates 25207412-25207703. In one example, the nuclease agent is a CRISPR/Cas system, the cell or subject is a mouse cell (e.g., a mouse liver cell) or a mouse subject, and the genomic safe harbor locus is selected from the following genomic locations: (i) chromosome 14, coordinates 103,450,397-103,451,396; (ii) chromosome 17, coordinates 15,226,387-15,227,386; and (iii) chromosome 4, coordinates 92,827,563-92,828,592. Alternatively, the cell or subject is a non-human animal cell (e.g., non-human animal liver cell) or subject, and the genomic safe harbor locus is selected from the corresponding genomic locations in the non-human animal. In such methods, the guide RNA can bind to the Cas protein and target the Cas protein to the guide RNA target sequence in the genomic safe harbor locus, the Cas protein can cleave the guide RNA target sequence (e.g., to create a cleavage site), the nucleic acid construct can be inserted into the genomic safe harbor locus to create a modified genomic safe harbor locus, and the product of interest (e.g., polypeptide of interest) can be expressed from the modified genomic safe harbor locus. In one specific example, the genomic safe harbor locus is human L-SH5 (chromosome 13, coordinates 77460242-77460537) or a corresponding region (e.g., orthologous or syntenic region) in a non-human animal, non-human mammal (e.g., non-human primate), or rodent, such as a rat or a mouse. In another specific example, the genomic safe harbor locus is human L-SH18 (chromosome 6, coordinates 170031084-170031382) or a corresponding region (e.g., orthologous or syntenic region) in a non-human animal, non-human mammal (e.g., non-human primate), or rodent, such as a rat or a mouse. In another specific example, the genomic safe harbor locus is human L-SH20 (chromosome 9, coordinates 25207412-25207703) or a corresponding region (e.g., orthologous or syntenic region) in a non-human animal, non-human mammal (e.g., non-human primate), or rodent, such as a rat or a mouse. In one specific example, the genomic safe harbor locus is mouse L-SH5 (chromosome 14, coordinates 103,450,397-103,451,396) or a corresponding region (e.g., orthologous or syntenic region) in a non-human animal, non-human mammal (e.g., non-human primate), or rodent, such as a rat. In another specific example, the genomic safe harbor locus is mouse L-SH18 (chromosome 17, coordinates 15,226,387-15,227,386) or a corresponding region (e.g., orthologous or syntenic region) in a non-human animal, non-human mammal (e.g., non-human primate), or rodent, such as a rat. In another specific example, the genomic safe harbor locus is mouse L-SH20 (chromosome 4, coordinates 92,827,563-92,828,592) or a corresponding region (e.g., orthologous or syntenic region) in a non-human animal, non-human mammal (e.g., non-human primate), or rodent, such as a rat.

In any of the above methods, the cells can be from any suitable species, such as eukaryotic cells or mammalian cells (e.g., non-human mammalian cells or human cells). A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples of cells include, but are not limited to, human cells, rodent cells, mouse cells, rat cells, and non-human primate cells. In a specific example, the cell is a human cell. Likewise, cells can be any suitable type of cell. In a specific example, the cell is a liver cell such as a hepatocyte (e.g., a human liver cell or human hepatocyte).

The cells can be isolated cells (e.g., in vitro), ex vivo cells, or can be in vivo within an animal (i.e., in a subject). In a specific example, the cell can be in vitro or ex vivo. In a specific example, the cell is in vivo (in a subject). Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatocytes (e.g., mouse, non-human primate, or human hepatocytes).

The cells provided herein can be normal, healthy cells, or can be diseased or mutant-bearing cells. For example, the cells may demonstrate a loss of function, e.g., a loss of enzyme function.

In some methods, the product of interest is a therapeutic product, and the subject is a subject in need of the therapeutic product. For example, the product of interest can be a therapeutic polypeptide (e.g., enzyme), such as a polypeptide that is lacking or deficient in a subject or a polypeptide whose activity is lacking or deficient in a subject. For example, the subject can comprise a mutation in their genome, wherein the mutation results in reduced activity or expression of an endogenous polypeptide having enzymatic activity, and the polypeptide of interest can encode a polypeptide having the enzymatic activity of a wild type polypeptide encoded by the gene in which the subject has a mutation that results in reduced activity or expression of the endogenous polypeptide. Alternatively, the product of interest can be a therapeutic RNA such as an antisense oligonucleotide or an RNAi agent, or a therapeutic polypeptide such as an antibody, an antigen-binding protein, an exogenous T cell receptor, or a chimeric antigen receptor (CAR), wherein the therapeutic product (e.g., therapeutic RNA or therapeutic polypeptide) treats a disease or condition in the subject.

The compositions disclosed herein (e.g., nucleic acid constructs encoding a product of interest, or nucleic acid constructs in combination with the nuclease agents (e.g., CRISPR/Cas systems) are useful for the treatment of a subject in need of the product of interest. Likewise, the compositions disclosed herein can be used for the preparation of a pharmaceutical composition or medicament for treating a subject in need thereof. The terms “treat,” “treated,” “treating,” and “treatment,” include the administration of the nucleic acid constructs disclosed herein (e.g., together with a nuclease agent disclosed herein) to subjects to prevent or delay the onset of the symptoms, complications, or biochemical indicia of a disease, alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

In some methods, a therapeutically effective amount of the nucleic acid construct or the composition comprising the nucleic acid construct or the combination of the nucleic acid construct and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311. In certain embodiments, the pharmaceutical compositions are non-pyrogenic.

The subject in any of the above methods can be from any suitable species, such as a eukaryote or a mammal. A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples of suitable species include, but are not limited to, humans, rodents, mice, rats, and non-human primates. In a specific example, the subject is a human.

Any genomic safe harbor locus capable of expressing a gene can be used in the methods described herein. Such loci are described in more detail elsewhere herein. In one specific example, the genomic safe harbor locus is human L-SH5 (chromosome 13, coordinates 77460242-77460537) or a corresponding region (e.g., orthologous or syntenic region) in a non-human animal, non-human mammal (e.g., non-human primate), or rodent, such as a rat or a mouse. In another specific example, the genomic safe harbor locus is human L-SH18 (chromosome 6, coordinates 170031084-170031382) or a corresponding region (e.g., orthologous or syntenic region) in a non-human animal, non-human mammal (e.g., non-human primate), or rodent, such as a rat or a mouse. In another specific example, the genomic safe harbor locus is human L-SH20 (chromosome 9, coordinates 25207412-25207703) or a corresponding region (e.g., orthologous or syntenic region) in a non-human animal, non-human mammal (e.g., non-human primate), or rodent, such as a rat or a mouse.

In a specific example, the genomic safe harbor locus is selected from the following genomic locations: (i) human chromosome 13, coordinates 77460242-77460537 (referred to herein as L-SH5) or a corresponding region (e.g., orthologous or syntenic region) in a non-human animal, non-human mammal (e.g., non-human primate), or rodent, such as a rat or a mouse; (ii) human chromosome 6, coordinates 170031084-170031382 (referred to herein as L-SH18) or a corresponding region (e.g., orthologous or syntenic region) in a non-human animal, non-human mammal (e.g., non-human primate), or rodent, such as a rat or a mouse; and (iii) human chromosome 9, coordinates 25207412-25207703 (referred to herein as L-SH20) or a corresponding region (e.g., orthologous or syntenic region) in a non-human animal, non-human mammal (e.g., non-human primate), or rodent, such as a rat or a mouse.

In a specific example, the genomic safe harbor locus is selected from the following genomic coordinates: (i) about 77460242 to about 77460537 on human chromosome 13 (corresponds to L-SH5) or a corresponding region (e.g., orthologous or syntenic region) in a non-human animal, non-human mammal (e.g., non-human primate), or rodent, such as a rat or a mouse; (ii) about 170031084 to about 170031382 on human chromosome 6 (corresponds to L-SH18) or a corresponding region (e.g., orthologous or syntenic region) in a non-human animal, non-human mammal (e.g., non-human primate), or rodent, such as a rat or a mouse; and (iii) about 25207412 to about 25207703 on human chromosome 9 (corresponds to L-SH20) or a corresponding region (e.g., orthologous or syntenic region) in a non-human animal, non-human mammal (e.g., non-human primate), or rodent, such as a rat or a mouse. The term “about” when referring to genomic coordinates means±20 base pairs. In other examples, the genomic safe harbor locus is near the regions identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In one specific example, the genomic safe harbor locus is human L-SH5 (chromosome 13, coordinates 77460242-77460537) or a corresponding region (e.g., orthologous or syntenic region) in a non-human animal, non-human mammal (e.g., non-human primate), or rodent, such as a rat or a mouse. For example, the genomic safe harbor locus can comprise the sequence set forth in SEQ ID NO: 39 or variants thereof which are located at the same position, or genetic locus, on a chromosome in humans or orthologous or syntenic regions in a non-human animal, non-human mammal (e.g., non-human primate), or rodent, such as a rat or a mouse. Syntenic regions are derived from a single ancestral genomic region. For example, syntenic regions can be from different organisms and are derived from speciation.

In another specific example, the genomic safe harbor locus is human L-SH18 (chromosome 6, coordinates 170031084-170031382) or a corresponding region (e.g., orthologous or syntenic region) in a non-human animal, non-human mammal (e.g., non-human primate), or rodent, such as a rat or a mouse. For example, the genomic safe harbor locus can comprise the sequence set forth in SEQ ID NO: 40 or variants thereof which are located at the same position, or genetic locus, on a chromosome in humans or orthologous or syntenic regions in a non-human animal, non-human mammal (e.g., non-human primate), or rodent, such as a rat or a mouse.

In another specific example, the genomic safe harbor locus is human L-SH20 (chromosome 9, coordinates 25207412-25207703) or a corresponding region (e.g., orthologous or syntenic region) in a non-human animal, non-human mammal (e.g., non-human primate), or rodent, such as a rat or a mouse. For example, the genomic safe harbor locus can comprise the sequence set forth in SEQ ID NO: 41 or variants thereof which are located at the same position, or genetic locus, on a chromosome in humans or orthologous or syntenic regions in a non-human animal, non-human mammal (e.g., non-human primate), or rodent, such as a rat or a mouse.

In one specific example, the genomic safe harbor locus corresponds to human L-SH5 (coordinates of about 77460242 to about 77460537 on chromosome 13) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to human L-SH18 (coordinates of about 170031084 to about 170031382 on chromosome 6) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means ±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to human L-SH20 (coordinates of about 25207412 to about 25207703 on chromosome 9) or a corresponding region (e.g., orthologous or syntenic region) in a non-human animal, non-human mammal (e.g., non-human primate), or rodent, such as a rat or a mouse, or variants thereof which are located at the same position, or genetic locus, on a chromosome in humans or orthologous or syntenic regions in a non-human animal, non-human mammal (e.g., non-human primate), or rodent, such as a rat or a mouse. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

Any genomic safe harbor locus capable of expressing a gene can be used in the methods described herein. Such loci are described in more detail elsewhere herein. In one specific example, the genomic safe harbor locus is mouse L-SH5 (chromosome 14, coordinates 103,450,397-103,451,396) or a corresponding region (e.g., orthologous or syntenic region) in a non-human animal, non-human mammal (e.g., non-human primate), or rodent, such as a rat. In another specific example, the genomic safe harbor locus is mouse L-SH18 (chromosome 17, coordinates 15,226,387-15,227,386) or a corresponding region (e.g., orthologous or syntenic region) in a non-human animal, non-human mammal (e.g., non-human primate), or rodent, such as a rat. In another specific example, the genomic safe harbor locus is mouse L-SH20 (chromosome 4, coordinates 92,827,563-92,828,592) or a corresponding region (e.g., orthologous or syntenic region) in a non-human animal, non-human mammal (e.g., non-human primate), or rodent, such as a rat.

In a specific example, the genomic safe harbor locus is selected from the following genomic locations: (i) mouse chromosome 14, coordinates 103,450,397-103,451,396 (referred to herein as mouse L-SH5) or a corresponding region (e.g., orthologous or syntenic region) in a non-human animal, non-human mammal (e.g., non-human primate), or rodent, such as a rat; (ii) mouse chromosome 17, coordinates 15,226,387-15,227,386 (referred to herein as mouse L-SH18) or a corresponding region (e.g., orthologous or syntenic region) in a non-human animal, non-human mammal (e.g., non-human primate), or rodent, such as a rat; and (iii) mouse chromosome 4, coordinates 92,827,563-92,828,592 (referred to herein as mouse L-SH20) or a corresponding region (e.g., orthologous or syntenic region) in a non-human animal, non-human mammal (e.g., non-human primate), or rodent, such as a rat.

In a specific example, the genomic safe harbor locus is selected from the following genomic coordinates: (i) about 103,450,397 to about 103,451,396 on mouse chromosome 14 (corresponds to mouse L-SH5) or a corresponding region (e.g., orthologous or syntenic region) in a non-human animal, non-human mammal (e.g., non-human primate), or rodent, such as a rat; (ii) about 15,226,387 to about 15,227,386 on mouse chromosome 17 (corresponds to mouse L-SH18) or a corresponding region (e.g., orthologous or syntenic region) in a non-human animal, non-human mammal (e.g., non-human primate), or rodent, such as a rat; and (iii) about 92,827,563 to about 92,828,592 on mouse chromosome 4 (corresponds to mouse L-SH20) or a corresponding region (e.g., orthologous or syntenic region) in a non-human animal, non-human mammal (e.g., non-human primate), or rodent, such as a rat. The term “about” when referring to genomic coordinates means±20 base pairs. In other examples, the genomic safe harbor locus is near the regions identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or +0.1 kb.

In one specific example, the genomic safe harbor locus is mouse L-SH5 (mouse chromosome 14, coordinates 103,450,397-103,451,396) or a corresponding region (e.g., orthologous or syntenic region) in a non-human animal, non-human mammal (e.g., non-human primate), or rodent, such as a rat. For example, the genomic safe harbor locus can comprise the sequence set forth in SEQ ID NO: 405 or variants thereof which are located at the same position, or genetic locus, on a chromosome in mice or orthologous or syntenic regions in a human or non-human animal, non-human mammal (e.g., non-human primate), or rodent, such as a rat. Syntenic regions are derived from a single ancestral genomic region. For example, syntenic regions can be from different organisms and are derived from speciation.

In another specific example, the genomic safe harbor locus is mouse L-SH18 (chromosome 17, coordinates 15,226,387-15,227,386) or a corresponding region (e.g., orthologous or syntenic region) in a non-human animal, non-human mammal (e.g., non-human primate), or rodent, such as a rat. For example, the genomic safe harbor locus can comprise the sequence set forth in SEQ ID NO: 406 or variants thereof which are located at the same position, or genetic locus, on a chromosome in mice or orthologous or syntenic regions in a human or non-human animal, non-human mammal (e.g., non-human primate), or rodent, such as a rat.

In another specific example, the genomic safe harbor locus is mouse L-SH20 (chromosome 4, coordinates 92,827,563-92,828,592) or a corresponding region (e.g., orthologous or syntenic region) in a non-human animal, non-human mammal (e.g., non-human primate), or rodent, such as a rat. For example, the genomic safe harbor locus can comprise the sequence set forth in SEQ ID NO: 407 or variants thereof which are located at the same position, or genetic locus, on a chromosome in mice or orthologous or syntenic regions in a human or non-human animal, non-human mammal (e.g., non-human primate), or rodent, such as a rat.

In one specific example, the genomic safe harbor locus corresponds to mouse L-SH5 (coordinates of about 103,450,397 to about 103,451,396 on mouse chromosome 14) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to mouse L-SH18 (coordinates of about 15,226,387 to about 15,227,386 on mouse chromosome 17) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

In another specific example, the genomic safe harbor locus corresponds to mouse L-SH20 (coordinates of about 92,827,563 to about 92,828,592 on mouse chromosome 4) or a corresponding region (e.g., orthologous or syntenic region) in a non-human animal, non-human mammal (e.g., non-human primate), or rodent, such as a rat, or variants thereof which are located at the same position, or genetic locus, on a chromosome in mice or orthologous or syntenic regions in a non-human animal, non-human mammal (e.g., non-human primate), or rodent, such as a rat. The term “about” when referring to genomic coordinates means±20 base pairs. In other examples, the genomic safe harbor locus is near the region identified by the above coordinates. The term “near” when referring to genomic coordinates means±5 kb, ±4 kb, ±3 kb, ±2 kb, ±1 kb, ±0.5 kb, ±0.4 kb, ±0.3 kb, ±0.2 kb, or ±0.1 kb.

The nucleic acid construct can be inserted into the target genomic locus by any means, including homologous recombination (HR) and non-homologous end joining (NHEJ) as described elsewhere herein. In a specific example, the nucleic acid construct is inserted by NHEJ (e.g., does not comprise a homology arm and is inserted by NHEJ).

In another specific example, the nucleic acid construct can be inserted via homology-independent targeted integration (e.g., directional homology-independent targeted integration). For example, the nucleic acid construct (i.e., the nucleic acid operably linked to a promoter, wherein the nucleic acid encodes a product of interest) can be flanked on each side by a target site for a nuclease agent (e.g., the same target site as in the target genomic locus, and the same nuclease agent being used to cleave the target site in the target genomic locus). The nuclease agent can then cleave the target sites flanking the nucleic acid construct (i.e., the nucleic acid operably linked to a promoter, wherein the nucleic acid encodes a product of interest). In a specific example, the nucleic acid construct is delivered AAV-mediated delivery, and cleavage of the target sites flanking the nucleic acid construct (i.e., the nucleic acid operably linked to a promoter, wherein the nucleic acid encodes a product of interest) can remove the inverted terminal repeats (ITRs) of the AAV. Removal of the ITRs can make it easier to assess successful targeting, because presence of the ITRs can hamper sequencing efforts due to the repeated sequences. In some methods, the target site in the target genomic locus (e.g., a gRNA target sequence including the flanking protospacer adjacent motif) is no longer present if the nucleic acid construct (i.e., the nucleic acid operably linked to a promoter, wherein the nucleic acid encodes a product of interest) is inserted into the target genomic locus in a first orientation but it is reformed if the nucleic acid construct (i.e., the nucleic acid operably linked to a promoter, wherein the nucleic acid encodes a product of interest) is inserted into the target genomic locus in the opposite orientation.

In any of the above methods, the nucleic acid construct encoding the product of interest can be administered simultaneously with the nuclease agent (e.g., CRISPR/Cas system) or not simultaneously (e.g., sequentially in any combination). For example, in a method comprising administering a composition comprising the nucleic acid construct and a nuclease agent, they can be administered separately. For example, the nucleic acid construct can be administered prior to the nuclease agent, subsequent to the nuclease agent, or at the same time as the nuclease agent.

In one example, the nucleic acid construct is administered about 4 hours, about 8 hours, about 12 hours, about 18 hours, about 1 day, about 2 days, about 3 days, about 4 days, about 5 days, about 6 days, or about 1 week prior to administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nuclease agent.

In one example, the nucleic acid construct is administered about 4 hours, about 8 hours, about 12 hours, about 18 hours, about 1 day, about 2 days, about 3 days, about 4 days, about 5 days, about 6 days, or about 1 week after administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nuclease agent.

Any suitable methods of administering nucleic acid constructs and nuclease agents to cells can be used, particularly methods of administering to the liver, and examples of such methods are described in more detail elsewhere herein. In methods of targeting a cell in vivo in a subject, the nucleic acid construct can be inserted in particular types of cells in the subject. The method and vehicle for introducing the nucleic acid construct and/or the nuclease agent into the subject can affect which types of cells in the subject are targeted. In some methods, for example, the nucleic acid construct is inserted into a target genomic locus (e.g., a genomic safe harbor locus as disclosed herein) in liver cells, such as hepatocytes. Methods and vehicles for introducing such constructs and nuclease agents into the subject (including methods and vehicles that target the liver or hepatocytes, such as lipid nanoparticle-mediated delivery and AAV-mediated delivery (e.g., rAAV8-mediated delivery) and intravenous injection), are disclosed in more detail elsewhere herein.

In any of the above methods, the nucleic acid construct and the nuclease agent (e.g., CRISPR/Cas system) can be administered using any suitable delivery system and known method. The nuclease agent components and nucleic acid construct (e.g., the guide RNA, Cas protein, and nucleic acid construct) can be delivered individually or together in any combination, using the same or different delivery methods as appropriate.

In methods in which a CRISPR/Cas system is used, a guide RNA can be introduced into or administered to a subject or cell, for example, in the form of an RNA (e.g., in vitro transcribed RNA, such as the modified guide RNAs disclosed herein) or in the form of a DNA encoding the guide RNA. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be introduced into a subject or cell in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)), such as a modified mRNA as disclosed herein, or DNA). Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

In one example, the Cas protein is introduced in the form of an mRNA (e.g., a modified mRNA as disclosed herein), and the guide RNA is introduced in the form of RNA such as a modified gRNA as disclosed herein (e.g., together within the same lipid nanoparticle). Guide RNAs can be modified as disclosed elsewhere herein. Likewise, Cas mRNAs can be modified as disclosed elsewhere herein.

In methods in which a nucleic acid construct is inserted following cleavage by a genome-editing system (e.g., a Cas protein), the genome-editing system (e.g., Cas protein) can cleave the target genomic locus to create a single-strand break (nick) or double-strand break, and the cleaved or nicked locus can be repaired by insertion of the nucleic acid construct via non-homologous end joining (NHEJ)-mediated insertion or homology-directed repair. Optionally, repair with the nucleic acid construct removes or disrupts the guide RNA target sequence(s) so that alleles that have been targeted cannot be re-targeted by the CRISPR/Cas reagents.

As explained in more detail elsewhere herein, the nucleic acid constructs can comprise deoxyribonucleic acid (DNA) or ribonucleic acid (RNA), they can be single-stranded or double-stranded, and they can be in linear or circular form. The nucleic acid constructs can be naked nucleic acids or can be delivered by viruses, such as AAV. In a specific example, the nucleic acid construct can be delivered via AAV and can be capable of insertion into the target genomic locus (e.g., a genomic safe harbor locus as described elsewhere herein) by non-homologous end joining (e.g., the nucleic acid construct can be one that does not comprise a homology arm).

Some nucleic acid constructs are capable of insertion by non-homologous end joining. In some cases, such nucleic acid constructs do not comprise a homology arm. For example, such nucleic acid constructs can be inserted into a blunt end double-strand break following cleavage with a Cas protein. In a specific example, the nucleic acid construct can be delivered via AAV and can be capable of insertion by non-homologous end joining (e.g., the nucleic acid construct can be one that does not comprise a homology arm).

In another example, the nucleic acid construct can be inserted via homology-independent targeted integration. For example, the nucleic acid construct (i.e., the nucleic acid operably linked to a promoter, wherein the nucleic acid encodes a product of interest) can be flanked on each side by a guide RNA target sequence (e.g., the same target site as in the target genomic locus, and the CRISPR/Cas reagent (Cas protein and guide RNA) being used to cleave the target site in the target genomic locus). The Cas protein can then cleave the target sites flanking the nucleic acid construct (i.e., the nucleic acid operably linked to a promoter, wherein the nucleic acid encodes a product of interest). In a specific example, the nucleic acid construct is delivered AAV-mediated delivery, and cleavage of the target sites flanking the nucleic acid construct (i.e., the nucleic acid operably linked to a promoter, wherein the nucleic acid encodes a product of interest) can remove the inverted terminal repeats (ITRs) of the AAV. In some methods, the target site in the target genomic locus (e.g., a guide RNA target sequence including the flanking protospacer adjacent motif) is no longer present if the nucleic acid construct (i.e., the nucleic acid operably linked to a promoter, wherein the nucleic acid encodes a product of interest) is inserted into the target genomic locus in a first orientation but it is reformed if the nucleic acid construct (i.e., the nucleic acid operably linked to a promoter, wherein the nucleic acid encodes a product of interest) is inserted into the target genomic locus in the opposite orientation.

The methods disclosed herein can comprise introducing or administering into a subject (e.g., an animal or mammal, such as a human) or cell a nucleic acid construct encoding a product of interest and optionally a nuclease agent such as CRISPR/Cas reagents, including in the form of nucleic acids (e.g., DNA or RNA), proteins, or nucleic-acid-protein complexes. “Introducing” or “administering” includes presenting to the cell or subject the molecule(s) (e.g., nucleic acid(s) or protein(s)) in such a manner that it gains access to the interior of the cell or to the interior of cells within the subject. The introducing can be accomplished by any means, and two or more of the components (e.g., two of the components, or all of the components) can be introduced into the cell or subject simultaneously or sequentially in any combination. For example, a Cas protein can be introduced into a cell or subject before introduction of a guide RNA, or it can be introduced following introduction of the guide RNA. As another example, a nucleic acid construct can be introduced prior to the introduction of a Cas protein and a guide RNA, or it can be introduced following introduction of the Cas protein and the guide RNA (e.g., the nucleic acid construct can be administered about 1, 2, 3, 4, 8, 12, 24, 36, 48, or 72 hours before or after introduction of the Cas protein and the guide RNA). See, e.g., US 2015/0240263 and US 2015/0110762, each of which is herein incorporated by reference in its entirety for all purposes. In addition, two or more of the components can be introduced into the cell or subject by the same delivery method or different delivery methods. Similarly, two or more of the components can be introduced into a subject by the same route of administration or different routes of administration.

A guide RNA can be introduced into a subject or cell, for example, in the form of an RNA (e.g., in vitro transcribed RNA) or in the form of a DNA encoding the guide RNA. Guide RNAs can be modified as disclosed elsewhere herein. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Cas RNAs can be modified as disclosed elsewhere herein. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a pluripotent cell, an embryonic stem (ES) cell, an adult stem cell, a developmentally restricted progenitor cell, an induced pluripotent stem (iPS) cell, or a one-cell stage embryo. For example, a suitable promoter can be active in a liver cell such as a hepatocyte. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery. In preferred embodiments, promotors are accepted by regulatory authorities for use in humans. In certain embodiments, promotors drive expression in a liver cell.

Molecules (e.g., Cas proteins or guide RNAs or nucleic acids encoding) introduced into the subject or cell can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or subject. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids and proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper components to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes.

In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a nucleic acid construct. In certain LNPs, the cargo can include an mRNA encoding a Cas nuclease, such as Cas9, a guide RNA or a nucleic acid encoding a guide RNA, and a nucleic acid construct. LNPs for use in the methods are described in more detail elsewhere herein.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or nucleic acid construct encoding a polypeptide of interest). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example as mRNA or protein, can ensure that the Cas/gRNA complex is only present and active for a short period of time and can reduce immunogenicity caused by peptides from the bacterially-derived Cas enzyme being displayed on the surface of the cell by MHC molecules. Such transient delivery can also reduce the possibility of off-target modifications.

Administration in vivo can be by any suitable route including, for example, systemic routes of administration such as parenteral administration, e.g., intravenous, subcutaneous, intra-arterial, or intramuscular. In a specific example, administration in vivo is intravenous.

Compositions comprising the guide RNAs and/or Cas proteins (or nucleic acids encoding the guide RNAs and/or Cas proteins) can be formulated using one or more physiologically and pharmaceutically acceptable carriers, diluents, excipients or auxiliaries. The formulation can depend on the route of administration chosen. Pharmaceutically acceptable means that the carrier, diluent, excipient, or auxiliary is compatible with the other ingredients of the formulation and not substantially deleterious to the recipient thereof. In a specific example, the route of administration and/or formulation or chosen for delivery to the liver (e.g., hepatocytes).

The methods disclosed herein can increase product of interest (e.g., polypeptide of interest) levels and/or product of interest (e.g., polypeptide of interest) activity levels in a cell or subject and can comprise measuring product of interest (e.g., polypeptide of interest) levels and/or activity levels in a cell or subject.

Some methods comprise expressing a therapeutically effective amount of the product of interest (e.g., polypeptide of interest). The specific level of expression required depends, for example, on the particular disease or condition to be treated

In some methods in which the subject did not express the product of interest (e.g., polypeptide of interest) prior to treatment, the method results in expression of the product of interest (e.g., polypeptide of interest) at a detectable level above zero, e.g., at a statistically significant level (e.g., a clinically relevant level).

Some methods comprise achieving a durable or sustained effect in a human, such as an at least at least 8 weeks, at least 24 weeks, for example, at least 1 year (52 weeks), or optionally at least 2 year effect, and in some embodiments, at least 3 year, at least 4 year, or at least 5 year effect. Some methods comprise achieving an effect (e.g., a therapeutic effect) in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased product of interest (e.g., polypeptide of interest) activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of product of interest (e.g., polypeptide of interest) in a human is achieved by at least 7 days, at least 14 days, or at least 28 days, optionally at least 56 days, at least 80 days, or at least 96 days. In additional methods, the method comprises maintaining product of interest (e.g., polypeptide of interest) activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the product of interest (e.g., polypeptide of interest) can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the product of interest (e.g., polypeptide of interest) can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the product of interest (e.g., polypeptide of interest) is maintained at a level higher than the expression or activity of the product of interest (e.g., polypeptide of interest) prior to treatment (i.e., the subject's baseline). In some methods, expression or activity of the product of interest (e.g., polypeptide of interest) is considered sustained if it is maintained at a therapeutically effective level of expression or activity. Relative durations, in other organisms, are understood based, e.g., on life span and developmental stages, are covered within the disclosure above. In some methods, expression or activity of the product of interest (e.g., polypeptide of interest) is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the product of interest (e.g., polypeptide of interest), e.g., weekly, monthly, particularly early after administration, e.g., within the first six months. Periodic measurements may establish that the effect on expression or activity is sustained at, e.g. 6 months after administration, one year after administration, or two years after administration.

In some methods, the expression or activity of the product of interest (e.g., polypeptide of interest) is at least 50% of the expression or activity of the product of interest (e.g., polypeptide of interest) at a peak level of expression measured for the human subject at 24 weeks after the administering. In some methods, the expression or activity of the product of interest (e.g., polypeptide of interest) is at least 50% of the expression or activity of the product of interest (e.g., polypeptide of interest) at a peak level of expression measured for the human subject at one year after the administering. In some methods, the expression or activity of the product of interest (e.g., polypeptide of interest) is at least 60% of the expression or activity of the product of interest (e.g., polypeptide of interest) at a peak level of expression measured for the human subject at 24 weeks after the administering. In some methods, expression or activity of the product of interest (e.g., polypeptide of interest) is at least 50% of the expression or activity of the product of interest (e.g., polypeptide of interest) at a peak level of expression measured for the human subject at two years after the administering. In some methods, the expression or activity of the product of interest (e.g., polypeptide of interest) is at least 60% of the expression or activity of the polypeptide at a peak level of expression measured for the human subject at 2 years after the administering. In some methods, the expression or activity of the product of interest (e.g., polypeptide of interest) is at least 60% of the expression or activity of the product of interest (e.g., polypeptide of interest) at a peak level of expression measured for the human subject at 24 weeks after the administering.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 2
Description of Sequences.

| SEQ ID NO | Type | Description |
|---|---|---|
| 1 | Protein | Cas9 Protein |
| 2 | DNA | Cas9 CDS |
| 3 | Protein | SV40 NLS v1 |
| 4 | Protein | SV40 NLS v2 |
| 5 | Protein | Nucleoplasmin NLS |
| 6 | RNA | crRNA Tail v1 |
| 7 | RNA | crRNA Tail v2 |
| 8 | RNA | TracrRNA v1 |
| 9 | RNA | TracrRNA v2 |
| 10 | RNA | TracrRNA v3 |
| 11 | RNA | gRNA Scaffold v1 |
| 12 | RNA | gRNA Scaffold v2 |
| 13 | RNA | gRNA Scaffold v3 |
| 14 | RNA | gRNA Scaffold v4 |
| 15 | RNA | gRNA Scaffold v5 |
| 16 | RNA | gRNA Scaffold v6 |
| 17 | RNA | gRNA Scaffold v7 |
| 18 | RNA | gRNA Scaffold v8 |
| 19 | DNA | Guide RNA Target Sequence Plus PAM v1 |
| 20 | DNA | Guide RNA Target Sequence Plus PAM v2 |
| 21 | DNA | Guide RNA Target Sequence Plus PAM v3 |
| 22 | DNA | L-SH5.1 gRNA Target Site |
| 23 | DNA | L-SH18.2 gRNA Target Site |
| 24 | DNA | L-SH20.1 gRNA Target Site |
| 25 | RNA | L-SH 5.1 Guide Sequence |
| 26 | RNA | L-SH18.2 Guide Sequence |
| 27 | RNA | L-SH20.1 Guide Sequence |
| 28 | RNA | L-SH5.1 sgRNA |
| 29 | RNA | L-SH18.2 sgRNA |
| 30 | RNA | L-SH20.1 sgRNA |
| 31 | Protein | T2A |
| 32 | Protein | P2A |
| 33 | Protein | E2A |
| 34 | Protein | F2A |
| 35 | DNA | L-SH4.2 gRNA Target Site |
| 36 | DNA | L-SH11 gRNA Target Site |
| 37 | DNA | L-SH14.2 gRNA Target Site |
| 38 | DNA | L-SH17 gRNA Target Site |
| 39 | DNA | L-SH5 Genomic Safe Harbor |
| 40 | DNA | L-SH18 Genomic Safe Harbor |
| 41 | DNA | L-SH20 Genomic Safe Harbor |
| 42 | DNA | L-SH5.2 gRNA Target Site |
| 43 | DNA | L-SH18.1 gRNA Target Site |
| 44 | DNA | L-SH20.2 gRNA Target Site |
| 45 | RNA | L-SH 5.2 Guide Sequence |
| 46 | RNA | L-SH18.1 Guide Sequence |
| 47 | RNA | L-SH20.2 Guide Sequence |
| 48 | RNA | L-SH5.2 sgRNA |
| 49 | RNA | L-SH18.1 sgRNA |
| 50 | RNA | L-SH20.2 sgRNA |
| 51-79 | DNA | Human L-SH5 gRNA Target Sites |
| 80-108 | DNA | Human L-SH18 gRNA Target Sites |
| 109-137 | DNA | Human L-SH20 gRNA Target Sites |
| 138-167 | DNA | Mouse L-SH5 gRNA Target Sites |
| 168-197 | DNA | Mouse L-SH18 gRNA Target Sites |
| 198-227 | DNA | Mouse L-SH20 gRNA Target Sites |
| 228-256 | RNA | Human L-SH5 gRNA Guide Sequences |
| 257-285 | RNA | Human L-SH18 gRNA Guide Sequences |
| 286-314 | RNA | Human L-SH20 gRNA Guide Sequences |
| 315-344 | RNA | Mouse L-SH5 gRNA Guide Sequences |
| 345-374 | RNA | Mouse L-SH18 gRNA Guide Sequences |
| 375-404 | RNA | Mouse L-SH20 gRNA Guide Sequences |
| 405 | DNA | Mouse Syntenic L-SH5 Genomic Safe Harbor |
| 406 | DNA | Mouse Syntenic L-SH18 Genomic Safe Harbor |
| 407 | DNA | Mouse Syntenic L-SH20 Genomic Safe Harbor |

### EXAMPLES

### Example 1. Identification of Liver Extragenic Safe Harbors for Gene Therapy Approaches

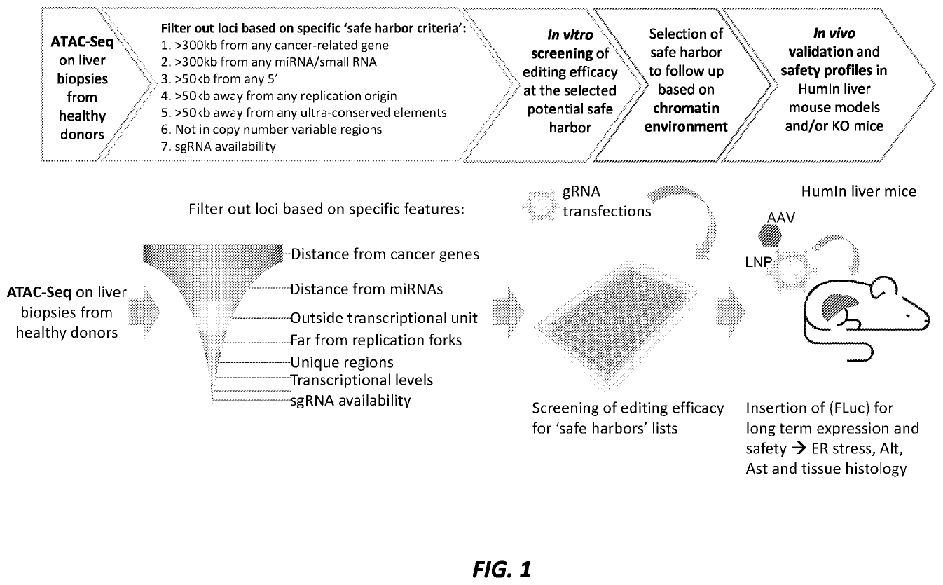

To identify extragenic genomic loci accessible in the liver, a systematic approach was used as shown in FIG. 1. To first identify accessible chromatin sites in the liver, we used ATAC-Seq datasets specifically from human liver biopsies, as chromatin states can largely diverge across different tissues and cell types. A total of 15,349 unique ATAC-Seq peaks were identified in healthy human liver biopsies. The compiled list of genomic loci was then filtered using the safe harbor criteria shown in Table 3.

TABLE 3
Identification of Putative Genomic Safe Harbors

| Genomic Safe Harbor Criteria | Passed | |
|---|---|---|
| 1. >300 kb from any cancer-related gene (724 genes) | 11,353 | (74%) |
| 2. >300 kb from any miRNA/small RNA (1,340 genes) | 11,468 | (75%) |
| 3. >50 kb from any 5′ | 172 | (1%) |
| 4. >50 kb away from any replication origin | 11,464 | (75%) |
| 5. >50 kb away from any ultra-conserved elements | 15,026 | (99%) |
| 6. Not in copy number variable regions (unique) | 5,744 | (37%) |

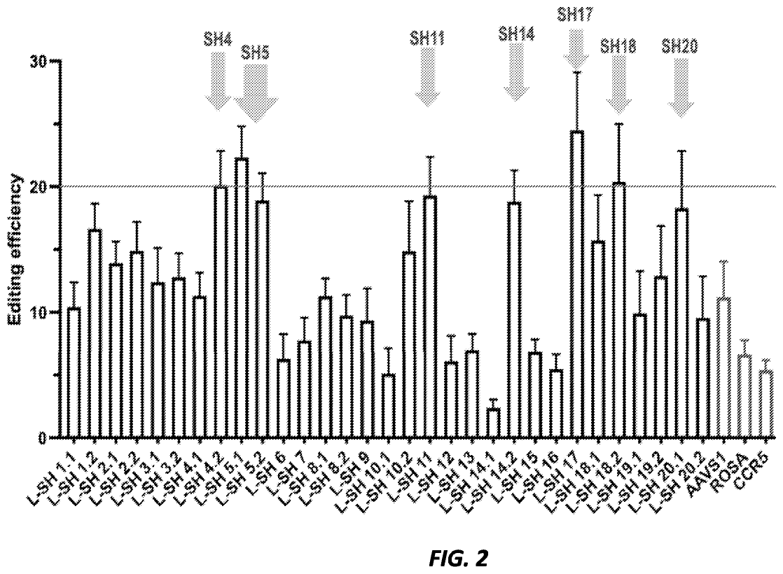

Out of the 15,349 ATAC-Seq peaks, 44 passed the criteria we used for genomic safe harbors. The list of potential safe harbors was then screened in primary human hepatocytes to determine editing efficiency for each locus compared to well-characterized gRNAs for two well-characterized liver intragenic loci (positive control) and a non-targeting gRNA (negative control). In the case of liver, we identified 44 ATAC-Seq peaks, for which we could design 33 gRNAs (high score, no/low off-targets) that work with Streptococcus pyogenes Cas9, covering 20 loci. Of these 33gRNAs, we identified 7 gRNAs with good editing efficiency and able to edit 7 potential safe harbors. See FIG. 2.

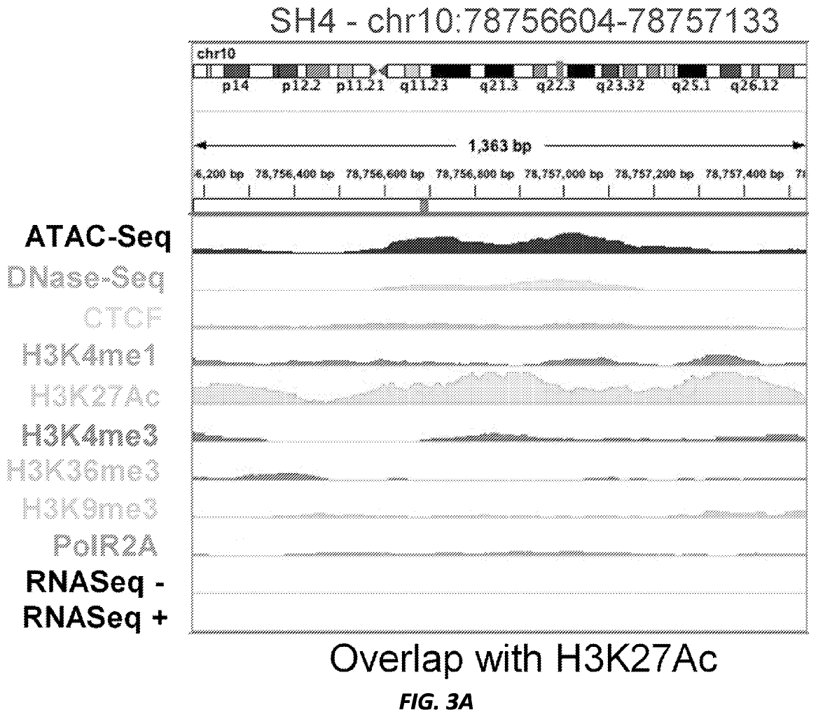

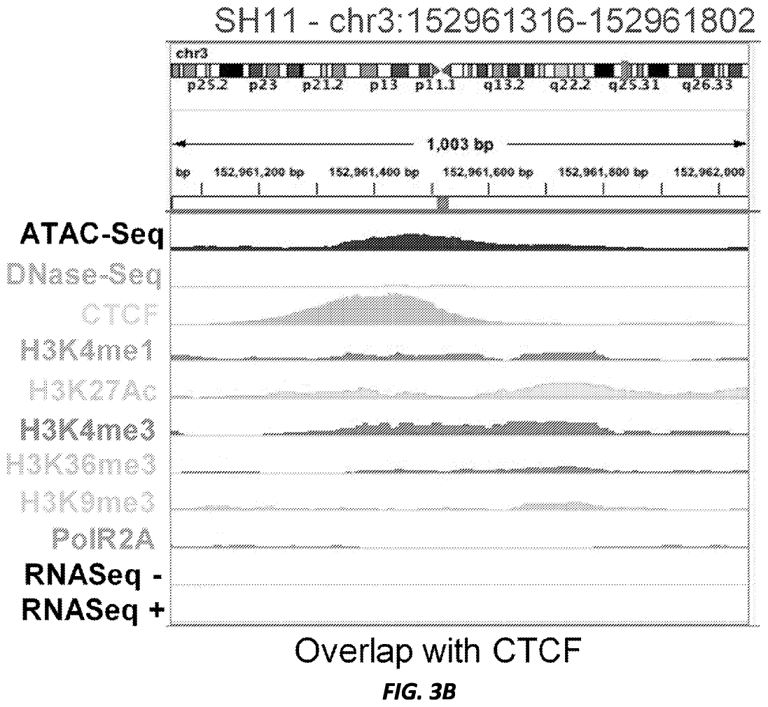

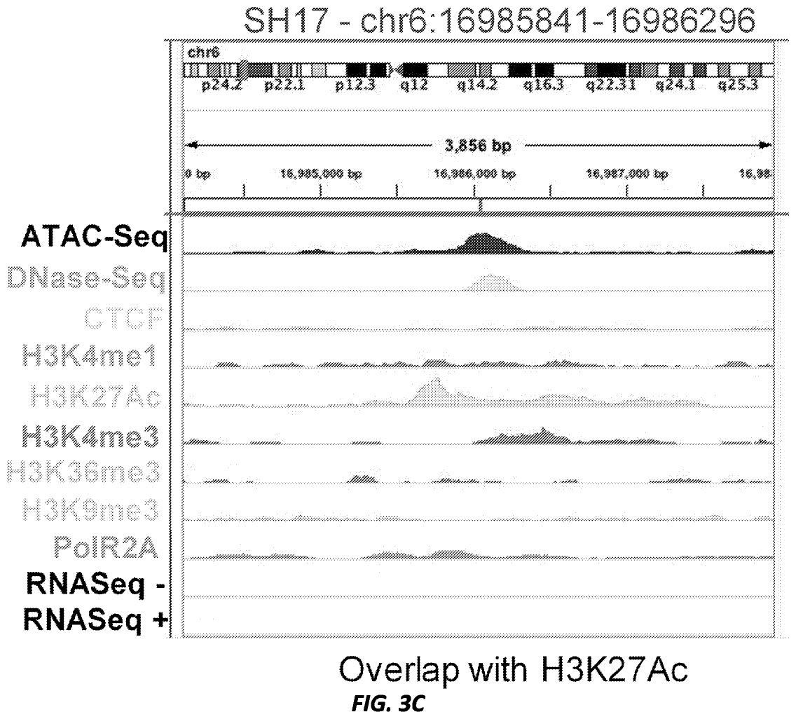

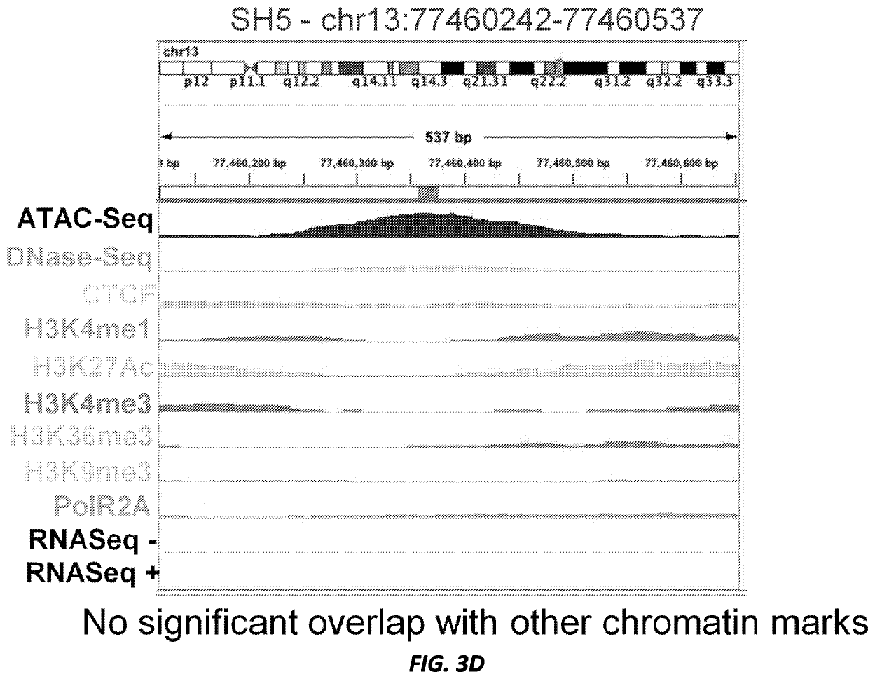

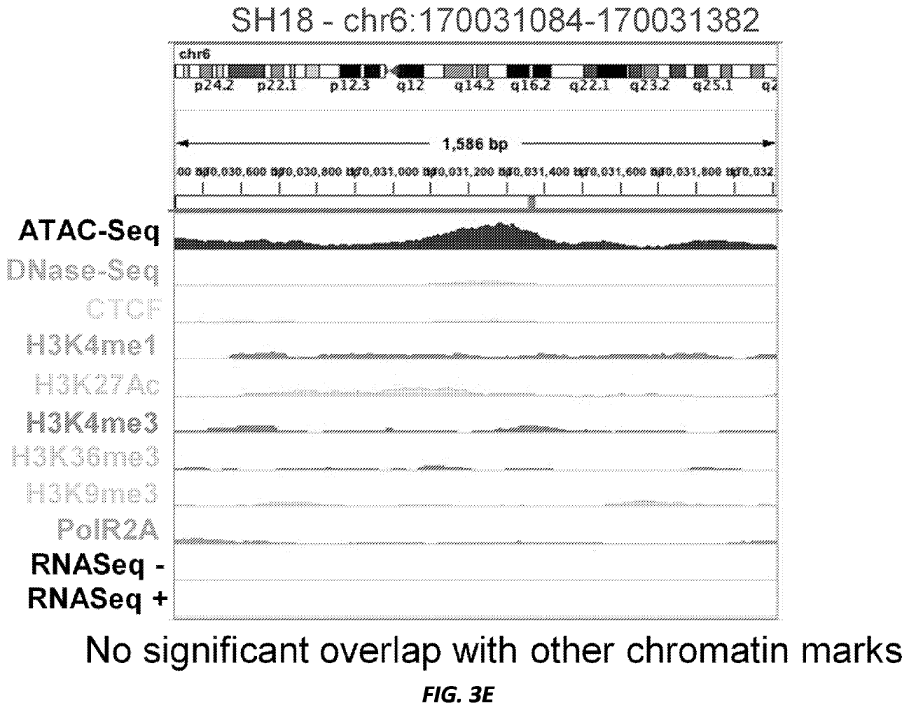

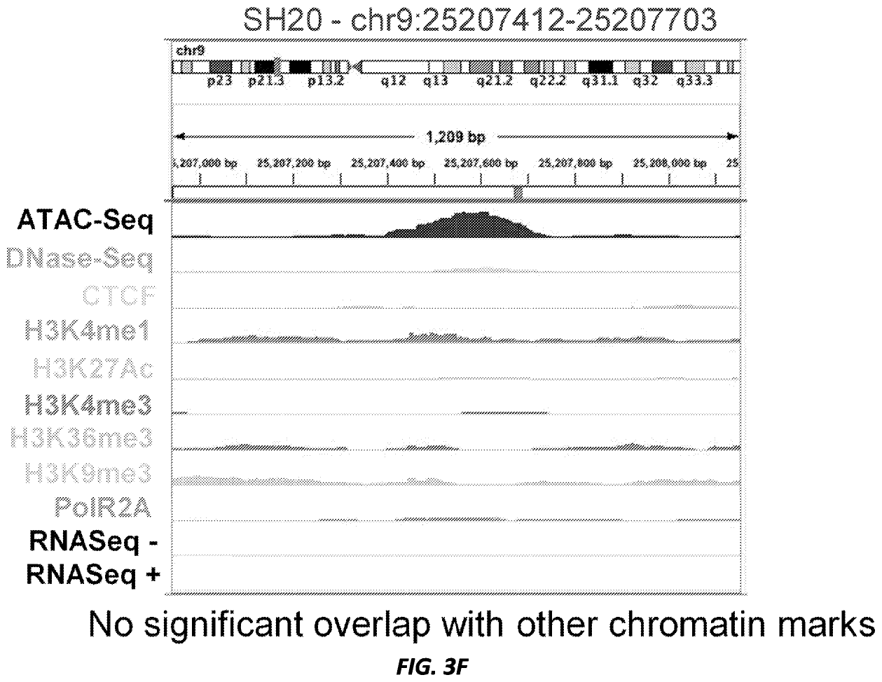

This list of loci passing the editing screening was then manually curated to analyze the chromatin environment based on Chip Seq data for chromatin marks to disqualify from the analysis any potential safe harbor that was falling in regions predicted to be regulatory regions (H3K4me1, H3K27ac, H3K4me3), heterochromatin regions (H3K9me3), or participating into chromatin organization (CTCF signals). See, e.g., FIGS. 3A-3C for loci that were disqualified and FIGS. 3D-3F for loci that were selected (summarized in Table 4).

TABLE 4
Candidate Liver Extragenic Genomic Safe Harbors.

| Potential | Genomic | | | |
|---|---|---|---|---|
| Safe | Location | gRNA | | Chromatin |
| Harbor | GRCh38/hg38 | Name | sgRNA Target Site | Status: |
| L-SH4 | chr10: 78756604- | L- | ACCACGCCTATGTAGACAAC | Overlap with |
| | 78757133 | SH4.2 | (SEQ ID NO: 35) | H3K27Ac |
| L-SH5 | chr13: 77460242- | L-SH | GCTTACATTTGCCACAGGCG | PERMISSIVE |
| | 77460537 | 5.1 | (SEQ ID NO: 22) | |
| L-SH11 | chr3: 152961316- | L-SH11 | GCCAAACTCAAACCAGTCCG | Overlap with |
| | 152961802 | | (SEQ ID NO: 36) | CTCF |
| L-SH14 | chr4: 99723588- | L-SH | TTGGCAAATCGTGTTGGCTG | Overlap with |
| | 99723981 | 14.2 | (SEQ ID NO: 37) | H3K27Ac |
| L-SH17 | chr6: 16985841- | L-SH17 | CCAGTTTATCGTAGAGCACA | Overlap with |
| | 16986296 | | (SEQ ID NO: 38) | H3K27Ac |
| L-SH18 | chr6: 170031084- | L- | GGCACCATCTGCGCCGAGCT | PERMISSIVE |
| | 170031382 | SH18.2 | (SEQ ID NO: 23) | |
| L-SH20 | chr9: 25207412- | L- | GGTCCAGCAAATACTAGATT | PERMISSIVE |
| | 25207703 | SH20.1 | (SEQ ID NO: 24) | |

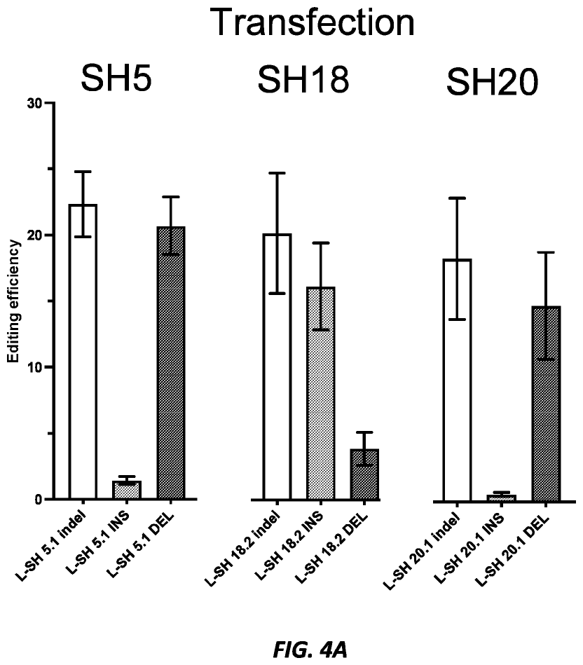

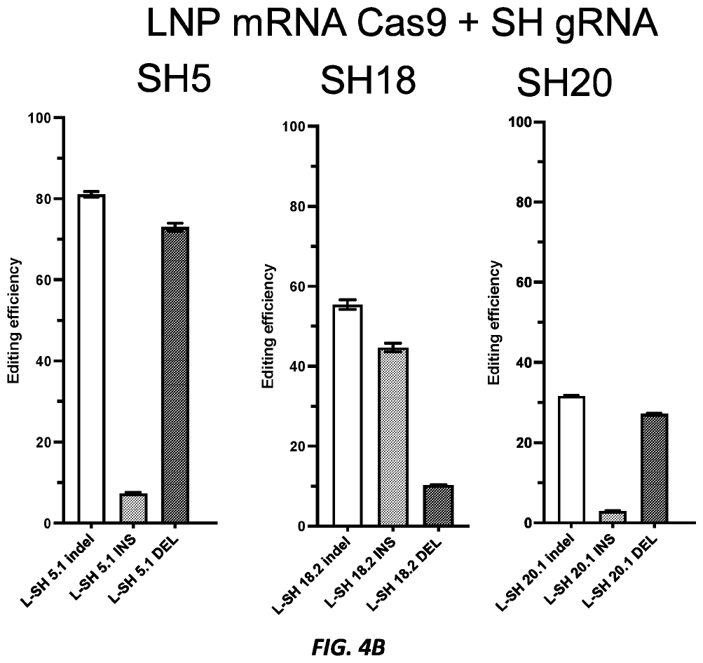

Three top candidate extragenic safe harbor loci were identified, as shown in Table 5. Editing efficiency in primary human hepatocytes, including an assessment of whether the repair resulted in insertions or deletions, after transfection of Cas9 mRNA and gRNA or following delivery in a lipid nanoparticle is shown in FIGS. 4A and 4B, respectively.

TABLE 5
Liver Extragenic Genomic Safe Harbors.

| Potential | Genomic | | sgRNA | SEQ |
|---|---|---|---|---|
| Safe | Location | gRNA | Target | ID |
| Harbor | GRCh38/hg38 | Name | Site | NO |
| L-SH5 | chr13: | L-SH5.1 | GCTTACATTT | 22 |
| | 77460242- | | GCCACAGGCG | |
| | 77460537 | | | |
| L-SH18 | chr6: | L-SH18.2 | GGCACCATCT | 23 |
| | 170031084- | | GCGCCGAGCT | |
| | 170031382 | | | |
| L-SH20 | chr9: | L-SH20.1 | GGTCCAGCAA | 24 |
| | 25207412- | | ATACTAGATT | |
| | 25207703 | | | |

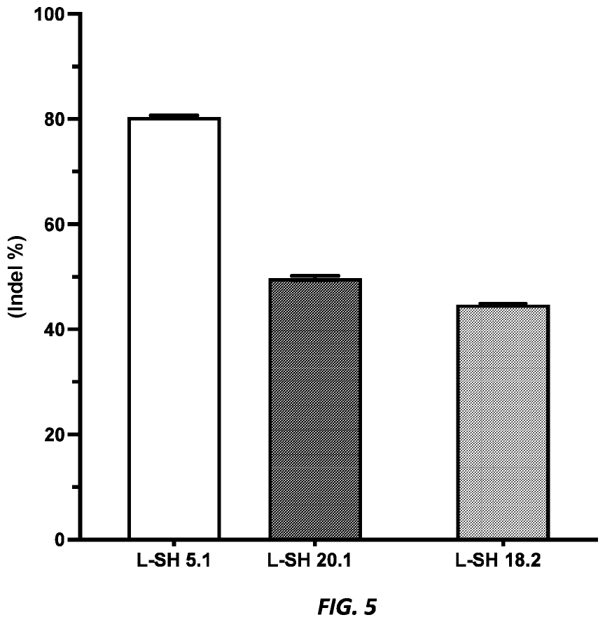

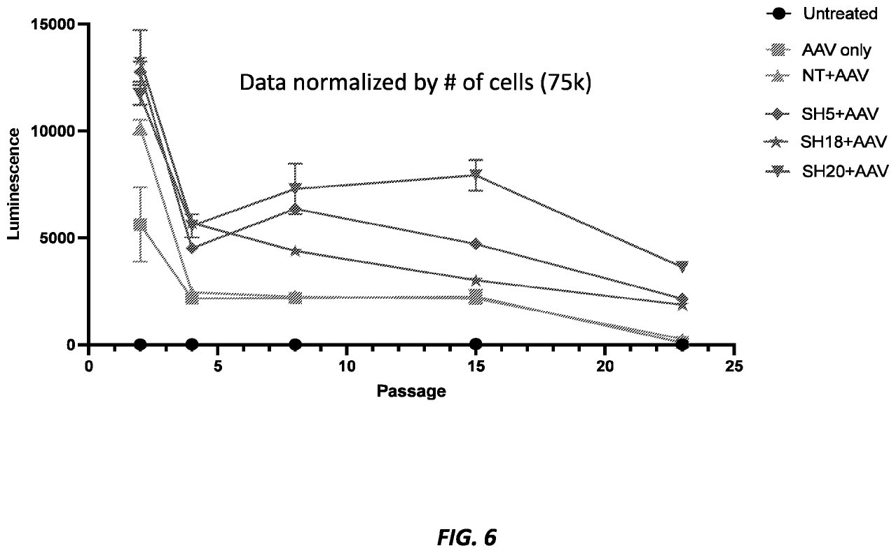

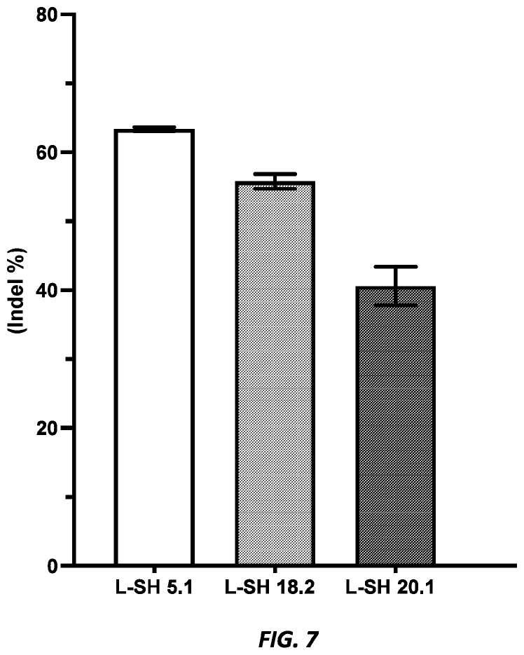

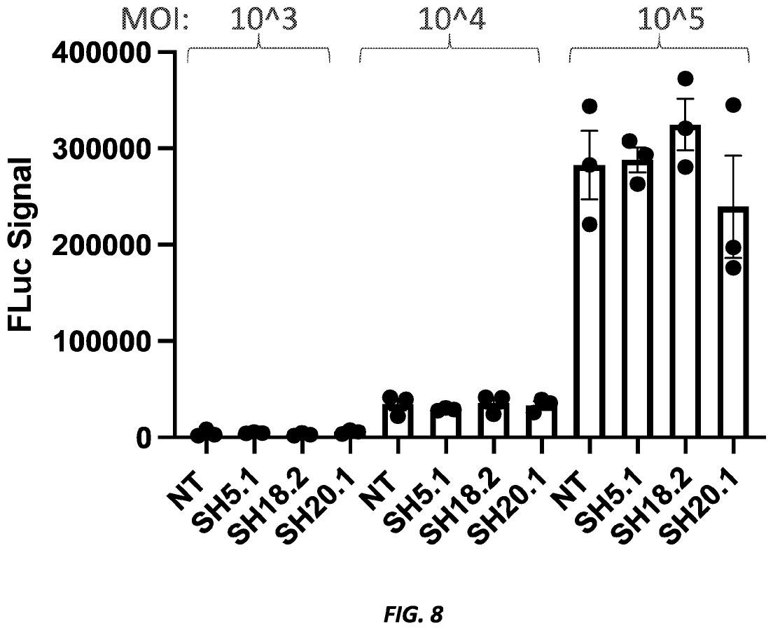

The top 3 candidates were then tested in combination with a nucleic acid construct for insertion into the L-SH5, L-SH18, and L-SH20 genomic loci. The nucleic acid construct included a firefly luciferase (FLuc) coding sequence operably linked to a CMV promoter, packaged in a recombinant AAV-DJ vector. The AAV-DJ construct and lipid nanoparticle comprising Cas9 mRNA and sgRNA were delivered to HepG2 cells, and editing efficiency was assessed as shown in FIG. 5. FLuc signal was also assessed relative to an untreated control and a negative control in which a non-targeting sgRNA was used. The results are shown in FIG. 6. The AAV-DJ construct and lipid nanoparticle comprising Cas9 mRNA and sgRNA were then delivered to primary human hepatocytes cells, and editing efficiency was assessed as shown in FIG. 7. FLuc signal was also assessed relative to a negative control in which a non-targeting sgRNA was used. The results for three different doses of AAV are shown in FIG. 8.

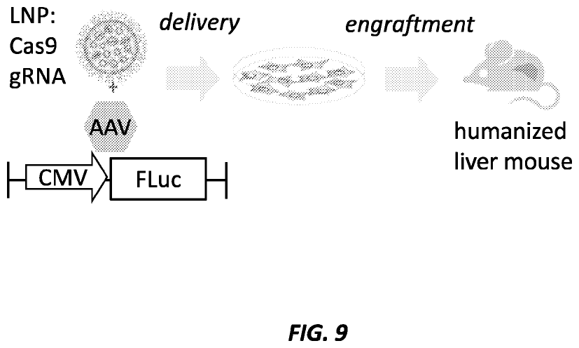

The top 3 candidates were then considered for in vivo validation in liver humanized mice (i.e., Fah (−/−) mice engrafted with primary human hepatocytes). See FIG. 9. Lipid nanoparticles (LNPs) including the CRISPR/Cas components (sgRNA and Cas9 mRNA) and recombinant AAV-DJ vector comprising an insertion template (CMV-FLuc) are administered to primary human hepatocytes, and mice are then engrafted with the primary human hepatocytes. Mice engrafted with untreated primary human hepatocytes are used as a first negative control. A second negative control includes a group of mice engrafted with primary human hepatocytes treated with recombinant AAV-DJ vector and LNP comprising Cas9 mRNA and a non-targeting sgRNA. Integration at each specific locus is assessed, and the following readouts are monitored: (i) long-term expression by MRI (up to 1 year); (ii) liver toxicity by specific ELISA (ALT, Ast, bilirubin); and (iii) gene expression changes by RNASeq.

The top 3 candidates are then considered for additional in vivo validation in liver humanized mice (i.e., Fah (−/−) mice engrafted with primary human hepatocytes). Lipid nanoparticles (LNPs) including the CRISPR/Cas components (sgRNA and Cas9 mRNA) and recombinant AAV-DJ vector comprising an insertion template (CMV-FLuc) are administered to Fah (−/−) mice engrafted with primary human hepatocytes. Untreated mice are a first negative control. A second negative control includes a group of mice treated with recombinant AAV-DJ vector and LNP comprising Cas9 mRNA and a non-targeting sgRNA. Integration at each specific locus is assessed, and the following readouts are monitored: (i) long-term expression by MRI (up to 1 year); (ii) liver toxicity by specific ELISA (ALT, Ast, bilirubin); and (iii) gene expression changes by RNASeq.

### Example 2. Safety Profile of Targeting Safe Harbor Loci in a Humanized Liver Mouse Model

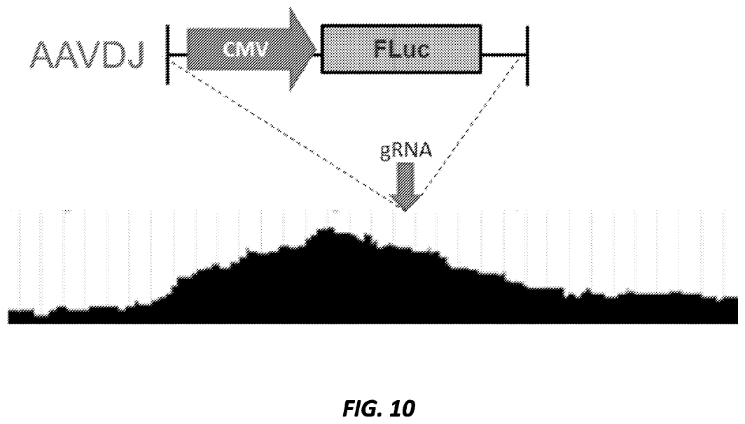

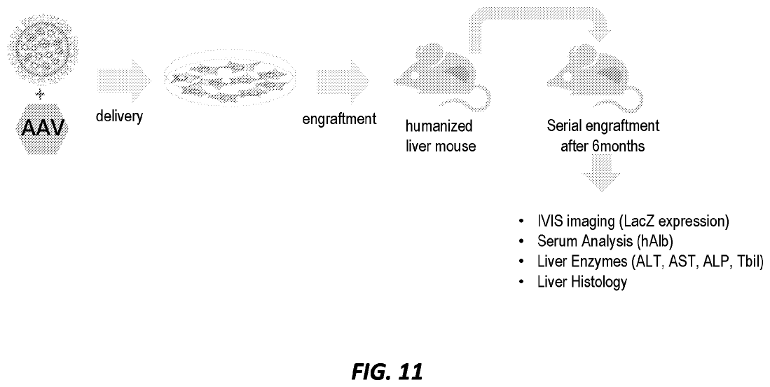

To validate the safety profile of targeting the selected potential safe harbor loci for therapeutic purposes, a transgene (FLuc) driven by a CMV promoter was inserted into these sites in primary human hepatocytes (PHH), as shown in FIG. 10, mimicking what would happen in human patients undergoing insertion of a therapeutic transgene in the liver. In turn, these modified PHH were engrafted in recipient FRG mice to establish humanized liver mouse models, as shown in FIG. 11.

The delivery of the expression cassettes to PHH was performed with AAV serotype DJ at MOI 10⁵ genome copies/cell. The cells were further treated with LNP-Cas9 mRNA and sgRNA targeting the loci at concentration of 1 g/mL to create a double strand break to facilitate the insertion.

After 4 days in culture, PHH were engrafted in FRG mice, allowing the repopulation of the mouse liver with the human counterpart. FRG mice are Fah (−/−), Rag-2(−/−) and interleukin 2 receptor common gamma chain (−/−). These triple mutant mice are immunodeficient at two loci and still retained the selective pressure provided by Fah deficiency. Fumarylacetoacetate hydrolase (Fah), a gene in the catabolic pathway for tyrosine, is deleted and mice are kept in healthy state by feeding them the drug 2-(2-nitro-4-trifluoro-methylbenzoyl)1,3-cyclohexedione (NTBC), which blocks the accumulation of the toxic metabolite and prevents liver damage. When transplanted with PHH, mice FRG mice are withdrawn of NTBC, thus causing mouse liver cells to be replaced with the human counterpart (carrying a wild type FAH function), which will repopulate the mouse liver.

This system was chosen to monitor long term expression of the transgene and to monitor liver physiology based on histology and hepatic liver serum chemistry, thus establishing the safety of such approach, following targeting of these loci.

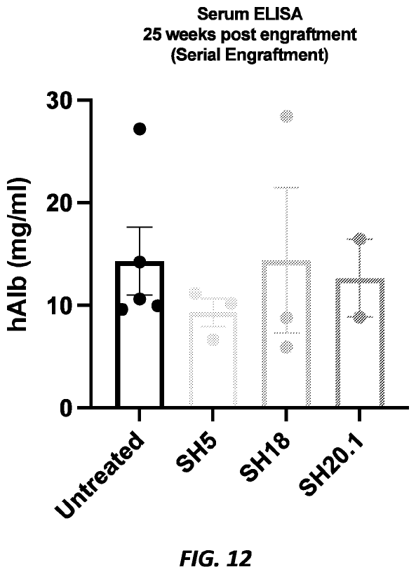

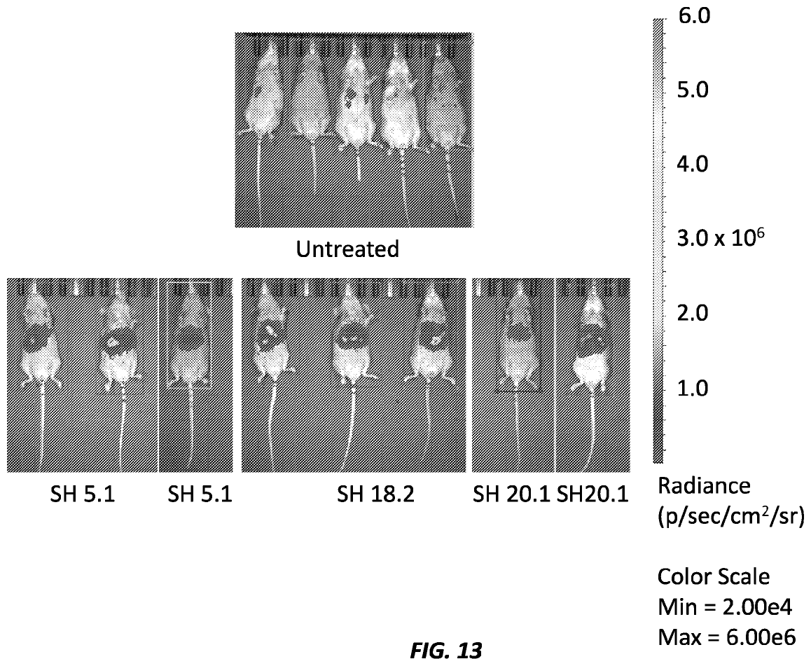

As shown in FIG. 12, high levels of human albumin (hAlb) were detected upon serial engraftment, indicating correct and productive engraftment. Twelve months after initial treatment, FLuc expression was assayed by IVIS imaging and a strong signal was detected, suggesting integration of the transgene (FIG. 13). Since the PHH rapidly replicate upon engraftment, episomal copies of AAV should be lost, as shown in the untreated group (top left FIG. 13).

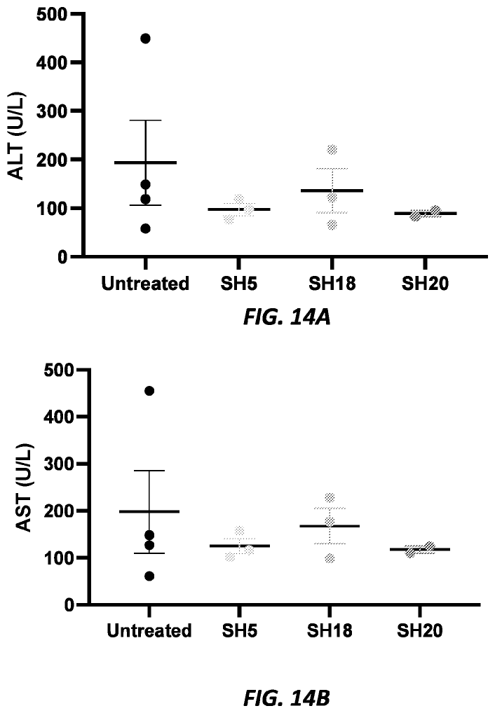

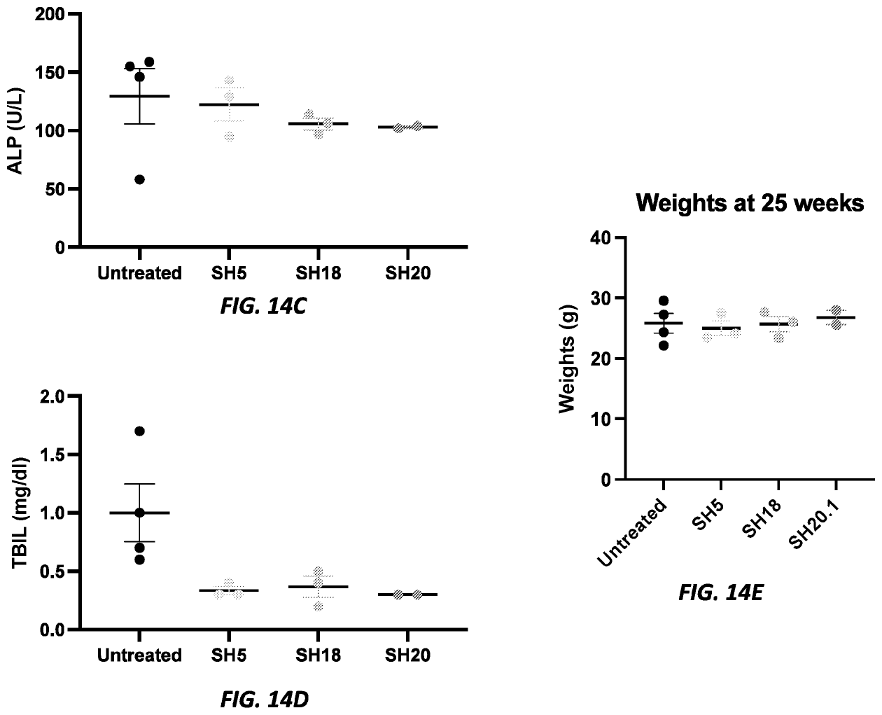

Serum was collected from individual mice to assess whether the liver chemistry was altered upon treatment. As shown in FIGS. 14A-14C, ALT, AST, and ALP, markers of liver functionality, were consistent among treatment and untreated groups, suggesting that no major detrimental effect was caused by targeting these loci. Bilirubin was reduced in the treatment groups, as shown in FIG. 14D. However, low levels of bilirubin have not been connected any medical conditions and no detrimental effect has been associated to this reduction in human patients. The cause for this reduction in bilirubin is not well understood. Moreover, no body weight differences were observed among treated and untreated groups, as shown in FIG. 14E.

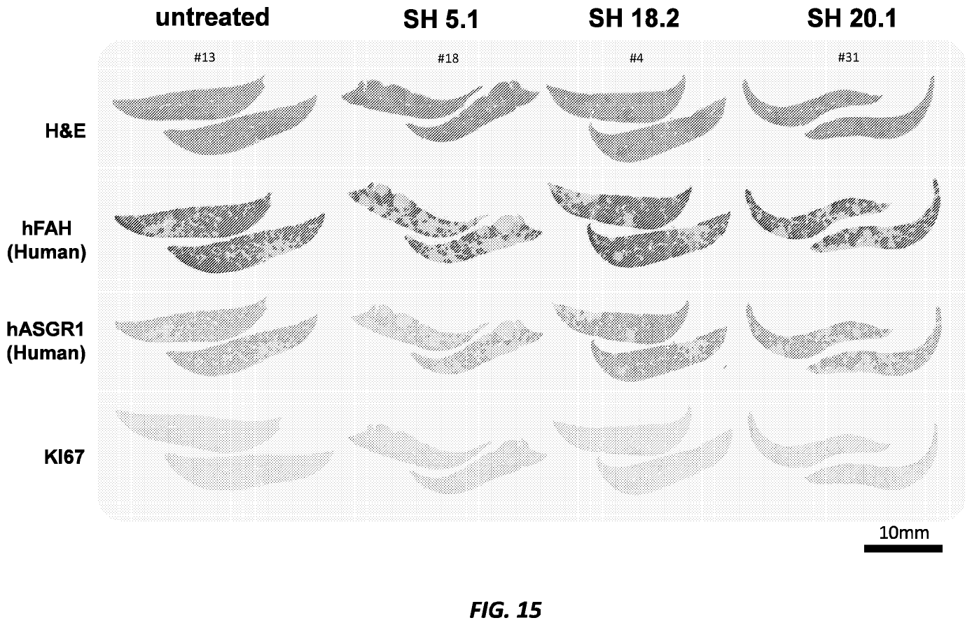

Since one of the major concerns of inserting exogenous DNA into the genomic DNA is the potential oncogenic effect, Ki67 was assayed as a marker of proliferation in the liver indicative of active oncogenic transformation. Ki67 did not produce any significant staining (FIG. 15, bottom row), suggesting no tumorigenesis as confirmed by H&E staining (FIG. 15, top row). In addition, staining for human ASGR1 and human FAH, two human liver-specific genes, showed a high degree of humanization of these mouse livers (FIG. 15, middle rows).

Taken together, these results show that engineering these loci with the insertion of a transgene driven by a CMV promoter has no detrimental effect on the mouse/liver physiology, thus establishing these loci as safe harbors for human therapeutics.

### Example 3. Identification of Syntenic Mouse Regions

To identify the syntenic mouse regions corresponding to the identified safe harbors, the human genomic coordinates were used in the Ensembl genome browser for comparative genomics. The synteny analysis relies on the identification of conserved order of genomic blocks between species. It was calculated from the pairwise genome alignments created by Ensembl, when both species have a chromosome-level assembly. The search was run in two phases: (1) search for alignment blocks that are in the same order in the two genomes; syntenic alignments that were closer than 200 kb were grouped into a synteny block; and (2) groups that are in synteny were linked, provided that no more than two non-syntenic groups were found between them and they were less than 3 Mb apart.

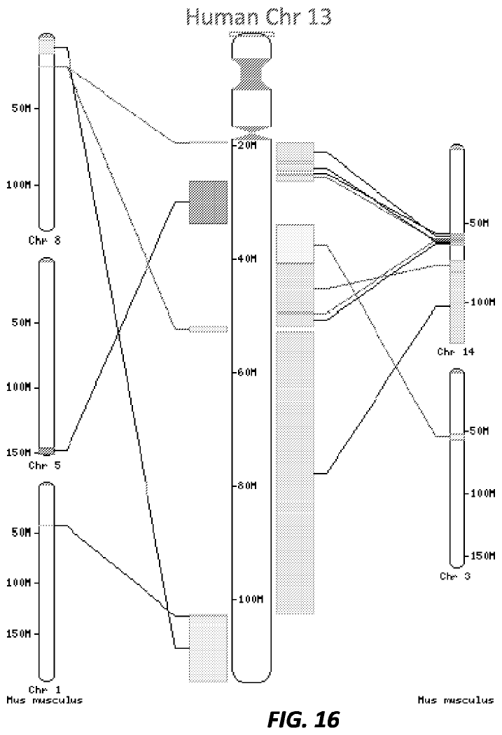

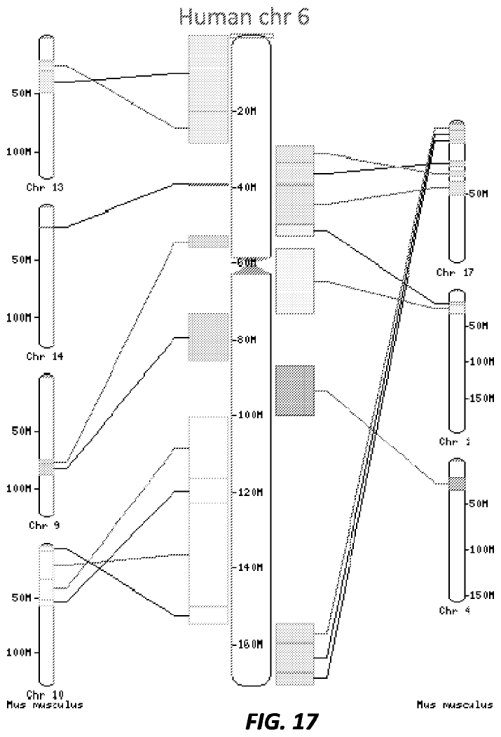

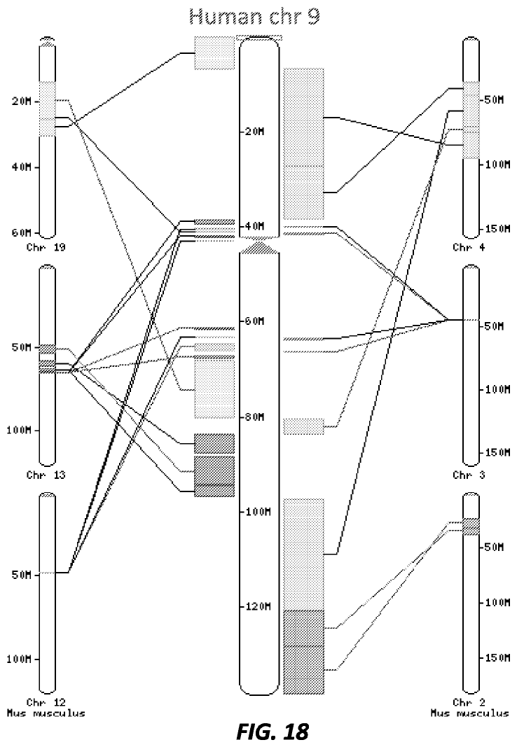

For all three human regions, we identified the mouse syntenic blocks as shown in FIGS. 16-18. The figures show the alignment blocks in between the human chromosome region containing the potential safe harbor (indicated by the arrow) and the corresponding mouse chromosome's block with same alignment order.

TABLE 6
Candidate Mouse Liver Extragenic Genomic Safe Harbors.

| Potential Safe Harbor | Mouse Genomic Location (GRCm38/mm10) |
|---|---|
| Syntenic Safe Harbor SH5 | chr14: 103, 450, 397-103, 451, 396 |
| Syntenic Safe Harbor SH18 | chr17: 15, 226, 387-15, 227, 386 |
| Syntenic Safe Harbor SH20 | chr4: 92, 827, 563-92, 828, 592 |

### Example 4. Identification of gRNAs

Guide RNAs targeting the human SH5, SH18, and SH20 genomic safe harbor sites (+/−5 kb) are provided below in Tables 7-9. Those in italics are within the genomic safe harbor loci (ATAC peaks). Guide RNAs targeting the mouse syntenic SH5, SH18, and SH20 genomic safe harbor sites (+/−5 kb) are provided below in Tables 10⁻¹². Those in italics are immediately adjacent to the genomic safe harbor loci (ATAC peaks).

TABLE 7
gRNAs Targeting Human SH5.

| | | SEQ | SEQ ID NO |
|---|---|---|---|
| | | ID | corresponding |
| Genomic Location | sgRNA Target Site | NO | guide sequence |
| NC_000013.11: 77462080-77462102 | CGTTATCGAGTATTGGGCGA | 51 | 228 |
| NC_000013.11: 77462079-77462101 | ACGTTATCGAGTATTGGGCG | 52 | 229 |
| NC_000013.11: 77462081-77462103 | GTTATCGAGTATTGGGCGAG | 53 | 230 |
| NC_000013.11: 77462073-77462095 | ATCATGACGTTATCGAGTAT | 54 | 231 |
| NC_000013.11: 77463717-77463739 | GTAGCCGTCACTTATTAGAT | 55 | 232 |
| NC_000013.11: 77462074-77462096 | TCATGACGTTATCGAGTATT | 56 | 233 |
| NC_000013.11: 77458147-77458169 | TATCTTGTACGATAGGGTCA | 57 | 234 |
| NC_000013.11: 77460391-77460413 | ACTGGCCAGTACACATCGCT | 58 | 235 |
| NC_000013.11: 77458595-77458617 | ATTGTTAATTGTAGTCCGGG | 59 | 236 |
| NC_000013.11: 77460386-77460408 | AACTCCCCAGCGATGTGTAC | 60 | 237 |
| NC_000013.11: 77462764-77462786 | CGCATGATGCTTGCTCTCAC | 61 | 238 |
| NC_000013.11: 77459946-77459968 | GTTAAAAGACGGGATTCCTT | 62 | 239 |
| NC_000013.11: 77464693-77464715 | GTATAGGTACTAACACTCCC | 63 | 240 |
| NC_000013.11: 77458143-77458165 | TTGTACGATAGGGTCAAGGA | 64 | 241 |
| NC_000013.11: 77459659-77459681 | ATGCAATGTGCTGATCCGGA | 65 | 242 |
| NC_000013.11: 77460355-77460377 | GCTTACATTTGCCACAGGCG | 22 | 25 |
| NC_000013.11: 77462085-77462107 | TCGAGTATTGGGCGAGGGGT | 66 | 243 |
| NC_000013.11: 77459660-77459682 | CATGCAATGTGCTGATCCGG | 67 | 244 |
| NC_000013.11: 77458144-77458166 | CTTGTACGATAGGGTCAAGG | 68 | 245 |
| NC_000013.11: 77460390-77460412 | CTGGCCAGTACACATCGCTG | 69 | 246 |
| NC_000013.11: 77464441-77464463 | GCATCGAAGGACACTAAAGC | 70 | 247 |
| NC_000013.11: 77459658-77459680 | TGCAATGTGCTGATCCGGAG | 71 | 248 |
| NC_000013.11: 77459738-77459760 | GAGATCAATCATGTACTAGG | 72 | 249 |
| NC_000013.11: 77459789-77459811 | CACGCTAACATAATATTCCC | 73 | 250 |
| NC_000013.11: 77459844-77459866 | ACTTACAATAAGACTCCTGC | 74 | 251 |
| NC_000013.11: 77460392-77460414 | CACTGGCCAGTACACATCGC | 75 | 252 |
| NC_000013.11: 77460715-77460737 | GCTAGAGGACATATCCTCTT | 76 | 253 |
| NC_000013.11: 77463670-77463692 | CAAGGTTGGTGGTTTCCGGC | 77 | 254 |
| NC_000013.11: 77458598-77458620 | CGCATTGTTAATTGTAGTCC | 78 | 255 |
| NC_000013.11: 77459790-77459812 | CTGGGAATATTATGTTAGCG | 79 | 256 |

TABLE 8
gRNAs Targeting Human SH18.

| | | SEQ | SEQ ID NO |
|---|---|---|---|
| | | ID | corresponding |
| Genomic Location | sgRNA Target Site | NO | guide sequence |
| NC_000006.12: 170032875-170032897 | GCACGGACCCGTTTACGGAG | 80 | 257 |
| NC_000006.12: 170033338-170033360 | ACGCTACGGAAGTCAACTGC | 81 | 258 |
| NC_000006.12: 170032874-170032896 | CACGGACCCGTTTACGGAGA | 82 | 259 |
| NC_000006.12: 170032880-170032902 | AATTTGCACGGACCCGTTTA | 83 | 260 |
| NC_000006.12: 170029268-170029290 | TCGGGCGGCACTGAACGCCC | 84 | 261 |
| NC_000006.12: 170026603-170026625 | AAATTCCACAGACGCGCACC | 85 | 262 |
| NC_000006.12: 170028678-170028700 | GCCGGGTTCTGCGTACACAT | 86 | 263 |
| NC_000006.12: 170032831-170032853 | ATCTGGACCCGTGCGCCAGC | 87 | 264 |
| NC_000006.12: 170026701-170026723 | AGATCGCATGCACGCCCAGC | 88 | 265 |
| NC_000006.12: 170034262-170034284 | GTGTAGTCAACCCTGCCCGA | 89 | 266 |
| NC_000006.12: 170032885-170032907 | AACGGGTCCGTGCAAATTCA | 90 | 267 |
| NC_000006.12: 170031376-170031398 | TGCAGCACGCAGTTCTTGCG | 91 | 268 |
| NC_000006.12: 170033497-170033519 | TGGGCACCTTGATTCGGTGC | 92 | 269 |
| NC_000006.12: 170034323-170034345 | AGGTCTCTTGTTGCACCCGA | 93 | 270 |
| NC_000006.12: 170031178-170031200 | CTGGCACGTCCCCGAAAATG | 94 | 271 |
| NC_000006.12: 170029404-170029426 | CATTGTACGACTAAGATTGA | 95 | 272 |
| NC_000006.12: 170029275-170029297 | GTTCAGTGCCGCCCGACAGA | 96 | 273 |
| NC_000006.12: 170034261-170034283 | TGTGTAGTCAACCCTGCCCG | 97 | 274 |
| NC_000006.12: 170031353-170031375 | GGCACCATCTGCGCCGAGCT | 23 | 26 |
| NC_000006.12: 170029120-170029142 | GGCACACCTGACGATGTCAA | 98 | 275 |
| NC_000006.12: 170031432-170031454 | CGGCCACCAGCTTTTGCCGG | 99 | 276 |
| NC_000006.12: 170032417-170032439 | GGGACTCTGGCCCTAACGTG | 100 | 277 |
| NC_000006.12: 170034126-170034148 | AGGTGCACTAGGTAGACTAG | 101 | 278 |
| NC_000006.12: 170029126-170029148 | AGGCCCCCTTTGACATCGTC | 102 | 279 |
| NC_000006.12: 170031354-170031376 | AGGCACCATCTGCGCCGAGC | 103 | 280 |
| NC_000006.12: 170032824-170032846 | GCGCACGGGTCCAGATGGAG | 104 | 281 |
| NC_000006.12: 170032838-170032860 | CTGGATACCAGCTGGCGCAC | 105 | 282 |
| NC_000006.12: 170033352-170033374 | GGTACTGGTGGCAAACGCTA | 106 | 283 |
| NC_000006.12: 170026601-170026623 | AGGTGCGCGTCTGTGGAATT | 107 | 284 |
| NC_000006.12: 170028679-170028701 | GCCCATGTGTACGCAGAACC | 108 | 285 |

TABLE 9
gRNAs Targeting Human SH20.

| | | | SEQ ID NO |
|---|---|---|---|
| | | SEQ | corresponding |
| Genomic Location | sgRNA Target Site | ID | guide sequence |
| NC_000009.12: 25209472-25209494 | ACCCGCTAGAATCTAGTCAT | 109 | 286 |
| NC_000009.12: 25204332-25204354 | AAGTAGGACGTTCCCGCATA | 110 | 287 |
| NC_000009.12: 25207601-25207623 | ATCCGACCTGCCTCGGGCTT | 111 | 288 |
| NC_000009.12: 25207665-25207687 | GGTCCAGCAAATACTAGATT | 24 | 27 |
| NC_000009.12: 25211734-25211756 | TCGAGAGCAAGACAACGCAA | 112 | 289 |
| NC_000009.12: 25212404-25212426 | CACTTGAAGATTCCGGATGA | 113 | 290 |
| NC_000009.12: 25207026-25207048 | CTACCAGTGGGCCTAAATAA | 114 | 291 |
| NC_000009.12: 25209154-25209176 | TAAGTGTCAGCTTACCAACG | 115 | 292 |
| NC_000009.12: 25209691-25209713 | GGGGCCAATAGCAAACTAAC | 116 | 293 |
| NC_000009.12: 25209440-25209462 | CCGAATCTATCAGTGCCTGG | 117 | 294 |
| NC_000009.12: 25209895-25209917 | CGAGCATATATGCTAGATTT | 118 | 295 |
| NC_000009.12: 25207585-25207607 | GTCGGATGCCAGGATTTCAA | 119 | 296 |
| NC_000009.12: 25209742-25209764 | TAGAACTCACGACATAGGGC | 120 | 297 |
| NC_000009.12: 25205551-25205573 | TTTAACTAAGGTTGCCGAAA | 121 | 298 |
| NC_000009.12: 25206179-25206201 | GAACGGATGCAACAAGACCA | 122 | 299 |
| NC_000009.12: 25208119-25208141 | GATGTGCTAACATCCTCCCG | 123 | 300 |
| NC_000009.12: 25206147-25206169 | AGTTTCTTTATCACGATCAT | 124 | 301 |
| NC_000009.12: 25206178-25206200 | GGAACGGATGCAACAAGACC | 125 | 302 |
| NC_000009.12: 25209471-25209493 | TCCTATGACTAGATTCTAGC | 126 | 303 |
| NC_000009.12: 25207023-25207045 | ATGCCATTATTTAGGCCCAC | 127 | 304 |
| NC_000009.12: 25207472-25207494 | AGTCTGACACGGACAAGTAC | 128 | 305 |
| NC_000009.12: 25207600-25207622 | CATCCGACCTGCCTCGGGCT | 129 | 306 |
| NC_000009.12: 25207835-25207857 | TCAGCACTGGTACATATATT | 130 | 307 |
| NC_000009.12: 25209443-25209465 | AGTCCGAATCTATCAGTGCC | 131 | 308 |
| NC_000009.12: 25204921-25204943 | ATATAGGATCCCAACAATAT | 132 | 309 |
| NC_000009.12: 25207662-25207684 | CCAGCAAATACTAGATTGGG | 133 | 310 |
| NC_000009.12: 25208132-25208154 | ACTCCATATTCTTCCCCGGG | 134 | 311 |
| NC_000009.12: 25212540-25212562 | AACGCACATCGTTTATATGG | 135 | 312 |
| NC_000009.12: 25209705-25209727 | ACTAACAGGATGCATGCAAG | 136 | 313 |
| NC_000009.12: 25204335-25204357 | TAGGACGTTCCCGCATAAGG | 137 | 314 |

TABLE 10
gRNAs Targeting Mouse SH5.

| | | | SEQ ID NO |
|---|---|---|---|
| | | SEQ | corresponding |
| | | ID | guide |
| Genomic Location | sgRNA Target Site | NO | sequence |
| NC_000080.6: 103453466-103453488 | GGGACCGATTTGGCCGTTAA | 138 | 315 |
| NC_000080.6: 103453470-103453492 | GTAACCATTAACGGCCAAAT | 139 | 316 |
| NC_000080.6: 103445660-103445682 | CACGGCCCTTCGATTGTTGA | 140 | 317 |
| NC_000080.6: 103451136-103451158 | AAGGATCGTTAGGTGATCGT | 141 | 318 |
| NC_000080.6: 103449387-103449409 | TACCTACCGGTCATATATCC | 142 | 319 |
| NC_000080.6: 103450385-103450407 | CACGAGGACGATTACCATAT | 143 | 320 |
| NC_000080.6: 103451135-103451157 | AGGATCGTTAGGTGATCGTC | 144 | 321 |
| NC_000080.6: 103451733-103451755 | CACGGGACGCTATTCTAAAC | 145 | 322 |
| NC_000080.6: 103451954-103451976 | CCATAATCAGGTCGTTTATC | 146 | 323 |
| NC_000080.6: 103453452-103453474 | ATCGGTCCCATAATAGGGTG | 147 | 324 |
| NC_000080.6: 103453458-103453480 | GGCCAAATCGGTCCCATAAT | 148 | 325 |
| NC_000080.6: 103454184-103454206 | CTCGGTTTACTAGGACAAAG | 149 | 326 |
| NC_000080.6: 103451743-103451765 | ATAGCGTCCCGTGATGTTTT | 150 | 327 |
| NC_000080.6: 103452860-103452882 | TCAGTATTGTACCGCTTTAG | 151 | 328 |
| NC_000080.6: 103453453-103453475 | AATCGGTCCCATAATAGGGT | 152 | 329 |
| NC_000080.6: 103453456-103453478 | ACCCTATTATGGGACCGATT | 153 | 330 |
| NC_000080.6: 103456002-103456024 | TGCACTTGCCCGTGTAACAC | 154 | 331 |
| NC_000080.6: 103445666-103445688 | GATCTACCATCAACAATCGA | 155 | 332 |
| NC_000080.6: 103449385-103449407 | CACCAGGATATATGACCGGT | 156 | 333 |
| NC_000080.6: 103452169-103452191 | GGTGGCTACGTTGGGCCCAC | 157 | 334 |
| NC_000080.6: 103453457-103453479 | GCCAAATCGGTCCCATAATA | 158 | 335 |
| NC_000080.6: 103446063-103446085 | ATCAGTTATAACAGCCTAGC | 159 | 336 |
| NC_000080.6: 103448477-103448499 | CCGCTTGCACTAGAGGGTCC | 160 | 337 |
| NC_000080.6: 103448481-103448503 | GACCCTCTAGTGCAAGCGGC | 161 | 338 |
| NC_000080.6: 103454001-103454023 | GATAGTTACTTTACTCTCCG | 162 | 339 |
| NC_000080.6: 103445651-103445673 | ATCGAAGGGCCGTGTTAAGA | 163 | 340 |
| NC_000080.6: 103450410-103450432 | AAAGAGGCTTCTCAACCCGC | 164 | 341 |
| NC_000080.6: 103452256-103452278 | ACGGACCATCATGCACGGCA | 165 | 342 |
| NC_000080.6: 103453539-103453561 | GTCCCCCGTGCCCTAGATCC | 166 | 343 |
| NC_000080.6: 103454766-103454788 | TCGAAGGACCAAACTCATAG | 167 | 344 |

TABLE 11
gRNAs Targeting Mouse SH18.

| | | | SEQ ID NO |
|---|---|---|---|
| | | SEQ | corresponding |
| | | ID | guide |
| Region-name | Target-Seq | NO | sequence |
| NC_000083.6: 15231200-15231222 | GTACGACCCATAAAAGTGCC | 168 | 345 |
| NC_000083.6: 15223879-15223901 | CTGTGAATACGCCTTGGCAC | 169 | 346 |
| NC_000083.6: 15226621-15226643 | CCTTCAACAGTCCCGCCCAT | 170 | 34 |
| NC_000083.6: 15223975-15223997 | CGTGTGCAGCCCGGTGCCGA | 171 | 348 |
| NC_000083.6: 15228435-15228457 | CACAAACGATGGGTAGTTTC | 172 | 349 |
| NC_000083.6: 15230895-15230917 | TCGCACACTAAACGAGGAAC | 173 | 350 |
| NC_000083.6: 15223873-15223895 | ATACGCCTTGGCACTGGATC | 174 | 351 |
| NC_000083.6: 15223966-15223988 | TGCCACGGGCCTTCGGCACC | 175 | 352 |
| NC_000083.6: 15224413-15224435 | GGAACTGTGATCCGTGCCAC | 176 | 353 |
| NC_000083.6: 15225620-15225642 | AGGCTACTATGATGCGGTAG | 177 | 354 |
| NC_000083.6: 15230901-15230923 | GTTTGATCGCACACTAAACG | 178 | 355 |
| NC_000083.6: 15231424-15231446 | CATGCTTTCCCGCAAGCGTT | 179 | 356 |
| NC_000083.6: 15231433-15231455 | CGGATGTGACCAAACGCTTG | 180 | 357 |
| NC_000083.6: 15221497-15221519 | GTGTGCCCCGTTTATTGGTG | 181 | 358 |
| NC_000083.6: 15223968-15223990 | AGCCCGGTGCCGAAGGCCCG | 182 | 359 |
| NC_000083.6: 15227111-15227133 | TTCTAGAGGATCAATACTCG | 183 | 360 |
| NC_000083.6: 15228396-15228418 | GGGTCGGCTTATTAAAGAAT | 184 | 361 |
| NC_000083.6: 15228404-15228426 | TTAATAAGCCGACCCCTATG | 185 | 362 |
| NC_000083.6: 15228728-15228750 | GCCGGTCTTCCTAGTACAAC | 186 | 363 |
| NC_000083.6: 15229298-15229320 | GCAACCTTCTTTCCGAGTAG | 187 | 364 |
| NC_000083.6: 15229316-15229338 | GTTGCTGATTGGAGCCGGCA | 188 | 365 |
| NC_000083.6: 15231186-15231208 | GGGTCGTACCTATGTCTCTC | 189 | 366 |
| NC_000083.6: 15231417-15231439 | CTTGCGGGAAAGCATGCTAC | 190 | 367 |
| NC_000083.6: 15231686-15231708 | TGTGTTGGCGCTGCCTCGAC | 191 | 368 |
| NC_000083.6: 15226620-15226642 | CTTCAACAGTCCCGCCCATG | 192 | 369 |
| NC_000083.6: 15226621-15226643 | CCCATGGGCGGGACTGTTGA | 193 | 370 |
| NC_000083.6: 15229808-15229830 | GCCCTAGTGCATATTGGTTA | 194 | 371 |
| NC_000083.6: 15231818-15231840 | TATGGCTACCGGAACTCTGT | 195 | 372 |
| NC_000083.6: 15231829-15231851 | CCGGTAGCCATAATTGCTCC | 196 | 373 |
| NC_000083.6: 15221492-15221514 | GCCATGTGTGCCCCGTTTAT | 197 | 374 |

TABLE 12
gRNAs Targeting Mouse SH20.

| | | | SEQ ID NO |
|---|---|---|---|
| | | SEQ | corresponding |
| | | ID | guide |
| Region-name | Target-Seq | NO | sequence |
| NC_000070.6: 92829703-92829725 | ATCACCGGTGCCAAAGGGCC | 198 | 375 |
| NC_000070.6: 92831933-92831955 | TCTACGCTATCCCATACTGA | 199 | 376 |
| NC_000070.6: 92829708-92829730 | ATGTAATCACCGGTGCCAAA | 200 | 377 |
| NC_000070.6: 92831347-92831369 | ATAGTGACTATGCAGTGCGA | 201 | 378 |
| NC_000070.6: 92828459-92828481 | GCCCCCTATCTTAAATTTAT | 202 | 379 |
| NC_000070.6: 92828606-92828628 | TGGTGTGCCCTTACATCATT | 203 | 380 |
| NC_000070.6: 92830393-92830415 | GGATTCCACCGGAGGCAAAC | 204 | 381 |
| NC_000070.6: 92828599-92828621 | GCAGGCACCCAATGATGTAA | 205 | 382 |
| NC_000070.6: 92831883-92831905 | ATGAAAGTACGTACAAACTG | 206 | 383 |
| NC_000070.6: 92832217-92832239 | GATCTAGGGCTGGATCACTT | 207 | 384 |
| NC_000070.6: 92829193-92829215 | TAGCCACTAGCATGTTTAAT | 208 | 385 |
| NC_000070.6: 92830388-92830410 | AGAATCCTGTTTGCCTCCGG | 209 | 386 |
| NC_000070.6: 92831309-92831331 | AGCAGTTCAGTATACCTTAA | 210 | 387 |
| NC_000070.6: 92828468-92828490 | TTAAGATAGGGGGCATGTTG | 211 | 388 |
| NC_000070.6: 92829709-92829731 | AATGTAATCACCGGTGCCAA | 212 | 389 |
| NC_000070.6: 92830401-92830423 | AGATCCATGGATTCCACCGG | 213 | 390 |
| NC_000070.6: 92831260-92831282 | TCCTGTCAAGCTGTACACAC | 214 | 391 |
| NC_000070.6: 92822580-92822602 | GAAAACCCTTGCCAGTAGTT | 215 | 392 |
| NC_000070.6: 92828577-92828599 | AGATATCACCTATGGTAGCT | 216 | 393 |
| NC_000070.6: 92829196-92829218 | CATCCAATTAAACATGCTAG | 217 | 394 |
| NC_000070.6: 92830562-92830584 | TTGCAATTACTTAGATGTAC | 218 | 395 |
| NC_000070.6: 92832701-92832723 | GCTATAATCACTCAATGTGT | 219 | 396 |
| NC_000070.6: 92832969-92832991 | GTTAGATTCCTAGGTTTCTA | 220 | 397 |
| NC_000070.6: 92826167-92826189 | AAGACCCCCCACTCCAGGGC | 221 | 398 |
| NC_000070.6: 92827111-92827133 | GACCTACAGTTAATGGTTTA | 222 | 399 |
| NC_000070.6: 92828598-92828620 | GGCAGGCACCCAATGATGTA | 223 | 400 |
| NC_000070.6: 92829016-92829038 | TTGTGGAGGGCAAATCACAC | 224 | 401 |
| NC_000070.6: 92829939-92829961 | CTTGCTGATTCTGGAATACG | 225 | 402 |
| NC_000070.6: 92830397-92830419 | TTTGCCTCCGGTGGAATCCA | 226 | 403 |
| NC_000070.6: 92830409-92830431 | GGAATCCATGGATCTAAGTT | 227 | 404 |

We claim:

1. A method of integrating a nucleic acid construct into a genomic safe harbor locus in a human cell, comprising administering to the human cell:
(a) a nuclease agent or one or more nucleic acids encoding the nuclease agent, wherein the nuclease agent targets a nuclease target site in the genomic safe harbor locus, wherein the genomic safe harbor locus is selected from the following genomic locations:
(i) genomic coordinates of about 77460242 to about 77460537 on human chromosome 13;
(ii) genomic coordinates of about 170031084 to about 170031382 on human chromosome 6; and
(iii) genomic coordinates of about 25207412 to about 25207703 on human chromosome 9; and
(b) the nucleic acid construct, wherein the nucleic acid construct comprises a nucleic acid operably linked to a promoter, wherein the nucleic acid encodes a product of interest,
wherein the nuclease agent cleaves the nuclease target site, and the nucleic acid construct is inserted into the genomic safe harbor locus.

2. A method of expressing a product of interest from a genomic safe harbor locus in a human cell, comprising administering to the human cell:
(a) a nuclease agent or one or more nucleic acids encoding the nuclease agent, wherein the nuclease agent targets a nuclease target site in the genomic safe harbor locus, wherein the genomic safe harbor locus is selected from the following genomic locations:
(i) genomic coordinates of about 77460242 to about 77460537 on human chromosome 13;
(ii) genomic coordinates of about 170031084 to about 170031382 on human chromosome 6; and
(iii) genomic coordinates of about 25207412 to about 25207703 on human chromosome 9; and
(b) a nucleic acid construct, wherein the nucleic acid construct comprises a nucleic acid operably linked to a promoter, wherein the nucleic acid encodes the product of interest,
wherein the nuclease agent cleaves the nuclease target site, the nucleic acid construct is inserted into the genomic safe harbor locus to create a modified genomic safe harbor locus, and the product of interest is expressed from the modified genomic safe harbor locus.

3. The method of claim 1 or 2, wherein the human cell is a liver cell.

4. The method of any preceding claim, wherein the human cell is a hepatocyte.

5. The method of any preceding claim, wherein the human cell is in vitro or ex vivo.

6. The method of any one of claims 1-4, wherein the human cell is in vivo in a subject.

7. A method of integrating a nucleic acid construct into a genomic safe harbor locus in a human cell in a human subject, comprising administering to the human subject:
(a) a nuclease agent or one or more nucleic acids encoding the nuclease agent, wherein the nuclease agent targets a nuclease target site in the genomic safe harbor locus, wherein the genomic safe harbor locus is selected from the following genomic locations:
(i) genomic coordinates of about 77460242 to about 77460537 on human chromosome 13;
(ii) genomic coordinates of about 170031084 to about 170031382 on human chromosome 6; and
(iii) genomic coordinates of about 25207412 to about 25207703 on human chromosome 9; and
(b) the nucleic acid construct, wherein the nucleic acid construct comprises a nucleic acid operably linked to a promoter, wherein the nucleic acid encodes a product of interest,
wherein the nuclease agent cleaves the nuclease target site, and the nucleic acid construct is inserted into the genomic safe harbor locus.

8. A method of expressing a product of interest from a genomic safe harbor locus in a human cell in a human subject, comprising administering to the human subject:
(a) a nuclease agent or one or more nucleic acids encoding the nuclease agent, wherein the nuclease agent targets a nuclease target site in the genomic safe harbor locus, wherein the genomic safe harbor locus is selected from the following genomic locations:
(i) genomic coordinates of about 77460242 to about 77460537 on human chromosome 13;
(ii) genomic coordinates of about 170031084 to about 170031382 on human chromosome 6; and
(iii) genomic coordinates of about 25207412 to about 25207703 on human chromosome 9; and
(b) a nucleic acid construct, wherein the nucleic acid construct comprises a nucleic acid operably linked to a promoter, wherein the nucleic acid encodes the product of interest,
wherein the nuclease agent cleaves the nuclease target site, the nucleic acid construct is inserted into the genomic safe harbor locus to create a modified genomic safe harbor locus, and the product of interest is expressed from the modified genomic safe harbor locus.

9. The method of claim 7 or 8, wherein the human cell is a liver cell.

10. The method of any one of claims 7-9, wherein the human cell is a hepatocyte.

11. The method of any preceding claim, wherein the nuclease agent comprises:
(a) a zinc finger nuclease (ZFN);
(b) a transcription activator-like effector nuclease (TALEN); or
(c) (i) a Cas protein or a nucleic acid encoding the Cas protein; and
(ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

12. The method of any one of claims 1-10, wherein the nuclease agent comprises:
(a) a Cas protein or a nucleic acid encoding the Cas protein; and
(b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

13. The method of claim 12, wherein the method comprises administering the guide RNA in the form of RNA.

14. The method of claim 13, wherein the guide RNA comprises at least one modification.

15. The method of claim 14, wherein the at least one modification comprises a 2′-O-methyl-modified nucleotide.

16. The method of claim 14 or 15, wherein the at least one modification comprises a phosphorothioate bond between nucleotides.

17. The method of any one of claims 12-16, wherein the guide RNA is a single guide RNA (sgRNA).

18. The method of any one of claims 12-17, wherein the Cas protein is a Cas9 protein.

19. The method of claim 18, wherein the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

20. The method of claim 18, wherein the Cas protein is derived from a Streptococcus pyogenes Cas9 protein.

21. The method of any one of claims 12-20, wherein the nucleic acid encoding the Cas protein is codon-optimized for expression in a mammalian cell or a human cell.

22. The method of any one of claims 12-21, wherein the method comprises administering the nucleic acid encoding the Cas protein, wherein the nucleic acid comprises an mRNA encoding the Cas protein.

23. The method of claim 22, wherein the mRNA encoding the Cas protein comprises at least one modification.

24. The method of any one of claims 12-23, wherein the Cas protein or the nucleic acid encoding the Cas protein and the guide RNA or the one or more DNAs encoding the guide RNA are associated with a lipid nanoparticle.

25. The method of any preceding claim, wherein the genomic safe harbor locus is selected from the following genomic locations:
(i) human chromosome 13, coordinates 77460242-77460537;
(ii) human chromosome 6, coordinates 170031084-170031382; and
(iii) human chromosome 9, coordinates 25207412-25207703.

26. The method of any one of claims 12-25, wherein the genomic safe harbor locus is genomic coordinates of about 77460242 to about 77460537 on human chromosome 13.

27. The method of any one of claims 12-26, wherein the genomic safe harbor locus is human chromosome 13, coordinates 77460242-77460537 or comprises, consists essentially of, or consists of the sequence set forth in SEQ ID NO: 39.

28. The method of claim 26 or 27, wherein:
(I) the DNA-targeting segment comprises at least 17, at least 18, at least 19, or at least 20 contiguous nucleotides of the sequence set forth in any one of SEQ ID NOS: 25, 45, and 228-256; and/or
(II) the DNA-targeting segment is at least 90% or at least 95% identical to the sequence set forth in any one of SEQ ID NOS: 25, 45, and 228-256; and/or
(III) the DNA-targeting segment comprises any one of SEQ ID NOS: 25, 45, and 228-256; and/or
(IV) the DNA-targeting segment consists of any one of SEQ ID NOS: 25, 45, and 228-256.

29. The method of claim 26 or 27, wherein:
(I) the DNA-targeting segment comprises at least 17, at least 18, at least 19, or at least 20 contiguous nucleotides of the sequence set forth in SEQ ID NO: 25; and/or
(II) the DNA-targeting segment is at least 90% or at least 95% identical to the sequence set forth in SEQ ID NO: 25.

30. The method of any one of claims 26-29, wherein the DNA-targeting segment comprises SEQ ID NO: 25.

31. The method of any one of claims 26-30, wherein the DNA-targeting segment consists of SEQ ID NO: 25.

32. The method of any one of claims 12-25, wherein the genomic safe harbor locus is genomic coordinates of about 170031084 to about 170031382 on human chromosome 6.

33. The method of any one of claims 12-25 and 32, wherein the genomic safe harbor locus is human chromosome 6, coordinates 170031084-170031382 or comprises, consists essentially of, or consists of the sequence set forth in SEQ ID NO: 40.

34. The method of claim 32 or 33, wherein:
(I) the DNA-targeting segment comprises at least 17, at least 18, at least 19, or at least 20 contiguous nucleotides of the sequence set forth in any one of SEQ ID NOS: 26, 46, and 257-285; and/or
(II) the DNA-targeting segment is at least 90% or at least 95% identical to the sequence set forth in any one of SEQ ID NOS: 26, 46, and 257-285; and/or
(III) the DNA-targeting segment comprises any one of SEQ ID NOS: 26, 46, and 257-285; and/or
(IV) the DNA-targeting segment consists of any one of SEQ ID NOS: 26, 46, and 257-285.

35. The method of claim 32 or 33, wherein:
(I) the DNA-targeting segment comprises at least 17, at least 18, at least 19, or at least 20 contiguous nucleotides of the sequence set forth in SEQ ID NO: 26; and/or
(II) the DNA-targeting segment is at least 90% or at least 95% identical to the sequence set forth in SEQ ID NO: 26.

36. The method of any one of claims 32-35, wherein the DNA-targeting segment comprises SEQ ID NO: 26.

37. The method of any one of claims 32-36, wherein the DNA-targeting segment consists of SEQ ID NO: 26.

38. The method of any one of claims 12-25, wherein the genomic safe harbor locus is genomic coordinates of about 25207412 to about 25207703 on human chromosome 9.

39. The method of any one of claims 12-25 and 38, wherein the genomic safe harbor locus is human chromosome 9, coordinates 25207412-25207703 or comprises, consists essentially of, or consists of the sequence set forth in SEQ ID NO: 41.

40. The method of claim 38 or 39, wherein:
(I) the DNA-targeting segment comprises at least 17, at least 18, at least 19, or at least 20 contiguous nucleotides of the sequence set forth in any one of SEQ ID NOS: 27, 47, and 286-314; and/or
(II) the DNA-targeting segment is at least 90% or at least 95% identical to the sequence set forth in any one of SEQ ID NOS: 27, 47, and 286-314; and/or
(III) the DNA-targeting segment comprises any one of SEQ ID NOS: 27, 47, and 286-314; and/or
(IV) the DNA-targeting segment consists of any one of SEQ ID NOS: 27, 47, and 286-314.

41. The method of claim 38 or 39, wherein:
(I) the DNA-targeting segment comprises at least 17, at least 18, at least 19, or at least 20 contiguous nucleotides of the sequence set forth in SEQ ID NO: 27; and/or
(II) the DNA-targeting segment is at least 90% or at least 95% identical to the sequence set forth in SEQ ID NO: 27.

42. The method of any one of claims 38-41, wherein the DNA-targeting segment comprises SEQ ID NO: 27.

43. The method of any one of claims 38-42, wherein the DNA-targeting segment consists of SEQ ID NO: 27.

44. The method of any one of claims 1-11, wherein the genomic safe harbor locus is genomic coordinates of about 77460242 to about 77460537 on human chromosome 13.

45. The method of any one of claims 1-11, wherein the genomic safe harbor locus is human chromosome 13, coordinates 77460242-77460537 or comprises, consists essentially of, or consists of the sequence set forth in SEQ ID NO: 39.

46. The method of any one of claims 1-11, wherein the genomic safe harbor locus is genomic coordinates of about 170031084 to about 170031382 on human chromosome 6.

47. The method of any one of claims 1-11, wherein the genomic safe harbor locus is human chromosome 6, coordinates 170031084-170031382 or comprises, consists essentially of, or consists of the sequence set forth in SEQ ID NO: 40.

48. The method of any one of claims 1-11, wherein the genomic safe harbor locus is genomic coordinates of about 25207412 to about 25207703 on human chromosome 9.

49. The method of any one of claims 1-11, wherein the genomic safe harbor locus is human chromosome 9, coordinates 25207412-25207703 or comprises, consists essentially of, or consists of the sequence set forth in SEQ ID NO: 41.

50. A method of integrating a nucleic acid construct into a genomic safe harbor locus in a mouse cell, comprising administering to the mouse cell:
(a) a nuclease agent or one or more nucleic acids encoding the nuclease agent, wherein the nuclease agent targets a nuclease target site in the genomic safe harbor locus, wherein the genomic safe harbor locus is selected from the following genomic locations:
(i) genomic coordinates of about 103,450,397 to about 103,451,396 on mouse chromosome 14;
(ii) genomic coordinates of about 15,226,387 to about 15,227,386 on mouse chromosome 17; and
(iii) genomic coordinates of about 92,827,563 to about 92,828,592 on mouse chromosome 4; and
(b) the nucleic acid construct, wherein the nucleic acid construct comprises a nucleic acid operably linked to a promoter, wherein the nucleic acid encodes a product of interest,
wherein the nuclease agent cleaves the nuclease target site, and the nucleic acid construct is inserted into the genomic safe harbor locus.

51. A method of expressing a product of interest from a genomic safe harbor locus in a mouse cell, comprising administering to the mouse cell:
(a) a nuclease agent or one or more nucleic acids encoding the nuclease agent, wherein the nuclease agent targets a nuclease target site in the genomic safe harbor locus, wherein the genomic safe harbor locus is selected from the following genomic locations:
(i) genomic coordinates of about 103,450,397 to about 103,451,396 on mouse chromosome 14;
(ii) genomic coordinates of about 15,226,387 to about 15,227,386 on mouse chromosome 17; and
(iii) genomic coordinates of about 92,827,563 to about 92,828,592 on mouse chromosome 4; and
(b) a nucleic acid construct, wherein the nucleic acid construct comprises a nucleic acid operably linked to a promoter, wherein the nucleic acid encodes the product of interest,
wherein the nuclease agent cleaves the nuclease target site, the nucleic acid construct is inserted into the genomic safe harbor locus to create a modified genomic safe harbor locus, and the product of interest is expressed from the modified genomic safe harbor locus.

52. The method of claim 50 or 51, wherein the mouse cell is a liver cell.

53. The method of any one of claims 50-52, wherein the mouse cell is a hepatocyte.

54. The method of any one of claims 50-53, wherein the mouse cell is in vitro or ex vivo.

55. The method of any one of claims 50-53, wherein the mouse cell is in vivo in a subject.

56. A method of integrating a nucleic acid construct into a genomic safe harbor locus in a mouse cell in a mouse subject, comprising administering to the mouse subject:
(a) a nuclease agent or one or more nucleic acids encoding the nuclease agent, wherein the nuclease agent targets a nuclease target site in the genomic safe harbor locus, wherein the genomic safe harbor locus is selected from the following genomic locations:
(i) genomic coordinates of about 103,450,397 to about 103,451,396 on mouse chromosome 14;
(ii) genomic coordinates of about 15,226,387 to about 15,227,386 on mouse chromosome 17; and
(iii) genomic coordinates of about 92,827,563 to about 92,828,592 on mouse chromosome 4; and
(b) the nucleic acid construct, wherein the nucleic acid construct comprises a nucleic acid operably linked to a promoter, wherein the nucleic acid encodes a product of interest,
wherein the nuclease agent cleaves the nuclease target site, and the nucleic acid construct is inserted into the genomic safe harbor locus.

57. A method of expressing a product of interest from a genomic safe harbor locus in a mouse cell in a mouse subject, comprising administering to the mouse subject:
(a) a nuclease agent or one or more nucleic acids encoding the nuclease agent, wherein the nuclease agent targets a nuclease target site in the genomic safe harbor locus, wherein the genomic safe harbor locus is selected from the following genomic locations:
(i) genomic coordinates of about 103,450,397 to about 103,451,396 on mouse chromosome 14;
(ii) genomic coordinates of about 15,226,387 to about 15,227,386 on mouse chromosome 17; and
(iii) genomic coordinates of about 92,827,563 to about 92,828,592 on mouse chromosome 4; and
(b) a nucleic acid construct, wherein the nucleic acid construct comprises a nucleic acid operably linked to a promoter, wherein the nucleic acid encodes the product of interest,
wherein the nuclease agent cleaves the nuclease target site, the nucleic acid construct is inserted into the genomic safe harbor locus to create a modified genomic safe harbor locus, and the product of interest is expressed from the modified genomic safe harbor locus.

58. The method of claim 56 or 57, wherein the mouse cell is a liver cell.

59. The method of any one of claims 56-58, wherein the mouse cell is a hepatocyte.

60. The method of any one of claims 50-59, wherein the nuclease agent comprises:
(a) a zinc finger nuclease (ZFN);
(b) a transcription activator-like effector nuclease (TALEN); or
(c) (i) a Cas protein or a nucleic acid encoding the Cas protein; and
(ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

61. The method of any one of claims 50-59, wherein the nuclease agent comprises:
(a) a Cas protein or a nucleic acid encoding the Cas protein; and
(b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

62. The method of claim 61, wherein the method comprises administering the guide RNA in the form of RNA.

63. The method of claim 62, wherein the guide RNA comprises at least one modification.

64. The method of claim 63, wherein the at least one modification comprises a 2′-O-methyl-modified nucleotide.

65. The method of claim 63 or 64, wherein the at least one modification comprises a phosphorothioate bond between nucleotides.

66. The method of any one of claims 61-65, wherein the guide RNA is a single guide RNA (sgRNA).

67. The method of any one of claims 61-66, wherein the Cas protein is a Cas9 protein.

68. The method of claim 67, wherein the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

69. The method of claim 67, wherein the Cas protein is derived from a Streptococcus pyogenes Cas9 protein.

70. The method of any one of claims 61-69, wherein the nucleic acid encoding the Cas protein is codon-optimized for expression in a mammalian cell or a mouse cell.

71. The method of any one of claims 61-70, wherein the method comprises administering the nucleic acid encoding the Cas protein, wherein the nucleic acid comprises an mRNA encoding the Cas protein.

72. The method of claim 71, wherein the mRNA encoding the Cas protein comprises at least one modification.

73. The method of any one of claims 61-72, wherein the Cas protein or the nucleic acid encoding the Cas protein and the guide RNA or the one or more DNAs encoding the guide RNA are associated with a lipid nanoparticle.

74. The method of any one of claims 50-73, wherein the genomic safe harbor locus is selected from the following genomic locations:
(i) mouse chromosome 14, coordinates 103,450,397-103,451,396;
(ii) mouse chromosome 17, coordinates 15,226,387-15,227,386; and
(iii) mouse chromosome 4, coordinates 92,827,563-92,828,592.

75. The method of any one of claims 61-74, wherein the genomic safe harbor locus is genomic coordinates of about 103,450,397 to about 103,451,396 on mouse chromosome 14.

76. The method of any one of claims 61-75, wherein the genomic safe harbor locus is mouse chromosome 14, coordinates 103,450,397-103,451,396 or comprises, consists essentially of, or consists of the sequence set forth in SEQ ID NO: 405.

77. The method of claim 75 or 76, wherein:
(I) the DNA-targeting segment comprises at least 17, at least 18, at least 19, or at least 20 contiguous nucleotides of the sequence set forth in any one of SEQ ID NOS: 315-344; and/or
(II) the DNA-targeting segment is at least 90% or at least 95% identical to the sequence set forth in any one of SEQ ID NOS: 315-344; and/or
(III) the DNA-targeting segment comprises any one of SEQ ID NOS: 315-344; and/or
(IV) the DNA-targeting segment consists of any one of SEQ ID NOS: 315-344.

78. The method of any one of claims 61-74, wherein the genomic safe harbor locus is genomic coordinates of about 15,226,387 to about 15,227,386 on mouse chromosome 17.

79. The method of any one of claims 61-74 and 78, wherein the genomic safe harbor locus is mouse chromosome 17, coordinates 15,226,387-15,227,386 or comprises, consists essentially of, or consists of the sequence set forth in SEQ ID NO: 406.

80. The method of claim 78 or 79, wherein:
(I) the DNA-targeting segment comprises at least 17, at least 18, at least 19, or at least 20 contiguous nucleotides of the sequence set forth in any one of SEQ ID NOS: 345-374; and/or
(II) the DNA-targeting segment is at least 90% or at least 95% identical to the sequence set forth in any one of SEQ ID NOS: 345-374; and/or
(III) the DNA-targeting segment comprises any one of SEQ ID NOS: 345-374; and/or
(IV) the DNA-targeting segment consists of any one of SEQ ID NOS: 345-374.

81. The method of any one of claims 61-74, wherein the genomic safe harbor locus is genomic coordinates of about 92,827,563 to about 92,828,592 on mouse chromosome 4.

82. The method of any one of claims 61-74 and 81, wherein the genomic safe harbor locus is mouse chromosome 4, coordinates 92,827,563-92,828,592 or comprises, consists essentially of, or consists of the sequence set forth in SEQ ID NO: 407.

83. The method of claim 81 or 82, wherein:
(I) the DNA-targeting segment comprises at least 17, at least 18, at least 19, or at least 20 contiguous nucleotides of the sequence set forth in any one of SEQ ID NOS: 375-404; and/or
(II) the DNA-targeting segment is at least 90% or at least 95% identical to the sequence set forth in any one of SEQ ID NOS: 375-404; and/or
(III) the DNA-targeting segment comprises any one of SEQ ID NOS: 375-404; and/or
(IV) the DNA-targeting segment consists of any one of SEQ ID NOS: 375-404.

84. The method of any one of claims 50-60, wherein the genomic safe harbor locus is genomic coordinates of about 103,450,397 to about 103,451,396 on mouse chromosome 14.

85. The method of any one of claims 50-60, wherein the genomic safe harbor locus is mouse chromosome 14, coordinates 103,450,397-103,451,396 or comprises, consists essentially of, or consists of the sequence set forth in SEQ ID NO: 405.

86. The method of any one of claims 50-60, wherein the genomic safe harbor locus is genomic coordinates of about 15,226,387 to about 15,227,386 on mouse chromosome 17.

87. The method of any one of claims 50-60, wherein the genomic safe harbor locus is mouse chromosome 17, coordinates 15,226,387-15,227,386 or comprises, consists essentially of, or consists of the sequence set forth in SEQ ID NO: 406.

88. The method of any one of claims 50-60, wherein the genomic safe harbor locus is genomic coordinates of about 92,827,563 to about 92,828,592 on mouse chromosome 4.

89. The method of any one of claims 50-60, wherein the genomic safe harbor locus is mouse chromosome 4, coordinates 92,827,563-92,828,592 or comprises, consists essentially of, or consists of the sequence set forth in SEQ ID NO: 407.

90. The method of any preceding claim, wherein the nucleic acid construct is administered simultaneously with the nuclease agent or the one or more nucleic acids encoding the nuclease agent.

91. The method of any one of claims 1-89, wherein the nucleic acid construct is not administered simultaneously with the nuclease agent or the one or more nucleic acids encoding the nuclease agent.

92. The method of claim 91, wherein the nucleic acid construct is administered prior to the nuclease agent or the one or more nucleic acids encoding the nuclease agent.

93. The method of claim 91, wherein the nucleic acid construct is administered after the nuclease agent or the one or more nucleic acids encoding the nuclease agent.

94. The method of any preceding claim, wherein the product of interest is a polypeptide of interest.

95. The method of claim 94, wherein the polypeptide of interest comprises a therapeutic polypeptide.

96. The method of claim 94 or 95, wherein the polypeptide of interest is a secreted polypeptide.

97. The method of claim 94 or 95, wherein the polypeptide of interest is an intracellular polypeptide.

98. The method of any preceding claim, wherein the promoter is active in liver cells.

99. The method of any preceding claim, wherein the promoter is a tissue-specific promoter.

100. The method of any one of claims 1-98, wherein the promoter is a constitutive promoter.

101. The method of any one of claims 1-99, wherein the promoter is an inducible promoter.

102. The method of any preceding claim, wherein the nucleic acid construct does not comprise a homology arm.

103. The method of claim 102, wherein the nucleic acid construct is inserted into the target genomic locus via non-homologous end joining.

104. The method of any one of claims 1-101, wherein the nucleic acid construct comprises homology arms.

105. The method of claim 104, wherein the nucleic acid construct is inserted into the target genomic locus via homology-directed repair.

106. The method of any preceding claim, wherein the nucleic acid construct is single-stranded DNA or double-stranded DNA.

107. The method of claim 106, wherein the nucleic acid construct is single-stranded DNA.

108. The method of any preceding claim, wherein the nucleic acid construct is in a nucleic acid vector or a lipid nanoparticle.

109. The method of claim 108, wherein the nucleic acid construct is in the nucleic acid vector.

110. The method of claim 109, wherein the nucleic acid vector is a viral vector.

111. The method of claim 109 or 110, wherein the nucleic acid vector is an adeno-associated viral (AAV) vector.

112. The method of claim 111, wherein the AAV vector is a single-stranded AAV (ssAAV) vector.

113. The method of claim 111 or 112, wherein the AAV vector is derived from an AAV8 vector, an AAV3B vector, an AAV5 vector, an AAV6 vector, an AAV7 vector, an AAV9 vector, an AAVrh.74 vector, an AAV-DJ vector, or an AAVhu.37 vector.

114. The method of any one of claims 111-113, wherein the AAV vector is a recombinant AAV8 (rAAV8) vector.

115. The method of claim 114, wherein the AAV vector is a single-stranded rAAV8 vector.

116. A cell made by the method of any preceding claim.

117. A human cell comprising a nucleic acid construct integrated into a genomic safe harbor locus,
wherein the nucleic acid construct comprises a nucleic acid operably linked to a promoter, wherein the nucleic acid encodes a product of interest, and
wherein the genomic safe harbor locus is selected from the following genomic locations:
(i) genomic coordinates of about 77460242 to about 77460537 on human chromosome 13;
(ii) genomic coordinates of about 170031084 to about 170031382 on human chromosome 6; and
(iii) genomic coordinates of about 25207412 to about 25207703 on human chromosome 9.

118. The human cell of claim 117, wherein the genomic safe harbor locus is selected from the following genomic locations:
(i) human chromosome 13, coordinates 77460242-77460537;
(ii) human chromosome 6, coordinates 170031084-170031382; and
(iii) human chromosome 9, coordinates 25207412-25207703.

119. The human cell of claim 117 or 118, wherein the genomic safe harbor locus is genomic coordinates of about 77460242 to about 77460537 on human chromosome 13.

120. The human cell of any one of claims 117-119, wherein the genomic safe harbor locus is human chromosome 13, coordinates 77460242-77460537 or comprises, consists essentially of, or consists of the sequence set forth in SEQ ID NO: 39.

121. The human cell of claim 117 or 118, wherein the genomic safe harbor locus is genomic coordinates of about 170031084 to about 170031382 on human chromosome 6.

122. The human cell of any one of claims 117, 118, and 121, wherein the genomic safe harbor locus is human chromosome 6, coordinates 170031084-170031382 or comprises, consists essentially of, or consists of the sequence set forth in SEQ ID NO: 40.

123. The human cell of claim 117 or 118, wherein the genomic safe harbor locus is genomic coordinates of about 25207412 to about 25207703 on human chromosome 9.

124. The human cell of any one of claims 117, 118, and 123, wherein the genomic safe harbor locus is human chromosome 9, coordinates 25207412-25207703 or comprises, consists essentially of, or consists of the sequence set forth in SEQ ID NO: 41.

125. A mouse cell comprising a nucleic acid construct integrated into a genomic safe harbor locus,
wherein the nucleic acid construct comprises a nucleic acid operably linked to a promoter, wherein the nucleic acid encodes a product of interest, and
wherein the genomic safe harbor locus is selected from the following genomic locations:
(i) genomic coordinates of about 103,450,397 to about 103,451,396 on mouse chromosome 14;
(ii) genomic coordinates of about 15,226,387 to about 15,227,386 on mouse chromosome 17; and
(iii) genomic coordinates of about 92,827,563 to about 92,828,592 on mouse chromosome 4.

126. The mouse cell of claim 125, wherein the genomic safe harbor locus is selected from the following genomic locations:
(i) mouse chromosome 14, coordinates 103,450,397-103,451,396;
(ii) mouse chromosome 17, coordinates 15,226,387-15,227,386; and
(iii) mouse chromosome 4, coordinates 92,827,563-92,828,592.

127. The mouse cell of claim 125 or 126, wherein the genomic safe harbor locus is genomic coordinates of about 103,450,397 to about 103,451,396 on mouse chromosome 14.

128. The mouse cell of any one of claims 125-127, wherein the genomic safe harbor locus is mouse chromosome 14, coordinates 103,450,397-103,451,396 or comprises, consists essentially of, or consists of the sequence set forth in SEQ ID NO: 405.

129. The mouse cell of claim 125 or 126, wherein the genomic safe harbor locus is genomic coordinates of about 15,226,387 to about 15,227,386 on mouse chromosome 17.

130. The mouse cell of any one of claims 125, 126, and 129, wherein the genomic safe harbor locus is mouse chromosome 17, coordinates 15,226,387-15,227,386 or comprises, consists essentially of, or consists of the sequence set forth in SEQ ID NO: 406.

131. The mouse cell of claim 125 or 126, wherein the genomic safe harbor locus is genomic coordinates of about 92,827,563 to about 92,828,592 on mouse chromosome 4.

132. The mouse cell of any one of claims 125, 126, and 131, wherein the genomic safe harbor locus is mouse chromosome 4, coordinates 92,827,563-92,828,592 or comprises, consists essentially of, or consists of the sequence set forth in SEQ ID NO: 407.

133. The cell of any one of claims 116-132, wherein the cell is a liver cell.

134. The cell of any one of claims 116-133, wherein the cell is a hepatocyte.

135. The cell of any one of claims 116-134, wherein the product of interest is expressed.

136. The cell of any one of claims 116-135, wherein the product of interest is a polypeptide of interest.

137. The cell of claim 136, wherein the polypeptide of interest comprises a therapeutic polypeptide.

138. The cell of claim 136 or 137, wherein the polypeptide of interest is a secreted polypeptide.

139. The cell of claim 136 or 137, wherein the polypeptide of interest is an intracellular polypeptide.

140. The cell of any one of claims 116-139, wherein the promoter is active in liver cells.

141. The cell of any one of claims 116-140, wherein the promoter is a tissue-specific promoter.

142. The cell of any one of claims 116-140, wherein the promoter is a constitutive promoter.

143. The cell of any one of claims 116-141, wherein the promoter is an inducible promoter.

144. A composition comprising a guide RNA or a DNA encoding a guide RNA, wherein the guide RNA comprises a DNA-targeting segment that targets a guide RNA target sequence in a genomic safe harbor locus and a protein-binding segment that binds to a Cas protein, wherein the genomic safe harbor locus is selected from the following genomic locations:
(i) genomic coordinates of about 77460242 to about 77460537 on human chromosome 13;
(ii) genomic coordinates of about 170031084 to about 170031382 on human chromosome 6; and
(iii) genomic coordinates of about 25207412 to about 25207703 on human chromosome 9.

145. The composition of claim 144, wherein the genomic safe harbor locus is selected from the following genomic locations:
(i) human chromosome 13, coordinates 77460242-77460537;
(ii) human chromosome 6, coordinates 170031084-170031382; and
(iii) human chromosome 9, coordinates 25207412-25207703.

146. The composition of claim 144 or 145, wherein the genomic safe harbor locus is genomic coordinates of about 77460242 to about 77460537 on human chromosome 13.

147. The composition of any one of claims 144-146, wherein the genomic safe harbor locus is human chromosome 13, coordinates 77460242-77460537 or comprises, consists essentially of, or consists of the sequence set forth in SEQ ID NO: 39.

148. The composition of claim 146 or 147, wherein:
(I) the DNA-targeting segment comprises at least 17, at least 18, at least 19, or at least 20 contiguous nucleotides of the sequence set forth in any one of SEQ ID NOS: 25, 45, and 228-256; and/or
(II) the DNA-targeting segment is at least 90% or at least 95% identical to the sequence set forth in any one of SEQ ID NOS: 25, 45, and 228-256; and/or
(III) the DNA-targeting segment comprises any one of SEQ ID NOS: 25, 45, and 228-256; and/or
(IV) the DNA-targeting segment consists of any one of SEQ ID NOS: 25, 45, and 228-256.

149. The composition of claim 146 or 147, wherein:
(I) the DNA-targeting segment comprises at least 17, at least 18, at least 19, or at least 20 contiguous nucleotides of the sequence set forth in SEQ ID NO: 25; and/or
(II) the DNA-targeting segment is at least 90% or at least 95% identical to the sequence set forth in SEQ ID NO: 25.

150. The composition of claim any one of claims 146-149, wherein the DNA-targeting segment comprises SEQ ID NO: 25.

151. The composition of any one of claims 146-150, wherein the DNA-targeting segment consists of SEQ ID NO: 25.

152. The composition of claim 144 or 145, wherein the genomic safe harbor locus is genomic coordinates of about 170031084 to about 170031382 on human chromosome 6.

153. The composition of any one of claims 144, 145 and 152, wherein the genomic safe harbor locus is human chromosome 6, coordinates 170031084-170031382 or comprises, consists essentially of, or consists of the sequence set forth in SEQ ID NO: 40.

154. The composition of claim 152 or 153, wherein:
(I) the DNA-targeting segment comprises at least 17, at least 18, at least 19, or at least 20 contiguous nucleotides of the sequence set forth in any one of SEQ ID NOS: 26, 46, and 257-285; and/or
(II) the DNA-targeting segment is at least 90% or at least 95% identical to the sequence set forth in any one of SEQ ID NOS: 26, 46, and 257-285; and/or
(III) the DNA-targeting segment comprises any one of SEQ ID NOS: 26, 46, and 257-285; and/or
(IV) the DNA-targeting segment consists of any one of SEQ ID NOS: 26, 46, and 257-285.

155. The composition of claim 152 or 153, wherein:
(I) the DNA-targeting segment comprises at least 17, at least 18, at least 19, or at least 20 contiguous nucleotides of the sequence set forth in SEQ ID NO: 26; and/or
(II) the DNA-targeting segment is at least 90% or at least 95% identical to the sequence set forth in SEQ ID NO: 26.

156. The composition of any one of claims 152-155, wherein the DNA-targeting segment comprises SEQ ID NO: 26.

157. The composition of any one of claims 152-156, wherein the DNA-targeting segment consists of SEQ ID NO: 26.

158. The composition of claim 144 or 145, wherein the genomic safe harbor locus is genomic coordinates of about 25207412 to about 25207703 on human chromosome 9.

159. The composition of any one of claims 144, 145, and 158, wherein the genomic safe harbor locus is human chromosome 9, coordinates 25207412-25207703 or comprises, consists essentially of, or consists of the sequence set forth in SEQ ID NO: 41.

160. The composition of claim 158 or 159, wherein:
(I) the DNA-targeting segment comprises at least 17, at least 18, at least 19, or at least 20 contiguous nucleotides of the sequence set forth in any one of SEQ ID NOS: 27, 47, and 286-314; and/or
(II) the DNA-targeting segment is at least 90% or at least 95% identical to the sequence set forth in any one of SEQ ID NOS: 27, 47, and 286-314; and/or
(III) the DNA-targeting segment comprises any one of SEQ ID NOS: 27, 47, and 286-314; and/or
(IV) the DNA-targeting segment consists of any one of SEQ ID NOS: 27, 47, and 286-314.

161. The composition of claim 158 or 159, wherein:
(I) the DNA-targeting segment comprises at least 17, at least 18, at least 19, or at least 20 contiguous nucleotides of the sequence set forth in SEQ ID NO: 27; and/or
(II) the DNA-targeting segment is at least 90% or at least 95% identical to the sequence set forth in SEQ ID NO: 27.

162. The composition of any one of claims 158-161, wherein the DNA-targeting segment comprises SEQ ID NO: 27.

163. The composition of any one of claims 158-162, wherein the DNA-targeting segment consists of SEQ ID NO: 27.

164. A composition comprising a guide RNA or a DNA encoding a guide RNA, wherein the guide RNA comprises a DNA-targeting segment that targets a guide RNA target sequence in a genomic safe harbor locus and a protein-binding segment that binds to a Cas protein, wherein the genomic safe harbor locus is selected from the following genomic locations:
(i) genomic coordinates of about 103,450,397 to about 103,451,396 on mouse chromosome 14;
(ii) genomic coordinates of about 15,226,387 to about 15,227,386 on mouse chromosome 17; and
(iii) genomic coordinates of about 92,827,563 to about 92,828,592 on mouse chromosome 4.

165. The composition of claim 164, wherein the genomic safe harbor locus is selected from the following genomic locations:
(i) mouse chromosome 14, coordinates 103,450,397-103,451,396;
(ii) mouse chromosome 17, coordinates 15,226,387-15,227,386; and
(iii) mouse chromosome 4, coordinates 92,827,563-92,828,592.

166. The composition of claim 164 or 165, wherein the genomic safe harbor locus is genomic coordinates of about 103,450,397 to about 103,451,396 on mouse chromosome 14.

167. The composition of any one of claims 164-166, wherein the genomic safe harbor locus is mouse chromosome 14, coordinates 103,450,397-103,451,396 or comprises, consists essentially of, or consists of the sequence set forth in SEQ ID NO: 405.

168. The composition of claim 166 or 167, wherein:
(I) the DNA-targeting segment comprises at least 17, at least 18, at least 19, or at least 20 contiguous nucleotides of the sequence set forth in any one of SEQ ID NOS: 315-344; and/or
(II) the DNA-targeting segment is at least 90% or at least 95% identical to the sequence set forth in any one of SEQ ID NOS: 315-344; and/or
(III) the DNA-targeting segment comprises any one of SEQ ID NOS: 315-344;
and/or (IV) the DNA-targeting segment consists of any one of SEQ ID NOS: 315-344.

169. The composition of claim 164 or 165, wherein the genomic safe harbor locus is genomic coordinates of about 15,226,387 to about 15,227,386 on mouse chromosome 17.

170. The composition of any one of claims 164, 165, and 169, wherein the genomic safe harbor locus is mouse chromosome 17, coordinates 15,226,387-15,227,386 or comprises, consists essentially of, or consists of the sequence set forth in SEQ ID NO: 406.

171. The composition of claim 169 or 170, wherein:
(I) the DNA-targeting segment comprises at least 17, at least 18, at least 19, or at least 20 contiguous nucleotides of the sequence set forth in any one of SEQ ID NOS: 345-374; and/or
(II) the DNA-targeting segment is at least 90% or at least 95% identical to the sequence set forth in any one of SEQ ID NOS: 345-374; and/or
(III) the DNA-targeting segment comprises any one of SEQ ID NOS: 345-374; and/or
(IV) the DNA-targeting segment consists of any one of SEQ ID NOS: 345-374.

172. The composition of claim 164 or 165, wherein the genomic safe harbor locus is genomic coordinates of about 92,827,563 to about 92,828,592 on mouse chromosome 4.

173. The composition of any one of claims 164, 165, and 172, wherein the genomic safe harbor locus is mouse chromosome 4, coordinates 92,827,563-92,828,592 or comprises, consists essentially of, or consists of the sequence set forth in SEQ ID NO: 407.

174. The composition of claim 172 or 173, wherein:
(I) the DNA-targeting segment comprises at least 17, at least 18, at least 19, or at least 20 contiguous nucleotides of the sequence set forth in any one of SEQ ID NOS: 375-404; and/or
(II) the DNA-targeting segment is at least 90% or at least 95% identical to the sequence set forth in any one of SEQ ID NOS: 375-404; and/or
(III) the DNA-targeting segment comprises any one of SEQ ID NOS: 375-404; and/or
(IV) the DNA-targeting segment consists of any one of SEQ ID NOS: 375-404.

175. The composition of any one of claims 144-174, wherein the composition comprises the DNA encoding the guide RNA.

176. The composition of claim 175, wherein the DNA encoding the guide RNA is in a nucleic acid vector.

177. The composition of claim 176, wherein the nucleic acid vector is a viral vector.

178. The composition of claim 176 or 177, wherein the nucleic acid vector is an adeno-associated viral (AAV) vector.

179. The composition of claim 178, wherein the AAV vector is a single-stranded AAV (ssAAV) vector.

180. The composition of claim 178 or 179, wherein the AAV vector is derived from an AAV8 vector, an AAV3B vector, an AAV5 vector, an AAV6 vector, an AAV7 vector, an AAV9 vector, an AAVrh.74 vector, an AAV-DJ vector, or an AAVhu.37 vector.

181. The composition of any one of claims 178-180, wherein the AAV vector is a recombinant AAV8 (rAAV8) vector.

182. The composition of claim 181, wherein the AAV vector is a single-stranded rAAV8 vector.

183. The composition of any one of claims 144-174, wherein the composition comprises the guide RNA in the form of RNA.

184. The composition of claim 183, wherein the guide RNA comprises at least one modification.

185. The composition of claim 184, wherein the at least one modification comprises a 2′-O-methyl-modified nucleotide.

186. The composition of claim 184 or 185, wherein the at least one modification comprises a phosphorothioate bond between nucleotides.

187. The composition of any one of claims 144-186, wherein the guide RNA is a single guide RNA (sgRNA).

188. The composition of any one of claims 144-187, further comprising the Cas protein or a nucleic acid encoding the Cas protein.

189. The composition of claim 188, wherein the composition comprises the Cas protein.

190. The composition of claim 188, wherein the composition comprises the nucleic acid encoding the Cas protein.

191. The composition of claim 190, wherein the nucleic acid encoding the Cas protein is codon-optimized for expression in a mammalian cell or a human cell.

192. The composition of claim 190 or 191, wherein the nucleic acid encoding the Cas protein comprises a DNA encoding the Cas protein.

193. The composition of claim 192, wherein the DNA encoding the guide RNA is in a nucleic acid vector.

194. The composition of claim 193, wherein the nucleic acid vector is a viral vector.

195. The composition of claim 193 or 194, wherein the nucleic acid vector is an adeno-associated viral (AAV) vector.

196. The composition of claim 195, wherein the AAV vector is a single-stranded AAV (ssAAV) vector.

197. The composition of claim 195 or 196, wherein the AAV vector is derived from an AAV8 vector, an AAV3B vector, an AAV5 vector, an AAV6 vector, an AAV7 vector, an AAV9 vector, an AAVrh.74 vector, an AAV-DJ vector, or an AAVhu.37 vector.

198. The composition of any one of claims 195-197, wherein the AAV vector is a recombinant AAV8 (rAAV8) vector.

199. The composition of claim 198, wherein the AAV vector is a single-stranded rAAV8 vector.

200. The composition of claim 190 or 191, wherein the nucleic acid encoding the Cas protein comprises an mRNA encoding the Cas protein.

201. The composition of claim 200, wherein the mRNA encoding the Cas protein comprises at least one modification.

202. The composition of any one of claims 144-201, wherein the Cas protein or the nucleic acid encoding the Cas protein and the guide RNA or the one or more DNAs encoding the guide RNA are associated with a lipid nanoparticle.

203. The composition of any one of claims 144-202, wherein the Cas protein is a Cas9 protein.

204. The composition of claim 203, wherein the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

205. The composition of claim 203, wherein the Cas protein is derived from a Streptococcus pyogenes Cas9 protein.

206. The composition of any one of claims 144-202, wherein the composition further comprises a nucleic acid construct, wherein the nucleic acid construct comprises a nucleic acid operably linked to a promoter, wherein the nucleic acid encodes a product of interest.

207. The composition of claim 206, wherein the product of interest is a polypeptide of interest.

208. The composition of claim 207, wherein the polypeptide of interest comprises a therapeutic polypeptide.

209. The composition of claim 207 or 208, wherein the polypeptide of interest is a secreted polypeptide.

210. The composition of claim 207 or 208, wherein the polypeptide of interest is an intracellular polypeptide.

211. The composition of any one of claims 206-210, wherein the promoter is active in liver cells.

212. The composition of any one of claims 206-211, wherein the promoter is a tissue-specific promoter.

213. The composition of any one of claims 206-211, wherein the promoter is a constitutive promoter.

214. The composition of any one of claims 206-211, wherein the promoter is an inducible promoter.

215. The composition of any one of claims 206-214, wherein the nucleic acid construct does not comprise a homology arm.

216. The composition of any one of claims 206-214, wherein the nucleic acid construct comprises homology arms.

217. The composition of any one of claims 206-216, wherein the nucleic acid construct is single-stranded DNA or double-stranded DNA.

218. The composition of claim 217, wherein the nucleic acid construct is single-stranded DNA.

219. The composition of any one of claims 206-218, wherein the nucleic acid construct is in a nucleic acid vector or a lipid nanoparticle.

220. The composition of claim 219, wherein the nucleic acid construct is in the nucleic acid vector.

221. The composition of claim 220, wherein the nucleic acid vector is a viral vector.

222. The composition of claim 220 or 221, wherein the nucleic acid vector is an adeno-associated viral (AAV) vector.

223. The composition of claim 222, wherein the AAV vector is a single-stranded AAV (ssAAV) vector.

224. The composition of claim 222 or 223, wherein the AAV vector is derived from an AAV8 vector, an AAV3B vector, an AAV5 vector, an AAV6 vector, an AAV7 vector, an AAV9 vector, an AAVrh.74 vector, an AAV-DJ vector, or an AAVhu.37 vector.

225. The composition of any one of claims 222-224, wherein the AAV vector is a recombinant AAV8 (rAAV8) vector.

226. The composition of claim 225, wherein the AAV vector is a single-stranded rAAV8 vector.

227. A nucleic acid comprising a genomic safe harbor locus comprising an integrated nucleic acid construct,
wherein the nucleic acid construct comprises a nucleic acid operably linked to a promoter, wherein the nucleic acid encodes a product of interest, and
wherein the genomic safe harbor locus is selected from the following genomic locations:
(i) genomic coordinates of about 77460242 to about 77460537 on human chromosome 13;
(ii) genomic coordinates of about 170031084 to about 170031382 on human chromosome 6; and
(iii) genomic coordinates of about 25207412 to about 25207703 on human chromosome 9.

228. The nucleic acid of claim 227, wherein the genomic safe harbor locus is selected from the following genomic locations:
(i) human chromosome 13, coordinates 77460242-77460537;
(ii) human chromosome 6, coordinates 170031084-170031382; and
(iii) human chromosome 9, coordinates 25207412-25207703.

229. The nucleic acid of claim 227 or 228, wherein the genomic safe harbor locus is genomic coordinates of about 77460242 to about 77460537 on human chromosome 13.

230. The nucleic acid of any one of claims 227-229, wherein the genomic safe harbor locus is human chromosome 13, coordinates 77460242-77460537 or comprises, consists essentially of, or consists of the sequence set forth in SEQ ID NO: 39.

231. The nucleic acid of claim 227 or 228, wherein the genomic safe harbor locus is genomic coordinates of about 170031084 to about 170031382 on human chromosome 6.

232. The nucleic acid of any one of claims 227, 228, and 231, wherein the genomic safe harbor locus is human chromosome 6, coordinates 170031084-170031382 or comprises, consists essentially of, or consists of the sequence set forth in SEQ ID NO: 40.

233. The nucleic acid of claim 227 or 228, wherein the genomic safe harbor locus is genomic coordinates of about 25207412 to about 25207703 on human chromosome 9.

234. The nucleic acid of any one of claims 227, 228, and 233, wherein the genomic safe harbor locus is human chromosome 9, coordinates 25207412-25207703 or comprises, consists essentially of, or consists of the sequence set forth in SEQ ID NO: 41.

235. A nucleic acid comprising a genomic safe harbor locus comprising an integrated nucleic acid construct,
wherein the nucleic acid construct comprises a nucleic acid operably linked to a promoter, wherein the nucleic acid encodes a product of interest, and
wherein the genomic safe harbor locus is selected from the following genomic locations:
(i) genomic coordinates of about 103,450,397 to about 103,451,396 on mouse chromosome 14;
(ii) genomic coordinates of about 15,226,387 to about 15,227,386 on mouse chromosome 17; and
(iii) genomic coordinates of about 92,827,563 to about 92,828,592 on mouse chromosome 4.

236. The nucleic acid of claim 235, wherein the genomic safe harbor locus is selected from the following genomic locations:
(i) mouse chromosome 14, coordinates 103,450,397-103,451,396;
(ii) mouse chromosome 17, coordinates 15,226,387-15,227,386; and
(iii) mouse chromosome 4, coordinates 92,827,563-92,828,592.

237. The nucleic acid of claim 235 or 236, wherein the genomic safe harbor locus is genomic coordinates of about 103,450,397 to about 103,451,396 on mouse chromosome 14.

238. The nucleic acid of any one of claims 235-237, wherein the genomic safe harbor locus is mouse chromosome 14, coordinates 103,450,397-103,451,396 or comprises, consists essentially of, or consists of the sequence set forth in SEQ ID NO: 405.

239. The nucleic acid of claim 235 or 236, wherein the genomic safe harbor locus is genomic coordinates of about 15,226,387 to about 15,227,386 on mouse chromosome 17.

240. The nucleic acid of any one of claims 235, 236, and 239, wherein the genomic safe harbor locus is mouse chromosome 17, coordinates 15,226,387-15,227,386 or comprises, consists essentially of, or consists of the sequence set forth in SEQ ID NO: 406.

241. The nucleic acid of claim 235 or 236, wherein the genomic safe harbor locus is genomic coordinates of about 92,827,563 to about 92,828,592 on mouse chromosome 4.

242. The nucleic acid of any one of claims 235, 236, and 241, wherein the genomic safe harbor locus is mouse chromosome 4, coordinates 92,827,563-92,828,592 or comprises, consists essentially of, or consists of the sequence set forth in SEQ ID NO: 407.

243. The nucleic acid of any one of claims 227-242, wherein the product of interest is a polypeptide of interest.

244. The nucleic acid of claim 243, wherein the polypeptide of interest comprises a therapeutic polypeptide.

245. The nucleic acid of claim 243 or 244, wherein the polypeptide of interest is a secreted polypeptide.

246. The nucleic acid of claim 243 or 244, wherein the polypeptide of interest is an intracellular polypeptide.

247. The nucleic acid of any one of claims 227-246, wherein the promoter is active in liver cells.

248. The nucleic acid of any one of claims 227-247, wherein the promoter is a tissue-specific promoter.

249. The nucleic acid of any one of claims 227-247, wherein the promoter is a constitutive promoter.

250. The nucleic acid of any one of claims 227-248, wherein the promoter is an inducible promoter.

251. A method of identifying one or more genomic safe harbor loci in a tissue or cell type of interest, comprising:
(a) identifying accessible genomic loci in the tissue or cell type of interest;
(b) selecting genomic loci identified in step (a) based on safety criteria, functional silencing criteria, and/or structural accessibility criteria; and
(c) selecting genomic loci identified in step (b) based on guide RNA availability, efficacy, and specificity.

252. The method of claim 251, wherein step (a) comprises identifying accessible genomic loci using an assay for transposase-accessible chromatin with high-throughput sequencing.

253. The method of claim 251 or 252, wherein step (a) comprises identifying accessible genomic loci using DNase I hypersensitive sites sequencing.

254. The method of any one of claims 251-253, wherein step (a) comprises identifying accessible genomic loci using an assay for transposase-accessible chromatin with high-throughput sequencing and DNase I hypersensitive sites sequencing.

255. The method of any one of claims 251-254, wherein step (b) comprises selecting genomic loci identified in step (a) based on safety criteria, functional silencing criteria, and structural accessibility criteria.

256. The method of any one of claims 251-255, wherein the safety criteria in step (b) comprise selecting genomic loci only if they are more than 300 kb from any cancer-related gene, more than 300 kb from any miRNA or small RNA, and more than 50 kb from the 5′ end of any gene.

257. The method of any one of claims 251-256, wherein the functional silencing criteria in step (b) comprise selecting genomic loci only if they are more than 50 kb from any replication origin and more than 50 kb from any ultra-conserved elements.

258. The method of any one of claims 251-257, wherein the structural accessibility criteria in step (b) comprise selecting genomic loci only if they are not in copy number variable regions.

259. The method of any one of claims 251-258, wherein efficacy in step (c) comprises editing efficiency in the tissue or cell type of interest.

260. The method of any one of claims 251-259, further comprising analyzing the chromatin environment of the genomic loci selected in step (c) for markers to disqualify any genomic locus that is in a region predicted to be a regulatory region, a heterochromatin region, a region participating in chromatin three-dimensional organization, or transcriptionally active region.

261. The method of claim 260, wherein the markers for the regulatory region comprise H3K4me1, H3K27ac, and H3K4me3.

262. The method of claim 260 or 261, wherein the markers for the heterochromatin region comprise H3K9me3.

263. The method of any one of claims 260-262, wherein the markers for the region participating in chromatin three-dimensional organization comprise CTCF.

264. The method of any one of claims 260-263, wherein the markers for the transcriptionally active region comprise H3K36me3, PolR2A, RNASeq−, and RNASeq+.

265. The method of any one of claims 251-264, wherein step (a) comprises identifying accessible genomic loci using an assay for transposase-accessible chromatin with high-throughput sequencing and DNase I hypersensitive sites sequencing,
wherein step (b) comprises selecting genomic loci identified in step (a) based on safety criteria, functional silencing criteria, and structural accessibility criteria, wherein the safety criteria in step (b) comprise selecting genomic loci only if they are more than 300 kb from any cancer-related gene, more than 300 kb from any miRNA or small RNA, and more than 50 kb from the 5′ end of any gene, wherein the functional silencing criteria in step (b) comprise selecting genomic loci only if they are more than 50 kb from any replication origin and more than 50 kb from any ultra-conserved elements, and wherein the structural accessibility criteria in step (b) comprise selecting genomic loci only if they are not in copy number variable regions, and
wherein the method further comprises analyzing the chromatin environment of the genomic loci selected in step (c) for markers to disqualify any genomic locus that is in a region predicted to be a regulatory region, a heterochromatin region, a region participating in chromatin three-dimensional organization, or a transcriptionally active region, wherein the markers for the regulatory region comprise H3K4me1, H3K27ac, and H3K4me3, wherein the markers for the heterochromatin region comprise H3K9me3, wherein the markers for the region participating in chromatin three-dimensional organization comprise CTCF, and wherein the markers for the transcriptionally active region comprise H3K36me3, PolR2A, RNASeq−, and RNASeq+.

266. The method of any one of claims 251-265, wherein the method is for identifying one or more genomic safe harbor loci in a human tissue or cell type of interest.

267. The method of any one of claims 251-266, wherein the tissue or cell type of interest is liver.

268. The method of any one of claims 251-266, wherein the tissue or cell type of interest is hematopoietic cells.
